(19) United States
(12) Patent Application Publication
(10) Pub. No.: US 2025/0215044 A1
(43) Pub. Date: Jul. 3, 2025

(54) Compounds and Methods for Inhibiting LPA

(71) Applicant: Ionis Pharmaceuticals, Inc., Carlsbad, CA (US)

(72) Inventors: Dieter Kubli, Santee, CA (US); Adam Mullick, Carlsbad, CA (US); Thazha P. Prakash, Carlsbad, CA (US); Eric E. Swayze, Encinitas, CA (US)

(73) Assignee: Ionis Pharmaceuticals, Inc., Carlsbad, CA (US)

(21) Appl. No.: 18/891,595

(22) Filed: Sep. 20, 2024

Related U.S. Application Data
(60) Provisional application No. 63/584,489, filed on Sep. 21, 2023.
(60) Provisional application No. 63/648,647, filed on May 16, 2024.

(51) Int. Cl. C07H 21/04; A61K 31/713

(52) U.S. Cl. CPC C07H 21/04; A61K 31/713

(57) ABSTRACT

Provided are oligomeric duplexes, oligomeric compounds and antisense agents, methods, and pharmaceutical compositions for reducing the amount or activity of LPA RNA in a cell or animal, and in certain instances reducing the amount of Lp(a) in a subject. Such oligomeric duplexes, oligomeric compounds and agents, methods, and pharmaceutical compositions are useful to treat or manage inflammatory, cardiovascular and/or metabolic diseases, disorders, or conditions.

### CROSS-REFERENCE TO RELATED APPLICATIONS

This application claims the benefit of priority to U.S. Provisional Application No. 63/584,489, filed Sep. 21, 2023, and U.S. Provisional Application No. 63/648,647, filed May 16, 2024, each of which is incorporated by reference herein in its entirety for any purpose.

### SEQUENCE LISTING

The present application is being filed concurrently with a Sequence Listing in electronic format. The Sequence Listing is provided as a file entitled BIOL0483SEQ.xml, created on Sep. 9, 2024, which is 1,236 KB in size. The contents of the electronic format of the sequence listing are incorporated herein by reference in their entirety.

### FIELD

Provided herein are oligomeric duplexes, oligomeric compounds, compositions, and uses therefor, including methods for modulating the amount and/or activity of ApoA expression, LPA RNA, and/or Lp(a), as well as methods for treating or managing inflammatory, cardiovascular and/or metabolic diseases, disorders or conditions, and in certain embodiments, ameliorating at least one symptom of an inflammatory, cardiovascular and/or metabolic disease, disorder or condition.

### BACKGROUND

The lipoprotein(a) [Lp(a)] particle comprises a unique LDL particle in which one apolipoprotein B (apoB) protein is linked via a disulfide bond to a single apolipoprotein(a) [apo(a)] protein. The apo(a) protein shares a high degree of homology with plasminogen, particularly within the kringle IV type 2 repetitive domain. Levels of circulating Lp(a) are inversely proportional to the number of kringle IV type 2 variable repeats present in the molecule and, as both alleles are co-expressed within individuals, can display heterozygous plasma isoform profiles among individuals (Kraft et al., Eur J Hum Genet, 1996; 4(2): 74-87). It is thought that the kringle repeat domain in apo(a) may be responsible for its pro-thrombotic and anti-fibrinolytic properties, potentially enhancing atherosclerotic progression. Apo(a) has been shown to preferentially bind oxidized phospholipids and potentiate vascular inflammation (Bergmark et al., J Lipid Res 2008; 49:2230-2239; Tsimikas et al., Circulation. 2009; 119(13):1711-1719). Further, studies suggest the Lp(a) particle may also stimulate endothelial permeability, induce plasminogen activator inhibitor type-1 expression, and activate macrophage interleukin-8 secretion (Koschinsky and Marcovina, Curr Opin Lipidol 2004; 15:167-174). Importantly, genetic association studies revealed that Lp(a) was an independent risk factor for myocardial infarction, stroke, peripheral vascular disease, and abdominal aortic aneurysm (Rifai et al., Clin Chem 2004; 50:1364-71; Ergou et al., JAMA 2009; 302:412-23; Kamstrup et al., Circulation 2008; 117:176-84). Further, the Precocious Coronary Artery Disease (PROCARDIS) study, Clarke et al. (Clarke et al., NEJM (2009)361; 2518-2528) described robust and independent associations between coronary heart disease and plasma Lp(a) concentrations; and Solfrizzi et al., suggested that increased serum Lp(a) may be linked to an increased risk for Alzheimer's Disease (AD) (Solfrizzi et al., J Neurol Neurosurg Psychiatry 2002, 72:732-736.

Examples of indirect apo(a) inhibitors for treating cardiovascular disease in a clinical setting include aspirin, Niaspan, Mipomersen, Anacetrapib, Epirotirome and Lomitapide which reduce plasma Lp(a) levels by 18%, 39%, 32%, 36%, 43% and 17%, respectively. Lp(a) apheresis has also been used in the clinic to reduce apo(a) containing Lp(a) particles. To date, proposed therapeutic strategies to treat cardiovascular disease by directly targeting apo(a) levels have been described (e.g., U.S. Pat. Nos. 5,877,022, 8,138,328, 8,673,632 and 7,259,150; U.S. Patent Publication No. US20040242516; International patent application publication nos. WO2005/000201, WO2003/014397, WO2013/177468, WO 2014/179625, WO2017/059223, WO2020/099476 and WO 2022/032288; and Merki et al., J Am Coll Cardiol 2011; 57:1611-1621., and Tsimikas et al. (Lancet. 2015 Oct. 10; 386:1472-83)). Although active therapeutics have been developed, none have been approved for commercial use. There remains an need for agents which can potently and selectively lower Lp(a) levels in subjects, including in patients at enhanced risk for cardiovascular events due to chronically elevated plasma Lp(a) levels. In particular, new therapeutic agents that enable safe, low frequency administration for treatment and prevention of cardiovascular disease are desired.

### SUMMARY

Provided herein are oligomeric duplexes, pharmaceutical compositions, and methods of use for reducing the amount or activity for reducing the amount or activity of LPA RNA, and reducing the expression of Apo(a) protein in a cell or subject and/or Lp(a) in a subject. In certain embodiments, a subject has a disease or disorder associated with misregulation of lipoproteins or a mutation in lipoprotein regulation pathway. In certain embodiments, the subject has elevated lipoprotein (a). In certain embodiments the subject has or is at risk for a severe a cardiovascular disease, disorder, or condition. In certain embodiments, the subject has or is at risk for a metabolic or inflammatory disease, disorder, or condition. In certain embodiments, agents useful for reducing the amount or activity of LPA RNA are oligomeric duplex, oligomeric compound, or composition as provided herein. In certain embodiments, agents useful for decreasing expression of Apo(a) or levels of lipoprotein (a) are oligomeric compounds, oligomeric duplexes, antisense agents, and/or RNAi agents.

Provided are modified oligonucleotides and compounds and compositions comprising them, including, but not limited to, antisense agents, oligomeric agents, oligomeric duplexes, and pharmaceutical compositions comprising modified oligonucleotides. In certain embodiments, a modified oligonucleotide provided herein comprises a nucleobase sequence at least 80% complementary to an equal length portion of an LPA nucleic acid. In certain embodiments, the modified oligonucleotide consists of 16 to 50, 16 to 30, 18 to 28, 16 to 25, or 18 to 23 linked nucleosides targeting LPA nucleic acid. In certain embodiments, a modified oligonucleotide provided herein comprises a sequence of nucleobases complementary to an equal length portion of the nucleobase sequence of SEQ ID NO:1 and/or SEQ ID NO: 2. In certain embodiments, provided oligomeric duplexes comprise a first oligomeric compound and a second oligomeric compound, wherein a first oligomeric compound comprises a modified oligonucleotide consisting of 18 to 28 linked nucleosides, wherein the nucleobase sequence of the first oligomeric compound comprises at least 18, at least 19, at least 20, at least 21, at least 22, or at least 23 nucleobases complementary to the nucleobase sequence of SEQ ID No: 2, wherein each of the nucleosides of the modified oligonucleotide comprises a modified sugar moiety or sugar surrogate and wherein no more than 18%, no more than 15%, no more than 14%, no more than 10%, or no more than 5% of the modified nucleosides in the first oligomeric compound comprises a 2′-fluoro sugar moiety; and wherein a second oligomeric compound comprises a modified oligonucleotide consisting of 16 to 26 contiguous linked nucleosides wherein the nucleobase sequence of the second oligomeric compound comprises at least 16, at least 17, at least 18, at least 19, at least 20, or at least 21 nucleobases of the nucleobase sequence of SEQ ID NO: 2, wherein each of the nucleosides of the second oligomeric compound comprises a modified sugar moiety or sugar surrogate and wherein no more than 18%, no more than 16%, no more than 14%, no more than 12%, or no more than 10%, of the modified nucleosides in the second oligomeric compound comprises a 2′-fluoro sugar moiety. In certain embodiments, a modified oligonucleotide provided herein comprises at least one modified sugar moiety and/or at least one modified internucleoside linkage. In certain embodiments, an oligomeric compound provided herein comprises a modified oligonucleotide comprising at least one 3′-fluoro-hexitol sugar moiety. In certain embodiments, an oligomeric compound provided herein comprises a modified oligonucleotide comprising at least one a 2′-deoxynucleoside. In certain embodiments, an oligomeric compound provided herein comprises a modified oligonucleotide comprising at least one a beta-D-deoxyxylosyl nucleoside. In certain embodiments, an oligomeric compound provided herein comprises a modified oligonucleotide comprising at least one a beta-D-deoxyribosyl nucleoside. In certain embodiments, an oligomeric compound provided herein comprises a modified oligonucleotide conjugated to a liver cell targeting agent. Modified oligonucleotides and compositions comprising them, including, but not limited to, oligomeric duplexes, oligomeric compounds, modified oligonucleotides, and composition described herein are useful for reducing or inhibiting LPA expression in a cell, organ, tissue, system, organism, or subject.

In certain embodiments provided are oligomeric compounds comprising a first modified oligonucleotide consisting of 18 to 28 linked nucleosides, wherein the nucleobase sequence comprises at least 18, at least 19, at least 20, at least 21, at least 22, or at least 23 nucleobases complementary to the nucleobase sequence of SEQ ID NO:2, wherein no more than 20%, no more than 18%, no more than 15%, no more than 10%, or no more than 5% of the modified nucleosides in the first modified oligonucleotide comprises a fluorine, and a second modified oligonucleotide consisting of 16 to 26 linked nucleosides, wherein the nucleobase sequence comprises at least 16, at least 17, at least 18, at least 19, at least 20, or at least 21 nucleobases of the nucleobase sequence of SEQ ID NOs:2, wherein no more than no more than 18%, no more than 16%, no more than 14%, no more than 12%, or no more than 10%, of the modified nucleosides in the second modified oligonucleotide comprises a fluorine, wherein the first modified oligonucleotide comprising a modified sugar motif and is paired with the second modified oligonucleotide comprising a sugar motif selected from: efyyyyyyyyyyyfyfyyyyyee and yyyyyyyyfffyyeyyyyyyy; efyyyyyyyyyyyfyfyyyyyee and eeyyyyyyfffyyeyyyyyee; efyyyfyyyyyyyfyyyyyyyee and yyyyyyyyfffyyeyyyyyyy; efyyyfyyyyyyyfyyyyyyyee and eeyyyyyyfffyyeyyyyyee; efyyydyyeyyyydydyyyyyee and yyyyyyyyfffyyeyyyyyyy; efyyyfyyyyyyyfyyyyyyyee and eeyyyyyydffyyeyyyyyee; efyyyfyyyyyyyfyyyyyyyee and eeyyyyyyfdfyyeyyyyyee; efyyyfyyyyyyyfyyyyyyyee and eeyyyyyyffdyyeyyyyyee; efyyyfyyyyyyyfyyyyyyyee and eeyyyyyyhffyyeyyyyyee; efyyyfyyyyyyyfyyyyyyyee and eeyyyyyyfhfyyeyyyyyee; efyyyfyyyyyyyfyyyyyyyee and eeyyyyyyffhyyeyyyyyee; efyyyfyyyyyyyfyyyyyyyee and eeyyyyyyfxfyyeyyyyyee; efyyyfyyyyyyyfyyyyyyyee and eeyyyyyyffxyyeyyyyyee; efyyyfyyyyyyyfyyyyyyyee and eeyyyyyyxffyyeyyyyyee; efyyydyyeyyyydydyyyyyee and eeyyyyyyfhfyyeyyyyyee; efyyydyyeyyyydydyyyyyee and eeyyyyyyfdfyyeyyyyyee; efyyydyyeyyyydydyyyyyee and eeyyyyyyffhyyeyyyyyee; efyyydyyeyyyydydyyyyyee and eeyyyyyyffdyyeyyyyyee; efyyydyyeyyyydydyyyyyee and eeyyyyyydffyyeyyyyyee; efyyydyyeyyyydydyyyyyee and eeyyyyyyhffyyeyyyyyee; efyyydyyeyyyydydyyyyyee and eeyyyyyyxffyyeyyyyyee; efyyydyyeyyyydydyyyyyee and eeyyyyyyfxfyyeyyyyyee; efyyydyyeyyyydydyyyyyee and eeyyyyyyffxyyeyyyyyee; efyyyyyyeyyyyfyfyyyyyee and eeyyyyyyfffyyeyyyyyee; efyyyfyyeyyyyfyyyyyyyee and eeyyyyyyfffyyeyyyyyee; efyyxfyyyyyyyfyyyyyyyee and eeyyyyyyfffyyeyyyyyee; efyyyfxyyyyyyfyyyyyyyee and eeyyyyyyfffyyeyyyyyee; and efyyyxyyyyyyyfyyyyyyyee and eeyyyyyyfffyyeyyyyyee; wherein each ‘e’ represents a 2′-MOE sugar moiety, each ‘y’ represents a 2′-OMe sugar moiety, each ‘f’ represents a 2′-F sugar moiety, each ‘h’ represents a 3′-fluoro-hexitol sugar moiety, each ‘d’ represents a 2′-deoxyribose sugar moiety, and each ‘x’ represents a 2′-deoxyxylose sugar moiety; and wherein all except 0, 1, or 2 modifications are identical to the sugar motif. In certain embodiments provided are oligomeric compounds comprising a first modified oligonucleotide comprising a sequence comprising at least 18, at least 19, or at least 20 nucleobases of any one of the sequences of SEQ ID NOs:5-6, or 15-48, or 132-140, and a second modified oligonucleotide comprising a sequence comprising at least 16, at least 17, or at least 18 nucleobases of any one of the sequences of SEQ ID NOs:52-82, or 141-142.

In certain embodiments provided are oligomeric compounds comprising a first modified oligonucleotide comprising a sequence selected from any one of SEQ ID NOs:5-6, or 15-48, or 132-140, and a second modified oligonucleotide comprising a sequence selected from any one of SEQ ID NOs:52-82, or 141-142. In certain embodiments provided are oligomeric duplexes consisting of a first oligomeric compound having a sequence selected from any one of SEQ ID NOs:5-6, or 15-48, or 132-140, and a second oligomeric compound having a sequence selected from any one of SEQ ID NOs:52-82, or 141-142.

Additionally provided are methods for reducing or inhibiting LPA expression, LPA RNA levels and/or Apo(a) protein levels and/or Lp(a) activity in a cell, tissue, organ, or subject. In certain embodiments, methods include contacting a cell or subject with a composition provided herein, comprising, e.g., an oligomeric duplex, oligomeric compound, modified oligonucleotide as described herein. In certain embodiments, the subject is a human who has or is at risk of having a cardiovascular, metabolic, or inflammatory disease, disorder, condition, or injury associated with increased lipoprotein a levels, misregulation of lipoprotein turnover or a mutation in LPA. In certain embodiments, the subject is a human who has or is at risk of having hypertriglyceridemia. In certain embodiments, the subject is a human who has or is at risk of having atherosclerotic cardiovascular disease (ASCVD) or coronary artery disease (CAD).

Provided herein are methods of treating a disease, disorder, condition, or injury associated with lipoprotein metabolism misregulation, regulation of lipoprotein turnover or a mutation in LPA. In certain embodiments, a method of treating a disease, disorder, condition or injury associated with lipoprotein metabolism misregulation, regulation of Lp(a) levels or a mutation in LPA comprises administering to a subject, e.g., a human subject, having, or at risk of having, a disease, disorder or condition associated with elevated Lp(a), a provided oligomeric duplex, oligomeric compound, or composition provided herein, wherein the disease, disorder, condition or injury is selected from a cardiovascular disease, disorder, condition, a metabolic disease, disorder, or condition, or an inflammatory disease disorder or condition. In certain embodiments, the subject has or is at risk for developing cardiovascular disease (CVD) coronary artery disease (CAD), hypercholesterolemia, myocardial infarction (MI), peripheral arterial disease (PAD), calcific aortic valve disease (CAVD), aortic stenosis, atherosclerotic cardiovascular disease (ASCVD), atherosclerosis, dyslipidemia, thrombosis, or stroke. In certain embodiments, methods of treating provided herein result in ameliorating (whether by reduced frequency, severity) a at least one symptom of a disease, disorder, condition, or injury associated with lipoprotein metabolism misregulation. In certain embodiments, methods of treating provided herein result in preventing, delay or postponing, or slowing the development or progression of at least one symptom of a disease, disorder or condition associated with elevated Lp(a).

Also provided are methods useful for ameliorating at least one symptom of a disorder associated with lipoprotein metabolism misregulation. In certain embodiments the disorder is severe hypertriglyceridemia, hyperlipidemia, dyslipidemia, and/or hyperlipoproteinemia. In certain embodiments, a symptom of hypertriglyceridemia, hyperlipidemia, dyslipidemia, and/or hyperlipoproteinemia include, but are not limited to, abdominal pain, physical fatigue, difficulty thinking, diarrhea, acute pancreatitis, eruptive xanthomas, lipemia retinalis, or hepatosplenomegaly, or a combination of two or more of the foregoing in the subject. In certain embodiments, methods provided herein for preventing, treating, ameliorating, delaying the onset of, or reducing frequency of at least one symptom of hypertriglyceridemia, hyperlipidemia, dyslipidemia, and/or hyperlipoproteinemia include administering to a subject, e.g., a human subject, having or at risk of having at least one symptom a composition provided herein, e.g., a modified oligonucleotide, oligomeric duplex, oligomeric compound, or pharmaceutical composition provided herein,

### DESCRIPTION OF THE DRAWINGS

FIGS. 1A-1C depict results of duration of inhibition of human LPA in transgenic mouse study described in Example 5C. FIG. 1A depicts results of apo(a) protein levels measured overtime by clinical chemistry analyzer; FIG. 1B depicts results of apo(a) protein levels measured overtime by ELISA assay; and FIG. 1C depicts results of liver LPA RNA analysis measured by RTPCR at the final timepoint in FIGS. 1A and 1B.

FIGS. 2A-2C depict results of duration of inhibition of human LPA in transgenic mouse study described in Example 5C. FIG. 2A depicts results of apo(a) protein levels measured overtime by clinical chemistry analyzer; FIG. 2B depicts results of apo(a) protein levels measured overtime by ELISA assay; and FIG. 2C depicts results of liver LPA RNA analysis measured by RTPCR at the final timepoint in FIGS. 2A and 2B.

FIGS. 3A-3B depict results of duration of inhibition of human LPA in transgenic mouse study described in Example 5C. FIG. 3A depicts results of apo(a) protein levels measured overtime by clinical chemistry analyzer; and FIG. 3B depicts results of apo(a) protein levels measured over time by ELISA assay.

FIGS. 4A-4B depict results of duration of inhibition of human LPA in transgenic mouse study described in Example 5C. FIG. 4A depicts results of apo(a) protein levels measured overtime by clinical chemistry analyzer; and FIG. 4B depicts results of apo(a) protein levels measured over time by ELISA assay.

FIGS. 5A-5C depict results of duration of inhibition of human LPA in transgenic mouse study described in Example 5C. FIGS. 5A-5B depict results of apo(a) protein levels measured over time by clinical chemistry analyzer; and FIG. 5C depicts results of apo(a) protein levels measured overtime by ELISA assay.

FIGS. 6A-6C depict results of duration of inhibition of human LPA in transgenic mouse study described in Example 5C. FIGS. 6A-6B depict results of apo(a) protein levels measured over time by clinical chemistry analyzer; and FIG. 6C depicts results of apo(a) protein levels measured over time by ELISA assay.

FIGS. 7A-7B depict results of duration of inhibition of human LPA in transgenic mouse study described in Example 5C. FIG. 7A depicts results of apo(a) protein levels measured overtime by clinical chemistry analyzer; and FIG. 7B depicts results of apo(a) protein levels measured over time by ELISA assay.

FIGS. 8A-8B depict reduction of human plasminogen (PLG) described in Example 7. FIG. 8A depicts results of PLG levels measured over time by RTPCR after treatment with duplex 1826573; and FIG. 8B depicts results of PLG levels measured over time by RTPCR after treatment with duplex 1840071.

FIGS. 9A-9D depict results of dose dependent inhibition ofhuman LPA RNA and apo(a) protein in transgenic mouse study described in Example 8. FIGS. 9A and 9C depict results of LPA RNA levels after two weeks of various doses of compound treatment. FIGS. 9B and 9D depict results of apo(a) protein levels measured after two weeks of various doses of compound treatment.

FIGS. 10A-10D depict results of inhibition of human LPA in a transgenic mouse study described in Example 9. FIGS. 10A-10D depict duration results of reduction of apo(a) protein levels measured over time by ELISA assay after a single 3 mg/kg dose of compound. Each of FIGS. 10A-10D represents three separate experiments, and FIGS. 10C and 10D are one experiment depicted in two graphs due to the number of compounds tested.

FIGS. 11A-11D depict inhibition of human LPA in transgenic mouse study described in Example 9. FIGS. 11A-11D depict duration results of reduction of apo(a) protein levels measured over time by ELISA assay after different doses of compound.

### DETAILED DESCRIPTION

It is to be understood that both the foregoing general description and the following detailed description are exemplary and explanatory only and are not restrictive. Herein, the use of the singular includes the plural unless specifically stated otherwise. Furthermore, the use of the term “including” as well as other forms, such as “includes” and “included,” is not limiting. Also, terms such as “element” or “component” encompass both elements and components comprising one unit and elements and components that comprise more than one subunit, unless specifically stated otherwise.

### Definitions

The following definitions are provided, along with additional definitions throughout the specification, for a complete understanding of the instant invention. Unless specific definitions are provided herein, nomenclature used in connection with, and procedures and techniques of, analytical chemistry, synthetic organic chemistry, and medicinal and pharmaceutical chemistry described herein are those well-known and commonly used in the art. Unless otherwise indicated, certain terms have the following meanings:

As used herein, a substituent at the “2′-position” means that the substituent is directly attached to the carbon at the 2′-position of a furanosyl sugar moiety.

As used herein, “2′-deoxynucleoside” means a nucleoside comprising a 2′-deoxyfuranosyl sugar moiety. Unless otherwise indicated, a 2′-deoxynucleoside is a 2′-β-D-deoxynucleoside which comprises a 2′-deoxyribosyl sugar moiety or a 2′-deoxyxylosyl sugar moiety, e.g., which is in the β-D configuration in naturally occurring deoxyribonucleic acid (DNA) or deoxyxylonucleic acid (dXNA). As used herein, “2′-deoxy sugar moiety” means a 2′-H(H) deoxyfuranosyl sugar moiety. Unless otherwise indicated, a 2′-deoxy sugar moiety is a 2′-deoxyribosyl sugar moiety or a 2′-deoxyxylosyl sugar moiety, which has the β-D stereochemical configuration in naturally occurring deoxyribonucleic acid (DNA, dXNA).

As used herein, “2′-deoxyribonucleoside” means a nucleoside comprising a 2′-deoxyribosyl sugar moiety, a modified sugar moiety. Unless otherwise indicated, a 2′-deoxyribose nucleoside is a 2′-β-D-deoxyribose nucleoside which comprises a 2′-β-D-deoxyribosyl sugar moiety, which is in the β-D configuration. A 2′-deoxyribonucleoside or a nucleoside comprising an unmodified 2′-deoxyribosyl sugar moiety may be abasic, comprise a modified nucleobase, or may comprise an RNA nucleobase (uracil).

As used herein, “2′-deoxyxylonucleoside” means a nucleoside comprising a 2′-deoxyxylosyl sugar moiety, a modified sugar moiety. As used herein, “2′-deoxyxylose sugar moiety” means a 2′-H(H) deoxyxylosyl sugar moiety, a modified sugar moiety. Unless otherwise indicated, a 2′-deoxyxylose sugar moiety herein is a 2′-β-D-deoxyxylosyl sugar moiety, which has the β-D stereochemical configuration. Unless otherwise indicated, a 2′-deoxyxylose nucleoside is a 2′-β-D-deoxyxylose nucleoside which comprises a 2′-β-D-deoxyxylosyl sugar moiety, which is in the β-D configuration. A 2′-deoxyxylose nucleoside or a nucleoside comprising an unmodified 2′-deoxyxylosyl sugar moiety may be abasic, comprise an unmodified or a modified nucleobase, including any nucleobase found in DNA or RNA.

As used herein, “2′-MOE” means a 2′-OCH₂CH₂OCH₃ group at the 2′-position of a furanosyl sugar moiety. A “2′-MOE sugar moiety” means a sugar moiety with a 2′-OCH₂CH₂OCH₃ group at the 2′-position of a furanosyl sugar moiety. Unless otherwise indicated, a 2′-MOE sugar moiety is in the 3-D-ribosyl stereochemical configuration. “MOE” means O-methoxyethyl.

As used herein, “2′-MOE nucleoside” or “2′-OCH₂CH₂OCH₃ nucleoside” means a nucleoside comprising a 2′-MOE sugar moiety (or 2′-OCH₂CH₂OCH₃ furanosyl sugar moiety).

As used herein, “2′-OMe” means a 2′-O-methyl or 2′-OCH₃ group at the 2′-position of a furanosyl sugar moiety. A “2′-OMe sugar moiety” means a sugar moiety with a 2′-O-methyl or a 2′-OCH₃ group at the 2′-position of a furanosyl sugar moiety. Unless otherwise indicated, a 2′-OMe sugar moiety is in the β-D-ribosyl stereochemical configuration.

As used herein, “2′-OMe nucleoside” means a nucleoside comprising a 2′-OMe sugar moiety.

As used herein, “2′-F” means a 2′-fluoro group at the 2′-position of a furanosyl sugar moiety. A “2′-F sugar moiety” means a sugar moiety with a 2′-F group at the 2′-position of a furanosyl sugar moiety. Unless otherwise indicated, a 2′-F sugar moiety is in the β-D-ribosyl configuration.

As used herein, “2′-F nucleoside” means a nucleoside comprising a 2′-F sugar moiety.

As used herein, “2′-substituted nucleoside” means a modified nucleoside comprising a 2′-substituted furanosyl sugar moiety. 2′-substituted nucleosides include, but are not limited to, a 2′-OMe nucleoside, a 2′-MOE nucleoside, a 2′-F nucleoside, a cEt nucleoside, and a LNA nucleoside.

As used herein, “2′-substitution” or “2′-substituted sugar moiety” means a modified furanosyl sugar moiety wherein the 2′-position is attached to at least one substituent other than H or OH. A 2′-substituted sugar moiety includes a bicyclic sugar moiety wherein the second ring is joined to the furanosyl ring at the 2′-position. 2′-substituted sugar moieties include, but are not limited to, 2′-OMe sugar moieties, 2′-MOE sugar moieties, 2′-F sugar moieties, cEt sugar moieties, and LNA sugar moieties.

As used herein, “5-methylcytosine” means a cytosine modified with a methyl group attached at the 5 position. A 5-methylcytosine is a modified nucleobase.

As used herein, “abasic nucleoside” means a modified nucleoside in which the sugar moiety is not attached to a nucleobase.

As used herein, “acyclic sugar surrogate nucleoside” means a nucleoside having Formula II, Formula III, or Formula IV:

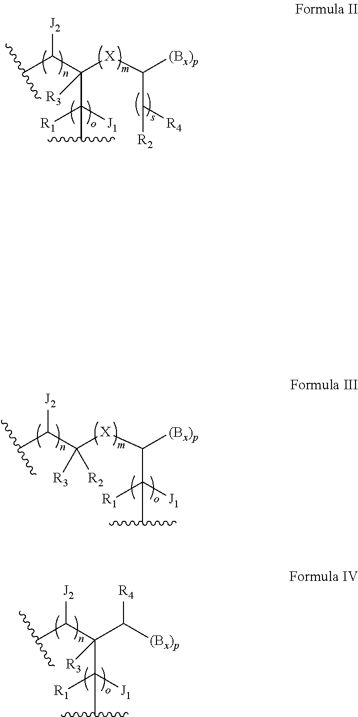

wherein
X is O, S, C(R₅R₆), N(E₁), NC(═O)-(E₁);
each J₁ and J₂ are independently H or C₁-C₆ alkyl;
n is 0, 1 or 2;
m is 0, 1, or 2;
p is 0 or 1;
o is 0 or 1;
s is 0 or 1;
R₁ is H, OH, halogen, C₁-C₆ alkyl, C₁-C₆ alkoxy, C₂-C₆ alkenyl, C₂-C₆ alkynyl, or (CH₂)_{q}R₈;
R₂, R₃, and R₄ are each independently H, OH, halogen, C₁-C₆ alkyl, C₁-C₆ alkoxy, C₂-C₆ alkenyl, C₂-C₆ alkynyl, S—CH₃, N(CH₃)(CH₃), OCH₂CH₂OCH₃, O-alkylamino, or (CH₂)_{q}R₈;
E₁ is H, C₁-C₆ alkyl or substituted C₁-C₆ alkyl;
R₅ and R₆ are independently H, OH, C₁-C₆ alkyl, or N(R₇); wherein if R₅ is OH, then R₆ is not OH; R₇ is H, C₁-C₆ alkyl, or C(═O)R₉, wherein R₉ is C₁-C₆ alkyl;
R₈ is OH, halogen, methoxy, ethoxy, azido, C₂-C₆ alkenyl, or C₂-C₆ alkynyl, and q is 1, 2, or 3; and
Bx is a nucleobase.

As used herein, “acyclic sugar surrogate” means the sugar moiety of an acyclic sugar surrogate nucleoside.

As used herein, “ameliorate” with reference to a symptom of a disease, means improvement in, or lessening, or preclusion of, at least one symptom of a disease. Amelioration may be reduction in severity or frequency of a symptom or the delayed onset, prevention of occurrence of, or slowing of progression in the severity or frequency of, a symptom. Progression, frequency, or severity indicators may be determined by subjective or objective measures known in the art and/or described herein.

As used herein, “antisense activity” means any detectable and/or measurable change attributable (whether directly and/or indirectly) to hybridization of an antisense oligonucleotide to a target nucleic acid. For example, compounds have antisense activity when they alter the amount or activity of a target nucleic acid by 25% or more in an in vitro assay; or, for example compounds have antisense activity when they alter the amount or activity of a target nucleic acid by 25% or more in an in vivo assay. Antisense activity may be assessed in a standard assay. Herein, antisense activity is a reduction or inhibition in the amount or expression of a target nucleic acid, or a protein encoded by such target nucleic acid compared to target nucleic acid levels or target protein levels in the absence of the oligonucleotide.

As used herein, “antisense agent” means an oligomeric agent comprising an antisense oligonucleotide.

As used herein, “antisense oligonucleotide” means an oligonucleotide having at least one region (a “targeting region”) that is complementary to a target nucleic acid (e.g., a target region). An antisense oligonucleotide may be paired with a second oligonucleotide (herein, a “sense oligonucleotide”) that is complementary to the antisense oligonucleotide (for example, forming an “oligomeric duplex”), may be an unpaired antisense oligonucleotide (herein, a single-stranded antisense oligonucleotide), or may be a “hairpin oligonucleotide” that has at least one region that is self-complementary.

As used herein, “bicyclic sugar” or “bicyclic sugar moiety” means a modified sugar moiety comprising a furanosyl sugar moiety and a second ring, wherein the second ring is formed via a bridge connecting two non-geminal atoms in the ring of the furanosyl sugar moiety, thereby forming a bicyclic structure. Examples of bicyclic sugar moieties include locked nucleic acid (LNA) sugar moieties and constrained ethyl (cEt) sugar moieties as defined herein.

As used herein, “bicyclic nucleoside” or “BNA” means a nucleoside comprising a bicyclic sugar moiety.

As used herein, “cell-targeting moiety” means a conjugate group or portion of a conjugate group that has affinity for a particular cell type or particular cell types. For example, a cell-targeting moiety may have affinity for a cell surface moiety, such as a cell surface receptor on a particular cell type.

As used herein, “cleavable moiety” means a group of atoms comprising at least one bond that is cleaved under physiological conditions, e.g., in a cell, in a subject. For example, a cleavable moiety cleaved inside a cell or sub-cellular compartment, such as an endosome or lysosome. A cleavable moiety may be cleaved by endogenous enzymes, such as nucleases. A cleavable moiety may be cleaved by endogenous change in conditions such as pH.

As used herein, “complementary nucleobase(s)” or “complementary” in reference to nucleobase(s) means nucleobases that form hydrogen bonds with another when two regions of linked nucleosides (e.g., an oligonucleotide and a target nucleic acid; two oligonucleotides). Complementary nucleobase pairs include, but are not limited to, adenine (A) and thymine (T), adenine (A) and uracil (U), cytosine (C) and guanine (G), 5-methylcytosine (^{m}C) and guanine (G). Certain modified nucleobases that are complementary to unmodified nucleobases or to other modified nucleobases are known in the art. For example, hypoxanthine, the nucleobase of the nucleoside inosine (I), can pair with adenine, cytosine, thymine, or uracil. Herein, hypoxanthine (I) is considered a complementary nucleobase to thymine (T), adenine (A), uracil (U), and cytosine (C).

As used herein, “complementary sequence(s)” or “complementary” in reference to sequence(s) refers to two nucleobase sequences in which some, a majority, or all the nucleobases in the two sequences are complementary nucleobases when the sequences are aligned. A “nucleobase sequence” means the order of contiguous nucleobases in a strand of linked nucleosides or a region thereof (e.g., an oligonucleotide or region thereof, or a target nucleic acid or region thereof) independent of any sugar or internucleoside linkage modification. Complementary nucleobase sequences may be nucleobase sequences of two separate strands of linked nucleosides or region thereof (e.g., an oligonucleotide and a region of a target nucleic acid); or complementary nucleobase sequences may be two regions of a single strand of linked nucleosides (e.g., self-complementary regions of a hairpin oligonucleotide). As used herein, when a first nucleobase sequence (e.g., an oligonucleotide) or region thereof is described as being complementary to a second nucleobase sequence (e.g., a target nucleic acid or another oligonucleotide), it means that a majority of nucleobases of the first nucleobase sequence or region thereof base pair with complementary nucleobases of the second nucleobase sequence when aligned. Not every pair of nucleobases in the aligned nucleobase sequences needs to be a base pair match for the two sequences to be “complementary.” Rather, some mismatches are tolerated. Where complementarity is expressed as a percent, such percent represents the percent of nucleobases within one nucleobase sequence that are complementary to nucleobases within an equal length second sequence when the sequences are aligned. Unless otherwise specified, “complementary” is assumed to be at least 70%. Complementary nucleobase sequences may be 75%, 80%, 85%, 90%, 95%, or 100% complementary. For example, if a nucleobase sequence of an oligonucleotide consisting of 20 nucleosides is 80% complementary to another nucleobase sequence, then 16 of the nucleobase pairs are complementary nucleobases, and there are 4 mismatches when the sequences are aligned. If a nucleobase sequence of an oligonucleotide consisting of 20 nucleosides is at least 80% complementary to another nucleobase sequence, then 16, 17, 18, 19, or 20 of the nucleobase pairs are complementary nucleobases, and there are 0-4 mismatches when the sequences are aligned. As used herein, “fully complementary” or “100% complementary” means that each nucleobase pair of two nucleobase sequences is complementary when the equal length sequences are aligned.

As used herein, “conjugate group” means a group of atoms including a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that when covalently bound to a molecule (e.g., an oligonucleotide) modifies one or more properties of such molecule compared to the same molecule lacking the conjugate moiety, wherein such properties include, but are not limited to pharmacodynamics, pharmacokinetics, stability, binding, absorption, tissue distribution, cellular distribution, cellular uptake, charge, and clearance.

As used herein, “constrained ethyl” or “cEt” or “cEt sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt sugar moiety.

As used herein a “cyclic sugar surrogate nucleoside” is a nucleoside having Formula I.

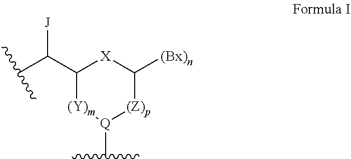

Wherein
J is H, C₁-C₆ alkyl, or C₂-C₆ alkenyl;
X is O, S, C(R₁R₂), N(R₃); or X is X₁—X₂, wherein X₁—X₂ is C(R₁)═C(R₂), C(R₁R₂)—C(R₁R₂), O—C(R₁R₂), C(R₁R₂)—O, S—C(R₁R₂), C(R₁R₂)—S, N(R₃)—C(R₁R₂), or C(R₁R₂)—N(R₃);
Y is C(R₁R₂); or Y₁-Y₂, wherein Y₁-Y₂, is C(R₁)═C(R₂) or C(R₁R₂)—C(R₁R₂);
Z is C(G₁G₂); or Z₁-Z₂, wherein Z₁-Z₂ is C(G₁)═C(R₁), C(R₁)═C(G₁); C(G₁G₂)-C(R₁R₂), C(R₁R₂)—C(G₁G₂); or Z₁—Z₂-Z₃, wherein Z₁—Z₂-Z₃ is C(G₁G₂)-C(R₁R₂)—C(R₁R₂) or C(R₁R₂)—C(R₁R₂)—C(G₁G₂);
Q is CH or N;
each R₁ and R₂ is independently H, OH, C₁-C₆ alkyl, or N(R₄); wherein if R₁ is OH, then R₂ is not OH;
each R₃ and R₄ is independently H, C₁-C₆ alkyl, or C(═O)R₅, wherein R₈ is C₁-C₆ alkyl;
each G₁ and G₂ is independently H, OH, halogen or O—[C(R₆)(R₇)]_{q}—[(C═O)₅—X^{G}]ⱼ—R₈; wherein if G₁ is OH, then G₂ is not OH;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is 0 or 1;
m is 0 or 1;
p is 0 or 1;
q is from 1 to 6;
s is 0 or 1;
j is 0 or 1;
Bx is a nucleobase; and
provided that if X is O, Z is C(G₁G₂), and Q is CH, then m is 1.

As used herein, “cyclic sugar surrogate” means the sugar moiety of a cyclic sugar surrogate nucleoside, and is represented by Formula Ia herein.

As used herein, “double-stranded” refers to hybridized or bound complementary regions, including those between two separate strands of linked nucleosides (e.g., an antisense oligonucleotide and a sense oligonucleotide) and those within a single strand of linked nucleosides (e.g., a hairpin oligonucleotide). Paired complementary regions of two separate strands of linked nucleosides form a duplex of the separate strands. Paired complementary regions of a single strand of linked nucleosides (i.e., a first region of the strand of linked nucleosides and a second region of the strand of linked nucleosides) form a “hairpin.”

As used herein, a “furanosyl sugar moiety” is a group of atoms that comprises a furanose ring and optional substituents, and is numbered according to the structure below, with optional additional substituents at any of the 1′, 2′, 3′, 4′, and 5′ positions.

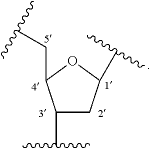 [SMILES: *CC1OC(*)CC1* ; "."]

As used herein, “hybridize” or “hybridization” means the process of two complementary regions of linked nucleosides (e.g., oligonucleotides, nucleic acids) annealing to form a double-stranded region. While not limited to a particular mechanism, the most common mechanism of hybridization involves hydrogen bonding, which may be Watson-Crick, Hoogsteen, or reversed Hoogsteen hydrogen bonding, between complementary nucleobases.

As used herein, “internucleoside linkage” means the covalent linkage between adjacent nucleosides in an oligonucleotide. As used herein, “unmodified internucleoside linkage” means a phosphodiester internucleoside linkage. As used herein, “modified internucleoside linkage” means any internucleoside linkage other than a phosphodiester internucleoside linkage. A “phosphorothioate internucleoside linkage” is a modified internucleoside linkage in which one of the non-bridging oxygen atoms of a phosphodiester internucleoside linkage is replaced with a sulfur atom. A “mesyl phosphoramidate internucleoside linkage” is a modified internucleoside linkage in which one of the non-bridging oxygen atoms of a phosphodiester internucleoside linkage is replaced with NS(═O)₂CH₃. Unless otherwise indicated, and in the context of linked nucleosides each comprising a furanosyl sugar moiety, an internucleoside linkage joins the 3′-carbon of one furanosyl sugar moiety to the 5′-carbon of the other furanosyl sugar moiety.

As used herein, “inverted nucleoside” means a nucleoside having a 3′ to 3′ and/or 5′ to 5′ internucleoside linkage.

As used herein, “linked nucleosides” are nucleosides that are connected in a contiguous sequence (i.e., nucleosides immediately adjacent to one another, no additional nucleosides are presented between those that are linked).

As used herein, a “mismatch” between two aligned strands of linked nucleosides means that two nucleobases at a specified position of the aligned nucleobase sequences are not complementary nucleobases as defined herein.

As used herein, “modified nucleoside” means a compound or subunit comprising a sugar moiety and optionally a nucleobase, wherein the sugar moiety is modified and/or the nucleobase is modified or absent.

As used herein, “modified sugar moiety” means a sugar moiety other than a β-D-ribosyl sugar moiety in RNA. A modified sugar moiety is selected from a modified furanosyl sugar moiety, a cyclic sugar surrogate, an acyclic sugar surrogate, or a sugar mimic. Modified sugar moieties include deoxyfuranosyl sugar moieties in the β-D-stereochemical configuration, and have one hydrogen at each of the 1′, 3′, and 4′ positions, an oxygen at the 3′ position, two hydrogens at the 5′ position, and two hydrogens (e.g., DNA, dXNA), and includes, e.g., deoxyribosyl, deoxyxylosyl, deoxylyxosyl, or deoxyarabinosyl sugar moiety; or a hydrogen and an OH (e.g., XNA) at the 2′ position in other modified furanosyl sugar moiety, and includes, e.g., a xylosyl, lyxosyl, or arabinosyl sugar moiety.

As used herein, a “modified nucleobase” means a nucleobase other than unmodified A, T, C, U, or G capable of pairing with at least one unmodified nucleobase. A “5-methylcytosine” is a modified nucleobase. Inosine (I) is a nucleoside comprising the modified nucleobase hypoxanthine.

As used herein, “motif” means a pattern of independently unmodified and/or independently modified sugar moieties, nucleobases, and/or internucleoside linkages in an oligonucleotide.

As used herein, “non-bicyclic modified sugar moiety” means a modified furanosyl sugar moiety comprising a modification, such as a substituent, that does not form a bridge between two atoms of the sugar to form a second ring. As used herein, “nucleobase” means an unmodified nucleobase or a modified nucleobase.

As used herein, “the nucleobase sequence of” a reference SEQ ID NO, refers only to the order of contiguous nucleobases provided in such SEQ ID NO, independent of any sugar or internucleoside linkage modifications and therefore, unless otherwise indicated, includes compounds wherein each sugar moiety and each internucleoside linkage, independently, is modified or unmodified, irrespective of the presence or absence of modifications indicated in the referenced SEQ ID NO.

As used herein, “nucleoside” means an “unmodified nucleoside” or a “modified nucleoside.”

As used herein, “oligomeric agent” means a compound or complex comprising or consisting of at least one modified oligonucleotide and optionally one or more additional associated features selected from: (a) one or more conjugate groups, which may be covalently attached directly or indirectly to any oligonucleotide of such oligomeric agent; (b) one or more terminal groups; and (c) one or more additional modified or unmodified oligonucleotides, each of which may be hybridized to or covalently linked to the at least one modified oligonucleotide and/or to each other. Herein, where two oligonucleotides are described as being covalently attached to one another, such attachment is other than through a direct internucleoside linkage. Thus, a single, unbranched oligonucleotide comprising only direct internucleoside linkages cannot be described as two separate covalently linked oligonucleotides.

As used herein, “oligomeric compound” means a compound comprising a modified oligonucleotide and optionally one or more covalently linked chemical features selected from one or more conjugate group and one or more terminal group.

As used herein, “oligonucleotide” means a strand of linked nucleosides, wherein, independently each nucleoside and/or independently each internucleoside linkage of the strand of linked nucleosides may be independently modified or unmodified. Unless otherwise indicated, oligonucleotides consist of 12-50 linked nucleosides. Unless otherwise indicated, no more than 10% of the nucleosides of an oligonucleotide are abasic nucleosides. As used herein, “modified oligonucleotide” means an oligonucleotide, wherein at least one nucleoside and/or internucleoside linkage is modified. As used herein, “unmodified oligonucleotide” means an oligonucleotide consisting of unmodified nucleosides linked by phosphodiester internucleoside linkages. An oligonucleotide may be paired with a second oligonucleotide that is complementary to the oligonucleotide to form an oligomeric duplex, or it may be unpaired.

As used herein, “pharmaceutical composition” means a mixture of substances suitable for administration to a subject. For example, a pharmaceutical composition may comprise an agent (e.g., an oligomeric agent, duplex, or antisense agent) and a sterile aqueous solution. A pharmaceutical composition may show activity in certain cell lines.

As used herein, “pharmaceutically acceptable carrier or diluent” means an ingredient in a pharmaceutical composition suitable for use in administering to a subject. Typically, a “carrier” or “diluent” lacks pharmacological activity but is desirable in preparing a pharmaceutical composition.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “RNA nucleoside” means a nucleoside comprising an unmodified RNA sugar moiety. An RNA nucleoside may comprise a modified or unmodified nucleobase. An RNA nucleoside may comprise a thymine nucleobase or a modified nucleobase, or may be an abasic nucleoside.

As used herein, “RNA sugar moiety” means an unmodified RNA sugar moiety

As used herein, “RNase H agent” means an antisense agent that acts, at least in part, through RNase H to modulate a target nucleic acid and/or protein encoded by a target nucleic acid. RNase H agents may be single-stranded, or RNase H agents may be double-stranded. RNase H compounds may comprise conjugate groups and/or terminal groups. RNase H agents may modulate the amount and/or activity of a target nucleic acid. The term RNase H agent excludes antisense agents that act principally through RISC/Ago2. An “RNAi agent” means an antisense agent that acts, at least in part, through RISC or Ago2 to modulate a target nucleic acid and/or a protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity of a target nucleic acid. The term RNAi agent excludes antisense agents that act through RNase H.

As used herein, “single-stranded” in reference to a nucleic acid (e.g., an oligonucleotide) means that the strand or region is unpaired; that is, the strand of linked nucleosides is not part of a duplex, not part of a double-stranded region. Single-stranded nucleic acids (e.g., single-stranded oligonucleotides) are capable of hybridizing to complementary nucleic acids to form duplexes, at which point they are no longer single-stranded.

As used herein, “stabilized phosphate moiety” means a 5′-phosphate analog that is metabolically more stable than a 5′-phosphate as naturally occurs on DNA or RNA.

As used herein, “stereorandom” or “stereorandom chiral center” in the context of a population of molecules of identical molecular formula means a chiral center that is not controlled during synthesis, or enriched following synthesis, for a particular absolute stereochemical configuration. The absolute stereochemical configuration of a chiral center can be controlled by using stereochemically-pure starting materials, e.g., using β-D-ribosyl nucleoside monomers for oligonucleotide synthesis. In contrast, the stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be the same as the number of molecules having the (R) configuration of the stereorandom chiral center (“racemic”). The stereorandom chiral center may not be racemic because one absolute configuration predominates following synthesis, e.g., due to the action of non-chiral reagents near the enriched stereochemistry of an adjacent sugar moiety. The stereorandom chiral center may be at the phosphorous atom of a stereorandom phosphorothioate or stereorandom mesyl phosphoramidate internucleoside linkage.

As used herein, a “strand” or “strand of linked nucleosides” means contiguous linked nucleosides connected via internucleoside linkages. A strand of linked nucleosides has a nucleobase sequence.

As used herein, “subject” means a human or a non-human animal.

As used herein, “sugar moiety” means an unmodified sugar moiety or a modified sugar moiety.

As used herein, “sugar mimic” means a group of atoms forming the portion of a nucleoside corresponding to the β-D-ribosyl sugar in RNA other than a modified furanosyl sugar moiety, a cyclic sugar surrogate, or an acyclic sugar surrogate.

As used herein, “sugar surrogate nucleoside” means a cyclic sugar surrogate nucleoside or an acyclic sugar surrogate nucleoside.

As used herein, “symptom” of a disease means any manifestation, indication, sign, or evidence of a disease. Symptoms include subjective and objective indicia of a disease and may be perceived, experienced, detected, observed, measured, and/or quantified. A symptom may be apparent upon diagnostic testing, and in certain instances only upon invasive diagnostic testing, including, but not limited to, post-mortem tests. A symptom may be an absence of a feature, such as failing to reach expected developmental milestones. Symptoms may include but are not limited to, angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen, and fever.

As used herein, “target nucleic acid” means a LPA nucleic acid that an antisense oligonucleotide is designed to affect. As used herein, “target RNA” means an LPA RNA transcript and includes pre-mRNA and/or mRNA unless otherwise specified.

As used herein, “target region” refers to a portion of a target nucleic acid that is complementary to the targeting region of an antisense oligonucleotide.

As used herein, “terminal group” means a chemical group or group of atoms that is covalently linked to a terminus of an oligonucleotide.

As used herein, “therapeutic index” means the ratio of a measure of toxicity or intolerability divided by a measure of potency or activity. Typically, the therapeutic index is expressed as the ratio between the concentration (or dose) at which a compound becomes toxic or induces unacceptable adverse effects (or the highest concentration or dose at which a compound is not toxic, or is tolerated, before it becomes toxic or induces unacceptable adverse effects) to a subject and the concentration (or dose) at which the compound is pharmacologically effective or produces the desired effect.

As used herein, “treating,” or “treatment,” with respect to a disease, means administering a compound or agent to a subject having or at risk for developing such disease. Treating a disease may result in amelioration of at least one symptom of such disease. Treatment may reduce, improve, and/or prevent one or more symptom(s) such that a symptom of the disease is diminished or is no longer apparent.

As used herein, “unmodified nucleobase” means unmodified adenine (A), unmodified thymine (T), unmodified cytosine (C), unmodified uracil (U), or unmodified guanine (G).

As used herein, an “unmodified nucleoside” means a compound or subunit comprising an unmodified sugar moiety and an unmodified nucleobase.

As used herein, “unmodified sugar moiety” means a 2′-OH(H) β-D-ribosyl sugar moiety, as found in RNA.

### EMBODIMENTS

1. An oligomeric duplex comprising a first oligomeric compound and a second oligomeric compound, wherein:
the first oligomeric compound comprises a first modified oligonucleotide consisting of 18 to 50 linked nucleosides, wherein the nucleobase sequence comprises a nucleobase sequence that is at least 80% identical to at least 18, at least 19, at least 20, at least 21, at least 22, or at least 23 contiguous nucleobases of the nucleobase sequence of any one of SEQ ID NO:83-87, wherein at least one but no more than 22%, no more than 20%, no more than 18%, no more than 15%, no more than 10%, or no more than 5% of the modified nucleosides in the first modified oligonucleotide comprises a 2′-F sugar moiety and/or an FHNA sugar surrogate, and
the second oligomeric compound comprises a second modified oligonucleotide consisting of 16 to 50 linked nucleosides, wherein the nucleobase sequence comprises a nucleobase sequence that is at least 80% identical to at least 16, at least 17, at least 18, at least 19, at least 20, or at least 21 contiguous nucleobases of the nucleobase sequence of any one of SEQ ID Nos: 99,100, or 112, wherein at least one but no more than 25%, no more than 20%, no more than 18%, no more than 16%, no more than 14%, no more than 12%, or no more than 10%, of the modified nucleosides in the second modified oligonucleotide comprises a 2′-F sugar moiety and/or an FHNA sugar surrogate;
wherein the first modified oligonucleotide and the second modified oligonucleotide are complementary to one another; and
wherein each of the nucleosides of the first modified oligonucleotide and the second modified oligonucleotide independently comprises a modified sugar moiety or sugar surrogate.

2. The oligomeric duplex of embodiment 1,
1) wherein the first modified oligonucleotide comprises at least one and no more than four modified nucleosides comprising a modified sugar moiety or sugar surrogate comprising a 2′-F sugar moiety and/or an FHNA sugar surrogate, and/or wherein the second modified oligonucleotide comprises at least one and no more than four modified nucleosides comprising a modified sugar moiety or sugar surrogate comprising a 2′-F sugar moiety and/or an FHNA sugar surrogate; and/or wherein the oligomeric duplex comprises at least one and no more than eight modified nucleosides comprising a modified sugar moiety or sugar surrogate comprising a 2′-F sugar moiety and/or an FHNA sugar surrogate; and/or
2) wherein at least 18 nucleosides of the first modified oligonucleotide, each independently, and at least 16 nucleosides of the second modified oligonucleotide, each independently, comprise a modified sugar moiety or sugar surrogate selected from a 2′-F sugar moiety, a 2′-MOE sugar moiety, a 2′-OMe sugar moiety, a DNA sugar moiety, and an FHNA sugar surrogate; and/or
3) wherein the nucleobase sequence of the first modified oligonucleotide comprises at least 18, at least 19, at least 20, at least 21, at least 22, or at least 23 contiguous nucleobases of the nucleobase sequence of any one of SEQ ID NOs:83-87, and wherein the nucleobase sequence of the second modified oligonucleotide comprises at least 16, at least 17, at least 18, at least 19, at least 20, or at least 21 contiguous nucleobases of the nucleobase sequence of any one of SEQ ID Nos:99, 100, or 112.

3. An oligomeric duplex comprising a first oligomeric compound and a second oligomeric compound, wherein:
a first oligomeric compound comprises a modified oligonucleotide consisting of 18 to 28 linked nucleosides, wherein the nucleobase sequence comprises at least 18, at least 19, at least 20, at least 21, at least 22, or at least 23 nucleobases complementary to the nucleobase sequence of SEQ ID NO:2, wherein no more than 20%, no more than 18%, no more than 15%, no more than 10%, or no more than 5% of the modified nucleosides in the first modified oligonucleotide comprises a fluorine, and a second oligomeric compound comprises a modified oligonucleotide consisting of 16 to 26 linked nucleosides, wherein the nucleobase sequence comprises at least 16, at least 17, at least 18, at least 19, at least 20, or at least 21 nucleobases of the nucleobase sequence of SEQ ID NOs:2, wherein no more than no more than 18%, no more than 16%, no more than 14%, no more than 12%, or no more than 10%, of the modified nucleosides in the second modified oligonucleotide comprises a fluorine; wherein each of the nucleosides of the first modified oligonucleotide independently and the second modified oligonucleotide independently comprises a modified sugar moiety or sugar surrogate independently selected from a 2′-F sugar moiety, a 2′-MOE sugar moiety, a 2′-OMe sugar moiety, a DNA sugar moiety, a deoxyxylose sugar moiety, and an FHNA;
wherein the first modified oligonucleotide comprises a stabilized phosphate group attached to the 5′-terminal nucleoside;
wherein one or more nucleosides of the first modified oligonucleotide comprise a 2′-MOE sugar moiety and one or more of the nucleosides of the first modified oligonucleotide comprise a 2′-OMe sugar moiety, and one or more of the nucleosides of the second modified oligonucleotide comprise a 2′-MOE sugar moiety, and one or more of the nucleosides of the second modified oligonucleotide comprise a 2′-OMe sugar moiety; and wherein the first modified oligonucleotide and the second modified oligonucleotide comprise at least one modified internucleoside linkage.

4. The oligomeric duplex of embodiment 3, wherein the first modified oligonucleotide comprises at least one and no more than four modified nucleosides comprising a modified sugar moiety or sugar surrogate comprising a fluorine, and/or wherein the second modified oligonucleotide comprises at least one and no more than four modified nucleosides comprising a modified sugar moiety or sugar surrogate comprising a fluorine; and/or wherein the oligomeric duplex comprises at least two and no more than eight modified nucleosides comprising a modified sugar moiety or sugar surrogate comprising a fluorine.

5. The oligomeric duplex of any one of embodiment 1-4, wherein a nucleoside comprising a modified sugar moiety or sugar surrogate comprising a fluorine of the first modified oligonucleotide is independently selected from one of:
i. the second nucleoside counting from the 5′ end,
ii. the second and fourteenth nucleosides counting from the 5′ end, or
iii. the second, sixth and fourteenth nucleosides counting from the 5′ end, or
iv. the second and sixteenth nucleosides counting from the 5′ end, or
v. the second, fourteenth and sixteenth nucleosides counting from the 5′ end, or
vi. the second, sixth, fourteenth, and sixteenth nucleosides counting from the 5′ end;
   wherein each modified sugar moiety or sugar surrogate comprising a fluorine is independently a 2′-fluoro sugar moiety or a 3′-fluoro-hexitol sugar moiety; and/or wherein a nucleoside comprising a modified sugar moiety or sugar surrogate comprising a fluorine of the second modified oligonucleotide is independently selected from one of:
i. the ninth and tenth nucleosides counting from the 5′ end, or
ii. the tenth and eleventh nucleosides counting from the 5′ end, or
iii. the ninth and eleventh nucleosides counting from the 5′ end, or
iv. the ninth, tenth and eleventh nucleosides counting from the 5′ end, or
v. the seventh, ninth, tenth and eleventh nucleosides counting from the 5′ end, or
vi. the seventh, ninth, and eleventh nucleosides counting from the 5′ end;
   wherein each modified sugar moiety or sugar surrogate comprising a fluorine is independently a 2′-fluoro sugar moiety or a 3′-fluoro-hexitol sugar moiety.

6. The oligomeric duplex of any one of embodiments 1-5, wherein one modified nucleoside of the first modified oligonucleotide comprising a fluorine comprises a 3′-fluoro-hexitol sugar moiety sugar surrogate.

7. The oligomeric duplex of any one of embodiments 1-5, wherein one or more nucleosides of the first modified oligonucleotide is a 2′-deoxynucleoside; and wherein optionally one or more nucleosides of the second modified oligonucleotide is a 2′-deoxynucleoside.

8. The oligomeric duplex of embodiment 7, wherein one or more nucleosides in a region of the sequence of the first modified oligonucleotide between and including the fifth nucleoside to the sixteenth nucleoside counting from the 5′ end of the first modified oligonucleotide is a 2′-deoxynucleoside.

9. The oligomeric duplex of embodiment 8, wherein the one or more 2′-deoxynucleosides in the first modified oligonucleotide is any of the fifth, sixth, seventh, fourteenth, and/or sixteenth nucleosides counting from the 5′ end of the first modified oligonucleotide, wherein the one or more 2′-deoxynucleosides is optionally at a position independently selected from one of:
i. the fifth, sixth or seventh nucleoside counting from the 5′ end, or
ii. the sixth and fourteenth nucleoside from the 5′ end, or
iii. the sixth and sixteenth nucleosides counting from the 5′ end, or
iv. the sixth, fourteenth and sixteenth nucleosides counting from the 5′ end, or
v. the fourteenth and sixteenth nucleosides counting from the 5′ end; and
   wherein optionally one or more 2′-deoxynucleosides is independently selected from one of the ninth, tenth and/or eleventh nucleoside counting from the 5′ end of the second modified oligonucleotide.

10. The oligomeric duplex of embodiment 9, wherein each 2′ deoxynucleoside independently comprises a 2′-2′-β-D-deoxyribose sugar moiety or a 2′-β-D-deoxyxylose sugar moiety.

11. The oligomeric duplex of any one of embodiments 1-10, wherein two of the 3′ terminal nucleosides of the first modified oligonucleotide comprise a two-nucleoside overhang, wherein the overhang nucleosides comprise one modified adenosine (A), two modified adenosine (AA), two modified uridine (UU) nucleosides, two modified inosine (II) nucleosides, or two modified nucleosides wherein one is an inosine and one is an adenosine (AI or IA).

12. The oligomeric duplex of embodiment 11, wherein each of the 5′- and/or 3′-terminal nucleosides of the first modified oligonucleotide comprise a 2′-MOE sugar moiety; and optionally, each of the 5′- and/or 3′-terminal nucleosides of the second modified oligonucleotide comprise a 2′-MOE sugar moiety.

13. The oligomeric duplex of embodiment 11, wherein one of the 5′-terminal nucleosides and/or two of the 3′-terminal nucleosides of the first modified oligonucleotide comprise a 2′-MOE sugar moiety; and optionally, two of the 5′-terminal nucleosides and/or two of the 3′-terminal nucleosides of the second modified oligonucleotide comprise a 2′-MOE sugar moiety.

14. The oligomeric duplex of any one of embodiments 1-13, wherein at least thirteen nucleosides, at least fourteen nucleosides, at least fifteen nucleosides, at least sixteen nucleosides, at least seventeen, at least eighteen nucleosides, or at least nineteen nucleosides of the first modified oligonucleotide comprise a 2′-OMe sugar moiety; and wherein at least thirteen nucleosides, at least fourteen nucleosides, at least fifteen nucleosides, at least sixteen nucleosides, at least seventeen nucleosides, at least eighteen nucleosides, or at least nineteen of the second modified oligonucleotide comprise a 2′-OMe sugar moiety.

15. The oligomeric duplex of any one of embodiments 1-14, wherein at least one nucleoside of the first modified oligonucleotide comprising a 2′-MOE sugar moiety is an internal nucleoside in a region of the sequence of the first modified oligonucleotide that is any of the ninth and/or tenth nucleosides counting from the 5′ end of the first modified oligonucleotide.

16. The oligomeric duplex of any one of embodiments 1-15, wherein the first oligomeric compound comprises a stabilized phosphate group attached to the 5′-terminal nucleoside, wherein the stabilized phosphate group comprises a methylene phosphonate, cyclopropyl phosphonate or a vinyl phosphonate.

17. The oligomeric duplex of any one of embodiments 1-16, wherein the first modified oligonucleotide and/or the second modified oligonucleotide comprises at least one modified internucleoside linkage, wherein at least one modified internucleoside linkage is a phosphorothioate internucleoside linkage.

18. The oligomeric duplex of embodiment 17, wherein each internucleoside linkage is independently selected from a phosphodiester internucleoside linkage and a phosphorothioate internucleoside linkage.

19. The oligomeric duplex of embodiment 17, wherein the internucleoside linkages between the first and second nucleosides and the second and third nucleosides counting from the 5′ end of the first modified oligonucleotide are phosphorothioate internucleoside linkages, and/or wherein the internucleoside linkages between the first and second nucleosides and the second and third nucleosides counting from the 3′ end of the first modified oligonucleotide are phosphorothioate internucleoside linkages; and
optionally, wherein the internucleoside linkages between the first and second nucleosides and the second and third nucleosides counting from the 5′ end of the second modified oligonucleotide are phosphorothioate internucleoside linkages, and/or wherein the internucleoside linkages between the first and second nucleosides and the second and third nucleosides counting from the 3′ end of the second modified oligonucleotide are phosphorothioate internucleoside linkages.

20. The oligomeric duplex of embodiment 1, wherein the first modified oligonucleotide comprises a modified sugar motif independently selected from one of efyyyfyyyyyyyfyfyyyyyyy, yfyyyfyyyyyyyfyfyyyyyyy, efyyydyyeyyyydydyyyyyee, efyyydyyyyyyyfyfyyyyyee, efyyydyyeyyyyfyfyyyyyee, ehyyyfyyyyyyyfyfyyyyyee, and ehyyyfyyeyyyyfyfyyyyyee, wherein each ‘e’ represents a 2′-MOE sugar moiety, each ‘y’ represents a 2′-OMe sugar moiety, each ‘f’ represents a 2′-F sugar moiety, each ‘h’ represents a 3′-fluoro-hexitol sugar moiety, and each ‘d’ represents a 2′-deoxy sugar moiety, and wherein all except 0, 1, or 2 modifications are identical to the sugar motif; and/or the second modified oligonucleotide comprises a modified sugar motif independently selected from one of yyyyyyfyfffyyyyyyyyyy and eeyyyyyyyffyyyyyyyyee, wherein each ‘e’ represents a 2′-MOE sugar moiety, each ‘y’ represents a 2′-OMe sugar moiety, and each ‘f’ represents a 2′-F sugar moiety, and wherein all except 0, 1, or 2 modifications are identical to the sugar motif.

21. The oligomeric duplex of embodiment 1, wherein the first modified oligonucleotide comprises a modified sugar motif independently selected from one of efyyyfyyyyyyyfyfyyyyyyy, yfyyyfyyyyyyyfyfyyyyyyy, efyyydyyeyyyydydyyyyyee, efyyydyyyyyyyfyfyyyyyee, efyyydyyeyyyyfyfyyyyyee, ehyyyfyyyyyyyfyfyyyyyee, and ehyyyfyyeyyyyfyfyyyyyee, wherein each ‘e’ represents a 2′-MOE sugar moiety, each ‘y’ represents a 2′-OMe sugar moiety, each ‘f’ represents a 2′-F sugar moiety, each ‘h’ represents a 3′-fluoro-hexitol sugar moiety, and each ‘d’ represents a 2′-deoxy sugar moiety; and/or the second modified oligonucleotide comprises a modified sugar motif independently selected from one of yyyyyyfyfffyyyyyyyyyy and eeyyyyyyyffyyyyyyyyee, wherein each ‘e’ represents a 2′-MOE sugar moiety, each ‘y’ represents a 2′-OMe sugar moiety, and each ‘f’ represents a 2′-F sugar moiety.

22. The oligomeric duplex of embodiment 21, wherein the first modified oligonucleotide comprises a modified sugar motif and is paired with the second modified oligonucleotide comprising a sugar motif selected from: efyyyfyyyyyyyfyfyyyyyyy and yyyyyyfyfffyyyyyyyyyy; yfyyyfYyyyyyyfyfyyyyyyy and yyyyyyfyfffyyyyyyyyyy; efyyydyyeyyyydydyyyyyee and eeyyyyyyyffyyyyyyyyee; efyyydyyyyyyyfyfyyyyyee and eeyyyyyyyffyyyyyyyyee; efyyydyyeyyyyfyfyyyyyee and eeyyyyyyyffyyyyyyyyee; ehyyyfyyeyyyyfyfyyyyyee and eeyyyyyyyffyyyyyyyyee; and ehyyyfyyyyyyyfyfyyyyyee and eeyyyyyyyffyyyyyyyyee.

23. The oligomeric duplex of embodiment 1-3, wherein the first modified oligonucleotide comprises a modified sugar motif independently selected from one of yfyyyfyyyyyyyfyfyyyyyyy, efyyydyyeyyyydydyyyyyee, efyyydyyyyyyyfyfyyyyyee, efyyydyyeyyyyfyfyyyyyee, ehyyyfyyyyyyyfyfyyyyee, ehyyyfyyeyyyyfyfyyyyyee, efyyyyyyyyyyyfyfyyyyyee, efyyyfyyyyyyyfyyyyyyyee, efyyyyyyeyyyyfyfyyyyee, efyyyfyyeyyyyfyyyyyyyee, efyyxfyyyyyyyfyyyyyyyee, efyyyfxyyyyyyfyyyyyyyee, and efyyyxyyyyyyyfyyyyyyyee, and/or the second modified oligonucleotide comprises a modified sugar motif independently selected from one of eeyyyyyyyffyyyyyyyyee, yyyyyyyyfffyyeyyyyyyy, eeyyyyyyfffyyeyyyyyee, eeyyyyyydffyyeyyyyyee, eeyyyyyyfdfyyeyyyyyee, eeyyyyyyffdyyeyyyyyee, eeyyyyyyhffyyeyyyyyee, eeyyyyyyfhfyyeyyyyyee, eeyyyyyyffhyyeyyyyyee, eeyyyyyyfxfyyeyyyyyee, eeyyyyyyffxyyeyyyyyee, and eeyyyyyyxffyyeyyyyyee; wherein each ‘e’ represents a 2′-MOE sugar moiety, each ‘y’ represents a 2′-OMe sugar moiety, each ‘f’ represents a 2′-F sugar moiety, each ‘h’ represents a 3′-fluoro-hexitol sugar moiety, each ‘d’ represents a 2′-deoxyribose sugar moiety, and each ‘x’ represents a 2′-deoxyxylose sugar moiety; and wherein all except 0, 1, or 2 modifications are identical to the sugar motif.

24. The oligomeric duplex of embodiment 1-3, wherein the first modified oligonucleotide comprises a modified sugar motif independently selected from one of efyyydyyeyyyydydyyyyyee, efyyyyyyyyyyfyfyyyyyee, efyyyfyyyyyyyfyyyyyyyee, efyyxfyyyyyyyfyyyyyyyee, and efyyyfxyyyyyyfyyyyyyyee, and/or the second modified oligonucleotide comprises a modified sugar motif independently selected from one of eeyyyyyyfffyyeyyyyyee and eeyyyyyyfdfyyeyyyyyee; wherein each ‘e’ represents a 2′-MOE sugar moiety, each ‘y’ represents a 2′-OMe sugar moiety, each ‘f’ represents a 2′-F sugar moiety, each ‘h’ represents a 3′-fluoro-hexitol sugar moiety, each ‘d’ represents a 2′-deoxyribose sugar moiety, and each ‘x’ represents a 2′-deoxyxylose sugar moiety; and wherein all except 0, 1, or 2 modifications are identical to the sugar motif.

25. The oligomeric duplex of embodiment 24, wherein the first modified oligonucleotide comprises a modified sugar motif and is paired with the second modified oligonucleotide comprising a sugar motif selected from: efyyyfyyyyyyyfyfyyyyyyy and yyyyyyfyfffyyyyyyyyyy; yfyyyfyyyyyyyfyfyyyyyy and yyyyyyfyfffyyyyyyyyyy; efyyydyyeyyyydydyyyyyee and eeyyyyyyyffyyyyyyyyee; efyyydyyyyyyyfyfyyyyyee and eeyyyyyyyffyyyyyyyyee; efyyydyyeyyyyfyfyyyyyee and eeyyyyyyyffyyyyyyyyee; ehyyyfyyyyyyyfyfyyyyyee and eeyyyyyyyffyyyyyyyyee; ehyyyfyyeyyyyfyfyyyyyee and eeyyyyyyyffyyyyyyyyee; efyyyyyyyyyyyfyfyyyyyee and yyyyyyyyfffyyeyyyyyyy; efyyyyyyyyyyyfyfyyyyyee and eeyyyyyyfffyyeyyyyyee; efyyyfyyyyyyyfyyyyyyyee and yyyyyyyyfffyyeyyyyyyy; efyyyfyyyyyyyfyyyyyyyee and eeyyyyyyfffyyeyyyyyee; efyyyyyyyyyyyfyfyyyyyee and eeyyyyyyyffyyyyyyyyee; efyyyfyyyyyyyfyyyyyyyee and eeyyyyyyyffyyyyyyyyee; efyyydyyeyyyydydyyyyyee and yyyyyyyyfffyyeyyyyyyy; efyyyfyyyyyyyfyyyyyyyee and eeyyyyyydffyyeyyyyyee; efyyyfyyyyyyyfyyyyyyyee and eeyyyyyyfdfyyeyyyyyee; efyyyfyyyyyyyfyyyyyyyee and eeyyyyyyffdyyeyyyyyee; efyyyfyyyyyyyfyyyyyyyee and eeyyyyyyhffyyeyyyyyee; efyyyfyyyyyyyfyyyyyyyee and eeyyyyyyfhfyyeyyyyyee; efyyyfyyyyyyyfyyyyyyyee and eeyyyyyyffhyyeyyyyyee; efyyyfyyyyyyyfyyyyyyyee and eeyyyyyyfxfyyeyyyyyee; efyyyfyyyyyyyfyyyyyyyee and eeyyyyyyffxyyeyyyyyee; efyyyfyyyyyyyfyyyyyyyee and eeyyyyyyxffyyeyyyyyee; efyyydyyeyyyydydyyyyyee and eeyyyyyyfhfyyeyyyyyee; efyyydyyeyyyydydyyyyyee and eeyyyyyyfdfyyeyyyyyee; efyyydyyeyyyydydyyyyyee and eeyyyyyyffhyyeyyyyyee; efyyydyyeyyyydydyyyyyee and eeyyyyyyffdyyeyyyyyee; efyyydyyeyyyydydyyyyyee and eeyyyyyydffyyeyyyyyee; efyyydyyeyyyydydyyyyyee and eeyyyyyyhffyyeyyyyyee; efyyydyyeyyyydydyyyyyee and eeyyyyyyxffyyeyyyyyee; efyyydyyeyyyydydyyyyyee and eeyyyyyyfxfyyeyyyyyee; efyyydyyeyyyydydyyyyyee and eeyyyyyyffxyyeyyyyyee; efyyyyyyeyyyyfyfyyyyyee and eeyyyyyyyffyyyyyyyyee; efyyyfyyeyyyyfyyyyyyyee and eeyyyyyyyffyyyyyyyyee; efyyyyyyeyyyyfyfyyyyyee and eeyyyyyyfffyyeyyyyyee; efyyyfyyeyyyyfyyyyyyyee and eeyyyyyyfffyyeyyyyyee; efyyxfyyyyyyyfyyyyyyyee and eeyyyyyyfffyyeyyyyyee; efyyyfxyyyyyyfyyyyyyyee and eeyyyyyyfffyyeyyyyyee; and efyyyxyyyyyyyfyyyyyyyee and eeyyyyyyfffyyeyyyyyee.

26. The oligomeric duplex of any one of embodiments 1-25, wherein the agent comprises a conjugate group comprises a liver cell-targeting moiety comprising a conjugate moiety and a conjugate linker.

27. The oligomeric duplex of embodiment 26, wherein the oligomeric duplex comprises a conjugate moiety that binds asialoglycoprotein receptor (ASGPR).

28. The oligomeric duplex of embodiment 27, wherein the conjugate moiety is selected from a GalNAc moiety.

29. The oligomeric duplex of embodiment 28, wherein the GalNAc conjugate moiety is selected from Table A.

30. The oligomeric duplex of embodiment 28, wherein the conjugate group consists of a GalNAc ligand and a conjugate linker.

31. The oligomeric duplex of embodiment 28, wherein the GalNAc ligand has the structure:

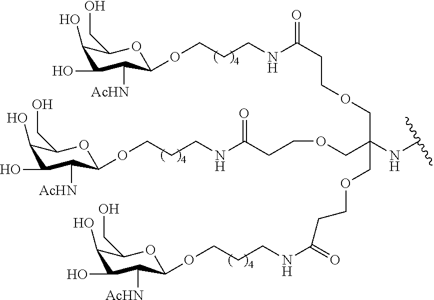

32. The oligomeric duplex of embodiment 30, wherein the conjugate group has the structure:

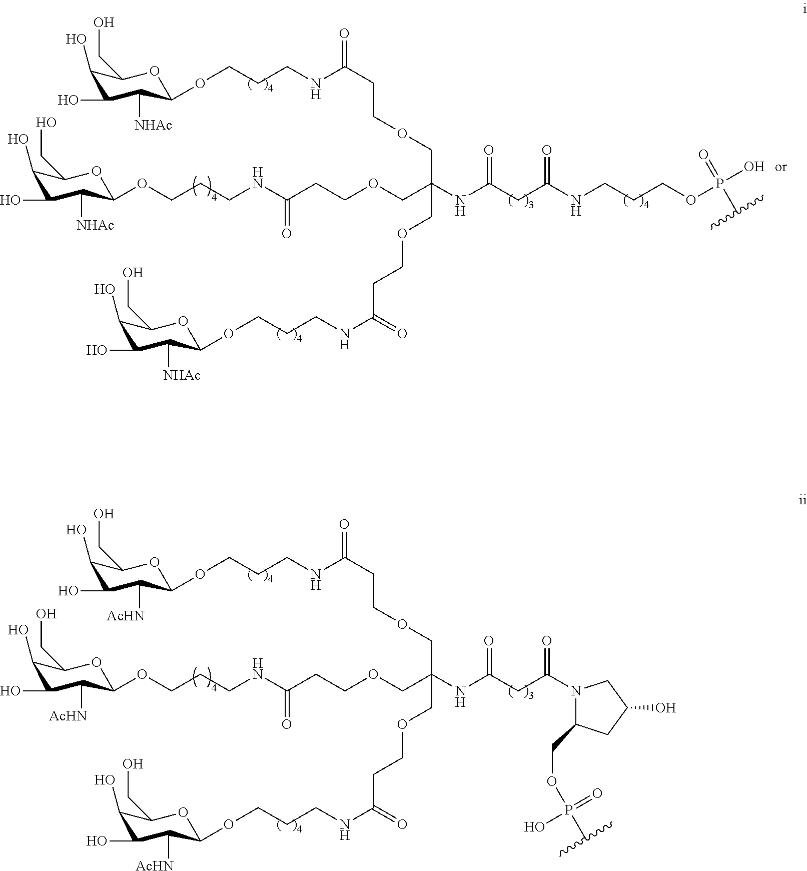

or an ion or salt thereof, wherein the conjugate linker is covalently connected to an oligonucleotide.

33. The oligomeric duplex of any one of embodiments 25-30, wherein the conjugate group is conjugated directly to the modified oligonucleotide, or the conjugate linker of the conjugate group consists of a single bond.

34. The oligomeric duplex of embodiment 30, wherein the conjugate group is conjugated to the 5′ end and/or the 3′ end of the modified oligonucleotide.

35. The oligomeric duplex of embodiment 34, wherein the conjugate group is attached to the 5′-terminal nucleoside of the second modified oligonucleotide or the 3′-terminal nucleoside of the second modified oligonucleotide.

36. The oligomeric duplex of embodiment 30, wherein the conjugate linker of the conjugate group is cleavable.

37. The oligomeric duplex of embodiment 36, wherein the conjugate linker comprises 1 to 3 linker-nucleosides.

38. The oligomeric duplex of embodiment 32, wherein the conjugate having the structure (i), or an ion or salt thereof is attached to the 5′-terminal nucleoside; and/or wherein the conjugate having the structure (ii) or an ion or salt thereof is attached to the 3′-terminal nucleoside of the modified oligonucleotide.

39. An oligomeric duplex comprising i) a first oligomeric compound comprising a modified oligonucleotide consisting of 16 to 50 linked nucleosides, wherein the nucleobase sequence comprises a nucleobase sequence that is at least 80% identical to at least 16 contiguous nucleobases of any one of SEQ ID NOs:83-87, wherein all except 0, 1, or 2 nucleobases are identical to the nucleobase sequence SEQ ID NOs:99, 100, or 112, and wherein each of the first modified oligonucleotide comprises a modified sugar moiety selected from: efyyyfyyyyyyyfyfyyyyyyy, yfyyyfyyyyyyyfyfyyyyyy, efyyydyyeyyyydydyyyyyee, efyyydyyyyyyyfyfyyyyyee, efyyydyyeyyyyfyfyyyyyee, ehyyyfyyyyyyyfyfyyyyyee, and ehyyyfyyeyyyyfyfyyyyyee, wherein each ‘e’ represents a 2′-MOE sugar moiety, each ‘y’ represents a 2′-OMe sugar moiety, each ‘f’ represents a 2′-F sugar moiety, each ‘h’ represents a 3′-fluoro-hexitol sugar moiety, and each ‘d’ represents a 2′-deoxy sugar moiety; and ii) a second oligomeric compound comprising a second modified oligonucleotide consisting of 14-50 linked nucleosides wherein the nucleobase sequence comprises a nucleobase sequence that is at least 80% identical to at least 14 contiguous nucleobases of any one of SEQ ID NOs: 83-87, wherein all except 0, 1, or 2 nucleobases are identical to the nucleobase sequence of SEQ ID NOs: 99, 100, or 112, and wherein each of the second modified oligonucleotide comprises a modified sugar motif selected from one of yyyyyyfyfffyyyyyyyyyy and eeyyyyyyyffyyyyyyyyee, wherein each ‘e’ represents a 2′-MOE sugar moiety, each ‘y’ represents a 2′-OMe sugar moiety, and each ‘f’ represents a 2′-F sugar moiety.

40. An oligomeric duplex comprising i) a first modified oligonucleotide consisting of 18 to 28 linked nucleosides, wherein the nucleobase sequence comprises at least 16 nucleobases complementary to SEQ ID NOs:2, and wherein all except 0, 1, or 2 nucleobases are identical to any one of SEQ ID NOs:3-48, or 132-140; and ii) a second modified oligonucleotide consisting of 16 to 26 linked nucleosides, wherein the nucleobase sequence comprises at least 15 nucleobases of SEQ ID NOs:2, and wherein all except 0, 1, or 2 nucleobases are identical to any one of SEQ ID NOs:49-82, or 141-142.

41. The oligomeric duplex of embodiment 40, wherein the sequence of the first modified oligonucleotide comprises at least 20, at least 21, at least 22 or 23 bases of the sequence of any one of SEQ ID NOs:3-48, or 132-140; and wherein the sequence of the second modified oligonucleotide comprises at least 18, at least 19, at least 20 or 21 bases of the sequence of any one of SEQ ID NO:49-82, or 141-142.

42. The oligomeric duplex of embodiment 40, wherein the sequence of the first modified oligonucleotide comprises the sequence of any one of SEQ ID NOs:3-48, or 132-140; and wherein the sequence of the second modified oligonucleotide comprises the sequence of any one of SEQ ID NO:49-82, or 141-142.

43. The oligomeric duplex of embodiment 40, wherein the sequence of the first modified oligonucleotide consists of the sequence of any one of SEQ ID NOs:3-48, or 132-140; and wherein the sequence of the second modified oligonucleotide consists of the sequence of any one of SEQ ID NO:49-82, or 141-142.

44. The oligomeric duplex of embodiment 40, comprising i) a first modified oligonucleotide consisting of 18 to 28 linked nucleosides, wherein the nucleobase sequence comprises at least 16 nucleobases complementary to SEQ ID NOs:2, and wherein all except 0, 1, or 2 nucleobases are identical to any one of SEQ ID NOs:5-6, or 15-48, or 132-140; and ii) a second modified oligonucleotide consisting of 16 to 26 linked nucleosides, wherein the nucleobase sequence comprises at least 15 nucleobases of SEQ ID NOs:2, and wherein all except 0, 1, or 2 nucleobases are identical to any one of SEQ ID NOs:52-82, or 141-142.

45. The oligomeric duplex of claim 44, wherein the sequence of the first modified oligonucleotide comprises at least 20, at least 21, at least 22 or 23 bases of the sequence of any one of SEQ ID NOs: 5-6, or 15-48, or 132-140; and wherein the sequence ofthe second modified oligonucleotide comprises at least 18, at least 19, at least 20 or 21 bases of the sequence of any one of SEQ ID NO:52-82, or 141-142.

46. The oligomeric duplex of claim 44, wherein the sequence of the first modified oligonucleotide comprises the sequence of any one of SEQ ID NOs: 5-6, or 15-48, or 132-140; and wherein the sequence of the second modified oligonucleotide comprises the sequence of any one of SEQ ID NO:52-82, or 141-142.

47. The oligomeric duplex of claim 44, wherein the sequence of the first modified oligonucleotide consists of the sequence of any one of SEQ ID NOs: 5-6, or 15-48, or 132-140; and wherein the sequence of the second modified oligonucleotide consists of the sequence of any one of SEQ ID NO:52-82, or 141-142.

48. An oligomeric duplex comprising any one the following antisense compound and sense compound pairs:

| Compound | Antisense | Sense |
|---|---|---|
| No. | Compound No. | Compound No. |
| 1749546 | 1749544 | 1749524 |
| 1764923 | 1764919 | 1749524 |
| 1792737 | 1792668 | 1792669 |
| 1792748 | 1792676 | 1792677 |
| 1792749 | 1792678 | 1792679 |
| 1792778 | 1792688 | 1792677 |
| 1792779 | 1792689 | 1792679 |
| 1792808 | 1792699 | 1792679 |
| 1792809 | 1792698 | 1792677 |
| 1792825 | 1792704 | 1792669 |
| 1792838 | 1792709 | 1792679 |
| 1792839 | 1792708 | 1792677 |
| 1792855 | 1792714 | 1792669 |
| 1792867 | 1792718 | 1792677 |
| 1792869 | 1792719 | 1792679 |
| 1820728 | 1814997 | 1814995 |
| 1820731 | 1814997 | 1814996 |
| 1820734 | 1814998 | 1814995 |
| 1820737 | 1814998 | 1814996 |
| 1824827 | 1819834 | 1819832 |
| 1824839 | 1819835 | 1819832 |
| 1824840 | 1819834 | 1819833 |
| 1824842 | 1819835 | 1819833 |
| 1826328 | 1819834 | 1792677 |
| 1826331 | 1819835 | 1792677 |
| 1826337 | 1814997 | 1792679 |
| 1826340 | 1814998 | 1792679 |
| 1826343 | 1792678 | 1814995 |
| 1826346 | 1814998 | 1792679 |
| 1826573 | 1819835 | 1826559 |
| 1829932 | 1819835 | 1821669 |
| 1829951 | 1819835 | 1821829 |
| 1829952 | 1819835 | 1821830 |
| 1829953 | 1819835 | 1821831 |
| 1829954 | 1819835 | 1821832 |
| 1829955 | 1819835 | 1821833 |
| 1829956 | 1819835 | 1821835 |
| 1829957 | 1819835 | 1821836 |
| 1829958 | 1819835 | 1821834 |
| 1829959 | 1792676 | 1821832 |
| 1829960 | 1792676 | 1821829 |
| 1829961 | 1792676 | 1821833 |
| 1829962 | 1792676 | 1821830 |
| 1829963 | 1792676 | 1821669 |
| 1829964 | 1792676 | 1821831 |
| 1829965 | 1792676 | 1821834 |
| 1829966 | 1792676 | 1821835 |
| 1829967 | 1792676 | 1821836 |
| 1831890 | 1826580 | 1792677 |
| 1831892 | 1826581 | 1792677 |
| 1831895 | 1826580 | 1819833 |
| 1831896 | 1826581 | 1819833 |
| 1831897 | 1826582 | 1814996 |
| 1831898 | 1826583 | 1814996 |
| 1839407 | 1839388 | 1826559 |
| 1839408 | 1839376 | 1826559 |
| 1839409 | 1839378 | 1826559 |
| 1839410 | 1839377 | 1814996 |
| 1839411 | 1839379 | 1814996 |
| 1839412 | 1839380 | 1814996 |
| 1839601 | 1826581 | 1826559 |
| 1840071 | 1792676 | 1839284 |
| 1840201 | 1838448 | 1838396 |
| 1840202 | 1838470 | 1838396 |
| 1840453 | 1838449 | 1838398 |
| 1840454 | 1838471 | 1838398 |
| 1840892 | 1838451 | 1838410 |
| 1840893 | 1838473 | 1838410 |
| 1841023 | 1838453 | 1838404 |
| 1841095 | 1838475 | 1838404 |
| 1841097 | 1838454 | 1838406 |
| 1841125 | 1838476 | 1838406 |
| 1841126 | 1838464 | 1838377 |
| 1841127 | 1838465 | 1838389 |
| 1841128 | 1838472 | 1838400 |
| 1841129 | 1838450 | 1838400 |
| 1841133 | 1838469 | 1838395 |
| 1841134 | 1838447 | 1838395 |
| 1841135 | 1838468 | 1838393 |
| 1841136 | 1838446 | 1838393 |
| 1841137 | 1838466 | 1838390 |
| 1843479 | 1792676 | 1841270 |
| 1843480 | 1792676 | 1841096 |
| 1843481 | 1792676 | 1841271 |
| 1843482 | 1792676 | 1841369 |
| 1858685 | 1838442 | 1838377 |
| 1859018 | 1838443 | 1838389 |
| 1859347 | 1838444 | 1838390 |
| 1859802 | 1838445 | 1838392 |
| 1861908 | 1838455 | 1838408 |
| 1861909 | 1838467 | 1838392 |
| 1861910 | 1838477 | 1838408 |

49. An oligomeric duplex comprising i) a first modified oligonucleotide consisting of 18 to 28 linked nucleosides, wherein the nucleobase sequence comprises at least 18 nucleobases complementary to SEQ ID NO: 2, and wherein the modified oligonucleotide comprises a sugar motif and internucleoside linkage motif selected from any one of:

| | |
|---|---|
| i | ehyyyfyyeyyyyfyfyyyyyee and ssooooooooooooooooooss, |
| ii | efyyydyyeyyyydydyyyyyee and ssooosooooooososooooss, |
| iii | efyyyyyyyyyyyfyfyyyyyee and ssooooooooooooooooooss, |
| iv | efyyyfyyyyyyyfyyyyyyyee and ssooooooooooooooooooss, |
| v | efyyyyyyeyyyyfyfyyyyyee and ssooooooooooooooooooss, |
| vi | efyyyfyyeyyyyfyyyyyyyee and ssooooooooooooooooooss, |
| vii | efyyxfyyyyyyyfyyyyyyyee and ssooooooooooooooooooss, |
| viii | efyyyfxyyyyyyfyyyyyyyee and ssooooooooooooooooooss, or |
| ix | efyyyxyyyyyyytyyyyyyyee and ssooooooooooooooooooss; |

and ii) a second modified oligonucleotide consisting of 16 to 26 linked nucleosides, wherein the nucleobase sequence comprises at least 16 nucleobases of SEQ ID NO: 2, the modified oligonucleotide comprises a sugar motif and an internucleoside linkage motif selected from any one of:

| | |
|---|---|
| i | eeyyyyyyyffyyyyyyyyee and ssooooooosooooooooss, |
| ii | yyyyyyyyfffyyeyyyyyyy and ssooooooooooooooooss, |
| iii | eeyyyyyyfffyyeyyyyyee and ssooooooooooooooooss, |
| iv | eeyyyyyydffyyeyyyyyee and ssoooooosoooooooooss, |
| v | eeyyyyyyfdfyyeyyyyyee and ssooooooosooooooooss, |
| vi | eeyyyyyyffdyyeyyyyyee and ssoooooooosoooooooss, |
| vii | eeyyyyyyhffyyeyyyyyee and ssooooooooooooooooss, |
| viii | eeyyyyyyfhfyyeyyyyyee and ssooooooooooooooooss, |
| ix | eeyyyyyyffhyyeyyyyyee and ssooooooooooooooooss, |
| x | eeyyyyyyfxfyyeyyyyyee and ssooooooooooooooooss, |
| xi | eeyyyyyyffxyyeyyyyyee and ssooooooooooooooooss, or |
| xii | eeyyyyyyxffyyeyyyyyee and ssooooooooooooooooss. |

50. An oligomeric duplex comprising A and B:

A. an oligomeric compound according to any one of the
following chemical notation:
(SEQ ID NO: 5)
vP-TesGfsGyoAyoGyoUdsAyoUyoGeoUyoGyoCyoCyoUdsCyoGdsAyoUyoAyoAyoCysAesAe,
(SEQ ID NO: 6)
vP-TesCfsCyoUyoCyoGdsAyoUyoAeoAyoCyoUyoCyoUdsGyoUdsCyoCyoAyoUyoUysAesAe,
(SEQ ID NO: 15)
vP-TesGfsCyoCyoUyoCyoGyoAyoUyoAyoAyoCyoUyoCfoUyoGfoUyoCyoCyoAyoUysAesAe,
(SEQ ID NO: 16)
vP-TesGfsCyoCyoUyoCfoGyoAyoUyoAyoAyoCyoUyoCfoUyoGyoUyoCyoCyoAyoUysAesAe,
(SEQ ID NO: 17)
vP-TesGfsGyoAyoGyoUyoAyoUyoGyoUyoGyoCyoCyoUfoCyoGfoAyoUyoAyoAyoCysAesAe,
(SEQ ID NO: 18)
vP-TesGfsGyoAyoGyoUfoAyoUyoGyoUyoGyoCyoCyoUfoCyoGyoAyoUyoAyoAyoCysAesAe,
(SEQ ID NO: 19)
vP-TesGfsGyoAyoGyoUyoAyoUyoGeoUyoGyoCyoCyoUfoCyoGfoAyoUyoAyoAyoCysAesAe,
(SEQ ID NO: 20)
vP-TesGfsGyoAyoGyoUfoAyoUyoGeoUyoGyoCyoCyoUfoCyoGyoAyoUyoAyoAyoCysAesAe,
(SEQ ID NO: 21)
vP-TesGfsCyoCyoUyoCyoGyoAyoUeoAyoAyoCyoUyoCfoUyoGfoUyoCyoCyoAyoUysAesAe,
(SEQ ID NO: 22)
vP-TesGfsCyoCyoUyoCfoGyoAyoUeoAyoAyoCyoUyoCfoUyoGyoUyoCyoCyoAyoUysAesAe,
(SEQ ID NO: 23)
vP-TesGfsGyoAyoGxoUfoAyoUyoGyoUyoGyoCyoCyoUfoCyoGyoAyoUyoAyoAyoCysAesAe,
(SEQ ID NO: 24)
vP-TesGfsGyoAyoGyoUfoAxoUyoGyoUyoGyoCyoCyoUfoCyoGyoAyoUyoAyoAyoCysAesAe,
(SEQ ID NO: 25)
vP-TesGfsGyoAyoGyoUxoAyoUyoGyoUyoGyoCyoCyoUfoCyoGyoAyoUyoAyoAyoCysAesAe,
(SEQ ID NO: 26)
vP-TesGfsCyoCyoUyoCfoGxoAyoUyoAyoAyoCyoUyoCfoUyoGyoUyoCyoCyoAyoUysAesAe,
(SEQ ID NO: 27)
vP-TesGfsCyoCyoUyoCxoGyoAyoUyoAyoAyoCyoUyoCfoUyoGyoUyoCyoCyoAyoUysAesAe,
(SEQ ID NO: 28)
vP-TesGfsCyoCyoUxoCfoGyoAyoUyoAyoAyoCyoUyoCfoUyoGyoUyoCyoCyoAyoUysAesAe,
(SEQ ID NO: 29)
vP-TesUfsGyoCyoCyoUfoCyoGyoAyoUyoAyoAyoCyoUfoCyoUyoGyoUyoCyoCyoAysAesAe,
(SEQ ID NO: 30)
vP-TesUfsGyoCyoCyoUyoCyoGyoAyoUyoAyoAyoCyoUfoCyoUfoGyoUyoCyoCyoAysAesAe,
(SEQ ID NO: 31)
vP-TesGfsUyoGyoCyoCfoUyoCyoGyoAyoUyoAyoAyoCfoUyoCyoUyoGyoUyoCyoCysAesAe,
(SEQ ID NO: 32)
vP-TesGfsUyoGyoCyoCyoUyoCyoGyoAyoUyoAyoAyoCfoUyoCfoUyoGyoUyoCyoCysAesAe,
(SEQ ID NO: 33)
vP-TesAfsUyoGyoUyoGfoCyoCyoUyoCyoGyoAyoUyoAfoAyoCyoUyoCyoUyoGyoUysAesAe,
(SEQ ID NO: 34)
vP-TesAfsUyoGyoUyoGyoCyoCyoUyoCyoGyoAyoUyoAfoAyoCfoUyoCyoUyoGyoUysAesAe,
(SEQ ID NO: 35)
vP-TesGfsUyoAyoUyoGfoUyoGyoCyoCyoUyoCyoGyoAfoUyoAyoAyoCyoUyoCyoUysAesAe,
(SEQ ID NO: 36)
vP-TesGfsUyoAyoUyoGyoUyoGyoCyoCyoUyoCyoGyoAfoUyoAfoAyoCyoUyoCyoUysAesAe,
(SEQ ID NO: 37)
vP-TesAfsGyoUyoAyoUfoGyoUyoGyoCyoCyoUyoCyoGfoAyoUyoAyoAyoCyoUyoCysAesAe,
(SEQ ID NO: 38)
vP-TesAfsGyoUyoAyoUyoGyoUyoGyoCyoCyoUyoCyoGfoAyoUfoAyoAyoCyoUyoCysAesAe,
(SEQ ID NO: 39)
vP-TesUfsAyoAyoCyoUyoCyoUyoGyoUyoCyoCyoAyoUfoUyoAfoCyoCyoAyoUyoGysAesAe,
(SEQ ID NO: 40)
vP-TesAfsUyoAyoAyoCyoUyoCyoUyoGyoUyoCyoCyoAfoUyoUfoAyoCyoCyoAyoUysAesAe,
(SEQ ID NO: 41)
vP-TesUfsGyoUyoGyoCyoCyoUyoCyoGyoAyoUyoAyoAfoCyoUfoCyoUyoGyoUyoCysAesAe,
(SEQ ID NO: 42)
vP-TesUfsGyoUyoGyoCfoCyoUyoCyoGyoAyoUyoAyoAfoCyoUyoCyoUyoGyoUyoCysAesAe,
(SEQ ID NO: 43)
vP-TesCfsUyoCyoGyoAyoUyoAyoAyoCyoUyoCyoUyoGfoUyoCfoCyoAyoUyoUyoAysAesAe,
(SEQ ID NO: 44)
vP-TesCfsUyoCyoGyoAfoUyoAyoAyoCyoUyoCyoUyoGfoUyoCyoCyoAyoUyoUyoAysAesAe,
(SEQ ID NO: 45)
vP-TesUfsCyoGyoAyoUyoAyoAyoCyoUyoCyoUyoGyoUfoCyoCfoAyoUyoUyoAyoCysAesAe,
(SEQ ID NO: 46)
vP-TesUfsCyoGyoAyoUfoAyoAyoCyoUyoCyoUyoGyoUfoCyoCyoAyoUyoUyoAyoCysAesAe,
(SEQ ID NO: 47)
vP-TesGfsAyoUyoAyoAyoCyoUyoCyoUyoGyoUyoCyoCfoAyoUfoUyoAyoCyoCyoAysAesAe,
and
(SEQ ID NO: 48)
vP-TesUfsGyoCyoCyoUdsCyoGyoAcoUyoAyoAyoCyoUdsCyoUdsGyoUyoCyoCyoAysAesAe;
wherein: A = an adenine nucleobase, C = a cytosine nucleobase,
G = a guanine nucleobase, T = a thymine nucleobase,U = a uracil
nucleobase, d = a 2′-2′-β-D-deoxyribosyl sugar moiety, x =
a 2′-β-D-deoxyxylosyl sugar moiety, e = a 2′-MOE sugar moiety,
f = a 2′-fluoro sugar moiety, h = a 3′-fluoro-hexitol sugar
moiety y = a 2′-OMe sugar moiety, o = a phosphodiester inter-
nucleoside linkage, s = a phosphorothioate internucleoside
linkage, and VP = a 5′ vinyl phosphonate moiety;
and
B. an oligomeric compound according to any one of the following
chemical notation:
(SEQ ID NO: 50)
[THA-GalNAc]-GesUesUyoAyoUyoCyoGyoAyoGyoGfsCfoAyoCyoAyoUyoAyoCyoUyoCysCe
sAe,
(SEQ ID NO: 51)
[THA-GalNAc]-AesAesUyoGyoGyoAyoCyoAyoGyoAfsGfoUyoUyoAyoUyoCyoGyoAyoGysGe
sAe,
(SEQ ID NO: 52)
[THA-GalNAc]-AysUysGyoGyoAyoCyoAyoGyoAfoGfoUfoUyoAyoUeoCyoGyoAyoGyoGysCy
sAy,
(SEQ ID NO: 53)
[THA-GalNAc]-AesUesGyoGyoAyoCyoAyoGyoAfoGfoUfoUyoAyoUeoCyoGyoAyoGyoGysCe
sAe,
(SEQ ID NO: 54)
GysUysUyoAyoUyoCyoGyoAyoGfoGfoCfoAyoCyoAcoUyoAyoCyoUyoCysCysAy-[HPPO-
GalNAc],
(SEQ ID NO: 55)
GesUesUyoAyoUyoCyoGyoAyoGfoGfoCfoAyoCyoAeoUyoAyoCyoUyoCysCesAe-[HPPO-
GalNAc],
(SEQ ID NO: 56)
[THA-GalNAc]-GesUesUyoAyoUyoCyoGyoAyoGfoGfoCfoAyoCyoAeoUyoAyoCyoUyoCysCe
sAe,
(SEQ ID NO: 57)
GesUesUyoAyoUyoCyoGyoAyoGdsGfoCfoAyoCyoAeoUyoAyoCyoUyoCysCesAe-[HPPO-
GalNAc],
(SEQ ID NO: 58)
GesUesUyoAyoUyoCyoGyoAyoGfoGdsCfoAyoCyoAcoUyoAyoCyoUyoCysCesAe-[HPPO-
GalNAc],
(SEQ ID NO: 59)
GesUesUyoAyoUyoCyoGyoAyoGfoGfoCdsAyoCyoAcoUyoAyoCyoUyoCysCesAe-[HPPO-
GalNAc],
(SEQ ID NO: 60)
GesUesUyoAyoUyoCyoGyoAyoGhoGfoCfoAyoCyoAcoUyoAyoCyoUyoCysCesAe-[HPPO-
GalNAc],
(SEQ ID NO: 61)
GesUesUyoAyoUyoCyoGyoAyoGfoGhoCfoAyoCyoAeoUyoAyoCyoUyoCysCesAe-[HPPO-
GalNAc],
(SEQ ID NO: 62)
GesUesUyoAyoUyoCyoGyoAyoGfoGfoChoAyoCyoAcoUyoAyoCyoUyoCysCesAe-[HPPO-
GalNAc],
(SEQ ID NO: 63)
GesUesUyoAyoUyoCyoGyoAyoGfoGxoCfoAyoCyoAcoUyoAyoCyoUyoCysCesAe-[HPPO-
GalNAc],
(SEQ ID NO: 64)
GesUesUyoAyoUyoCyoGyoAyoGxoGfoCfoAyoCyoAcoUyoAyoCyoUyoCysCesAe-[HPPO-
GalNAc],
(SEQ ID NO: 65)
GesUesUyoAyoUyoCyoGyoAyoGxoGfoCfoAyoCyoAeoUyoAyoCyoUyoCysCesAe-[HPPO-
GalNAc],
(SEQ ID NO: 66)
[THA-GalNAc]-GesUesUyoAyoUyoCyoGyoAyoGfoGdsCfoAyoCyoAeoUyoAyoCyoUyoCysCe
sAe,
(SEQ ID NO: 67)
[THA-GalNAc]-UesGesGyoAyoCyoAyoGyoAyoGfoUfoUfoAyoUyoCeoGyoAyoGyoGyoCysAe
sAe,
(SEQ ID NO: 68)
[THA-GalNAc]-GesGesAyoCyoAyoGyoAyoGyoUfoUfoAfoUyoCyoGeoAyoGyoGyoCyoAysCe
sAe,
(SEQ ID NO: 69)
[THA-GalNAc]-AesCesAyoGyoAyoGyoUyoUyoAfoUfoCfoGyoAyoGeoGyoCyoAyoCyoAysUe
sAe,
(SEQ ID NO: 70)
[THA-GalNAc]-AesGesAyoGyoUyoUyoAyoUyoCfoGfoAfoGyoGyoCeoAyoCyoAyoUyoAysCe
sAe,
(SEQ ID NO: 71)
[THA-GalNAc]-GesAesGyoUyoUyoAyoUyoCyoGfoAfoGfoGyoCyoAeoCyoAyoUyoAyoCysUe
sAe,
(SEQ ID NO: 72)
[THA-GalNAc]-CesAesUyoGyoGyoUyoAyoAyoUfoGfoGfoAyoCyoAeoGyoAyoGyoUyoUysAe
sAe,
(SEQ ID NO: 73)
[THA-GalNAc]-AesUesGyoGyoUyoAyoAyoUyoGfoGfoAfoCyoAyoGeoAyoGyoUyoUyoAysUe
sAe,
(SEQ ID NO: 74)
[THA-GalNAc]-GesAesCyoAyoGyoAyoGyoUyoUfoAfoUfoCyoGyoAeoGyoGyoCyoAyoCysAe
sAe,
(SEQ ID NO: 75)
[THA-GalNAc]-UesAesAyoUyoGyoGyoAyoCyoAfoGfoAfoGyoUyoUeoAyoUyoCyoGyoAysGe
sAe,
(SEQ ID NO: 76)
[THA-GalNAc]-GesUesAyoAyoUyoGyoGyoAyoCfoAfoGfoAyoGyoUeoUyoAyoUyoCyoGysAe
sAe,
(SEQ ID NO: 77)
[THA-GalNAc]-UesGesGyoUyoAyoAyoUyoGyoGfoAfoCfoAyoGyoAeoGyoUyoUyoAyoUysCe
sAe,
(SEQ ID NO: 78)
[THA-GalNAc]-GesUesUyoAyoUyoCyoGyoAyoGfoGfoCdsAyoCyoAeoUyoAyoCyoUyoCysCe
sAe,
(SEQ ID NO: 79)
[THA-GalNAc]-GesUesUyoAyoUyoCyoGyoAyoGdsGfoCfoAyoCyoAcoUyoAyoCyoUyoCysCe
sAe,
(SEQ ID NO: 80)
[THA-GalNAc]-GesUesUyoAyoUyoCyoGyoAyoGfoGhoCfoAyoCyoAcoUyoAyoCyoUyoCysCe
sAe,
(SEQ ID NO: 81)
[THA-GalNAc]-GesUesUyoAyoUyoCyoGyoAyoGfoGfoChoAyoCyoAcoUyoAyoCyoUyoCysCe
sAe,
and
(SEQ ID NO: 82)
UesGesGyoAyoCyoAyoGyoAyoGyoUfsUfoAyoUyoCyoGyoAyoGyoGyoCysAesAe-[HPPO-
GalNAc];

wherein:

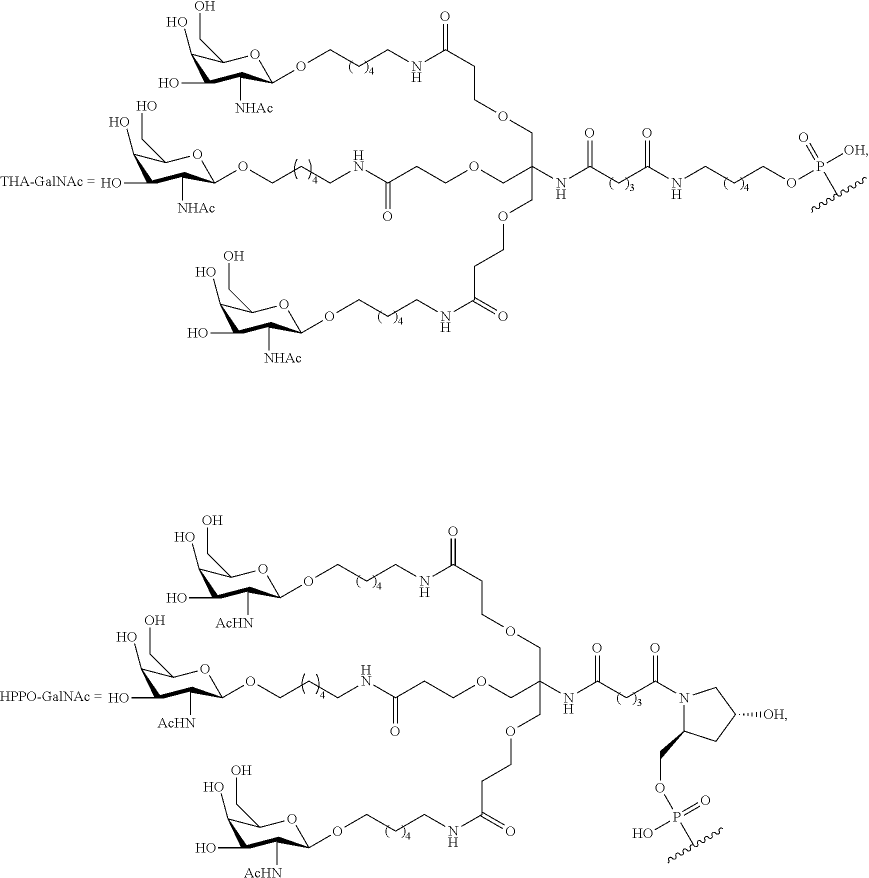

A=an adenine nucleobase, C=a cytosine nucleobase, G=a guanine nucleobase, T=a thymine nucleobase, U=a uracil nucleobase, d=a 2′-2′-β-D-deoxyribosyl sugar moiety, x=a 2′-β-D-deoxyxylosyl sugar moiety, e=a 2′-MOE sugar moiety, f=a 2′-fluoro sugar moiety, h=a 3′-fluoro-hexitol sugar moiety y=a 2′-OMe sugar moiety, o=a phosphodiester internucleoside linkage, and s=a phosphorothioate internucleoside linkage.

51. An oligomeric duplex according to the chemical structure of Compound 1829960 (SEQ ID NO: 5 and SEQ ID NO: 58), or an ion or salt thereof.

52. The oligomeric duplex of embodiment 51, which is the sodium salt or potassium salt.

53. An oligomeric duplex according to the chemical structure of Compound 1829960 sodium salt (SEQ ID NO: 5 and SEQ ID NO: 58).

54. An oligomeric duplex according to the chemical structure of Compound 1840071 (SEQ ID NO: 5 and SEQ ID NO: 66), or an ion or salt thereof.

55. The oligomeric duplex of embodiment 54, which is the sodium salt or potassium salt.

56. An oligomeric duplex according to the chemical structure of Compound 1840071 sodium salt (SEQ ID NO: 5 and SEQ ID NO: 66).

57. An oligomeric duplex according to the chemical structure of Compound 1839410 (SEQ ID NO: 26 and SEQ ID NO: 53), or an ion or salt thereof.

58. The oligomeric duplex of embodiment 57, which is the sodium salt or potassium salt.

59. An oligomeric duplex according to the chemical structure of Compound 1839410 sodium salt (SEQ ID NO: 26 and SEQ ID NO: 53).

60. An oligomeric duplex according to the chemical structure of Compound 1839412 (SEQ ID NO: 28 and SEQ ID NO: 53), or an ion or salt thereof.

61. The oligomeric duplex of embodiment 60, which is the sodium salt or potassium salt.

62. An oligomeric duplex according to the chemical structure of Compound 1839412 sodium salt (SEQ ID NO: 28 and SEQ ID NO: 53).

63. An oligomeric duplex according to the chemical structure of Compound 1840453 (SEQ ID NO: 31 and SEQ ID NO: 68), or an ion or salt thereof.

64. The oligomeric duplex of embodiment 63, which is the sodium salt or potassium salt.

65. An oligomeric duplex according to the chemical structure of Compound 1840453 sodium salt (SEQ ID NO: 31 and SEQ ID NO: 68)

66. An oligomeric duplex according to the chemical structure of Compound 1841135 (SEQ ID NO: 45 and SEQ ID NO: 76), or an ion or salt thereof.

67. The oligomeric duplex of embodiment 66, which is the sodium salt or potassium salt.

68. An oligomeric duplex according to the chemical structure of Compound 1841135 sodium salt (SEQ ID NO: 45 and SEQ ID NO: 76).

69. An oligomeric duplex according to the chemical structure of Compound 1841136 (SEQ ID NO: 46 and SEQ ID NO: 76), or an ion or salt thereof.

70. The oligomeric duplex of embodiment 69, which is the sodium salt or potassium salt.

71. An oligomeric duplex according to the chemical structure of Compound 1841136 sodium salt (SEQ ID NO: 46 and SEQ ID NO: 76).

72. A pharmaceutical composition comprising the oligomeric duplex of any one of embodiments 1-60 and a pharmaceutically acceptable diluent or carrier.

73. The pharmaceutical composition of embodiment 72, wherein the pharmaceutically acceptable diluent is water or phosphate-buffered saline.

74. The pharmaceutical composition of embodiment 72, wherein the pharmaceutical composition consists essentially of the oligomeric duplex and water or phosphate-buffered saline.

75. A method of decreasing the amount of LPA RNA or Lipoprotein(a) protein in a cell, tissue, organ, or subject, comprising contacting the cell, tissue, organ, or subject with the oligomeric duplex of any one of embodiments 1-71, or the pharmaceutical composition of any one of embodiments 72-74.

76. The method of embodiment 75, wherein the cell is a liver cell.

77. A method comprising administering to a subject an oligomeric duplex of any one of embodiments 1-71, or the pharmaceutical composition of any one of embodiments 72-74; wherein the subject has or is at risk for developing cardiovascular disease (CVD) coronary artery disease (CAD), hypercholesterolemia, myocardial infarction (MI), peripheral arterial disease (PAD), calcific aortic valve disease (CAVD), aortic stenosis, atherosclerotic cardiovascular disease (ASCVD), atherosclerosis, dyslipidemia, thrombosis, or stroke.

78. A method of preventing or treating a cardiovascular disease, disorder, condition, a metabolic disease, disorder, or condition, and/or an inflammatory disease disorder or condition in a subject, comprising administering to a subject having, or at risk of having, a cardiovascular, metabolic, and/or inflammatory disease, disorder, or condition, an oligomeric duplex of any one of embodiments 1-71, or the pharmaceutical composition of any one of embodiments 72-74; wherein the disease, disorder, condition or injury is hypertriglyceridemia, lipidemia (e.g., hyperlipidemia), dyslipidemia (e.g., atherogenic dyslipidemia, diabetic dyslipidemia, or mixed dyslipidemia), hyperlipoproteinemia, coronary artery disease, metabolic syndrome, acute coronary syndrome, aortic valve stenosis, aortic valve calcification, aortic valve regurgitation, aortic dissection, retinal artery occlusion, cerebrovascular disease, mesenteric ischemia, superior mesenteric artery occlusion, restenosis, renal artery stenosis, angina, cerebrovascular atherosclerosis, cerebrovascular disease, or venous thrombosis.

79. A method of decreasing the amount of LPA RNA and/or Lipoprotein(a) protein in the liver of a subject having or at risk of developing a disease, disorder or condition associated with elevated Lp(a), comprising administering to a subject having, or at risk of having, a disease, disorder or condition associated with lipoprotein(a) metabolism misregulation, an oligomeric duplex of any one of embodiments 1-71, or the pharmaceutical composition of any one of embodiments 72-74; wherein the disease, disorder, condition or injury is a cardiovascular disease, disorder, condition, a metabolic disease, disorder, or condition, and/or an inflammatory disease disorder or condition.

80. The method of any one of embodiments 75-79, wherein the amount of LPA RNA and/or lipoprotein(a) protein in liver and/or plasma of the subject is decreased.

81. The method of any one of embodiments 75-79, wherein the disease, disorder or condition is hypertriglyceridemia or atherosclerotic cardiovascular disease (ASCVD) or coronary artery disease (CAD).

82. The method of embodiment 81, wherein at least one symptom of a disease, disorder or condition associated with elevated lipoprotein(a) is episodes of abdominal pain, physical fatigue, difficulty thinking, diarrhea, recurrent acute pancreatitis, eruptive cutaneous xanthomata, and hepatosplenomegaly or a combination thereof.

83. The method of any one or embodiments 75-79, wherein the method prevents or protects against progression of atherosclerotic cardiovascular disease (ASCVD) or coronary artery disease (CAD).

84. The method of any one of embodiments 75-79, wherein administering of the oligomeric duplex of any one of embodiments 1-71, or the pharmaceutical composition of any one of embodiments 72-74 improves hypertriglyceridemia, hyperlipidemia, dyslipidemia hyperlipoproteinemia abdominal pain, physical fatigue, difficulty thinking, diarrhea, acute pancreatitis, eruptive xanthomas, lipemia retinalis, or hepatosplenomegaly, or a combination of two or more of the foregoing in the subject.

85. The method of any one of embodiments 75-79, wherein administering of the oligomeric duplex of any one of embodiments 1-71, or the pharmaceutical composition of any one of embodiments 72-74 is parenteral.

86. The method of any one of embodiments 75-79, wherein administering of the oligomeric duplex of any one of embodiments 1-71, or the pharmaceutical composition of any one of embodiments 72-74 is subcutaneous.

87. The method of any one of embodiments 75-79, wherein administering of the oligomeric duplex of any one of embodiments 1-71, or the pharmaceutical composition of any one of embodiments 72-74 is co-administering with a second agent.

88. The method of any one of embodiment 74-78, wherein administering of the oligomeric duplex of any one of embodiments 1-71, or the pharmaceutical composition of any one of embodiments 72-74 and the agent are administered concomitantly.

89. Use of the oligomeric duplex of any one of embodiments 1-71, or the pharmaceutical composition of any one of embodiments 72-74 for treating or preventing a disease, disorder or condition associated with lipoprotein metabolism misregulation or postponing a symptom of a disease, disorder or condition associated with elevated lipoprotein(a).

90. Use of the oligomeric duplex of any one of embodiments 1-71, or the pharmaceutical composition of any one of embodiments 72-74 in the manufacture of a medicament for treating or preventing a cardiovascular disease, disorder, condition, a metabolic disease, disorder, or condition, or an inflammatory disease disorder or condition.

91. An oligomeric duplex of any one of embodiments 1-71, or the pharmaceutical composition of any one of embodiments 72-74 for use in decreasing the amount of LPA RNA or Lipoprotein(a) protein in a cell, tissue, organ, or subject, comprising contacting the cell, tissue, organ, or subject.

92. The oligomeric duplex for use of embodiment 91, wherein the cell is a liver cell.

93. An oligomeric duplex of any one of embodiments 1-71, or the pharmaceutical composition of any one of embodiments 72-74 for use in method of treating cardiovascular disease (CVD) coronary artery disease (CAD), hypercholesterolemia, myocardial infarction (MI), peripheral arterial disease (PAD), calcific aortic valve disease (CAVD), aortic stenosis, atherosclerotic cardiovascular disease (ASCVD), atherosclerosis, dyslipidemia, thrombosis, or stroke.

94. An oligomeric duplex of any one of embodiments 1-71, or the pharmaceutical composition of any one of embodiments 72-74 for use in a method of preventing or treating a cardiovascular disease, disorder, condition, a metabolic disease, disorder, or condition, and/or an inflammatory disease disorder or condition in a subject having, or at risk of having, a cardiovascular, metabolic, and/or inflammatory disease, disorder, or condition; wherein the disease, disorder, condition or injury is hypertriglyceridemia, lipidemia (e.g., hyperlipidemia), dyslipidemia (e.g., atherogenic dyslipidemia, diabetic dyslipidemia, or mixed dyslipidemia), hyperlipoproteinemia, coronary artery disease, metabolic syndrome, acute coronary syndrome, aortic valve stenosis, aortic valve calcification, aortic valve regurgitation, aortic dissection, retinal artery occlusion, cerebrovascular disease, mesenteric ischemia, superior mesenteric artery occlusion, restenosis, renal artery stenosis, angina, cerebrovascular atherosclerosis, cerebrovascular disease, or venous thrombosis.

95. An oligomeric duplex of any one of embodiments 1-71, or the pharmaceutical composition of any one of embodiments 72-74 for use in a method of decreasing the amount of LPA RNA and/or Lipoprotein(a) protein in the liver of a subject having or at risk of developing a disease, disorder or condition associated with elevated Lp(a) in a subject having, or at risk of having, a disease, disorder or condition associated with lipoprotein(a) metabolism misregulation; wherein the disease, disorder, condition or injury is a cardiovascular disease, disorder, condition, a metabolic disease, disorder, or condition, and/or an inflammatory disease disorder or condition.

96. The oligomeric duplex or composition for use of any one of embodiments 91-95, wherein the amount of LPA RNA and/or lipoprotein(a) protein in liver and/or plasma of the subject is decreased.

97. The oligomeric duplex or composition for use of any one of embodiments 91-95, wherein the disease, disorder or condition is hypertriglyceridemia or atherosclerotic cardiovascular disease (ASCVD) or coronary artery disease (CAD).

98. The oligomeric duplex or composition for use of embodiment 97, wherein at least one symptom of a disease, disorder or condition associated with elevated lipoprotein(a) is episodes of abdominal pain, physical fatigue, difficulty thinking, diarrhea, recurrent acute pancreatitis, eruptive cutaneous xanthomata, and hepatosplenomegaly or a combination thereof.

99. The oligomeric duplex or composition for use of any one of embodiments 91-95, wherein the method prevents or protects against progression of atherosclerotic cardiovascular disease (ASCVD) or coronary artery disease (CAD).

100. The oligomeric duplex or composition for use of any one of embodiments 91-95, wherein administering of the oligomeric duplex or the pharmaceutical composition improves hypertriglyceridemia, hyperlipidemia, dyslipidemia hyperlipoproteinemia abdominal pain, physical fatigue, difficulty thinking, diarrhea, acute pancreatitis, eruptive xanthomas, lipemia retinalis, or hepatosplenomegaly, or a combination of two or more of the foregoing in the subject.

101. The oligomeric duplex or composition for use of any one of embodiments 91-95, wherein administering of the oligomeric duplex or the pharmaceutical composition is parenteral.

102. The oligomeric duplex or composition for use of any one of embodiments 91-95, wherein administering of the oligomeric duplex or the pharmaceutical composition is subcutaneous.

103. The oligomeric duplex or composition for use of any one of embodiments 91-95, wherein administering of the oligomeric duplex or the pharmaceutical composition is co-administering with a second agent.

104. The oligomeric duplex or composition for use of any one of embodiments 91-95, wherein administering of the oligomeric duplex or the pharmaceutical composition and the agent are administered concomitantly.

### I. Oligonucleotides

Provided herein are oligomeric duplexes and oligomeric compounds comprising a modified antisense oligonucleotide (e.g., an antisense oligomeric compound) complementary to LPA RNA and a modified sense oligonucleotide (e.g., a sense oligomeric compound) complementary to an antisense oligomeric compound. Modified antisense and/or sense oligonucleotides comprise at least one modified nucleoside (comprising a modified sugar moiety and/or a modified nucleobase) and/or at least one modified internucleoside linkage. Examples of certain modified nucleosides and modified internucleoside linkages suitable for use in modified antisense and/or sense oligonucleotides are described herein.

### A. Modified Nucleosides

Modified nucleosides comprise a modified sugar moiety, a modified nucleobase, or a combination thereof. In certain embodiments, modified nucleosides comprising the following modified sugar moieties and/or the following modified nucleobases may be incorporated into modified antisense oligonucleotides and/or sense oligonucleotides described herein.

### 1. Modified Sugar Moieties

Modified sugar moieties include modified furanosyl sugar moieties, cyclic sugar surrogates, acyclic sugar surrogates, and sugar mimics. In certain embodiments, modified sugar moieties are non-bicyclic modified furanosyl sugar moieties. In certain embodiments, modified sugar moieties are bicyclic or tricyclic furanosyl sugar moieties. In certain embodiments, modified sugar moieties are sugar surrogates. Sugar surrogates may comprise one or more substitutions corresponding to those of other types of modified sugar moieties.

In certain embodiments, modified sugar moieties are non-bicyclic modified furanosyl sugar moieties comprising one or more substituent groups including, but not limited to, substituents at the 2′, 3′, 4′, and/or 5′ positions, as numbered based on ribose:

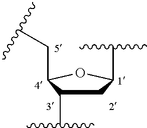

In certain embodiments, the modified furanosyl sugar moiety is a ribosyl sugar moiety that is not an unmodified sugar moiety (i.e., an unmodified RNA). In certain embodiments, the modified furanosyl sugar moiety is a xylosyl, lyxosyl, or arabinosyl sugar moiety. In certain embodiments, the modified furanosyl sugar moiety is a deoxyribosyl, deoxyxylosyl, deoxylyxosyl, or deoxyarabinosyl sugar moiety. In certain embodiments, the modified furanosyl sugar moiety is a deoxyxylosyl, or deoxyribosyl sugar moiety.
In certain embodiments, non-bicyclic modified sugar moieties are 2′-substituted sugar moieties and comprise a substituent group at the 2′-position. In certain embodiments one or more non-bridging substituent of non-bicyclic modified sugar moieties is branched. Examples of substituent groups suitable for the 2′-position of modified sugar moieties include but are not limited to: 2′-F, 2′-OCH₃ (“OMe” or “O-methyl”), and 2′-O(CH₂)₂OCH₃ (“MOE” or “O-methoxyethyl”). In certain embodiments, 2′-substituent groups are selected from: halo, allyl, amino, azido, SH, CN, OCN, CF₃, OCF₃, C₁-C₁₀ alkoxy, C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, O(CH₂)₂ON(CH₃)₂ (“DMAOE”), or 2′-O(CH₂)₂O(CH₂)₂N(CH₃)₂ (“DMAEOE”). Synthetic methods for some of these 2′-substituent groups may be found, e.g., in Cook et al., U.S. Pat. No. 6,531,584; Cook et al., U.S. Pat. No. 5,859,221; and Cook et al., U.S. Pat. No. 6,005,087. Certain embodiments of these 2′-substituent groups may be further substituted with one or more substituent groups independently selected from: halo, cyano, OR^{a2}, NO₂, NH₂, NHR^{a2}, N(R^{a2})₂, C₁-C₆ alkyl, C₁-C₆ haloalkyl, C₂-C₆ alkenyl, C₂-C₆ alkynyl, C₃-C₁₀ cycloalkyl, C₆-C₁₀ aryl, heteroaryl, heterocyclyl, C₁-C₆ alkylene-NH₂, C₁-C₆alkylene-NHR^{a2}, C₁-C₆ alkylene-N(R^{a2})₂, C(O)R^{a3}, C(O)OR^{a3}, C(O)NHR^{a3}, C(O)N(C₁-C₄alkyl)R^{a3}, SR^{a3}, S(O)₂R^{a3}, S(O)R^{a3}, NHC(O)R^{a3}, N(C₁-C₄alkyl)C(O)R^{a3} NHS(O)R^{a3}, N(C₁-C₄alkyl)S(O)R^{a3}, NHS(O)₂R^{a3}, and N(C₁-C₄ alkyl)S(O)₂R^{a3}; each R^{a2} is independently selected from C₂-C₆ alkyl, C₂-C₆ alkenyl, C₂-C₆ alkynyl, C₃-C₁₀ cycloalkyl, C₆-C₁₀ aryl, heteroaryl, and heterocyclyl; each R^{a3} is independently hydrogen, OH, C₁-C₆ alkyl, C₁-C₆ haloalkyl, C₃-C₁₀ cycloalkyl, C₆-C₁₀ aryl, heteroaryl, or heterocyclyl. In certain embodiments, a sugar moiety comprises two of the above substituents at the 2′-position. In certain embodiments, a sugar moiety comprises a 2′-fluoro and a second 2′-substituent.

In certain embodiments, a 2′-substituted sugar moiety comprises a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted sugar moiety comprises a non-bridging 2′-substituent group selected from: F, OCF₃, OCH₃, OCH₂CH₂OCH₃, O(CH₂)₂SCH₃, O(CH₂)₂₀N(CH₃)₂, O(CH₂)₂O(CH₂)₂N(CH₃)₂, O(CH₂)₂₀N(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, a 2′-substituted sugar moiety comprises a non-bridging 2′-substituent group selected from: F, OCF₃, OCH₃, OCH₂CH₂OCH₃, O(CH₂)₂SCH₃, O(CH₂)₂₀N(CH₃)₂, O(CH₂)₂O(CH₂)₂N(CH₃)₂, and OCH₂C(═O)—N(H)CH₃ (“NMA”).

In certain embodiments, a 2′-substituted sugar moiety comprises a 2′-substituent group selected from: F, OCH₃, and OCH₂CH₂OCH₃.

In certain embodiments, modified furanosyl sugar moieties and nucleosides incorporating such modified furanosyl sugar moieties are further defined by stereochemical configuration. For example, a 2′-deoxyfuranosyl sugar moiety (i.e., 2′-(H)H furanosyl sugar moiety) may be in seven isomeric configurations other than the naturally occurring β-D-deoxyribosyl configuration. Such modified sugar moieties are described in, e.g., WO 2020/072991, incorporated by reference herein. A 2′-modified sugar moiety has an additional stereocenter at the 2′-position relative to a 2′-deoxyfuranosyl sugar moiety; therefore, such sugar moieties have a total of sixteen possible stereochemical configurations. Modified furanosyl sugar moieties described herein are in the β-D-ribosyl stereochemical configuration unless otherwise specified. In certain embodiments, the modified furanosyl sugar moiety is a β-D-xylosyl, β-D-lyxosyl, or β-D-arabinosyl sugar moiety. In certain embodiments, the modified furanosyl sugar moiety is a β-D-deoxyribosyl, β-D-deoxyxylosyl, R-D-deolyxosyl, or D-D-deoxyarabinosyl sugar moiety. In certain embodiments, the modified furanosyl sugar moiety is a β-D-deoxyxylosyl, or β-D-deoxyribosyl sugar moiety.

In certain embodiments, non-bicyclic modified sugar moieties comprise a substituent group at the 4′-position. Examples of substituent groups suitable for the 4′-position of modified sugar moieties include, but are not limited to, alkoxy (e.g., methoxy), alkyl, and those described in Manoharan et al., WO 2015/106128, which is incorporated herein by reference.

In certain embodiments, non-bicyclic modified sugar moieties comprise a substituent group at the 3′-position. Examples of substituent groups suitable for the 3′-position of modified sugar moieties include, but are not limited to, alkoxy (e.g., methoxy), alkyl (e.g., methyl, ethyl).

In certain embodiments, non-bicyclic modified sugar moieties comprise a substituent group at the 5′-position. Examples of substituent groups suitable for the 5′-position of modified sugar moieties include, but are not limited to, vinyl, alkoxy (e.g., methoxy), alkynyl, allyl, and alkyl (e.g., methyl (R or S), ethyl (R or S)).

In certain embodiments, non-bicyclic modified sugar moieties comprise more than one non-bridging sugar substituent, for example, 2′-F-5′-methyl sugar moieties, such as described in Migawa et al., US 2010/0190837, which is incorporated herein by reference; or alternative 2′- and 5′-modified sugar moieties as described in Rajeev et al., US 2013/0203836, which is incorporated herein by reference.

Certain modified sugar moieties are bicyclic sugar moieties and comprise a substituent that bridges two atoms of the furanosyl ring to form a second ring. In certain embodiments, the bicyclic sugar moiety comprises a bridge between the 4′ and the 2′ furanose ring atoms. Examples of such 4′ to 2′ bridging sugar substituents include, but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof, 4′-C(CH₃)(CH₃)—O-2′ and analogs thereof, 4′-CH₂—N(OCH₃)-2′ and analogs thereof, 4′-CH₂—O—N(CH₃)-2′, 4′-CH₂—C(H)(CH₃)-2′, 4′-CH₂—C(═CH₂)-2′ and analogs thereof, 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl. Representative U.S. patents that teach the preparation of such bicyclic sugar moieties include, but are not limited to: Imanishi et al., U.S. Pat. No. 7,427,672; Swayze et al., U.S. Pat. No. 7,741,457; Swayze et al., U.S. Pat. No. 8,022,193; Seth et al., U.S. Pat. No. 8,278,283; Prakash et al., U.S. Pat. No. 8,278,425; and Seth et al., U.S. Pat. No. 8,278,426, each of which are incorporated herein by reference.

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, C(Rₐ)═C(R_{b}), C(Rₐ)═N—, C(═NRₐ)—, —C(═O)—, —C(═S)—, —O—, —Si(R^{a})₂—, —S(═O)ₓ—, and N(Rₐ)—;
wherein:
x is 0, 1, or 2;
n is 1, 2, 3, or 4;
each R^{a} and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

In certain embodiments, the bicyclic sugar moiety comprises a bridge between the 5′ and the 3′ furanose ring atoms. Examples of such 5′ to 3′ bridging sugar substituents include, but are not limited to, 5′-(CH₂)₂-3′ (bcDNA), 5′-(CH₂)₃-3′ (bc^{4,3}DNA), 5′-C(F)═CH—CH₂-3′, and 5′-CH₂—CHQ-3′, wherein Q is an attachment to an internucleoside linkage.

Additional bicyclic sugar moieties are known in the art, see, for example: Wan, et al., J. Medicinal Chemistry, 2016, 59, 9645-9667; Wengel et al., U.S. Pat. No. 8,080,644; Ramasamy et al., U.S. Pat. No. 6,525,191; Seth et al., U.S. Pat. No. 7,547,684; and Seth et al., U.S. Pat. No. 7,666,854, which are each incorporated herein by reference.

In certain embodiments, bicyclic sugar moieties and nucleosides incorporating such bicyclic sugar moieties are further defined by stereochemical configuration. For example, an LNA nucleoside (described herein) may be in the α-L configuration or in the β-D configuration.

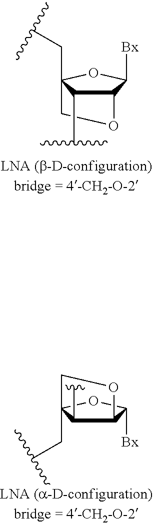

α-L-methyleneoxy (4′-CH₂—O-2′) or α-L-LNA bicyclic nucleosides have been incorporated into oligonucleotides that showed antisense activity (Frieden et al. Nucleic Acids Res. 2003, 21, 6365-6372). The addition of locked nucleic acids to siRNAs has been shown to increase siRNA stability in serum, and to reduce off-target effects (Elmén, J. et al. Nucleic Acids Res. 2005, 33(1), 439-447; Mook, O. R. et al. Mol. Canc. Ther. 2007, 6(3), 833-843; Grunweller, A. et al. Nucleic Acids Res. 2003, 31(12), 3185-3193). Herein, general descriptions of bicyclic nucleosides include both stereochemical configurations. When the positions of specific bicyclic nucleosides (e.g., LNA or cEt) are identified in exemplified embodiments herein, they are in the β-D stereochemical configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates, selected from cyclic sugar surrogates and acyclic sugar surrogates.

A cyclic sugar surrogate is represented by Formula Ia:

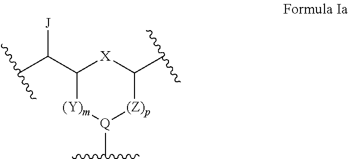

Wherein:
J is H, C₁-C₆ alkyl, or C₂-C₆ alkenyl;
X is O, S, C(R₁R₂), N(R₃), C(R₁)═C(R₂), C(R₁R₂)—C(R₁R₂), O—C(R₁R₂), C(R₁R₂)—O, S—C(R₁R₂), C(R₁R₂)—S, N(R₃)—C(R₁R₂), or C(R₁R₂)—N(R₃);
Y is C(R₁R₂), C(R₁)═C(R₂), or C(R₁R₂)—C(R₁R₂);
Z is C(G₁G₂), C(G₁)═C(R₁), C(R₁)═C(G₁), C(G₁G₂)-C(R₁R₂), C(R₁R₂)—C(G₁G₂), C(G₁G₂)-C(R₁R₂)—C(R₁R₂), or C(R₁R₂)—C(R₁R₂)—C(G₁G₂);
Q is CH or N;
each R₁ and R₂ is independently H, OH, C₁-C₆ alkyl, or N(R₄); wherein if R₁ is OH, then R₂ is not OH;
each R₃ and R₄ is independently H, C₁-C₆ alkyl, or C(═O)R₅, wherein R₅ is C₁-C₆ alkyl;
each G₁ and G₂ is independently H, OH, halogen or O—[C(R₆)(R₇)]_{q}—[(C═O)ₛ—X^{G}]ⱼ—R₈; wherein if G₁ is OH, then G₂ is not OH;
each R₆ and R₇ is, independently, H, halogen, C₁-C₆ alkyl or substituted C₁-C₆ alkyl;
each X^{G} is O, S or N(E₁);
R₈ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
   m is 0 or 1;
p is 0 or 1;
q is from 1 to 6;
s is 0 or 1;
j is 0 or 1; and
with the proviso that if X is O, Z is C(G₁G₂), and Q is CH, then m is 1.

In certain embodiments, the oxygen atom of a sugar moiety is replaced, e.g., with a sulfur, carbon, or nitrogen atom (X is S, C(R₁R₂), or N(R₃)). In certain such embodiments, such modified sugar moieties also comprise bridging and/or non-bridging substituents as described herein. For example, certain sugar surrogates comprise a 4′-sulfur atom and a substitution at the 2′-position and/or the 5′ position.

In certain embodiments, sugar surrogates comprise rings having other than 5 atoms. For example, in certain embodiments, a sugar surrogate comprises a six-membered tetrahydropyran (“THP”), where X is O—C(R₁R₂), p is 1, Z is C(G₁G₂), and m is 0. Such tetrahydropyrans may be further modified or substituted. Nucleosides comprising such modified tetrahydropyrans include but are not limited to hexitol nucleic acid (“HNA”), altritol nucleic acid (G₁=OH; G₂=H; “ANA”), and fluoro HNA:

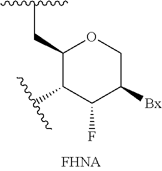

(G₁=F; G₂=H; “FHNA”, see e.g., Egli, M. et al. J. Am. Chem. Soc. 2011, 133(41), 16642-16649; Swayze et al., U.S. Pat. No. 8,088,904; and Swayze et al., U.S. Pat. No. 8,440,803); FHNA can also be referred to as a F-THP or 3′-fluoro tetrahydropyran or 3′-FHNA), each of which are incorporated herein by reference.

In certain embodiments, sugar surrogates comprise rings having more than 5 atoms and more than one heteroatom. For example, nucleosides comprising morpholino sugar moieties and their use in oligonucleotides have been reported. As used here, the term “morpholino” means a sugar surrogate having Formula Ia, above, wherein X is O, Y and Z are each CH₂, and Q is N. In certain embodiments, a morpholino is modified, for example by adding or altering various substituent groups from the above morpholino structure. Such sugar surrogates are referred to herein as “modified morpholinos.”

In certain embodiments, sugar surrogates are acyclic sugar surrogates and have Formula IIa or IIIa:

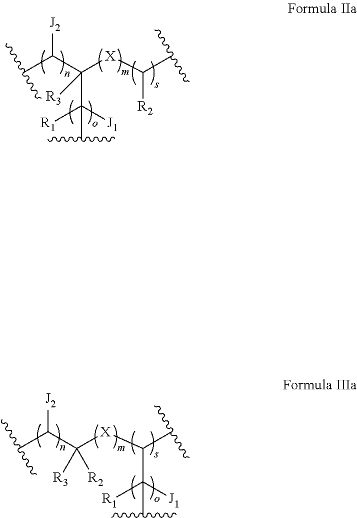

Wherein:
X is O, S, C(R₄R₅), N(E₁), N(E₁)-C(═O);
each J₁ and J₂ is independently H or C₁-C₆ alkyl;
n is 0, 1 or 2;
m is 0, 1, or 2;
o is 0 or 1;
s is 0 or 1;
R₁ is H, OH, halogen, C₁-C₆ alkyl, C₁-C₆ alkoxy, C₂-C₆ alkenyl, C₂-C₆ alkynyl, or (CH₂)_{q}R₇
R₂ and R₃ are each independently H, OH, halogen, C₁-C₆ alkyl, C₁-C₆ alkoxy, C₂-C₆ alkenyl, C₂-C₆ alkynyl, S—CH₃, N(CH₃)(CH₃), OCH₂CH₂OCH₃, O-alkylamino, or (CH₂)_{q}R₇;
E₁ is H, C₁-C₆ alkyl or substituted C₁-C₆ alkyl;
R₄ and R₅ are independently H, OH, C₁-C₆ alkyl, or N(R₆); wherein if R₄ is OH, then R₅ is not OH; R₆ is H, C₁-C₆ alkyl, or C(═O)R₈, wherein R₈ is C₁-C₆ alkyl;
R₇ is OH, halogen, methoxy, ethoxy, azido, and C₂-C₆ alkenyl, or C₂-C₆ alkynyl, and q is 1, 2, or 3.

In certain embodiments, acyclic sugar surrogates are the “unlocked” sugar structure of UNA (unlocked nucleic acid) nucleosides. Representative U.S. publications that teach the preparation of UNA include, but are not limited to, U.S. Patent Publication No. 2011/0313020.

In certain embodiments, acyclic sugar surrogates are the glycerol as found in GNA (glycol nucleic acid) nucleosides, having Formula IIa wherein n is 1, m and o are 0, s is 1, and J₂, R₂, and R₃ are each H, or the butyl as found in acyclic butyl nucleic acid, having Formula IIa wherein n is 2, m and o are 0, s is 1, and 12, R₂, and R₃ are each H. In certain embodiments, acyclic sugar surrogates are also known as “C₃ spacers” and have Formula IIa wherein n and o are 1; m and s are 0, and J₁, J₂, R₁, and R₃ are each H.

Further acyclic sugar surrogates include those described in Manoharan et al., U.S. Pat. No. 10,913,767; US patent publication US 2021/0238595; and PCT publication WO 2023/109940.

In certain embodiments, modified oligonucleotides include one or more sugar mimic, in which a group of atoms other than a “furanosyl sugar moiety” or a “sugar surrogate” form the portion of a nucleoside corresponding to the β-D-ribosyl sugar in RNA. In certain embodiments, a sugar mimic is a portion of the backbone of a peptide nucleic acid, while the remainder of the backbone of the peptide nucleic acid is an internucleoside linkage. Representative U.S. patents that teach the preparation of PNA compounds include, but are not limited to, U.S. Pat. Nos. 5,539,082; 5,714,331; and 5,719,262.

### 2. Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more abasic nucleoside. In certain embodiments, modified oligonucleotides contain only nucleosides comprising nucleobases. In certain embodiments, modified oligonucleotides comprise one or more inosine nucleosides (i.e., nucleosides comprising a hypoxanthine nucleobase). In certain embodiments modified oligonucleotides comprise one or more nucleosides comprising a 5-methylcytosine.

Unless otherwise indicated, modified adenine has structure (I):

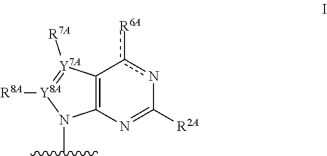

wherein: R_{2A} is H, C₁-C₆ alkyl, substituted C₁-C₆ alkyl, C₁-C₆ thioalkyl, or substituted C₁-C₆ thioalkyl, C₁-C₆ alkyloxy, or substituted C₁-C₆ alkyloxy; R^{6A} is H, N(R^{a})(R^{b}), oxo, acetyl, formyl, or O-phenyl; Y^{7A} is N and R_{7A} is absent or is C₁-C₆ alkyl; or Y^{7A} is C and R_{7A} is H, C₁-C₆ alkyl, or N(R^{a})(R^{b}); Y^{8A} is N and R_{8A} is absent, or Y^{8A} is C and R^{8A} is H, a halogen, OH, C₁-C₆ alkyl, or substituted C₁-C₆ alkyl; R^{a} and R^{b} are each independently H, C₁-C₆ alkyl, substituted C₁-C₆ alkyl, C₁-C₆ alkenyl, substituted C₁-C₆ alkenyl, acetyl, or formyl, or together form a 5-7-membered heterocycle; excluding where Y^{7A} is N and R_{7A} is absent; Y^{8A} is C, R^{8A} is H, R^{2A} is H, and R^{6A} is NH₂ (unmodified adenine).

Unless otherwise indicated, modified guanine has structure (II):

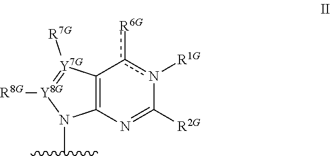

wherein: R_{2G} is N(R^{a})(R^{b}); R_{6G} is oxo and R_{1G} is H, or R_{6G} is O—C₁-C₆ alkyl or S—C₁-C₆ alkyl and R^{1G} is absent; Y^{7G} is N and R^{7G} is absent or is C₁-C₆ alkyl; or Y^{7G} is C and R^{7G} is H, C₁-C₆ alkyl, or N(R^{a})(R^{b}); Y^{8G} is N and R_{8G} is absent, or Y^{8G} is C and R^{8G} is H, a halogen, OH, C₁-C₆ alkyl, or substituted C₁-C₆ alkyl; R^{a} and R^{b} are independently H, C₁-C₆ alkyl, substituted C₁-C₆ alkyl, C₁-C₆ alkenyl, substituted C₁-C₆ alkenyl, acetyl, or formyl, or together form a 5-7-membered heterocycle; excluding where Y^{7G} is N and R^{7G} is absent; Y^{8G} is C, R^{8G} is H, R^{2G} is NH₂, and R^{6G} is ═O (unmodified guanine).

Unless otherwise indicated, modified thymine or modified uracil has structure (III):

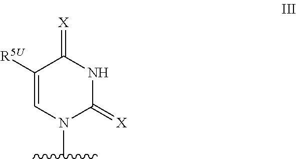

wherein: X is O or S and R^{5U} is H, OH, halogen, O—C₁-C₂₀ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl, C₁-C₁₂ alkynyl, or substituted C₁-C₁₂ alkynyl; wherein if each X is O, R^{5U} is not H or CH₃ (unmodified uracil and unmodified thymine, respectively).

Unless otherwise indicated, modified cytosine has structure (IV):

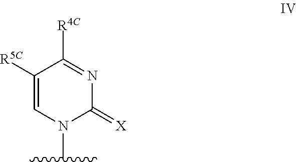

wherein: X is O or S; R^{4C} is N(R^{a})(R^{b}); R^{5C} is H, OH, halogen, O—C₁-C₁₂ alkyl, O—C₁-C₁₂ substituted alkyl, C₁-C₁₂ alkyl, substituted C₁-C₁₂ alkyl, C₁-C₁₂ alkenyl, or substituted C₁-C₁₂ alkenyl; R^{a} and R^{b} are independently H, C₁-C₆ alkyl, substituted C₁-C₆ alkyl, C₁-C₆ alkenyl, substituted C₁-C₆ alkenyl, C₁-C₁₂ alkynyl, substituted C₁-C₁₂ alkynyl, acetyl, or formyl, or together form a 5-7-membered heterocycle; excluding where X is O, R^{4C} is NH₂ and R^{5C} is H (unmodified cytosine).

In certain embodiments, modified nucleobases of a modified oligonucleotide are selected from: 5-substituted pyrimidines, 6-azapyrimidines, alkyl or alkynyl substituted pyrimidines, alkyl substituted purines, and N-2, N-6, and O-6 substituted purines. In certain embodiments, modified nucleobases are selected from: 5-methylcytosine, hypoxanthine, 1-methylpseudouridine, 2-aminopropyladenine, 5-hydroxymethyl cytosine, xanthine, 2-aminoadenine, 6-N-methylguanine, 6-N-methyladenine, 2-propyladenine, 2-thiouracil, 2-thiothymine and 2-thiocytosine, 5-propynyl (—C° C.—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo (particularly 5-bromo), 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine, and 2-pyridone. Further nucleobases include those disclosed in Englisch, U. et al., Angew. Chem. Int. Ed. 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Preparation of certain of the above noted modified nucleobases, as well as other modified nucleobases are known in the art and can be readily identified in publications that include without limitation, Rogers et al., U.S. Pat. No. 5,134,066; Benner et al., U.S. Pat. No. 5,432,272; Matteucci et al., U.S. Pat. No. 5,502,177; Froehler et al., U.S. Pat. No. 5,594,121; and Cook et al., U.S. Pat. No. 5,681,941.

In certain embodiments, each nucleobase of a modified oligonucleotide is selected from unmodified A, unmodified G, unmodified C, unmodified T, unmodified U, and ^{m}C. In certain embodiments, each nucleobase of a modified oligonucleotide is selected from unmodified A, unmodified G, unmodified C, unmodified T, unmodified U, ^{m}C, or hypoxanthine.

### 3. Modified Internucleoside Linkages

In certain embodiments, oligomeric compounds comprising a modified antisense oligonucleotide (e.g., an antisense oligomeric compound) and/or a modified sense oligonucleotide (e.g., a sense oligomeric compound) provided herein comprise or consist of a modified oligonucleotide comprising at least one modified internucleoside linkage. The naturally occurring internucleoside linkage of RNA and DNA is a 3′ to 5′ phosphodiester linkage. Herein, all internucleoside linkages between furanosyl sugar moieties are 3′ to 5′ internucleoside linkages unless otherwise indicated. In certain embodiments, nucleosides of modified oligonucleotides are linked together using one or more modified internucleoside linkages. The two main classes of internucleoside linkages are defined by the presence or absence of a phosphorus atom. Representative phosphorus-containing internucleoside linkages include but are not limited to phosphates, which contain a phosphodiester bond (“P═O”) (also referred to as unmodified linkages), phosphotriesters, methylphosphonates, phosphoramidates, phosphorothioates (“P═S”), and phosphorodithioates (“HS—P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—), siloxane (—O—SiH₂—O—), and N,N′-dimethylhydrazine (—CH₂—N(CH₃)—N(CH₃)—). Modified internucleoside linkages, compared to naturally occurring phosphodiester linkages, may be used to alter, typically increase, nuclease resistance of the oligonucleotide.

In certain embodiments, a modified internucleoside linkage is any of those described in WO 2021/030778, incorporated by reference herein. In certain embodiments, a modified internucleoside linkage has the formula:

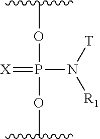

wherein independently for each internucleoside linkage of the modified oligonucleotide:
X is selected from O and S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
R₃ is selected from an aryl, a substituted aryl, CH₃, N(CH₃)₂, OCH₃, and a conjugate group;
R₄ is selected from OCH₃, OH, C₁-C₆ alkyl, substituted C₁-C₆ alkyl, and a conjugate group; and
R₅ is selected from OCH₃, OH, C₁-C₆ alkyl, and substituted C₁-C₆ alkyl.

In certain embodiments, a modified oligonucleotide comprises a mesyl phosphoramidate linkage having a formula:

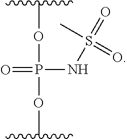 [SMILES: *OP(=O)(NS(C)(=O)=O)O*]

Certain internucleoside linkages having reduced charge (referred to as “neutral internucleoside linkages”) have been described. Such neutral internucleoside linkages include, without limitation, phosphotriesters, methylphosphonates, MMI (3′-CH₂—N(CH₃)—O—5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP) (see U.S. Pat. No. 9,926,556), and thioformacetal (3′-S—CH₂—O—5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, a modified oligonucleotide comprises an internucleoside linkage comprising a triazole, alkyne, or cyclic guanidine moiety. In certain embodiments, a modified oligonucleotide comprises an internucleoside linkage having a formula:

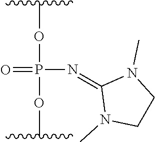 [SMILES: *OP(=O)(N=C1N(C)CCN1C)O*]

which may be stereorandom, or may be enriched for the Rp or Sp configuration.

In certain embodiments, internucleoside linkages are not 3′-to-5′ internucleoside linkages.

In certain embodiments, modified oligonucleotides comprise one or more inverted nucleoside, where a sugar moiety is linked 3′ to 3′ and/or 5′ to 5′, as shown below:

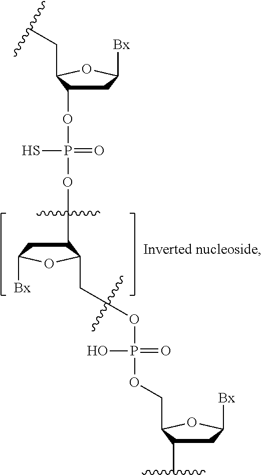

wherein each Bx independently represents any nucleobase.

In certain embodiments, an inverted nucleoside is terminal (i.e., the last nucleoside on one end of an oligonucleotide) and so only one internucleoside linkage depicted above will be present. In certain embodiments, additional features (e.g., a conjugate group) are attached to the inverted nucleoside. Such terminal inverted nucleosides may be attached to either or both ends of an oligonucleotide.

In certain embodiments, inverted nucleosides lack a nucleobase (are abasic nucleosides). In certain such embodiments, additional features (e.g., a conjugate group) are attached to the inverted abasic nucleoside. A terminal inverted nucleoside may be attached to either or both ends of an oligonucleotide.

In certain embodiments, nucleosides are linked 2′ to 5′ rather than the 3′ to 5′ linkage. Such a linkage is illustrated below.

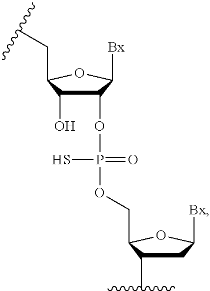

wherein each Bx represents any nucleobase.

In certain embodiments, a bicyclic sugar moiety may be linked via an atom on the non-furanosyl ring. In certain such embodiments, a bicyclic sugar moiety is linked 7′ to 5′, as shown below:

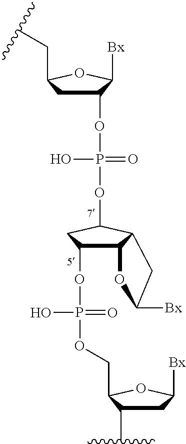

In certain embodiments, internucleoside linkages have at least one chiral center. In such embodiments, a chiral atom can be prepared as a racemic mixture, or as separate enantiomers. Representative internucleoside linkages having a chiral center include but are not limited to alkylphosphonates, mesyl phosphoramidates, and phosphorothioates.

The mesyl phosphoramidate internucleoside linkage comprises a chiral center. In certain embodiments, modified oligonucleotides comprising (Rp) and/or (Sp) mesyl phosphoramidates comprise one or more of the following formulas, respectively, wherein “Bx” indicates a nucleobase:

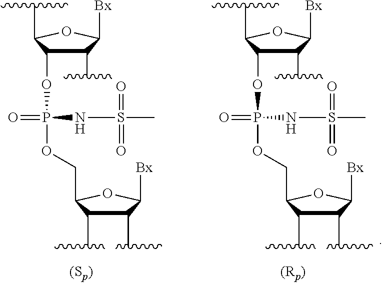

The phosphorothioate internucleoside linkage comprises a chiral center. In certain embodiments, modified oligonucleotides comprising (Rp) and/or (Sp) phosphorothioates comprise one or more of the following formulas, respectively, wherein “Bx” indicates a nucleobase:

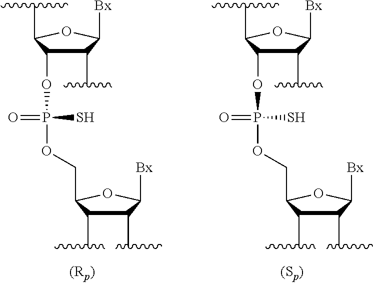

Modified oligonucleotides comprising internucleoside linkages having a chiral center may be prepared as populations of modified oligonucleotides comprising stereorandom internucleoside linkages, or as populations of modified oligonucleotides comprising internucleoside linkages containing chiral centers in particular stereochemical configurations. In certain embodiments, populations of modified oligonucleotides comprise one or more phosphorothioate internucleoside linkages wherein all the phosphorothioate internucleoside linkages are stereorandom. In certain embodiments, populations of modified oligonucleotides comprise one or more mesyl phosphoramidate internucleoside linkages wherein all the mesyl phosphoramidate internucleoside linkages are stereorandom. Such modified oligonucleotides can be generated using synthetic methods that result in random selection of the stereochemical configuration of each phosphorothioate and/or mesyl phosphoramidate linkage. Nonetheless, each individual phosphorothioate and/or mesyl phosphoramidate of each individual oligonucleotide molecule has a defined stereoconfiguration. In certain embodiments, populations of modified oligonucleotides are enriched for modified oligonucleotides comprising one or more particular phosphorothioate and/or mesyl phosphoramidate internucleoside linkages in a particular, independently selected stereochemical configuration. In certain embodiments, the particular configuration of the particular phosphorothioate and/or mesyl phosphoramidate linkage is present in at least 65% of the molecules in the population. In certain embodiments, the particular configuration of the particular phosphorothioate and/or mesyl phosphoramidate linkage is present in at least 70% of the molecules in the population. In certain embodiments, the particular configuration of the particular phosphorothioate and/or mesyl phosphoramidate linkage is present in at least 80% of the molecules in the population. In certain embodiments, the particular configuration of the particular phosphorothioate and/or mesyl phosphoramidate linkage is present in at least 90% of the molecules in the population. In certain embodiments, the particular configuration of the particular phosphorothioate and/or mesyl phosphoramidate linkage is present in at least 99% of the molecules in the population. Such chirally enriched populations of modified oligonucleotides can be generated using synthetic methods known in the art, e.g., methods described in Oka, N., et al. J. Am. Chem. Soc. 2003, 125, 8307-8317; Wan, W. B., et al. Nucleic Acids Res. 2014, 42, 13456, and WO 2017/015555.

As used herein, “chirally enriched” in reference to a population means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom as defined herein. Populations of molecules having multiple chiral centers within each molecule may contain one or more stereorandom chiral centers. In certain embodiments, the molecules are modified oligonucleotides. In certain embodiments, the chiral center is at the phosphorous atom of a phosphorothioate internucleoside linkage. In certain embodiments, the chiral center is at the phosphorous atom of a mesyl phosphoramidate internucleoside linkage. In certain embodiments, a population of modified oligonucleotides is enriched for modified oligonucleotides having at least one indicated phosphorothioate and/or mesyl phosphoramidate in the (Sp) configuration. In certain embodiments, a population of modified oligonucleotides is enriched for modified oligonucleotides having at least one phosphorothioate and/or mesyl phosphoramidate in the (Rp) configuration. Unless otherwise indicated, chiral internucleoside linkages of modified oligonucleotides described herein may be stereorandom or in a particular stereochemical configuration. In certain embodiments, the chiral center is at positions 1′, 2′, 3′, and/or 4′ of a furanosyl sugar moiety. In certain embodiments, each chiral center of each furanosyl sugar moiety is enriched such that the sugar moieties have the β-D ribosyl stereochemical configuration. In certain embodiments, modified oligonucleotides of a population are enriched for β-D ribosyl sugar moieties, and all the phosphorothioate internucleoside linkages are stereorandom and all the mesyl phosphoramidate internucleoside linkages are stereorandom. In certain embodiments, modified oligonucleotides of a population are enriched for R-D ribosyl sugar moieties, at least one particular phosphorothioate internucleoside linkage in a particular stereochemical configuration is enriched, and all the mesyl phosphoramidate internucleoside linkages are stereorandom. In certain embodiments, modified oligonucleotides of a population are enriched for β-D ribosyl sugar moieties, at least one particular mesyl phosphoramidate internucleoside linkage in a particular stereochemical configuration is enriched, and all the phosphorothioate internucleoside linkages are stereorandom. In certain embodiments, modified oligonucleotides of a population are enriched for both β-D ribosyl sugar moieties and at least one, particular phosphorothioate internucleoside linkage in a particular stereochemical configuration and at least one particular mesyl phosphoramidate internucleoside linkage in a particular stereochemical configuration is enriched.

### B. Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In certain such embodiments, the modified, unmodified, and differently modified sugar moieties, nucleobases, and/or internucleoside linkages of a modified oligonucleotide define a pattern or motif.

In certain embodiments, the patterns of sugar moieties, nucleobases, and internucleoside linkages are each independent of one another. Thus, a modified oligonucleotide may be described by its sugar motif, nucleobase motif, and/or internucleoside linkage motif (as used herein, nucleobase motif describes the modifications to the nucleobases independent of the nucleobase sequence).

### 1. Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein. In certain embodiments, the sugar moiety of at least one nucleoside of an antisense oligomeric compound is a modified sugar moiety. In certain embodiments, the sugar moiety of at least one nucleoside of a sense oligomeric compound is a modified sugar moiety.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification. In certain embodiments, every other nucleoside of a uniformly modified oligonucleotide comprises the same 2′-modification, resulting in an alternating 2′-modifications. In certain embodiments, neighboring nucleosides comprise different 2′-modification, and every other nucleoside of a uniformly modified oligonucleotide comprises the same 2′-modification, resulting in a uniform, alternating 2′-modification motif.

In certain embodiments, at least one nucleoside of a modified oligonucleotide comprises a 2′-OMe sugar moiety. In certain embodiments, at least 8 nucleosides comprise 2′-OMe sugar moieties. In certain embodiments, at least 10 nucleosides comprise 2′-OMe sugar moieties. In certain embodiments, at least 12 nucleosides comprise 2′-OMe sugar moieties. In certain embodiments, at least 13 nucleosides comprise 2′-OMe sugar moieties. In certain embodiments, at least 14 nucleosides comprise 2′-OMe sugar moieties. In certain embodiments, at least 15 nucleosides comprise 2′-OMe sugar moieties. In certain embodiments, at least 16 nucleosides comprise 2′-OMe sugar moieties. In certain embodiments, at least 17 nucleosides comprise 2′-OMe sugar moieties. In certain such embodiments, at least 18 nucleosides comprise 2′-OMe sugar moieties. In certain such embodiments, at least 20 nucleosides comprise 2′-OMe sugar moieties.

In certain embodiments, at least one nucleoside of a modified oligonucleotide comprises a 2′-F sugar moiety (i.e., a 2′-F modified nucleoside). In certain embodiments, at least 2 nucleosides comprise 2′-F sugar moieties. In certain embodiments, at least 3 nucleosides comprise 2′-F sugar moieties. In certain embodiments, 4 nucleosides comprise a 2′-F sugar moiety. In certain embodiments, at least one, but not more than four nucleosides comprise a 2′-F sugar moiety. In certain embodiments, 1 or 2 nucleosides comprise 2′-F sugar moieties. In certain embodiments, 1-3 nucleosides comprise 2′-F sugar moieties. In certain embodiments, only one nucleoside comprises a 2′-F sugar moiety. In certain embodiments, an antisense oligomeric compound comprises 2 to 4 non-contiguous 2′-F modified nucleosides. In certain embodiments, 4 nucleosides of an antisense oligomeric compound are 2′-F modified nucleosides and none of those 2′-F modified nucleosides are contiguous. In certain embodiments, 1, 2, 3, or 4 nucleosides of an antisense oligomeric compound are 2′-F modified nucleosides and each of those 2′-F modified nucleosides are non-contiguous. In certain such embodiments at least fifteen of the remainder of the nucleosides are 2′-OMe modified nucleosides. In certain embodiments, one nucleoside of an antisense oligomeric compound is a 2′-F modified nucleoside and at least fifteen of the remainder of the nucleosides are 2′-OMe modified nucleosides.

In certain embodiments, at least one nucleoside of a modified oligonucleotide and an oligomeric duplex comprises a 2′-deoxy sugar moiety that has no additional modifications. In certain embodiments, at least one nucleoside of a modified oligonucleotide and an oligomeric duplex comprises a 2′-deoxyribosyl sugar moiety that has no additional modifications. In certain embodiments, at least one nucleoside comprises a 2′-deoxyribosyl sugar moiety. In certain embodiments, at least 2 nucleosides comprise a 2′-deoxyribosyl sugar moiety. In certain embodiments, at least 3 nucleosides comprise a 2′-deoxyribosyl sugar moiety. In certain embodiments, at least 4 nucleosides comprise a 2′-deoxyribosyl sugar moiety. In certain embodiments, one nucleoside comprises a 2′-deoxyribosyl sugar moiety. In certain embodiments, 1 or 3 nucleosides comprise a 2′-deoxyribosyl sugar moiety. In certain embodiments, 1-3 nucleosides comprise a 2′-deoxyribosyl sugar moiety. In certain embodiments, three nucleosides comprise a 2′-deoxyribosyl sugar moiety. In certain embodiments, 1, 2, 3, or 4 nucleosides of an antisense oligomeric compound are a 2′-deoxyribosyl sugar modified nucleoside and each 2′-deoxyribosyl modified nucleoside is non-contiguous. In certain embodiments, 1, or 3 nucleosides of an antisense oligomeric compound are a 2′-deoxyribosyl sugar modified nucleoside and each 2′-deoxyribosyl modified nucleoside is non-contiguous. In certain embodiments, no nucleosides of a sense oligomeric compound are a 2′-deoxyribosyl sugar modified nucleoside. In certain embodiments, three nucleosides of an antisense oligomeric compound are 2′-deoxyribosyl sugar modified nucleosides and no nucleoside of a sense oligomeric compound is a 2′-deoxyribosyl modified nucleoside. In certain embodiments, three nucleosides of an antisense oligomeric compound are 2′-deoxyribosyl sugar modified nucleosides and one nucleoside of a sense oligomeric compound is a 2′-deoxyribosyl modified nucleoside. In certain embodiments, one nucleosides of an antisense oligomeric compound are 2′-deoxyribosyl sugar modified nucleosides and no nucleoside of a sense oligomeric compound is a 2′-deoxyribosyl modified nucleoside.

In certain embodiments, at least one nucleoside of a modified oligonucleotide comprises a 2′-deoxyxylosyl sugar moiety that has no additional modifications In certain embodiments, at least one nucleoside comprises a 2′-deoxyxylosyl sugar moiety. In certain embodiments, at least 2 nucleosides comprise a 2′-deoxyxylosyl sugar moiety. In certain embodiments, at least 3 nucleosides comprise a 2′-deoxyxylosyl sugar moiety. In certain embodiments, at least 4 nucleosides comprise a 2′-deoxyxylosyl sugar moiety. In certain embodiments, one nucleoside comprises a 2′-deoxyxylosyl sugar moiety. In certain embodiments, 1 or 3 nucleosides comprise a 2′-deoxyxylosyl sugar moiety. In certain embodiments, 1-3 nucleosides comprise a 2′-deoxyxylosyl sugar moiety. In certain embodiments, three nucleosides comprise a 2′-deoxyxylosyl sugar moiety. In certain embodiments, 1, 2, 3, or 4 nucleosides of an antisense oligomeric compound are a 2′-deoxyxylosyl sugar modified nucleoside and each 2′-deoxyxylosyl modified nucleoside is non-contiguous. In certain embodiments, 1, or 3 nucleosides of an antisense oligomeric compound are a 2′-deoxyxylosyl sugar modified nucleoside and each 2′-deoxyxylosyl modified nucleoside is non-contiguous. In certain embodiments, no nucleosides of a sense oligomeric compound are a 2′-deoxyxylosyl sugar modified nucleoside. In certain embodiments, three nucleosides of an antisense oligomeric compound are 2′-deoxyxylosyl sugar modified nucleosides and no nucleoside of a sense oligomeric compound is a 2′-deoxyxylosyl modified nucleoside. In certain embodiments, one nucleoside of an antisense oligomeric compound is 2′-deoxyxylosyl sugar modified nucleosides and no nucleoside of a sense oligomeric compound is a 2′-deoxyxylosyl modified nucleoside. In certain embodiments, no nucleoside of an antisense oligomeric compound is 2′-deoxyxylosyl sugar modified nucleosides and one nucleoside of a sense oligomeric compound is a 2′-deoxyxylosyl modified nucleoside.

In certain embodiments, at least one nucleoside of a modified oligonucleotide comprises a 2′-deoxy sugar moiety selected from a 2′-deoxyxylosyl and a 2′-deoxyribosyl that has no additional modifications. In certain embodiments, at least one nucleoside comprises a 2′-deoxy sugar moiety. In certain embodiments, at least 2 nucleosides comprise a 2′-deoxy sugar moiety. In certain embodiments, at least 3 nucleosides comprise a 2′-deoxy sugar moiety. In certain embodiments, at least 4 nucleosides comprise a 2′-deoxy sugar moiety. In certain embodiments, one nucleoside comprises a 2′-deoxy sugar moiety. In certain embodiments, 1 or 3 nucleosides comprise a 2′-deoxy sugar moiety. In certain embodiments, 1-3 nucleosides comprise a 2′-deoxy sugar moiety. In certain embodiments, three nucleosides comprise a 2′-deoxy sugar moiety. In certain embodiments, 1, 2, 3, or 4 nucleosides of an antisense oligomeric compound are a 2′-deoxy sugar modified nucleoside and each 2′-deoxy modified nucleoside is non-contiguous. In certain embodiments, 1, or 3 nucleosides of an antisense oligomeric compound are a 2′-deoxy sugar modified nucleoside and each 2′-deoxy modified nucleoside is non-contiguous. In certain embodiments, no nucleosides of a sense oligomeric compound are a 2′-deoxy sugar modified nucleoside. In certain embodiments, three nucleosides of an antisense oligomeric compound are 2′-deoxy sugar modified nucleosides and no nucleoside of a sense oligomeric compound is a 2′-deoxy modified nucleoside. In certain embodiments, one nucleoside of an antisense oligomeric compound is 2′-deoxy sugar modified nucleosides and no nucleoside of a sense oligomeric compound is a 2′-deoxy modified nucleoside. In certain embodiments, no nucleoside of an antisense oligomeric compound is 2′-deoxy sugar modified nucleosides and one nucleoside of a sense oligomeric compound is a 2′-deoxy modified nucleoside.

In certain embodiments, three nucleosides of an antisense oligomeric compound are 2′-deoxy sugar modified nucleosides and one nucleoside of a sense oligomeric compound is a 2′-deoxy modified nucleoside. In certain embodiments, one nucleoside of an antisense oligomeric compound is 2′-deoxy sugar modified nucleosides and one nucleoside of a sense oligomeric compound is a 2′-deoxy modified nucleoside. In certain embodiments, three nucleosides of an antisense oligomeric compound are 2′-deoxyribose sugar modified nucleosides and one nucleoside of a sense oligomeric compound is a 2′-deoxyxylose modified nucleoside. In certain embodiments, one nucleoside of an antisense oligomeric compound is 2′-deoxyribose sugar modified nucleosides and one nucleoside of a sense oligomeric compound is a 2′-deoxyxylose modified nucleoside. In certain embodiments, one nucleoside of an antisense oligomeric compound is 2′-deoxyxylose sugar modified nucleosides and one nucleoside of a sense oligomeric compound is a 2′-deoxyribose modified nucleoside.

In certain embodiments, at least one nucleoside of a modified oligonucleotide comprises a sugar surrogate moiety. In certain embodiments, at least one nucleoside of a modified oligonucleotide comprises a sugar surrogate moiety comprising FHNA. In certain embodiments, at least one nucleoside comprises a sugar surrogate. In certain embodiments, at least two nucleosides comprise a sugar surrogate. In certain embodiments, only one nucleoside comprises a sugar surrogate. In certain embodiments, two nucleosides of an antisense oligomeric compound are sugar surrogates and each of those surrogates are non-contiguous. In certain embodiments, at least one nucleoside comprises an FHNA sugar surrogate. In certain embodiments, at least two nucleosides comprise an FHNA sugar surrogate. In certain embodiments, only one nucleoside comprises an FHNA sugar surrogate. In certain embodiments, two nucleosides of an antisense oligomeric compound are FHNA sugar surrogates and each of those surrogates are non-contiguous. In certain embodiments, one nucleoside of an antisense oligomeric compound in an FHNA, located at position 2 from the 5′ end of the antisense oligonucleotide.

In certain embodiments, a sugar moiety of an antisense oligomeric compound is modified, wherein the modified sugar modifications and/or sugar surrogate is selected from 2′-F, a 3′-fluoro-hexitol, 2′-MOE, 2′-OMe, 2′-deoxyxylosyl and 2′-deoxyribosyl. In certain embodiments, a sugar motif (from 5′ to 3′) of the antisense oligomeric compound is selected from efyyydyyeyyyydydyyyyyee, efyyydyyyyyyyfyfyyyyyee, efyyydyyeyyyyfyfyyyyyee, ehyyyfyyyyyyyfyfyyyyyee, ehyyyfyyeyyyyfyfyyyyyee, efyyyyyyyyyyyfyfyyyyyee, efyyyfyyyyyyyfyyyyyyyee, efyyyyyyeyyyyfyfyyyyyee, efyyyfyyeyyyyfyyyyyyyee, efyyxfyyyyyyyfyyyyyyyee, efyyyfxyyyyyyfyyyyyyyee, and efyyyxyyyyyyyfyyyyyyyee, wherein each ‘e’ represents a 2′-MOE sugar moiety, each ‘y’ represents a 2′-OMe sugar moiety, each ‘f’ represents a 2′-F sugar moiety, each ‘h’ represents a 3′-fluoro-hexitol sugar moiety, each ‘d’ represents a 2′-deoxyribose sugar moiety, and each ‘x’ represents a 2′-deoxyxylose sugar moiety. In certain embodiments, a sugar motif (from 5′ to 3′) of a sense oligomeric compound is selected from among: eeyyyyyyyffyyyyyyyyee, yyyyyyyyfffyyeyyyyyyy, eeyyyyyyfffyyeyyyyyee, eeyyyyyydffyyeyyyyyee, eeyyyyyyfdfyyeyyyyyee, eeyyyyyyffdyyeyyyyyee, eeyyyyyyhffyyeyyyyyee, eeyyyyyyfhfyyeyyyyyee, eeyyyyyyffhyyeyyyyyee, eeyyyyyyfxfyyeyyyyyee, eeyyyyyyffxyyeyyyyyee, and eeyyyyyyxffyyeyyyyyee, wherein each ‘e’ represents a 2′-MOE sugar moiety, each ‘y’ represents a 2′-OMe sugar moiety, each ‘f’ represents a 2′-F sugar moiety, each ‘h’ represents a 3′-fluoro-hexitol sugar moiety, each ‘d’ represents a 2′-deoxyribose sugar moiety, and each ‘x’ represents a 2′-deoxyxylose sugar moiety.

### 2. Nucleobase Motifs

In certain embodiments, oligonucleotides comprise modified and/or unmodified nucleobases arranged along the oligonucleotide or region thereof in a defined pattern or motif. In certain embodiments, at least one nucleobase is modified. In certain embodiments, none of the nucleobases are modified. In certain embodiments, at least one purine and/or at least pyrimidine is modified. In certain embodiments, at least one adenine is modified. In certain embodiments, at least one guanine is modified. In certain embodiments, at least one thymine is modified. In certain embodiments, at least one uracil is modified. In certain embodiments, at least one cytosine is modified. In certain embodiments, at least one of the cytosine nucleobases in a modified oligonucleotide is 5-methylcytosine. In certain embodiments, all the cytosine nucleobases are 5-methylcytosines and all the other nucleobases of the modified oligonucleotide are unmodified nucleobases. In certain embodiments, one or two of the cytosine nucleobases are 5-methylcytosines and all the other nucleobases of the modified oligonucleotide are unmodified nucleobases. In certain embodiments, each nucleobase is selected from 5-methylcytosine, unmodified cytosine, unmodified thymine, unmodified uracil, unmodified adenine, unmodified guanine, and unmodified hypoxanthine. In certain embodiments, each nucleobase is selected from 5-methylcytosine, unmodified cytosine, unmodified thymine, unmodified uracil, unmodified adenine, unmodified guanine, and unmodified hypoxanthine. In certain embodiments, each nucleobase is selected from 5-methylcytosine, unmodified cytosine, unmodified thymine, unmodified adenine, and unmodified guanine. In certain embodiments, each nucleobase is selected from 5-methylcytosine, unmodified cytosine, unmodified thymine, unmodified adenine, and unmodified guanine. In certain embodiments, each nucleobase is selected from unmodified cytosine, unmodified thymine, unmodified uracil, unmodified adenine, and unmodified guanine. In certain embodiments, each nucleobase is selected from unmodified cytosine, unmodified thymine, unmodified adenine, and unmodified guanine.

### 3. Internucleoside Linkage Motifs

In certain embodiments, oligonucleotides comprise modified and unmodified internucleoside linkages arranged along the oligonucleotide or region thereof in a defined pattern or motif. In certain embodiments, each internucleoside linkage is a phosphodiester internucleoside linkage. In certain embodiments, each internucleoside linkage of a modified oligonucleotide is a phosphorothioate internucleoside linkage. In certain embodiments, each internucleoside linkage of a modified oligonucleotide is independently selected from a phosphorothioate internucleoside linkage, a mesyl phosphoramidate internucleoside linkage, and a phosphodiester internucleoside linkage. In certain embodiments, each internucleoside linkage of a modified oligonucleotide is independently selected from a phosphorothioate internucleoside linkage and a phosphodiester internucleoside linkage. In certain embodiments, each internucleoside linkage of a modified oligonucleotide is independently selected from a mesyl phosphoramidate internucleoside linkage and a phosphorothioate internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, each mesyl phosphoramidate internucleoside linkage is independently selected from a stereorandom mesyl phosphoramidate, a (Sp) mesyl phosphoramidate, and a (Rp) mesyl phosphoramidate.

In certain embodiments, the modified antisense oligonucleotide has an internucleoside linkage motif independently selected from (5′ to 3′) of: ssooooooooooooooooooss, ssooosooooooososooooss, and ssooosooooooooooooooss, wherein each “s” represents a phosphorothioate internucleoside linkage and each “o” represents a phosphodiester internucleoside. In certain embodiments, the modified sense oligonucleotide has an internucleoside linkage motif independently selected from (5′ to 3′) of: ssooooooooooooooooss, ssooooooosooooooooss, ssoooooosoooooooooss, and ssoooooooosoooooooss, wherein each “s” represents a phosphorothioate internucleoside linkage, and each “o” represents a phosphodiester internucleoside.

Provided oligomeric compounds comprise one or more modifications, (e.g., a modified sugar moiety, a modified nucleobase, a modified internucleoside linkage), and/or combinations thereof, incorporated into a modified oligonucleotide. In certain embodiments, a modified oligonucleotide is characterized by modification motif(s) and overall length. In certain embodiments, such parameters are each independent of one another. Thus, unless otherwise indicated, each internucleoside linkage of a modified oligonucleotide having one or more modified sugar moiety and/or sugar motif, independently, is modified or unmodified and may or may not follow the modification pattern of the sugar modifications or sugar motif. For example, internucleoside linkages within a region of a modified oligonucleotide comprising certain sugar modifications may be the same or different from one another and may be the same or different from the internucleoside linkages of the region of the modified oligonucleotide comprising different sugar modifications. Likewise, such modified oligonucleotides may comprise one or more modified nucleobase independent of the pattern of the sugar modifications or sugar motif and independent of the internucleoside linkages or internucleoside linkage motif. Unless specifically indicated, all modifications are independent of nucleobase sequence.

### C. Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were evaluated for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, modified oligonucleotides (including antisense oligomeric compounds) comprise 16 linked nucleosides having no more than 1 to 3 mismatches to a target sequence. In certain embodiments, modified oligonucleotides (including antisense oligomeric compounds) comprise 17 linked nucleosides having no more than 1 to 3 mismatches to a target sequence. In certain embodiments, modified oligonucleotides (including antisense oligomeric compounds) comprise 18 linked nucleosides having no more than 1 to 3 mismatches to a target sequence. In certain embodiments, modified oligonucleotides (including antisense oligomeric compounds) comprise 19 linked nucleosides having no more than 1 to 3 mismatches to a target sequence. In certain embodiments, modified oligonucleotides (including antisense oligomeric compounds) comprise 20 linked nucleosides having no more than 1 to 3 mismatches to a target sequence. In certain embodiments, modified oligonucleotides (including antisense oligomeric compounds) comprise 21 linked nucleosides having no more than 1 to 3 mismatches to a target sequence. In certain embodiments, modified oligonucleotides (including antisense oligomeric compounds) comprise 22 linked nucleosides having no more than 1 to 3 mismatches to a target sequence. In certain embodiments, modified oligonucleotides (including antisense oligomeric compounds) comprise 23 linked nucleosides having no more than 1 to 3 mismatches to a target sequence.

In certain embodiments, modified oligonucleotides (including antisense oligomeric compounds) consist of 16 linked nucleosides. In certain embodiments, modified oligonucleotides (including antisense oligomeric compounds) consist of 17 linked nucleosides. In certain embodiments, modified oligonucleotides (including antisense oligomeric compounds) consist of 18 linked nucleosides. In certain embodiments, modified oligonucleotides (including antisense oligomeric compounds) consist of 19 linked nucleosides. In certain embodiments, modified oligonucleotides (including antisense oligomeric compounds) consist of 20 linked nucleosides. In certain embodiments, modified oligonucleotides (including antisense oligomeric compounds) consist of 21 linked nucleosides. In certain embodiments, modified oligonucleotides (including antisense oligomeric compounds) consist of 22 linked nucleosides. In certain embodiments, modified oligonucleotides (including antisense oligomeric compounds) consist of 23 linked nucleosides.

In certain embodiments, antisense oligomeric compounds consist of 16-30 linked nucleosides. In certain embodiments, antisense oligomeric compounds consist of 17-25 linked nucleosides. In certain embodiments, antisense oligomeric compounds consist of 17-23 linked nucleosides. In certain embodiments, antisense oligomeric compounds consist of 17-21 linked nucleosides. In certain embodiments, antisense oligomeric compounds consist of 18-30 linked nucleosides. In certain embodiments, antisense oligomeric compounds consist of 20-30 linked nucleosides. In certain embodiments, antisense oligomeric compounds consist of 21-30 linked nucleosides. In certain embodiments, antisense oligomeric compounds consist of 23-30 linked nucleosides. In certain embodiments, antisense oligomeric compounds consist of 18-25 linked nucleosides. In certain embodiments, antisense oligomeric compounds consist of 20-22 linked nucleosides. In certain embodiments, antisense oligomeric compounds consist of 21-23 linked nucleosides. In certain embodiments, antisense oligomeric compounds consist of 23-24 linked nucleosides. In certain embodiments, antisense oligomeric compounds consist of 20 linked nucleosides. In certain embodiments, antisense oligomeric compounds consist of 21 linked nucleosides. In certain embodiments, antisense oligomeric compounds consist of 22 linked nucleosides. In certain embodiments, antisense oligomeric compounds consist of 23 linked nucleosides.

In certain embodiments, sense oligomeric compounds consist of 15-30 linked nucleosides. In certain embodiments, sense oligomeric compounds consist of 16-25 linked nucleosides. In certain embodiments, sense oligomeric compounds consist of 16-23 linked nucleosides. In certain embodiments, sense oligomeric compounds consist of 16-21 linked nucleosides. In certain embodiments, sense oligomeric compounds consist of 16-30 linked nucleosides. In certain embodiments, sense oligomeric compounds consist of 18-30 linked nucleosides. In certain embodiments, sense oligomeric compounds consist of 19-30 linked nucleosides. In certain embodiments, sense oligomeric compounds consist of 16-25 linked nucleosides. In certain embodiments, sense oligomeric compounds consist of 18-25 linked nucleosides. In certain embodiments, sense oligomeric compounds consist of 18-20 linked nucleosides. In certain embodiments, sense oligomeric compounds consist of 19-21 linked nucleosides. In certain embodiments, sense oligomeric compounds consist of 18 linked nucleosides. In certain embodiments, sense oligomeric compounds consist of 19 linked nucleosides. In certain embodiments, sense oligomeric compounds consist of 20 linked nucleosides. In certain embodiments, sense oligomeric compounds consist of 21 linked nucleosides.

### D. Nucleobase Sequence

In certain embodiments, modified oligonucleotides (e.g., oligomeric compounds) are further described by their nucleobase sequence. In certain embodiments oligonucleotides of oligomeric compounds have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 85%, at least 90%, at least 95%, or 100% complementary to the second oligonucleotide or nucleic acid, such as a target nucleic acid. In certain embodiments a first oligomeric compound comprises a modified oligonucleotide consisting of 18 to 50 linked nucleosides, wherein the nucleobase sequence of the first oligomeric compound comprises at least 18, at least 19, at least 20, at least 21, at least 22, or at least 23 nucleobases of the nucleobase sequence of any one of SEQ ID NOs: 85-98, or 143-144. In certain embodiments a second oligomeric compound comprises a modified oligonucleotide consisting of 18 to 50 linked nucleosides, wherein the nucleobase sequence of the second oligomeric compound comprises at least 16, at least 17, at least 18, at least 19, at least 20, or at least 21 contiguous nucleobases of the nucleobase sequence of any one of SEQ ID NOs: 99-112 or 145-146.

### II. Conjugates

In certain embodiments, provided herein are oligomeric compounds comprising one or more modified oligonucleotide and one or more conjugate groups. In certain embodiments, an oligomeric compound optionally further comprises one or more terminal groups. Conjugate groups comprise or consist of a conjugate moiety and a conjugate linker. A conjugate group may be attached at the 3′ end and/or the 5′ end of an oligonucleotide and/or at any internal position. In certain embodiments, conjugate groups are attached through a modified sugar moiety or a modified internucleoside linkage. In certain embodiments, oligomeric compounds comprise a modified oligonucleotide, a cell-targeting moiety, and a conjugate linker.

### A. Conjugate Groups

In certain embodiments, a conjugate group comprises a conjugate moiety and a conjugate linker.

### Conjugate Moieties

In certain embodiments, a conjugate moiety modifies one or more properties of an attached oligonucleotide, including but not limited to pharmacodynamics, pharmacokinetics, stability, binding, absorption, tissue distribution, cellular distribution, cellular uptake, charge, and clearance. In certain embodiments, a conjugate moiety imparts a new property on the attached oligonucleotide.

In certain embodiments, a conjugate moiety comprises or consists of a cell-targeting moiety. In certain embodiments, a cell-targeting moiety is capable of binding the cell-surface receptor or the cell-surface moiety. In certain embodiments, an agent comprising a cell-targeting moiety is capable of being internalized when it interacts with or binds the cell-surface receptor or the cell-surface moiety. In certain embodiments, a cell-targeting moiety comprises a liver cell targeting moiety or a liver cell ligand. In certain embodiments, a liver cell-targeting moiety consists of a cell-targeting moiety having affinity for the hepatic asialoglycoprotein receptor (ASGP-R). In certain embodiments, the cell-targeting moiety comprises more than one ligand, and each ligand has affinity for the ASGP-R. In certain embodiments, each ligand is a carbohydrate. In certain embodiments, each ligand is independently selected from galactose, N-acetyl galactosamine (GalNAc), mannose, glucose, glucosamine, and fucose.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29 or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, each ligand is N-acetyl galactosamine (GalNAc). In certain embodiments, the cell-targeting moiety comprises one GalNAc ligand. In certain embodiments, the cell-targeting moiety comprises two GalNAc ligands. In certain embodiments, the cell-targeting moiety comprises three GalNAc ligands. In certain embodiments, the cell-targeting moiety comprises a GalNAc ligand cluster. In certain embodiments, the cell-targeting moiety comprises a three GalNAc ligand cluster. In certain embodiments, the cell-targeting moiety is any one of those described in U.S. Pat. No. 9,127,276, the entire contents of which is incorporated herein by reference. In certain embodiments, a conjugate groups comprises a cell-targeting moiety selected from any one of the formula set forth in TABLE A:

TABLE A
Conjugate Groups

| | |
|---|---|
| 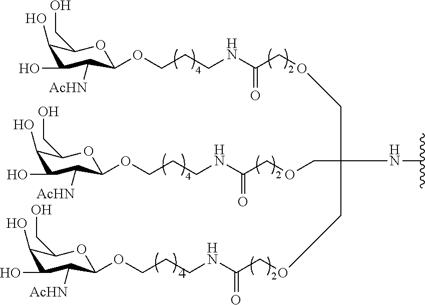 | GalNAc3-7 |
| 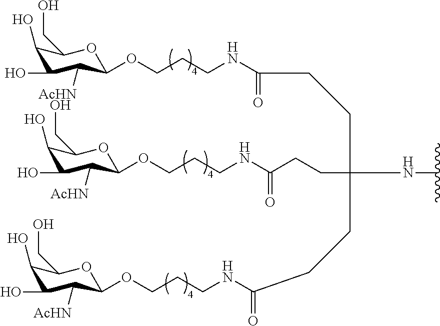 | GalNAc3-10 |
| 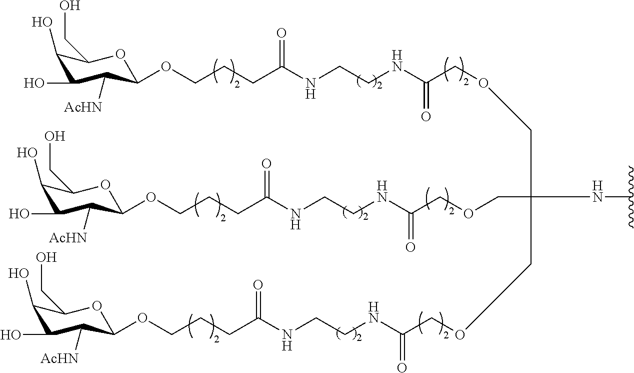 | GalNAc3-1 |
| 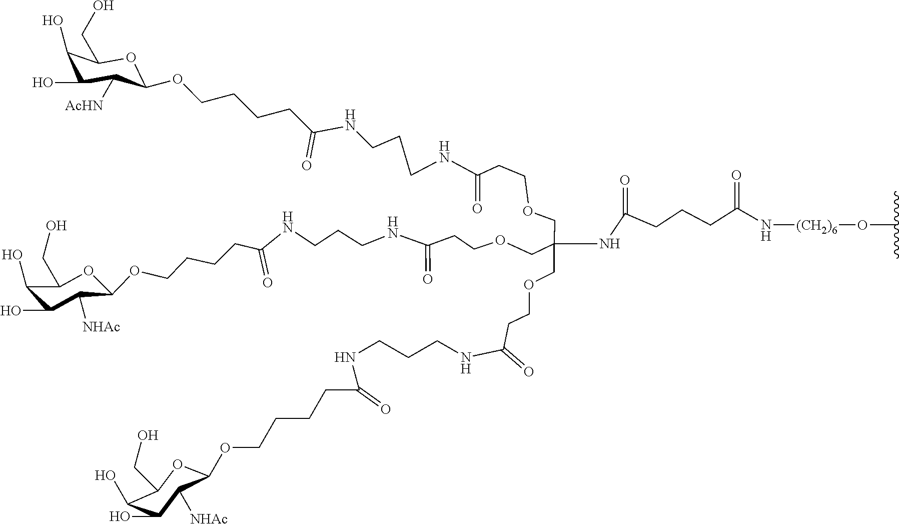 | GalNAc3-3 |
| 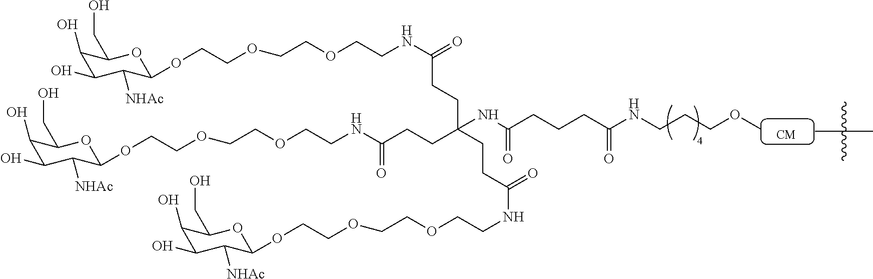 | GalNAc3-23 (with a cleavable linker moiety) |
| 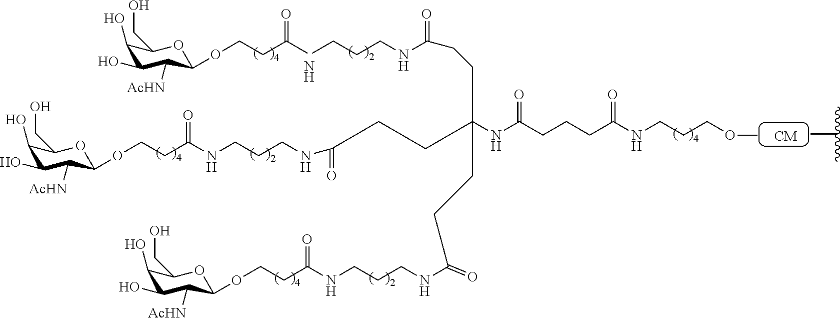 | GalNAc3-8 (with a cleavable linker moiety) |
| 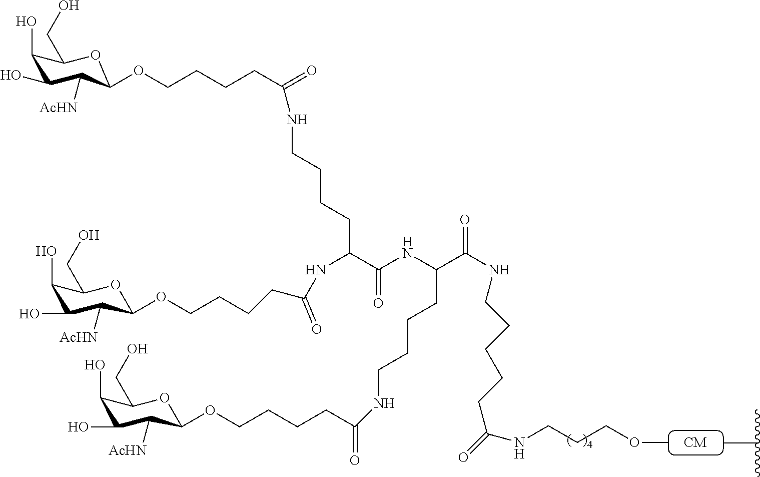 | GalNAc3-5 (with a cleavable linker moiety) |
| 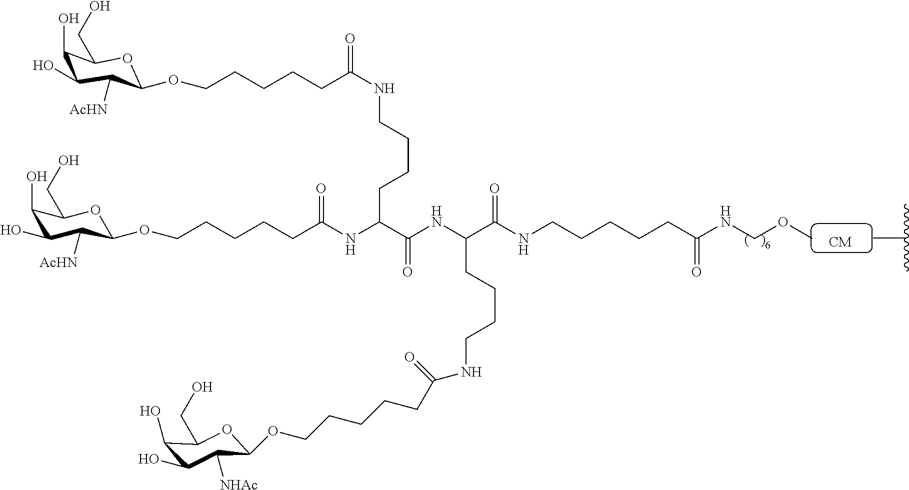 | GalNAc3-13 (with a cleavable linker) |
| 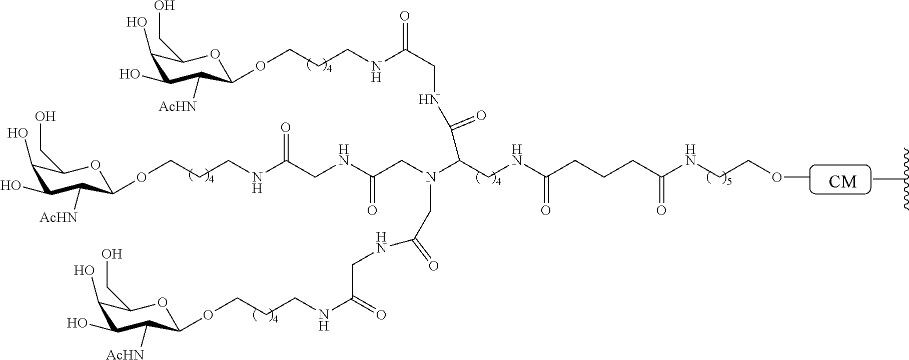 | GalNAc3-6 (with a cleavable linker moiety) |
| 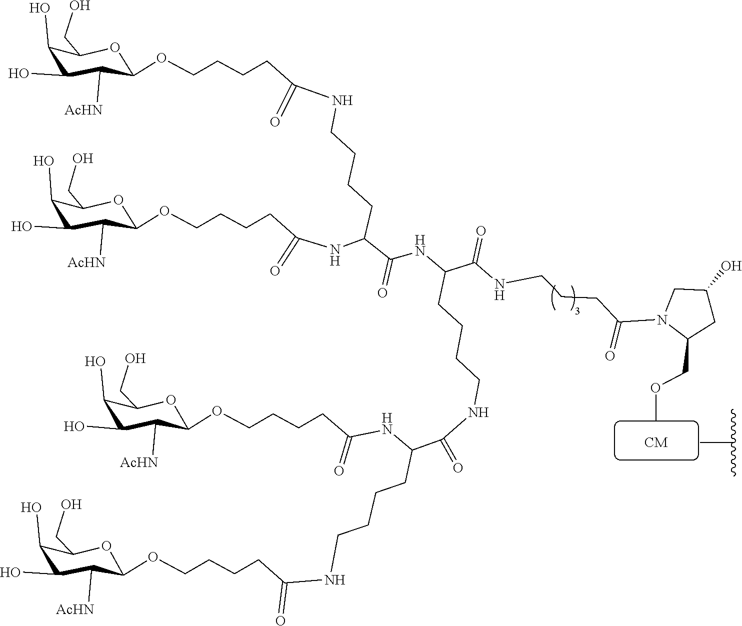 | GalNAc4-11 (with a cleavable linker moiety) |
| 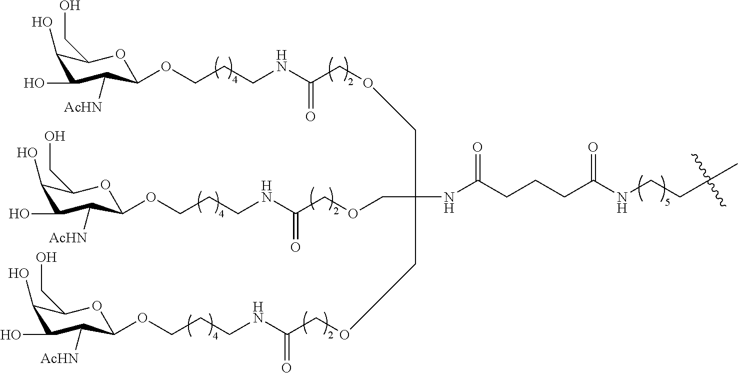 | LICA-1 |

### Conjugate Linkers

In certain embodiments, oligomeric compounds comprise an oligonucleotide and a conjugate group, wherein the conjugate group comprises a conjugate moiety and a conjugate linker. In certain embodiments, the conjugate linker links the conjugate moiety to the oligonucleotide. In certain embodiments, the conjugate linker is a single chemical bond (i.e., the conjugate moiety is attached directly to an oligonucleotide through a single bond). In certain embodiments, the conjugate linker comprises one or more atoms. In certain embodiments, the conjugate linker comprises a chemical group. In certain embodiments, the conjugate linker comprises a chain structure, such as a hydrocarbyl chain, or an oligomer of repeating units such as ethylene glycol, nucleosides, or amino acid units. In certain embodiments, the oligonucleotide is a modified oligonucleotide.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises one or more groups selected from alkyl, amino, oxo, amide and ether groups. In certain embodiments, the conjugate linker comprises one or more groups selected from alkyl and amide groups. In certain embodiments, the conjugate linker comprises one or more groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described herein, are bifunctional linking moieties, e.g., those known in the art to be useful for attaching conjugate moieties to parent compounds, such as the oligonucleotides provided herein. In general, a bifunctional linking moiety comprises at least two functional groups. One of the functional groups is selected to react with a particular site on a parent compound and the other is selected to react with a peptide extender. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

In certain embodiments, conjugate linkers comprise chemical groups that are formed upon a reaction between a first functional group and a second functional group. In certain embodiments, a modified oligonucleotide is attached to the first functional group during synthesis, and a conjugate moiety is attached to a second functional group during synthesis. Then, the two compounds are mixed under specific conditions to yield the final oligomeric compound. Such reactions that are compatible with both oligonucleotide and peptide chemistry have been previously described and are often called “bioconjugation” reactions. These reactions include strain promoted azido-alkyne cycloaddition (SPAAC), copper-catalyzed click reaction (CuAAC), active ester conjugation to an amino modified oligonucleotide, maleimide-thiol Michael addition, ketol/hydroxylamine ligation, the Staudinger ligation, reductive amination, thio ether formation, disulfide formation, reductive alkylation, catalyst-free N-arylation, sulfur fluoride exchange click reaction (SuFEx), and inverse demand Diels Alder reaction. Certain such reactions are described in, e.g., Jbara, et al., “Oligonucleotide Bioconjugation with Bifunctional Palladium Reagents”, Angew. Chem. Int. Ed. 2021, 60(21)₁₂₁₀₉-12115; Dong, et al., “Sulfur(VI) Fluoride Exchange (SuFEx): Another Good Reaction for Click Chemistry,” Angew. Chem. Int. Ed. 2014, 53(36):9430-9448.4; Zhang, et al., “Arylation Chemistry for Bioconjugation,” Angew. Chem. Int. Ed. Engl. 2019; 58(15): 4810-4839; Walsh, et al., “Site-selective modification strategies in antibody-drug conjugates” Chem. Soc. Rev., 2021, 50: 1305-1353; Tiefenbrunn, et al., “Chemoselective ligation techniques: modem applications of time-honored chemistry”, Biopolymers, 2010, 94(1):95-106; Drake, et al., Bioconjug. Chem. 2014, 25(7):1331-1341; Bode, Acc. Chem. Res., 2017, 50, 9, 2104-2115; J. Magano, B. Bock, et al, Org. Proc. Res. Dev. 2014, 18:142-151; Craig S. McKay and M. G. Finn, “Click Chemistry in Complex Mixtures: Bioorthogonal Bioconjugation”, Chemistry & Biology 2014; Mitchell P. Christy et al., Org. Lett. 2020, 22: 2365; Ren et al., Angew. Chem. Int. Ed. Engl. 2009, 48, 9658-9662; Rohrbacher, F. et al., Helv. Chim. Acta. 2018, 101; Baalmaan, et al, “A Bioorthogonal Click Chemistry Toolbox for Targeted Synthesis of Branched and Well-Defined Protein-Protein Conjugates”, Angew. Chem. Int. Ed. 2020 (59): 12885-12893; Lang, et al, “Biorthogonal Reactions for Labeling Proteins”, J. Am. Chem. Soc, 2014, 9(1):16-20; Nair, et al., “The Thiol-Michael Addition Click Reaction: A Powerful and Widely Used Tool in Materials Chemistry”, Chem. Mater. 2013 26(1):724-744; Kalia and Raines, “Hydrolytic Stability of Hydrazones and Oximes”, Angew. Chem. Int. Ed., 2008, 47:7523-7526.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-5 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine, or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) an oligonucleotide consisting of 18-30 nucleosides and (2) a conjugate linker comprising 1-10 linker-nucleosides that are contiguous with the nucleosides of the oligonucleotide. The total number of contiguous linked nucleosides in such an oligomeric compound is more than 30. Alternatively, an oligomeric compound may comprise an oligonucleotide consisting of 18-30 nucleosides and no conjugate linker. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate moiety to be cleaved from the oligonucleotide. For example, in certain circumstances oligomeric compounds comprising a particular conjugate moiety are better taken up by a particular cell type, but once the oligomeric compound has been taken up, it is desirable that the conjugate moiety be cleaved to release the unconjugated or parent oligonucleotide. Thus, certain conjugate linkers may comprise one or more cleavable moieties.

In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among an amide, an ester, an ether, one or both esters of a phosphodiester, a phosphate ester, a carbamate, or a disulfide. In certain embodiments, a cleavable bond is one or both esters of a phosphodiester. In certain embodiments, a cleavable moiety comprises a phosphate or phosphodiester. In certain embodiments, the cleavable moiety is a phosphodiester linkage between an oligonucleotide and a conjugate moiety.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

In certain embodiments, oligomeric compounds described herein comprise an oligonucleotide linked to a conjugate moiety by a conjugate linker, wherein the oligomeric compound is prepared using Click chemistry known in the art. Compounds have been prepared using Click chemistry wherein alkynyl phosphonate internucleoside linkages on an oligomeric compound attached to a solid support are converted into the 1,2,3-triazolylphosphonate internucleoside linkages and then cleaved from the solid support (Krishna et al., J. Am. Chem. Soc. 2012, 134(28), 11618-11631), which is incorporated by reference herein in its entirety. Additional conjugate linkers suitable for use in several embodiments are prepared by Click chemistry described in “Click Chemistry for Biotechnology and Materials Science” Ed. Joerg Laham, Wiley 2009, which is incorporated by reference herein in its entirety.

In certain embodiments, compounds comprise an oligonucleotide, a cell-targeting moiety, and a conjugate linker. In certain embodiments, oligomeric compounds comprise an oligonucleotide, a hepatic asialoglycoprotein receptor (ASGP-R) ligand, and a conjugate linker. In certain embodiments, oligomeric compounds comprise an oligonucleotide, a N-acetyl galactosamine (GalNAc) ligand, and a conjugate linker. In certain embodiments, oligomeric compounds comprise an oligonucleotide, a GalNAc trimer, a branching group, a conjugate linker, and optionally modifications to the GalNAc ligands. In certain embodiments, oligomeric compounds comprise an oligonucleotide, two or more GalNAc ligands, a branching group, a conjugate linker, and optionally modifications to the GalNAc ligands. In certain embodiments, a conjugate linker connects GalNAc ligand to an oligonucleotide.

In certain embodiments, two or more GalNAc ligands are covalently connected to a conjugate linker, and the conjugate linker is covalently connected to the 3′ end of an oligonucleotide. In certain embodiments, a three GalNAc cluster is covalently connected to a conjugate linker, and the conjugate linker is covalently connected to the 3′ end of an oligonucleotide. In certain embodiments, two or more GalNAc ligands are covalently connected to a conjugate linker, and the conjugate linker is covalently connected to the 5′ end of an oligonucleotide. In certain embodiments, a three GalNAc cluster is covalently connected to a conjugate linker, and the conjugate linker is covalently connected to the 5′ end of an oligonucleotide. In certain embodiments, two or more GalNAc ligands are covalently connected to a conjugate linker, and the conjugate linker is covalently connected to an internal position of an oligonucleotide. In certain embodiments, a three GalNAc cluster is covalently connected to a conjugate linker, and the conjugate linker is covalently connected to an internal position of an oligonucleotide. In certain embodiments, an internal position of an oligonucleotide is a 2′-position of a modified sugar moiety of a nucleoside within the internal region of an oligonucleotide that is not the 5′ terminal nucleoside or the 3′ terminal nucleoside. In certain embodiments, an internal position of an oligonucleotide is a modified internucleoside linkage of the oligonucleotide.

In certain embodiments, a sense oligomeric compound is conjugated to a THA-GalNAc conjugate group attached to the 5′-OH of the oligonucleotide. The structure of THA-GalNAc is:

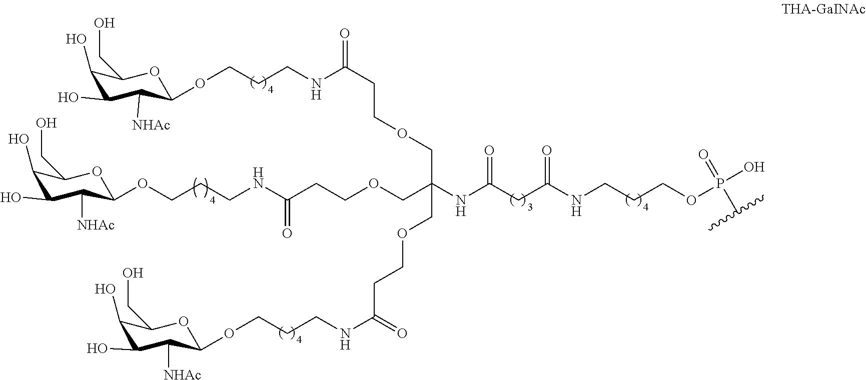

In certain embodiments a sense oligomeric compound is conjugated to a HPPO-GalNAc conjugate group attached to the 3′-OH of the oligonucleotide. The structure of HPPO-GalNAc is:

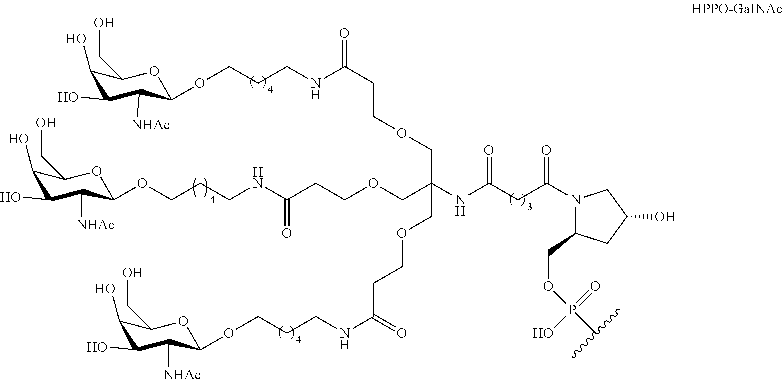

### B. Certain Terminal Groups

Examples of a terminal group include, but are not limited to, a conjugate group, a capping group, a phosphate moiety, a protecting group, a modified or unmodified nucleoside, and two or more nucleosides that are independently modified or unmodified, wherein one or more groups is attached to either or both ends of an oligonucleotide. In certain embodiments, one or more terminal groups is attached to either or both ends of an oligonucleotide. In certain embodiments, one or more terminal groups is attached at the 3′ and/or 5′-end of the oligonucleotide. In certain embodiments, one or more terminal groups is attached at the 3′-end of the oligonucleotide. In certain embodiments, one or more terminal groups is attached at the 5′-end of the oligonucleotide. In certain embodiments, one or more terminal groups is attached at the 3′-end of the oligonucleotide and one or more terminal groups is attached at the 5′-end of the oligonucleotide. In certain embodiments, a terminal group is attached at the 3′ and/or 5′-end of the oligonucleotide. In certain embodiments, a terminal group is attached at the 3′-end of the oligonucleotide. In certain embodiments, a terminal group is attached near the 3′-end of the oligonucleotide. In certain embodiments, a terminal group is attached at the 5′-end of the oligonucleotide. In certain embodiments, a terminal group is attached near the 5′-end of the oligonucleotide. In certain embodiments, a terminal group is attached at the 3′-end of the oligonucleotide and a terminal group is attached at the 5′-end of the oligonucleotide.

In certain embodiments, an oligomeric compound comprises one or more terminal groups. In certain embodiments, an oligomeric compound comprises a terminal group comprising a stabilized 5′-phosphate. Stabilized 5′-phosphates include, but are not limited to 5′-phosphonates, including, but not limited to 5′-vinylphosphonate, 5′-methylphosphonate. In certain embodiments, aterminal group comprises one or more abasic sugar moieties. In certain embodiments, a terminal group comprises one or more inverted sugar moieties and/or inverted nucleosides. In certain embodiments, a terminal group comprises one or more 2′-linked nucleosides or sugar moieties. In certain embodiments, the 2′-linked terminal group is an abasic sugar moiety. In certain embodiments, an antisense oligomeric compound comprises a vinylphosphonate. In certain embodiments, each antisense oligomeric compound has a vinyl phosphonate moiety on the 5′-end (5′-VP).

### III. Target Nucleic Acids

### A. LPA

In certain embodiments, an oligomeric compound comprises or consist of a modified oligonucleotide comprising a targeting region that is complementary to an equal-length target region of a target nucleic acid, wherein the target nucleic acid is LPA. In certain embodiments, LPA nucleic acid has the nucleobase sequence set forth in SEQ ID NO: 1 (the complement of GenBank Accession No. NM_005577.4) or to a region of human LPA comprising nucleobases 486-530 of GenBank Accession No. NM_005577.4: GCAUCCAUGGUAAUGGACAGAGUUAUCGAGGCACAUACUCCACCA (SEQ ID NO: 2), or to both. In certain embodiments, contacting a cell with an oligomeric duplex comprising an oligomeric compound comprising a modified oligonucleotide that is complementary to an equal-length target region of SEQ ID NO: 1 and/or SEQ ID NO: 2 inhibits LPA RNA in the cell, and in certain embodiments inhibits apo(a) protein or Lp(a) produced in the cell. In certain embodiments, the oligomeric compound comprises of a modified oligonucleotide. In certain embodiments, the oligomeric compound comprises a modified oligonucleotide and a conjugate group. In certain embodiments, the oligomeric compound comprises a modified oligonucleotide and one or more terminal group(s). In certain embodiments, the oligomeric compound comprises a modified oligonucleotide and a conjugate group and one or more terminal group(s). In certain embodiments, an oligomeric compound comprises an antisense oligonucleotide comprising a targeting region that is complementary to a region of human LPA comprising SEQ ID NO: 2.

In certain embodiments, antisense oligonucleotides provided herein are complementary to a target region of a LPA nucleic acid over the entire length of the modified oligonucleotide. In certain embodiments, antisense oligonucleotides are at least 99%, at least 95%, at least 90%, at least 85%, or at least 80% complementary to an equal length portion of the LPA nucleic acid. In certain embodiments, antisense oligonucleotides are at least 80% complementary to a target region of the LPA nucleic acid over the entire length of the antisense oligonucleotide and comprise a targeting region that is 100% or fully complementary to the target region of the LPA nucleic acid.

In certain embodiments, a targeting region is from 6 to 20, 10 to 18, 14 to 18, 16 to 20, or 18 to 20 nucleobases in length. In certain embodiments, the targeting region comprises or consists of at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases. In certain embodiments, the targeting region comprises or consists of at least 8, at least 9, at least 10, at least 11, at least 12, at least 13, at least 14, at least 15, at least 16, at least 17, at least 18, at least 19, at least 20, at least 21, at least 22, or 23 contiguous nucleobases. In certain embodiments, the targeting region constitutes at least 75%, at least 80%, at least 85%, at least 90%, or at least 95% of the nucleosides of the antisense oligonucleotide. In certain embodiments, the targeting region constitutes all the nucleosides of the antisense oligonucleotide. In certain embodiments, the targeting region of the antisense oligonucleotide is at least 99%, at least 95%, at least 90%, at least 85%, or at least 80% complementary to a target region of the LPA nucleic acid. In certain embodiments, the targeting region of the antisense oligonucleotide is 100% complementary to a target region of the LPA nucleic acid.

In certain embodiments, antisense oligonucleotides comprise one or more mismatches relative to the target region of the LPA nucleic acid. In certain embodiments, antisense activity against the target is reduced by such a mismatch, and activity against a non-target is reduced. In certain embodiments, activity against the non-target is reduced by a greater amount than activity against the target. Thus, in certain embodiments selectivity of the antisense oligonucleotides is improved. In certain embodiments, antisense oligonucleotides are at least 80% complementary to the target region of the LPA nucleic acid over the entire length of the antisense oligonucleotide and comprise no more than one to three mismatches with the LPA nucleic acid. In certain embodiments, antisense oligonucleotides comprise a targeting region that is at least 80% complementary to a target region of the LPA nucleic acid over the entire length of the targeting region, and the targeting region comprises no more than one to three mismatches with the target region. In certain embodiments, antisense oligonucleotides comprise a targeting region that is at least 80%, at least 85%, at least 90%, at least 95%, or 100% complementary to a target region of the LPA nucleic acid over the entire length of the targeting region. In certain embodiments, additional mismatches may be present at a terminus or at both termini of the antisense oligonucleotide, outside of the targeting region. In certain embodiments, a mismatch is specifically positioned within an antisense oligonucleotide. In certain embodiments, a mismatch is at position 3, 4, 5, 6, 7, 8, 9, 10, 11, or 12 from the 5′-end of the antisense oligonucleotide. In certain embodiments, a mismatch is at position 11, 10, 9, 8, 7, 6, 5, 4, 3, or 2 from the 31-end of the antisense oligonucleotide. In certain embodiments, a mismatch is at position 1, 2, 3, or 4 from the 5′-end of the antisense oligonucleotide. In certain embodiments, a mismatch is at position 4, 3, 2, or 1 from the 31-end of the antisense oligonucleotide.

### B. Target Nucleic Acids in Certain Tissues

In certain embodiments, oligomeric compounds comprise or consist of a modified oligonucleotide comprising a targeting region that is complementary to a target region in a LPA nucleic acid, wherein the LPA nucleic acid is expressed in a pharmacologically relevant tissue. In certain embodiments, the LPA nucleic acid is expressed in the liver.

### C. Oligonucleotide Sequences

Provided herein are oligomeric compounds comprising modified oligonucleotides complementary to a target region in a LPA nucleic acid, such as, for example, a human LPA nucleic acid, such as SEQ ID NO: 1 (GENBANK Accession No. NM_005577.4) or SEQ ID NO: 2 (a region of human LPA comprising nucleobases 486-530 of GenBank Accession No. NM_005577.4), or to both and compositions comprising such oligomeric compounds. In certain embodiments, a modified oligonucleotide has a nucleobase sequence comprising or consisting of a targeting region that is at least 80%, at least 85%, at least 90%, at least 95%, or at least 99% complementary to a region of SEQ ID NOs: 1 and/or 2. In certain embodiments, a modified oligonucleotide has a complementary region that is at least 80%, at least 85%, at least 90%, at least 95%, or at least 99% complementary to a targeting region that is at least 80%, at least 85%, at least 90%, at least 95%, or at least 99% complementary to a target region of SEQ ID NOs: 1 and/or 2. In certain embodiments, a modified oligonucleotide has a targeting region that is 100% complementary to a target region of SEQ ID NOs: 1 and/or 2. In certain embodiments, a modified oligonucleotide has a nucleobase sequence comprising or consisting of a complementary region that is at least 80%, at least 85%, at least 90%, at least 95%, or at least 99% complementary to a targeting region that is 100% complementary to a target region of SEQ ID NOs: 1 and/or 2. In certain embodiments, a modified antisense oligonucleotide has a nucleobase sequence comprising or consisting of any of SEQ ID NO: 3-48, or 132-140, or 83-98, or 143-144. In certain embodiments, a second modified sense oligonucleotide has a nucleobase sequence comprising or consisting of a complementary region that is at least 80%, at least 85%, at least 90%, at least 95%, or at least 99% complementary to the first modified antisense oligonucleotide, which is 100% complementary to the modified antisense oligonucleotide. In certain embodiments, a modified sense oligonucleotide has a nucleobase sequence comprising or consisting of any of SEQ ID NO: 49-82, 141-142, 99-112 or 145-146.

### IV. Oligomeric Duplexes

In certain embodiments, an oligomeric compound provided herein comprises a modified oligonucleotide having a nucleobase sequence complementary to a sequence in a LPA target nucleic acid paired with a second oligomeric compound to form an oligomeric duplex. Such oligomeric duplex comprises a first oligomeric compound comprising a modified oligonucleotide having a portion complementary to a sequence in a LPA target nucleic acid and a second oligomeric compound comprising a modified oligonucleotide having a portion complementary to the first oligomeric compound. In certain embodiments, the first oligomeric compound of an oligomeric duplex comprises or consists of (1) a first modified oligonucleotide and optionally a conjugate group and/or terminal group; and the second oligomeric compound of the oligomeric duplex comprises or consists of (2) a second modified oligonucleotide and optionally a terminal group and/or a conjugate group. Either or both oligomeric compounds of an oligomeric duplex may comprise a conjugate group. Either or both oligomeric compounds of an oligomeric duplex may comprise a terminal group. The oligonucleotides of each oligomeric compound of an oligomeric duplex may include non-complementary or unpaired overhanging nucleosides. In certain embodiments the non-complementary or unpaired overhanging nucleosides are adenosine or thymine. In certain embodiments the non-complementary or unpaired overhanging nucleosides include inosine. In certain embodiments, the two oligonucleotides have at least one mismatch relative to one another. In certain embodiments, the oligomeric duplex is an antisense agent.

In certain embodiments, an oligomeric duplex comprises: a first oligomeric compound comprising a first modified oligonucleotide consisting of 18 to 28 linked nucleosides, wherein the nucleobase sequence of the first modified oligonucleotide comprises at least 18, at least 19, at least 20, at least 21, at least 22, or at least 23 nucleobases of the nucleobase sequence of any one of SEQ ID NOs: 3-48, or 132-140, or 83-98, or 143-144; and a second oligomeric compound comprising a second modified oligonucleotide consisting of 15 to 25 linked nucleosides, wherein the nucleobase sequence of the second modified oligonucleotide comprises at least 16, at least 17, at least 18, at least 19, at least 20, or at least 21 nucleobases of the nucleobase sequence of any one of SEQ ID NOs: 49-82, 141-142, 99-112 or 145-146; and wherein each of the nucleosides of the first modified oligonucleotide comprises a modified sugar moiety or sugar surrogate and wherein no more than 22%, no more than 20%, no more than 18%, no more than 15%, no more than 10%, or no more than 5% of the modified nucleosides in the first modified oligonucleotide comprises a 2′-F modification and each of the nucleosides of the second modified oligonucleotide comprises a modified sugar moiety or sugar surrogate and wherein no more than 25%, no more than 20%, no more than 18%, no more than 16%, no more than 14%, no more than 12%, or no more than 10%, of the modified nucleosides in the second modified oligonucleotide comprises a 2′-F modification. In certain embodiments, the oligomeric duplex is an antisense agent. In certain embodiments, the first oligomeric compound of the oligomeric duplex is an antisense agent, wherein the first modified oligonucleotide is an antisense oligomeric compound. In certain embodiments, the second oligomeric compound of the oligomeric duplex is a sense agent, wherein the second modified oligonucleotide is a sense oligomeric compound. In certain embodiments, the first modified oligonucleotide is an antisense RNAi oligonucleotide wherein no more than 22%, no more than 20%, no more than 18%, no more than 16%, no more than 14%, no more than 12%, no more than 10%, or no more than 7%, of the modified nucleosides in the oligomeric duplex comprise a modified sugar moiety comprising a 2′-F modification. In certain embodiments, the second modified oligonucleotide is a sense RNAi oligonucleotide. In certain embodiments, the nucleobase sequence of the second modified oligonucleotide is at least 90%, 95% or 100% complementary to the nucleobase sequence of an equal length portion of the first modified oligonucleotide.

In certain embodiments, an oligomeric duplex comprises a first oligomeric compound comprising a first modified oligonucleotide consisting of 18 to 28 linked nucleosides, wherein the nucleobase sequence of the first modified oligonucleotide comprises at least 18, at least 19, at least 20, at least 21, at least 22, or 23 nucleobases of the nucleobase sequence of any one of SEQ ID NO: 3-48, or 132-140, or 83-98, or 143-144; and a second oligomeric compound comprising a second modified oligonucleotide consisting of 15 to 25 linked nucleosides, wherein the nucleobase sequence of the second modified oligonucleotide comprises at least 16, at least 17, at least 18, at least 19, at least 20, or at least 21 nucleobases of the nucleobase sequence of any one of SEQ ID NOs: 49-82, 141-142, 99-112 or 145-146, and wherein each of the nucleosides of the first modified oligonucleotide comprises a modified sugar moiety or sugar surrogate and the first modified oligonucleotide comprises at least one modified nucleoside and no more than four modified nucleosides of the first modified oligonucleotide comprises a 2′-F modification, and each of the nucleosides of the second modified oligonucleotide comprises a modified sugar moiety or sugar surrogate and at least one modified nucleoside and no more than four modified nucleosides of the second modified oligonucleotide comprises a 2′-F modification. In certain embodiments, the first oligomeric compound is an antisense agent, wherein the first modified oligonucleotide is an antisense oligomeric compound. In certain embodiments, the second oligomeric compound is a sense agent, wherein the second modified oligonucleotide is a sense oligomeric compound. In certain embodiments, the first modified oligonucleotide is an antisense RNAi oligonucleotide. In certain embodiments, the second modified oligonucleotide is a sense RNAi oligonucleotide. In certain embodiments, the nucleobase sequence of the second modified oligonucleotide comprises a complementary region of at least 10, at least 11, at least 12, at least 13, at least 14, at least 15, at least 16, at least 17, at least 18, at least 19, at least 20, or 21 nucleobases that is 100% complementary to the nucleobase sequence of an equal portion of the first modified oligonucleotide. In certain embodiments, the nucleobase sequence of the second modified oligonucleotide is at least 80%, at least 85%, at least 90%, at least 95%, at least 98%, at least 99%, or 100% complementary to the nucleobase sequence of an equal portion of the first modified oligonucleotide. In certain embodiments, the oligomeric duplex is an antisense agent. In certain embodiments, the nucleobase sequence of the second modified oligonucleotide comprises a complementary region of at least 10, at least 11, at least 12, at least 13, at least 14, at least 15, at least 16, at least 17, at least 18, at least 19, at least 20, or 21 nucleobases that is 100% complementary to the nucleobase sequence of an equal portion of the first modified oligonucleotide; and the nucleobase sequence of the second modified oligonucleotide is at least 80%, at least 85%, at least 90%, at least 95%, at least 98%, at least 99%, or 100% complementary to the nucleobase sequence of an equal portion of the first modified oligonucleotide. In certain embodiments, the oligomeric duplex is an antisense agent, wherein no more than three nucleosides, no more than four nucleosides, no more than five nucleosides, no more than six nucleosides, no more than seven nucleosides, or no more than eight nucleosides in the oligomeric duplex comprise a modified sugar moiety or sugar surrogate comprising a fluorine.

In certain embodiments, an oligomeric duplex comprises a first oligomeric compound comprising a first modified oligonucleotide consisting of 18 to 28 linked nucleosides, wherein the nucleobase sequence of the first modified oligonucleotide comprises at least 10, at least 11, at least 12, at least 13, at least 14, at least 15, at least 16, at least 17, at least 18, at least 19, at least 20, or at least 21 contiguous nucleobases of the nucleobase sequence of any one of SEQ ID NO: 3-48, or 132-140, or 83-98, or 143-144; and a second oligomeric compound comprising a second modified oligonucleotide consisting of 15 to 25 linked nucleosides, wherein the nucleobase sequence of the second modified oligonucleotide comprises at least 10, at least 11, at least 12, at least 13, at least 14, at least 15, at least 16, at least 17, at least 18, at least 19, or at least 20 contiguous nucleobases the nucleobase sequence of any one of SEQ ID NO: 49-82, 141-142, 99-112 or 145-146; wherein each of the nucleosides of the first modified oligonucleotide independently and the second modified oligonucleotide independently comprises a modified sugar moiety or sugar surrogate independently selected from 2′-F, 2′-MOE, 2′-OMe, 2′-deoxyribosyl, 2′-deoxyxylosyl, and 3′-fluoro-hexitol, wherein at least one modified nucleoside and no more than four modified nucleosides of the first modified oligonucleotide comprises a 2′-F modification, and at least one modified nucleoside and no more than four modified nucleosides of the second modified oligonucleotide comprises a 2′-F modification. In certain embodiments, the first oligomeric compound is an antisense agent, wherein the first modified oligonucleotide is an antisense oligomeric compound. In certain embodiments, the second oligomeric compound is a sense agent, wherein the second modified oligonucleotide is a sense oligomeric compound. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent wherein no more than three nucleosides, no more than four nucleosides, no more than five nucleosides, no more than six nucleosides, no more than seven nucleosides, or no more than eight nucleosides in the oligomeric duplex comprise a modified sugar moiety or sugar surrogate comprising a fluorine.

In certain embodiments, an oligomeric duplex comprises a first oligomeric compound comprising a first modified oligonucleotide consisting of 18 to 28 linked nucleosides, wherein the nucleobase sequence of the first modified oligonucleotide comprises at least 10, at least 11, at least 12, at least 13, at least 14, at least 15, at least 16, at least 17, at least 18, at least 19, at least 20, or at least 21 nucleobases of the sequence of any one of SEQ ID NO: 3-48 or 132-140; and a second oligomeric compound comprising a second modified oligonucleotide consisting of 15 to 25 linked nucleosides, wherein the nucleobase sequence of the second modified oligonucleotide comprises at least 10, at least 11, at least 12, at least 13, at least 14, at least 15, at least 16, at least 17, at least 18, at least 19, or at least 20 contiguous nucleobases of the sequence of any one of SEQ ID NO: 49-82, 141-142; wherein each of the nucleosides of the first modified oligonucleotide independently and the second modified oligonucleotide independently comprises a modified sugar moiety or sugar surrogate independently selected from 2′-F, 2′-MOE, 2′-OMe, 2′-deoxyribosyl, 2′-deoxyxylosyl, and 3′-fluoro-hexitol wherein only one nucleoside only two nucleosides or only three nucleosides of the first modified oligonucleotide are a 2′-deoxynucleoside and no nucleosides or one nucleoside of the second modified oligonucleotide are 2′-deoxynucleoside; wherein one two or three of the modified sugar moiety and/or sugar surrogate comprises a 2′-F modification in the first modified oligonucleotide, and two modified nucleosides of the second modified oligonucleotide comprises a 2′-F modification. In certain embodiments, the first oligomeric compound is an antisense agent, wherein the first modified oligonucleotide is an antisense oligomeric compound comprising a 5′ terminal group. In certain embodiments, the second oligomeric compound is a sense agent, wherein the second modified oligonucleotide is a sense oligomeric compound optionally conjugated to a cell targeting moiety. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent wherein no more than three nucleosides, no more than four nucleosides, or no more than five nucleosides, in the oligomeric duplex comprise a modified sugar moiety or sugar surrogate comprising a fluorine.

In certain embodiments, an oligomeric duplex comprises a first oligomeric compound comprising a first modified oligonucleotide consisting of 18 to 28 linked nucleosides, wherein the nucleobase sequence of the first modified oligonucleotide comprises at least 18, at least 19, at least 20, at least 21 or at least 22 nucleobases of the nucleobase sequence of any one of SEQ ID NO: 5-6 or 15-48 or 132-140 and a second oligomeric compound comprising a second modified oligonucleotide consisting of 15 to 25 linked nucleosides, wherein the nucleobase sequence of the second modified oligonucleotide comprises at least 15, at least 16, at least 17, at least 18, at least 19, or at least 20 nucleobases the nucleobase sequence of any one of SEQ ID NO: 52-82, or 141-142; wherein each of the nucleosides of the first modified oligonucleotide independently and the second modified oligonucleotide independently comprises a modified sugar moiety or sugar surrogate independently selected from 2′-F, 2′-MOE, 2′-OMe, 2′-deoxyribosyl, 2′-deoxyxylosyl, and 3′-fluoro-hexitol, wherein only one nucleoside of the first modified oligonucleotides comprises a 3′-fluoro-hexitol and no nucleosides of the second modified oligonucleotide comprise 3′-fluoro-hexitol or wherein no nucleoside of the first modified oligonucleotides comprises a 3′-fluoro-hexitol and only one nucleoside of the second modified oligonucleotide comprise 3′-fluoro-hexitol; wherein one, two or three of the modified sugar moiety and/or sugar surrogate comprises a 2′-F modification in the first modified oligonucleotide, and two, or three modified nucleosides of the second modified oligonucleotide comprises a 2′-F modification. In certain embodiments, the first oligomeric compound is an antisense agent, wherein the first modified oligonucleotide is an antisense oligomeric compound comprising a 5′ terminal group. In certain embodiments, the second oligomeric compound is a sense agent, wherein the second modified oligonucleotide is a sense oligomeric compound optionally conjugated to a cell targeting moiety. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent wherein no more than five nucleosides, no more than six nucleosides, no more than seven nucleosides or no more than eight nucleosides, in the oligomeric duplex comprise a modified sugar moiety or sugar surrogate comprising a fluorine.

In certain embodiments, an oligomeric duplex comprises a first oligomeric compound comprising a first modified oligonucleotide, wherein the first modified oligonucleotide consists of 21-23 linked nucleosides and has sequence comprising at least a 19-bp sequence of any one of SEQ ID NOs: 5-6, or 15-48, or 132-140 having 0, 1, 2 or 3 nucleosides that are different from the corresponding nucleotide in any of SEQ ID NOs: 5-6, or 15-48, or 132-140; and a second oligomeric compound comprising a second modified oligonucleotide wherein the second modified oligonucleotide consists of 19-21 linked nucleosides and has sequence comprising at least a 17-bp sequence of any one of SEQ ID NOs: 52-82 or 141-142, having 0, 1, 2 or 3 nucleosides that are different from the corresponding nucleoside in any of SEQ ID NOs: 52-82, or 141-142, wherein each of the nucleosides of the first modified oligonucleotide independently and the second modified oligonucleotide independently comprises a modified sugar moiety or sugar surrogate independently selected from 2′-F, 2′-MOE, 2′-OMe, 2′-deoxyribosyl, 2′-deoxyxylosyl, and 3′-fluoro-hexitol, wherein one, two or three nucleosides or no nucleosides of the first modified oligonucleotide is 2′-deoxynucleoside and one nucleoside or no nucleosides of the second modified oligonucleotide are 2′-deoxynucleoside; wherein one or two or three of the modified sugar moiety and/or sugar surrogate comprises a 2′-F modification in the first modified oligonucleotide, and two, or three modified nucleosides of the second modified oligonucleotide comprises a 2′-F modification. In certain embodiments, the first oligomeric compound is an antisense agent, wherein the first modified oligonucleotide is an antisense oligomeric compound comprising a 5′ terminal group. In certain embodiments, the second oligomeric compound is a sense agent, wherein the second modified oligonucleotide is a sense oligomeric compound optionally conjugated to a cell targeting moiety. In certain embodiments, the first modified oligonucleotide is an antisense RNAi oligonucleotide, and the second modified oligonucleotide is a sense RNAi oligonucleotide. In certain embodiments, the sequence of the second modified oligonucleotide is at least 95% or 100% complementary to the nucleobase sequence of an equal length portion of the first modified oligonucleotide. In certain embodiments, the oligomeric duplex is an antisense agent, wherein at least one modified nucleoside and no more than five nucleosides, no more than six nucleosides, no more than seven nucleosides or no more than eight nucleosides, in the oligomeric duplex comprise a modified sugar moiety or sugar surrogate comprising a fluorine. In certain embodiments, one or two or three of the modified sugar moiety and/or sugar surrogate comprises a 2′-F modification in the first modified oligonucleotide, and two or three modified nucleosides of the second modified oligonucleotide comprises a 2′-F modification, and no more than six nucleosides in the oligomeric duplex comprise a modified sugar moiety or sugar surrogate comprising a fluorine. In certain embodiments, two or three of the modified sugar moiety and/or sugar surrogate comprises a 2′-F modification in the first modified oligonucleotide, and two or three modified nucleosides of the second modified oligonucleotide comprises a 2′-F modification. In certain embodiments, the oligomeric duplex comprises one or two unpaired nucleosides at either or both ends, forming one or two overhang ends. In certain embodiments an overhang end is one or two nucleosides of the antisense oligomeric compound. In certain embodiments an overhang end is one or two 3′-nucleosides of the antisense oligomeric compound. In certain embodiments the last two 3′-nucleosides of the antisense oligomeric compound are overhang nucleosides not paired with the sense oligomeric compound. In certain embodiments the last one or two 3′-unpaired overhang nucleosides comprise an adenine nucleobase. In certain embodiments the last one or two 3′-unpaired overhang nucleosides comprise a thymine nucleobase. In certain embodiments the last one or two 3′-unpaired overhang nucleosides comprise a uridine nucleobase. In certain embodiments the last one or two 3′-unpaired overhang nucleosides comprise an inosine nucleobase. In certain embodiments, the oligomeric duplex is an antisense agent wherein no more than four nucleosides, no more than five nucleosides, no more than six nucleosides or no more than seven nucleosides, in the oligomeric duplex comprise a modified sugar moiety or sugar surrogate comprising a fluorine.

In certain embodiments, an oligomeric duplex comprises a first oligomeric compound comprising a first modified oligonucleotide, wherein the first modified oligonucleotide consists of 21-23 linked nucleosides and has a sequence comprising at least a 19-bp sequence of any one of SEQ ID NOs: 5-6, or 15-48, or 132-140 having 0, 1, 2 or 3 mismatches to SEQ ID NO: 2; and a second oligomeric compound comprising a second modified oligonucleotide, wherein the second modified oligonucleotide consists of 19-21 linked nucleosides and has a sequence comprising at least a 19-bp sequence of any one of SEQ ID NOs: 52-82, or 141-142 having 0, 1, 2 or 3 mismatches to the first modified oligonucleotide. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 85%, 90%, 95% or 100% complementary to the nucleobase sequence of an equal length portion of the first modified oligonucleotide. In certain embodiments, the oligomeric duplex is an antisense agent wherein each of the nucleosides of the first modified oligonucleotide independently and the second modified oligonucleotide independently comprises a modified sugar moiety or sugar surrogate independently selected from 2′-F, 2′-MOE, 2′-OMe, 2′-deoxyribosyl, 2′-deoxyxylosyl, and 3′-fluoro-hexitol, and wherein at least one modified nucleoside and no more than three modified nucleosides of the first modified oligonucleotide comprises a 2′-F modification, and at least one modified nucleoside and no more than three modified nucleosides of the second modified oligonucleotide comprises a 2′-F modification. In certain embodiments, the oligomeric duplex comprises one or two unpaired nucleosides at either or both ends, forming one or two overhang ends. In certain embodiments an overhang end is one or two nucleosides of the antisense oligomeric compound. In certain embodiments an overhang end is one or two 3′-nucleosides of the antisense oligomeric compound. In certain embodiments the last two 3′-nucleosides of the antisense oligomeric compound are overhang nucleosides not paired with the sense oligomeric compound. In certain embodiments the last one or two 3′-unpaired overhang nucleosides comprise an adenine nucleobase. In certain embodiments the last one or two 3′-unpaired overhang nucleosides comprise a thymine nucleobase. In certain embodiments the last one or two 3′-unpaired overhang nucleosides comprise a uridine nucleobase. In certain embodiments the last one or two 3′-unpaired overhang nucleosides comprise inosine nucleobase. In certain embodiments the last two 3′-unpaired overhang nucleosides comprise a thymine nucleobase and an inosine nucleobase. In certain embodiments the last two 3′-unpaired overhang nucleosides comprise 5′ to 3′ a thymine nucleobase and an inosine nucleobase. In certain embodiments the last two 3′-unpaired overhang nucleosides comprise 5′ to 3′ an inosine nucleobase and a thymine nucleobase. In certain embodiments the last two 3′-unpaired overhang nucleosides comprise 5′ to 3′ an adenine nucleobase and an inosine nucleobase. In certain embodiments the last two 3′-unpaired overhang nucleosides comprise 5′ to 3′ an inosine nucleobase and an adenine nucleobase.

In certain embodiments, an oligomeric duplex comprises a first oligomeric compound comprising a first modified oligonucleotide consisting of 19 to 25 linked nucleosides and a second oligomeric compound comprising a second modified oligonucleotide consisting of 16 to 24 linked nucleosides, wherein the nucleobase sequence of the first modified oligonucleotide and the nucleobase sequence of the second modified oligonucleotide each comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleosides of any of the following pairs selected from a first oligomeric compound selected from any one of SEQ ID NOs: 3-48, or 132-140 or 83-98, or 143-144 and a second oligomeric compound selected from any one of SEQ ID NOS: 52-82, or 141-142. In certain embodiments, the first oligomeric compound is an antisense agent. In certain embodiments, the first modified oligonucleotide is an antisense oligomeric compound. In certain embodiments, the second oligomeric compound is a sense agent. In certain embodiments, the second modified oligonucleotide is a sense oligomeric compound. In certain embodiments, the first modified oligonucleotide is an antisense RNAi oligonucleotide. In certain embodiments, the second oligomeric compound is a sense agent. In certain embodiments, the second modified oligonucleotide is a sense RNAi oligonucleotide.

In certain embodiments, an oligomeric duplex comprises a first oligomeric compound comprising a first modified oligonucleotide consisting of 21 or 23 linked nucleosides and a second oligomeric compound comprising a second modified oligonucleotide consisting of 19 or 21 linked nucleosides, wherein the sequences of the first modified oligonucleotide and second modified oligonucleotide consist of any of the following pairs of selected from a first oligomeric compound selected from any one of SEQ ID NOs: 5, 6, or 15-48, or 132-140 and a second oligomeric compound selected from any one of SEQ ID NOS: 52-82, or 141-142. In certain embodiments, the first oligomeric compound is an antisense agent. In certain embodiments, the first modified oligonucleotide is an antisense oligomeric compound. In certain embodiments, the second oligomeric compound is a sense agent. In certain embodiments, the second modified oligonucleotide is a sense oligomeric compound. In certain embodiments, the first modified oligonucleotide is an antisense RNAi oligonucleotide. In certain embodiments, the second oligomeric compound is a sense agent. In certain embodiments, the second modified oligonucleotide is a sense RNAi oligonucleotide. In certain embodiments, the oligomeric duplex comprises one or two unpaired nucleosides at either or both ends, forming one or two overhang ends. In certain embodiments an overhang end is one or two 3′-nucleosides of the antisense oligomeric compound. In certain embodiments the last two 3′-nucleosides of the antisense oligomeric compound are overhang nucleosides not paired with the sense oligomeric compound. In certain embodiments the last one or two 3′-unpaired overhang nucleosides comprise an adenine nucleobase. In certain embodiments the last one or two 3′-unpaired overhang nucleosides comprise a thymine nucleobase. In certain embodiments the last one or two 3′-unpaired overhang nucleosides comprise a uridine nucleobase. In certain embodiments the last one or two 3′-unpaired overhang nucleosides comprise an inosine nucleobase. In certain embodiments the antisense oligomeric compound comprises a 5′-terminal group. In certain embodiments the sense strand comprises a conjugate group attached at the 5′ or 3′ end of the sense oligomeric compound.

In any of the oligomeric duplexes described herein, at least one nucleoside of the first modified oligonucleotide and/or the second modified oligonucleotide comprises a modified sugar moiety. Examples of suitable modified sugar moieties include, but are not limited to, a bicyclic sugar moiety, such as a 2′-4′ bridge selected from —O—CH₂—; and —O—CH(CH₃)—, and a non-bicyclic sugar moiety, such as a 2′-MOE sugar moiety, a 2′-F sugar moiety, or a 2′-OMe sugar moiety. In certain embodiments, at least one nucleoside of the first modified oligonucleotide and/or the second modified oligonucleotide comprises a modified 2′-deoxyribosyl or 2′-deoxyxylosyl, sugar moiety. In certain embodiments, at least one nucleoside of the first modified oligonucleotide and/or the second modified oligonucleotide comprises a modified 3′-fluoro-hexitol sugar moiety. In certain embodiments, at least 80%, at least 90%, or 100% of the nucleosides of the first modified oligonucleotide and/or the second modified oligonucleotide comprises a modified sugar moiety independently selected from 2′-F, 2′-MOE, 2′-OMe, 2′-deoxyribosyl, 2′-deoxyxylosyl, and 3′-fluoro-hexitol. In certain embodiments, at least 80%, at least 90%, or 100% of the nucleosides of the first modified oligonucleotide and the second modified oligonucleotide comprises a modified sugar moiety independently selected from 2′-F, 2′-MOE, 2′-OMe, 2′-deoxyribosyl, and 2′-deoxyxylosyl.

In certain embodiments, in an oligomeric duplex provided herein, the first modified oligonucleotide the second modified oligonucleotide comprise a modified sugar moiety and/or sugar surrogate. In certain embodiments, in an oligomeric duplex provided herein, a sugar motif (from 5′ to 3′) of the first modified oligonucleotide comprising a modified sugar motif is paired with the second modified oligonucleotide comprising a sugar motif (from 5′ to 3′) selected from: efyyyyyyyyyyyfyfyyyyyee and yyyyyyyyfffyyeyyyyyyy; efyyyyyyyyyyyfyfyyyyyee and eeyyyyyyfffyyeyyyyyee; efyyyfyyyyyyyfyyyyyyyee and yyyyyyyyfffyyeyyyyyyy; efyyyfyyyyyyyfyyyyyyyee and eeyyyyyyfffyyeyyyyyee; efyyydyyeyyyydydyyyyyee and yyyyyyyyfffyyeyyyyyyy; efyyyfyyyyyyyfyyyyyyyee and eeyyyyyydffyyeyyyyyee; efyyyfyyyyyyyfyyyyyyyee and eeyyyyyyfdfyyeyyyyyee; efyyyfyyyyyyyfyyyyyyyee and eeyyyyyyffdyyeyyyyyee; efyyyfyyyyyyyfyyyyyyyee and eeyyyyyyhffyyeyyyyyee; efyyyfyyyyyyyfyyyyyyyee and eeyyyyyyfhfyyeyyyyyee; efyyyfyyyyyyyfyyyyyyyee and eeyyyyyyffhyyeyyyyyee; efyyyfyyyyyyyfyyyyyyyee and eeyyyyyyfxfyyeyyyyyee; efyyyfyyyyyyyfyyyyyyyee and eeyyyyyyffxyyeyyyyyee; efyyyfyyyyyyyfyyyyyyyee and eeyyyyyyxffyyeyyyyyee; efyyydyyeyyyydydyyyyyee and eeyyyyyyfhfyyeyyyyyee; efyyydyyeyyyydydyyyyyee and eeyyyyyyfdfyyeyyyyyee; efyyydyyeyyyydydyyyyyee and eeyyyyyyffhyyeyyyyyee; efyyydyyeyyyydydyyyyyee and eeyyyyyyffdyyeyyyyyee; efyyydyyeyyyydydyyyyyee and eeyyyyyydffyyeyyyyyee; efyyydyyeyyyydydyyyyyee and eeyyyyyyhffyyeyyyyyee; efyyydyyeyyyydydyyyyyee and eeyyyyyyxffyyeyyyyyee; efyyydyyeyyyydydyyyyyee and eeyyyyyyfxfyyeyyyyyee; efyyydyyeyyyydydyyyyyee and eeyyyyyyffxyyeyyyyyee; efyyyyyyeyyyyfyfyyyyyee and eeyyyyyyfffyyeyyyyyee; efyyyfyyeyyyyfyyyyyyyee and eeyyyyyyfffyyeyyyyyee; efyyxfyyyyyyyfyyyyyyyee and eeyyyyyyfffyyeyyyyyee; efyyyfxyyyyyyfyyyyyyyee and eeyyyyyyfffyyeyyyyyee; and efyyyxyyyyyyyfyyyyyyyee and eeyyyyyyfffyyeyyyyyee; wherein each ‘e’ represents a 2′-MOE sugar moiety, each ‘y’ represents a 2′-OMe sugar moiety, each ‘f’ represents a 2′-F sugar moiety, each ‘h’ represents a 3′-fluoro-hexitol sugar moiety, each ‘d’ represents a 2′-deoxyribose sugar moiety, and each ‘x’ represents a 2′-deoxyxylose sugar moiety; and wherein all except 0, 1, or 2 modifications independently on each of the first and second modified oligonucleotides are identical to the sugar motif.

In certain embodiments, in an oligomeric duplex provided herein, at least one internucleoside linkage of the first modified oligonucleotide and/or the second modified oligonucleotide comprises a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end ofthe second modified oligonucleotide comprises a phosphorothioate linkage.

In certain embodiments, in an oligomeric duplex provided herein, each internucleoside linkage of the first modified oligonucleotide is independently selected from a phosphodiester and a phosphorothioate, internucleoside linkage, and each internucleoside linkage of the second modified oligonucleotide is independently selected from a phosphodiester and a phosphorothioate, internucleoside linkage.

In certain embodiments, in an oligomeric duplex provided herein, at least one linkage of the antisense oligomeric compound is a modified linkage. In certain embodiments, in an oligomeric duplex provided herein, an internucleoside linkage of the first modified oligonucleotide is modified, wherein the 5′-most internucleoside linkage (i.e., linking the first nucleoside from the 5′-end to the second nucleoside from the 5′-end) is modified. In certain embodiments, in an oligomeric duplex provided herein, the internucleoside linkage motif (from 5′ to 3′) of the first modified oligonucleotide is selected from 5′-ssooooooooooooooooooss-3′, 5′-ssooosooooooososooooss-3′, and 5′-ssooosooooooooooooooss-3′, each “s” is a phosphorothioate internucleoside linkage and each “o” is a phosphodiester internucleoside linkage. In certain embodiments, in an oligomeric duplex provided herein, an internucleoside linkage of the second modified oligonucleotide is modified, wherein the 5′-most internucleoside linkage (i.e., linking the first nucleoside from the 5′-end to the second nucleoside from the 5′-end) is modified. In certain embodiments, in an oligomeric duplex provided herein, the internucleoside linkage motif (from 5′ to 3′) of the second modified oligonucleotide is selected from (from 5′ to 3′) of: 5′-ssooooooooooooooooss-3′, 5′-ssooooooosooooooooss-3′, 5′-ssoooooosoooooooooss-3′, and 5′-ssoooooooosoooooooss-3′, wherein each ‘o’ represents a phosphodiester internucleoside linkage and each ‘s’ represents a phosphorothioate internucleoside linkage. In certain embodiments, the two 5′-most internucleoside linkages are modified. In certain embodiments, the first one or 2 internucleoside linkages from the 3′-end are modified. In certain embodiments, the modified internucleoside linkage is a phosphorothioate linkage.

In certain embodiments, in an oligomeric duplex provided herein, at least one nucleobase of the first modified oligonucleotide and/or at least one nucleobase of the second modified oligonucleotide is a modified nucleobase. In certain embodiments, the modified nucleobase is methylcytosine. In certain embodiments, the modified nucleobase 15 inosine.

In certain embodiments, in an oligomeric duplex provided herein, the first oligomeric compound comprises a terminal group comprising a stabilized phosphate group attached to the 5′ position of the 5′-most nucleoside. In certain embodiments, the stabilized phosphate group comprises a cyclopropyl phosphonate or an (E)-vinyl phosphonate. In certain embodiments, the stabilized phosphate group is an (E)-vinyl phosphonate.

In certain embodiments, in an oligomeric duplex provided herein, the first modified oligonucleotide optionally is attached to a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group is attached to the first modified oligonucleotide at an internal position. In certain embodiments, the conjugate group is attached to the first modified oligonucleotide through a 2′-modification of a furanosyl sugar moiety. In certain embodiments, the conjugate group is attached to the first modified oligonucleotide through a modified internucleoside linkage. In certain embodiments, the conjugate group comprises N-acetyl galactosamine.

In any of the oligomeric duplexes described herein, the second modified oligonucleotide optionally is attached to a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group is attached to the second modified oligonucleotide at an internal position. In certain embodiments, the conjugate group is attached to the second modified oligonucleotide through a 2′-modification of a furanosyl sugar moiety. In certain embodiments, the conjugate group is attached to the second modified oligonucleotide through a modified internucleoside linkage. In certain embodiments, the conjugate group comprises N-acetyl galactosamine.

In certain embodiments, an oligomeric duplex comprises an oligomeric compound, which is an antisense agent described herein. In certain embodiments, an antisense agent, which is an oligomeric duplex described herein, is an RNAi agent capable of reducing the amount of LPA RNA through the activation of RISC/Ago2.

In certain embodiments, an oligomeric agent comprises at least two oligomeric duplexes linked together. In certain embodiments, an oligomeric agent comprises two oligomeric duplexes wherein at least one oligomeric duplex is targeted to LPA RNA as described herein. In certain embodiments, an oligomeric agent comprises two or more of the same oligomeric duplex, which is any of the oligomeric duplexes described herein. In certain embodiments, the two or more oligomeric duplexes are covalently linked together. In certain embodiments, the second modified oligonucleotides of the two or more oligomeric duplexes are covalently linked together. In certain embodiments, the second modified oligonucleotides of two or more oligomeric duplexes are covalently linked together at their 3′ ends. In certain embodiments, the second modified oligonucleotides of two or more oligomeric duplexes are covalently linked together at the 3′ end of one to the 5′ end of the other. In certain embodiments, the two or more oligomeric duplexes are covalently linked together by a glycol linker, such as a tetraethylene glycol linker. A structure of oligomeric duplexes covalently linked by a glycol linker is described in, e.g., Alterman, et al., Nature Biotech., 37:844-894, 2019. In certain embodiments, a first modified oligonucleotide of a first oligomeric duplex is covalently linked to a second modified oligonucleotide of a second oligomeric duplex and a first modified oligonucleotide of the second oligomeric duplex is covalently linked to a second modified oligonucleotide of the first oligomeric duplex (see, e.g., PCT International Patent Application Publication WO2020/065602 for a description of an example of a structure of linked oligomeric duplexes).

### V. Methods and Uses

### 1. Antisense Activity

In certain embodiments, oligomeric duplexes provided herein comprise an oligomeric compound that is capable of hybridizing to a target nucleic acid, resulting in at least one antisense activity; such oligomeric duplexes and oligomeric compounds are antisense agents.

In certain antisense activities, hybridization of an antisense oligomeric compound to a target nucleic acid results in recruitment of a protein that cleaves the target nucleic acid. For example, in certain antisense activities, an antisense agent or a portion of an antisense agent is loaded into an RNA-induced silencing complex (RISC), ultimately resulting in cleavage of the target nucleic acid. For example, certain antisense agents result in cleavage of the target nucleic acid by Argonaute. Antisense agents that are loaded into RISC are RNAi agents. RNAi agents may be double-stranded (siRNA or dsRNAi) or single-stranded (ssRNA). In certain embodiments, RNAi agents are capable of RISC-mediated modulation of a target nucleic acid in a cell. In certain embodiments, such compounds reduce or inhibit the amount or activity of a target nucleic acid by 25% or more in the standard in vitro assay. In certain embodiments, RNAi agents selectively affect one or more target nucleic acid. Such RNAi agents comprise a nucleobase sequence that hybridizes to one or more target nucleic acid, resulting in one or more desired antisense activity. In certain embodiments, an RNAi agent does not hybridize to one or more non-target nucleic acid or does not hybridize to one or more non-target nucleic acid in such a way that results in significant undesired antisense activity.

In certain embodiments, hybridization of an antisense agent to a target nucleic acid results in inhibition of a binding interaction between the target nucleic acid and a protein or other nucleic acid (e.g., miRNA, lncRNA, sncRNA). In certain embodiments, hybridization of an antisense agent to a target nucleic acid results in modulation of translation of the target nucleic acid. In certain embodiments, hybridization of an oligomeric compound to a target nucleic acid results in an increase in the amount or activity of a target nucleic acid. In certain embodiments, hybridization of an antisense oligonucleotide to a target nucleic acid results in increased translation of the target nucleic acid. In certain embodiments, hybridization of an antisense oligonucleotide to a target nucleic acid results in reduced translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of a nucleic acid or protein and/or a phenotypic change in a cell or animal.

Certain embodiments provide compositions and methods for reducing LPA RNA levels. Certain embodiments provide compositions and methods for reducing apo(a) and/or Lp(a) levels. In certain embodiments, reducing apo(a) levels in a tissue, organ and/or subject improves the ratio of LDL to HDL or the ratio of TG to HDL. In certain embodiments, provided are methods for using an oligomeric antisense agent targeted to an apo(a) nucleic acid for modulating the expression of apo(a) in a subject. In certain embodiments, expression of apo(a) is reduced.

In certain embodiments, inhibition of apo(a) or Lp(a) expression occurs in a cell, tissue, or organ. In certain embodiments, inhibition of apo(a) or Lp(a) occurs in a cell, tissue, or organ in a subject. In certain embodiments, inhibition is a reduction in apo(a) mRNA level. In certain embodiments, inhibition is a reduction in apo(a) protein level. In certain embodiments, both apo(a) mRNA and protein levels are reduced. In certain embodiments, inhibition is a reduction in Lp(a) level. Such reduction may occur in a time-dependent or in a dose-dependent manner.

In certain embodiments, a method of modulating expression of LPA or modulating apo(a) protein or Lp(a) in a cell comprises contacting the cell with an oligomeric duplex comprising or consisting of an oligomeric compound comprising a modified oligonucleotide having a targeting region complementary to a target region of a LPA nucleic acid. In certain embodiments, a method of inhibiting expression of LPA or inhibiting apo(a) protein or Lp(a) protein in a cell comprises contacting the cell with an oligomeric duplex comprising or consisting of an oligomeric compound comprising a modified oligonucleotide having a nucleobase sequence complementary to a target region of a LPA nucleic acid. In certain embodiments, the cell is a hepatocyte.

In certain embodiments, provided herein are methods of inhibiting or reducing LPA expression, LPA RNA levels and/or apo(a) protein or Lp(a) levels and/or activity, in a subject having, or at risk of having, a disease, disorder, condition or injury associated with LPA and/or apo(a) protein or Lp(a), such as a disease, disorder, condition or injury associated with inflammatory cardiovascular disease, wherein the method includes administering to the subject an oligomeric duplex comprising or consisting of an oligomeric antisense agent comprising a modified oligonucleotide targeting a LPA nucleic acid thereby inhibiting expression of LPA nucleic acid in the subject. In certain embodiments, expression of LPA nucleic acid is inhibited. In certain embodiments, administering an oligomeric duplex inhibits LPA expression, LPA RNA levels and/or apo(a) protein or Lp(a) levels and/or activity in the plasma/serum blood. In certain embodiments, administering an oligomeric duplex inhibits or reduces LPA expression, LPA RNA levels, and/or apo(a) protein or Lp(a) levels, and/or activity in the liver. In certain embodiments, administering such an oligomeric duplex inhibits or reduces LPA expression, LPA RNA levels and/or apo(a) protein or Lp(a) levels and/or activity in the plasma/serum blood and the liver of the subject. In certain embodiments, detectable amount of the LPA RNA may be reduced by at least 10%, at least 20%, at least 30%, at least 40%, at least 50%, at least 60%, at least 70%, at least 80%, or at least 90%. In certain embodiments, an oligomeric compound comprising or consisting of a modified oligonucleotide comprising a targeting region complementary to a target region of SEQ ID NO: 1 or SEQ ID NO: 2 is capable of decreasing or reducing a detectable amount of a apo(a) protein or Lp(a) in a cell, organ or tissue, e.g., the liver of the subject, when the compound is administered to the cell, a tissue, and/or subject. In certain embodiments, detectable amount of the apo(a) protein or Lp(a) may be reduced by at least 10%, at least 20%, at least 30%, at least 40%, at least 50%, at least 60%, at least 70%, at least 80%, or at least 90%.

In some embodiments an oligomeric duplex comprising or consisting of an oligomeric antisense agent has LPA RNA and/or apo(a) protein or Lp(a) reduction activity, and, in particular embodiments, liver LPA RNA and/or apo(a) protein or Lp(a) reduction activity, that is comparable to or greater than the LPA RNA and/or apo(a) protein or Lp(a) reduction activity of a comparator agent. In certain embodiments, the comparator agent is an agent that comprises a comparator modified oligonucleotide having a targeting region complementary to a target region of LPA. In certain embodiments, the comparator modified oligonucleotide is complementary to the same or a similar target region as the modified oligonucleotide of the oligomeric compound. In certain embodiments, the comparator modified oligonucleotide is complementary to a different target region from the modified oligonucleotide of the oligomeric compound. In some embodiments, the amount of LPA RNA is reduced by at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% in a cell (e.g., hepatocyte), organ (e.g., livery), or subject (e.g., animal) that has been contacted with or administered an oligomeric duplex provided herein (or a composition comprising such an oligomeric duplex) compared to a control (e.g., a cell, organ, tissue, system or subject that has not been contacted with or administered the oligomeric duplex, or was contacted with or administered a control substance (e.g., PBS)). In some embodiments, the percentage of LPA RNA decrease or reduction in a cell (e.g., a hepatocyte), organ (e.g., a livery), tissue, system, or subject (e.g., animal) contacted with or administered an oligomeric duplex or composition provided herein is 0.1% to 30% greater than or less than, 0.1% to 25% greater than or less than, 0.10% to 20% greater than or less than, 0.1% to 15% greater than or less than, 0.1% to 10% greater than or less than, or 0.1% to 5% greater than or less than, 0.1% to 1% greater than or less than, 5% to 40% greater than, 5% to 35% greater than, 10% to 40% greater than, at least 5% greater than, at least 10% greater than, at least 15% greater than, at least 20% greater than, at least 25% greater than, or at least 30% greater than the percentage of LPA RNA decrease or reduction in a cell (e.g., a cardiomyocyte), organ (e.g., a heart), tissue, system or subject (e.g., animal) contacted with or administered the same concentration or dose of a comparator agent.

### B. Therapeutic Indications, Methods

Atherosclerotic cardiovascular disease is highly prevalent and continues to be the highest cause of mortality worldwide despite the widespread use of low-density lipoprotein (LDL)-lowering therapies. Though LDL-lowering therapies reduce the risk of major cardiac events, residual cardiovascular risk encountered in some patients with low LDL levels implies other mechanisms of cardiovascular pathology. Over the last decade, compelling evidence from epidemiological studies and meta-analyses, Mendelian randomization studies, and genome wide association studies have shown that elevated serum Lp(a) concentration is associated with a higher risk of coronary artery disease and atherosclerosis-related disorders (Clarke et al., N. Engl. J. Med., Vol. 361:2518-2528, 2009; Kamstrup etal., JAMA, Vol. 301:2331-2339, 2009; Nordestgaard et al., European Heart Journal, Vol. 31:2844-2853, 2010; Helgadottir et al., J. Am. Coll. Cardiol, Vol. 60:722-729, 2012; Thanassoulis etal., J. Am. Coll. Cardiol., Vol. 55:2491-2498, 2010; Kamstrup et al., J. Am. Coll. Cardiol., Vol. 63:470-477, 2014; Kral et al., Journal of Cardiology, Vol. 118:656-661, 2016; Thanassoulis et al., J. Lipid Res., Vol. 57: 917-924, 2016; Tsimikas et al., J. Am. Coll. Cardiol., Vol. 69:692-711, 2017). It has been noted that this risk relationship is continuous and becomes proportionally more impactful with higher Lp(a) levels, and the association persists after correction for other lipid parameters (Emerging Risk Factors Collaboration, JAMA, Vol. 302:412-423, 2009). LPA gene mutations can increase LPA RNA levels, result in abnormal apo(a) cleavage that leads to increased apo(a) levels (and Lp(a) levels), or decreased degradation and clearance, and/or abnormal interactions between Lp(a) and other proteins or other endogenous or exogenous substances (e.g., plasminogen receptor) such that Lp(a) level is increased, or degradation is decreased. A condition associated with high Lp(a) levels may be relatively insensitive to lifestyle changes and common statin drugs, and therefore hard to treat.

Subjects with high Lp(a) levels are at a significant risk of disease (Lippi et al., Clinica Chimica Acta, 2011, 412:797-801; Solfrizz et al.). For example, subjects will Lp(a) levels greater than ≥75 nanomoles/liter (nmol/L) or ≥30 mg/dL are considered to have increased risk for various diseases. In many subjects with high Lp(a) levels, current treatments cannot reduce Lp(a) levels to safe levels. Apo(a) plays a key role in the formation of Lp(a), hence reducing apo(a) can reduce Lp(a) and prevent, treat, or ameliorate a disease associated with Lp(a). In certain embodiments, treatment with oligomeric duplexes and methods provided herein is indicated for a subject with elevated apo(a) levels and/or Lp(a) levels. In certain embodiments, the subject has apo(a) levels≥10 mg/dL, ≥20 mg/dL, ≥30 mg/dL, ≥40 mg/dL, ≥50 mg/dL, ≥60 mg/dL, ≥70 mg/dL, ≥80 mg/dL, ≥90 mg/dL or ≥100 mg/dL. In certain embodiments, the subject has Lp(a) levels≥10 mg/dL, ≥15 mg/dL, ≥20 mg/dL, ≥25 mg/dL, ≥30 mg/dL, ≥35 mg/dL, ≥40 mg/dL, ≥50 mg/dL, ≥60 mg/dL, ≥70 mg/dL, ≥80 mg/dL, ≥90 mg/dL, ≥100 mg/dL, ≥110 mg/dL, ≥120 mg/dL, ≥130 mg/dL, ≥140 mg/dL, ≥150 mg/dL, ≥160 mg/dL, ≥170 mg/dL, ≥175 mg/dL, ≥180 mg/dL, ≥190 mg/dL, ≥200 mg/dL. In certain embodiments, a subject has apo(a) level greater than the upper limit of normal, e.g. wherein the subject has apo(a) levels≥30 mg/dL, ≥35 mg/dL, ≥40 mg/dL, ≥50 mg/dL, ≥60 mg/dL, ≥70 mg/dL, ≥80 mg/dL, ≥90 mg/dL, ≥100 mg/dL, ≥110 mg/dL, ≥120 mg/dL, ≥130 mg/dL, ≥140 mg/dL, ≥150 mg/dL, ≥160 mg/dL, ≥170 mg/dL, ≥175 mg/dL, ≥180 mg/dL, ≥190 mg/dL, ≥200 mg/dL.

In certain embodiments, provided herein are methods for preventing, treating, or delaying or preventing the development or progression of, diseases, disorders, conditions or injuries associated with LPA and/or apo(a) protein or Lp(a) increased levels, wherein the method comprises administering to a subject an oligomeric duplex described herein (e.g., an antisense oligomeric compound comprising or consisting of a modified oligonucleotide comprising a targeting region complementary to a target region of a LPA nucleic acid). Also provided are methods of ameliorating, preventing, or delaying the onset of, one or more symptoms associated with diseases, disorders, conditions or injuries associated with LPA or apo(a) protein and/or Lp(a), wherein the method comprises administering to a subject an oligomeric compound comprising or consisting of a modified oligonucleotide having a nucleobase sequence complementary to a nucleobase sequence in a LPA nucleic acid.

A Lp(a)-associated condition may be, e.g., a cardiovascular condition, a metabolic condition, an inflammatory condition. In certain embodiments an Lp(a)-associated condition is an inflammatory, cardiovascular, or metabolic disease or disorder. In certain embodiments, an Lp(a) associated condition or lipoprotein metabolism disorder is a cardiometabolic disorder. In some embodiments, an oligomeric duplex comprising or consisting of an oligomeric antisense agent described herein is used to treat a subject with a cardiovascular disease (CVD) such as hypertriglyceridemia and/or other condition selected from lipidemia (e.g., hyperlipidemia), dyslipidemia (e.g., atherogenic dyslipidemia, diabetic dyslipidemia, or mixed dyslipidemia), hyperlipoproteinemia, hyperapobetalipoproteinemia, chronic heart disease, including coronary artery disease (CAD) or any symptoms or conditions associated with a CVD, e.g., hypercholesterolemia (e.g., statin-resistant hypercholesterolemia, heterozygous or homozygous familial hypercholesterolemia) myocardial infarction (MI), acute coronary syndrome, mesenteric ischemia, superior mesenteric artery occlusion, restenosis, renal artery stenosis, angina peripheral arterial disease (PAD), calcific aortic valve disease (CAVD), aortic stenosis, aortic valve regurgitation, aortic dissection, retinal artery occlusion, atherosclerotic cardiovascular disease (ASCVD), atherosclerosis, dyslipidemia, thrombosis, cerebrovascular atherosclerosis, cerebrovascular disease, or stroke.

Thus, in certain embodiments, a method comprises administering to a subject an oligomeric duplex comprising or consisting of an oligomeric antisense agent comprising a targeting region complementary to a target region of a LPA nucleic acid. In certain embodiments, the subject has or is at risk for developing a cardiovascular, metabolic and/or inflammatory injury, disease, condition, or disorder. In certain embodiments, the subject has or is at risk for developing cardiovascular disease (CVD) such as hypertriglyceridemia and/or other condition selected from lipidemia (e.g., hyperlipidemia), dyslipidemia (e.g., atherogenic dyslipidemia, diabetic dyslipidemia, or mixed dyslipidemia), hyperlipoproteinemia, hyperapobetalipoproteinemia, chronic heart disease, including coronary artery disease (CAD) or any symptoms or conditions associated with a CVD, e.g., hypercholesterolemia (e.g., statin-resistant hypercholesterolemia, heterozygous or homozygous familial hypercholesterolemia) myocardial infarction (MI), acute coronary syndrome, mesenteric ischemia, superior mesenteric artery occlusion, restenosis, renal artery stenosis, angina peripheral arterial disease (PAD), calcific aortic valve disease (CAVD), aortic stenosis, aortic valve regurgitation, aortic dissection, retinal artery occlusion, atherosclerotic cardiovascular disease (ASCVD), atherosclerosis, dyslipidemia, thrombosis, cerebrovascular atherosclerosis, cerebrovascular disease, or stroke. In certain embodiments, at least one symptom of the cardiovascular injury, disease, condition, or disorder is ameliorated. In certain embodiments, the at least one symptom is selected from include, but are not limited to, angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen, and fever. In certain embodiments, administration of an oligomeric duplex comprising or consisting of an oligomeric antisense agent provided herein to the subject reduces or delays the onset or progression of at least one at least one symptom of aortic stenosis.

Certain embodiments provide compositions and methods for preventing, delaying, slowing the progression of apo(a) related diseases, disorders, and conditions in a subject in need thereof. Certain embodiments provide compositions and methods for ameliorating apo(a) related diseases, disorders, and conditions in a subject in need thereof. Certain embodiments provide compositions and methods for preventing, delaying, slowing the progression of Lp(a) related diseases, disorders, and conditions in a subject in need thereof. Certain embodiments provide compositions and methods for ameliorating Lp(a) related diseases, disorders, and conditions in a subject in need thereof. In certain embodiments, such diseases, disorders, and conditions include inflammatory, cardiovascular and/or metabolic diseases, disorders, and conditions.

In certain embodiments, provided are methods of treating an individual with an apo(a) related disease, disorder or condition comprising administering a therapeutically effective amount of one or more pharmaceutical compositions as described herein. In certain embodiments, the individual has elevated apo(a) levels. In certain embodiments, provided are methods of treating an individual with an Lp(a) related disease, disorder or condition comprising administering a therapeutically effective amount of one or more pharmaceutical compositions as described herein. In certain embodiments, the individual has elevated Lp(a) levels. In certain embodiments, the individual has an inflammatory, cardiovascular and/or metabolic disease, disorder, or condition. In certain embodiments, administration of a therapeutically effective amount of an oligomeric duplex is accompanied by monitoring of apo(a) or Lp(a) levels. In certain embodiments, administration of an oligomeric duplex is accompanied by monitoring of markers of inflammatory, cardiovascular and/or metabolic disease, or other disease process associated with the expression of apo(a), to determine an individual's response to an oligomeric duplex. An individual's response to administration of an oligomeric duplex can be used by a physician to determine the amount and duration of therapeutic intervention with an oligomeric duplex.

In certain embodiments, provided herein are methods of treating a subject comprising administering one or more pharmaceutical compositions as described herein. In certain embodiments, a method of treating comprises administering an oligomeric duplex for inhibiting or reducing expression of LPA nucleic acid, such as RNA, in a subject having or at risk of a disease, injury, condition or disorder associated with LPA, comprising administering to the subject an oligomeric duplex comprising or consisting of a modified oligonucleotide having a targeting region complementary to a target region of a LPA nucleic acid, thereby inhibiting or reducing expression of LPA nucleic acid in the subject. In certain embodiments, the individual has an apo(a) related disease. In certain embodiments, the individual has an Lp(a) related disease. In certain embodiments, the individual has an inflammatory, cardiovascular and/or a metabolic disease, disorder, or condition.

In certain embodiments, a method of treating a subject comprises administering to a subject an oligomeric duplex comprising or consisting of an oligomeric antisense agent comprising or consisting of a modified oligonucleotide having a targeting region complementary to a target region of a LPA nucleic acid, thereby treating the subject. In certain embodiments, the subject has or is at risk for developing cardiovascular, metabolic, and/or inflammatory disease or disorder. In certain embodiments, administering the therapeutically effective amount of the oligomeric duplex improves one or more symptoms of the cardiovascular, metabolic, and/or inflammatory disease or disorder in the subject. In certain embodiments, at least one symptom of the cardiovascular, metabolic, and/or inflammatory disease or disorder is ameliorated. In certain embodiments, administration of a pharmaceutical composition comprising an oligomeric duplex to the subject reduces or delays the onset or progression of at least one or more symptoms.

In certain embodiments cardiovascular diseases, disorders or conditions include, but are not limited to, aortic stenosis, aneurysm (e.g., abdominal aortic aneurysm), angina, arrhythmia, atherosclerosis, cerebrovascular disease, coronary artery disease, coronary heart disease, dyslipidemia, hypercholesterolemia, hyperlipidemia, hypertension, hypertriglyceridemia, myocardial infarction, peripheral vascular disease (e.g., peripheral artery disease, peripheral artery occlusive disease), retinal vascular occlusion, stroke, elevated Lp(a) associated CVD risk, recurrent cardiovascular events with elevated Lp(a), calcific aortic valve stenosis associated with high Lp(a), and the like. Certain embodiments provide compositions and methods for preventing, delaying, slowing the progression of aortic stenosis. Certain embodiments provide compositions and methods for ameliorating aortic stenosis.

In certain embodiments, an oligomeric duplex modulates physiological markers or phenotypes of a cardiovascular disease, disorder, or condition. For example, administration of an oligomeric duplex to a human can decrease Lp(a), LDL and cholesterol levels compared to untreated subjects. In certain embodiments, modulation of the physiological markers or phenotypes can be associated with inhibition of apo(a) by an oligomeric duplex. In certain embodiments, physiological markers of the cardiovascular disease, disorder or condition can be quantifiable. For example, Lp(a), LDL or cholesterol levels can be measured and quantified by, for example, standard lipid tests. For such markers, in certain embodiments, the marker can be decreased by about 5, 10, 15, 20, 25, 30, 35, 40, 45, 50, 55, 60, 65, 70, 75, 80, 85, 90, 95 or 99%, or a range defined by any two of these values.

Provided herein are methods for treating a symptom associated with a cardiovascular disease, disorder, or condition in a subject in need thereof. disease, disorder. In certain embodiments, treating results in reducing the rate of onset of a symptom associated with the cardiovascular disease, disorder, or condition. In certain embodiments, treating results in reducing the severity of a symptom associated with the cardiovascular or condition. A cardiovascular disease, disorder or condition can be characterized by numerous physical symptoms. Any symptom known to one of skill in the art to be associated with the cardiovascular disease, disorder or condition can be prevented, treated, ameliorated, or otherwise modulated with the compounds and methods described herein. In certain embodiments, the symptom can be any of, but not limited to, angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen or fever.

In certain embodiments, a method of treatment of a subject comprises administering to the subject an oligomeric duplex comprising or consisting of an oligomeric antisense agent having a targeting region complementary to a target region of a LPA nucleic acid, thereby treating the subject. In certain embodiments, the subject has or is at risk for developing a cardiovascular disease, e.g., an atherosclerotic cardiovascular disease, an atherosclerotic cerebrovascular disease, a hyperlipidaemia, wherein the disease is associated with elevated levels of Lp(a)-containing particles. In certain embodiments, treatment is to prevent and/or reduce the risk of suffering from and/or treat stroke, atherosclerosis, thrombosis calcific aortic stenosis such as calcific aortic stenosis, ischaemic stroke, coronary artery disease, peripheral arterial disease, abdominal aortic aneurysm, heart failure secondary to ischaemic cardiomyopathy, or familial hypercholesterolaemia, wherein the disease is associated with elevated levels of Lp(a)-containing particles.

In certain embodiments treatment with an oligomeric duplex is to prevent and/or reduce the risk of suffering from and/or treat or cardiovascular disease such as coronary heart disease and any other disease or pathology associated with elevated levels of Lp(a)-containing particles. In certain embodiments treatment is to prevent and/or reduce a risk of suffering from and/or treat, a cardiovascular disease, e.g., an atherosclerotic cardiovascular disease, an atherosclerotic cerebrovascular disease, a hyperlipidaemia, wherein the disease is associated with elevated levels of Lp(a)-containing particles. In certain embodiments, treatment is to prevent and/or reduce the risk of suffering from and/or treat stroke, atherosclerosis, thrombosis calcific aortic stenosis such as calcific aortic stenosis, ischaemic stroke, coronary artery disease, peripheral arterial disease, abdominal aortic aneurysm, heart failure secondary to ischaemic cardiomyopathy, or familial hypercholesterolaemia, wherein the disease is associated with elevated levels of Lp(a)-containing particles.

In certain embodiments treatment with an oligomeric duplex results in reducing the risk of a cardiovascular event in a patient with atherosclerotic cardiovascular disease. In certain embodiments the cardiovascular event is cardiovascular death, myocardial infarction, stroke, and/or coronary revascularization. In certain embodiments, a subject has a history of coronary revascularization, a history of coronary artery bypass grafting, a diagnosis of coronary artery disease, a diagnosis of atherosclerotic cerebrovascular disease, a diagnosis of peripheral artery disease, and/or a history of myocardial infarction. In certain embodiments, administration of a pharmaceutical composition comprising an oligomeric duplex comprising or consisting of an oligomeric antisense agent to the subject reduces or delays the onset or progression of at least one of stroke, atherosclerosis, thrombosis calcific aortic stenosis such as calcific aortic stenosis, ischaemic stroke, coronary artery disease, peripheral arterial disease, abdominal aortic aneurysm, heart failure secondary to ischaemic cardiomyopathy, or familial hypercholesterolaemia. In certain embodiments, administration of a pharmaceutical composition comprising an oligomeric duplex comprising or consisting of an oligomeric antisense agent to the subject reduces or delays the onset or progression of at least one of angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen, and fever, or a combination thereof.

In certain embodiments treatment with an oligomeric duplex is to ameliorate one or more symptoms of cardiovascular disease such as coronary heart disease and/or other disease or pathology associated with elevated levels of Lp(a) containing particles. In certain embodiments treatment is to ameliorate symptoms associated with a cardiovascular disease such as coronary artery disease, carotid artery disease, peripheral artery disease, myocardial infarction, cerebrovascular disease, stroke, aortic valve stenosis, stable or unstable angina, atrial fibrillation, heart failure, hyperlipidemia, heterozygous familial hypercholesterolemia, or homozygous familial hypercholesterolemia. In certain embodiments a subject is diagnosed with or at risk of a cardiovascular disease; diagnosed myocardial infarction, acute coronary syndrome. In certain embodiments, the symptoms include, but are not limited to, angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen, and fever. Certain embodiments provide a method of reducing at least one symptom of aortic stenosis.

In certain embodiments, administration of a pharmaceutical composition comprising an oligomeric duplex comprising or consisting of an oligomeric antisense agent to the subject ameliorates or reduces one or more symptoms of at least one of stroke, atherosclerosis, thrombosis calcific aortic stenosis such as calcific aortic stenosis, ischaemic stroke, coronary artery disease, peripheral arterial disease, abdominal aortic aneurysm, heart failure secondary to ischaemic cardiomyopathy, or familial hypercholesterolaemia. In certain embodiments, administration of a pharmaceutical composition comprising an oligomeric duplex comprising or consisting of an oligomeric antisense agent to the subject ameliorates one or more symptoms of angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen, and fever, or a combination thereof.

In certain embodiments, metabolic diseases, disorders, or conditions include, but are not limited to, hyperglycemia, prediabetes, diabetes (type I and type II), obesity, insulin resistance, metabolic syndrome, and diabetic dyslipidemia.

In certain embodiments, an oligomeric duplex modulates physiological markers or phenotypes of a metabolic disease, disorder, or condition. For example, administration of an oligomeric duplex to humans can decrease glucose and insulin resistance levels in those subjects compared to untreated subjects. In certain embodiments, the modulation of the physiological markers or phenotypes can be associated with inhibition of apo(a) by an oligomeric duplex. In certain embodiments, physiological markers of the metabolic disease, disorder or condition can be quantifiable. For example, glucose levels or insulin resistance can be measured and quantified by standard tests known in the art. For such markers, in certain embodiments, the marker can be decreased by about 5, 10, 15, 20, 25, 30, 35, 40, 45, 50, 55, 60, 65, 70, 75, 80, 85, 90, 95 or 99%, or a range defined by any two of these values. In another example, insulin sensitivity can be measured and quantified by standard tests known in the art. For such markers, in certain embodiments, the marker can be increase by about 5, 10, 15, 20, 25, 30, 35, 40, 45, 50, 55, 60, 65, 70, 75, 80, 85, 90, 95 or 99%, or a range defined by any two of these values.

Provided herein are methods for treating a symptom associated with the metabolic disease, disorder, or condition in a subject in need thereof. In certain embodiments, treating results in reducing the rate of onset of a symptom associated with the metabolic disease, disorder, or condition. In certain embodiments, treating results in reducing the severity of a symptom associated with the metabolic disease, disorder, or condition. A metabolic disease, disorder or condition can be characterized by numerous physical symptoms. Any symptom known to one of skill in the art to be associated with the metabolic disease, disorder or condition can be prevented, treated, ameliorated, or otherwise modulated with the compounds and methods described herein. In certain embodiments, a symptom can be any of, but not limited to, excessive urine production (polyuria), excessive thirst and increased fluid intake (polydipsia), blurred vision, unexplained weight loss and lethargy.

In certain embodiments, inflammatory diseases, disorders or conditions may overlap with cardiometabolic disorders or conditions, and include, but are not limited to, elevated Lp(a) associated CVD risk, recurrent cardiovascular events with elevated Lp(a), aortic stenosis (e.g., calcific aortic valve stenosis associated with high Lp(a)), coronary artery disease (CAD), Alzheimer's Disease and thromboembolic diseases, disorder or conditions. Certain thromboembolic diseases, disorders or conditions include, but are not limited to, stroke, thrombosis, myocardial infarction, and peripheral vascular disease.

In certain embodiments, an oligomeric duplex modulates physiological markers or phenotypes of an inflammatory disease, disorder, or condition. For example, administration of an oligomeric duplex to a human can decrease inflammatory cytokine or other inflammatory markers levels in compared to untreated subjects. In certain embodiments, the modulation of the physiological markers or phenotypes can be associated with inhibition of apo(a) by an oligomeric duplex. In certain embodiments, physiological markers of the inflammatory disease, disorder or condition can be quantifiable. For example, cytokine levels can be measured and quantified by standard tests known in the art. For such markers, in certain embodiments, the marker can be decreased by at least about 5%, 10%, 15%, 20%, 25%, 30%, 35%, 40%, 45%, 50%, 55%, 60%, 65%, 70%, 75%, 80%, 85%, 90%, 95% or 99%, or a range defined by any two of these values.

Provided herein are methods for treating a symptom associated with an inflammatory disease, disorder, or condition in a subject in need thereof. In certain embodiments, treating results in reducing the rate of onset of a symptom associated with the inflammatory disease, disorder, or condition. In certain embodiments, treating results in reducing the severity of a symptom associated with the inflammatory disease, disorder, or condition.

In certain embodiments, an oligomeric duplex has greater LPA RNA and/or protein reduction activity (i.e., greater specificity of action) in a target cell/organ/tissue/system (e.g., hepatocytes, liver) LPA than a non-target (e.g., plasminogen). For example, in some embodiments, administration of an oligomeric duplex provided herein reduces the amount or activity of LPA RNA and/or protein at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to a control and has no, or a non-significant, effect on (e.g., reduction in) the amount or activity of plasminogen.

In certain embodiments, administration of an oligomeric duplex results in reduction of LPA expression by at least about 15%, 20%, 25%, 30%, 35%, 40%, 45%, 50%, 55%, 60%, 65%, 70%, 75%, 80%, 85%, 90%, 95% or 99%, or a range defined by any two of these values. In certain embodiments, apo(a) expression is reduced to at least ≤100 mg/dL, ≤90 mg/dL, ≤80 mg/dL, ≤70 mg/dL, ≤60 mg/dL, ≤50 mg/dL, ≤40 mg/dL, ≤30 mg/dL, ≤20 mg/dL or ≤10 mg/dL. In certain embodiments, administration of an oligomeric duplex results in reduction of apo(a) by at least about 15%, 20%, 25%, 30%, 35%, 40%, 45%, 50%, 55%, 60%, 65%, 70%, 75%, 80%, 85%, 90%, 95% or 99%, or a range defined by any two of these values. In certain embodiments, Lp(a) containing particles in serum is reduced to at least ≤200 mg/dL, ≤190 mg/dL, ≤180 mg/dL, ≤175 mg/dL, ≤170 mg/dL, ≤160 mg/dL, ≤150 mg/dL, ≤140 mg/dL, ≤130 mg/dL, ≤120 mg/dL, ≤110 mg/dL, ≤100 mg/dL, ≤90 mg/dL, ≤80 mg/dL, ≤70 mg/dL, ≤60 mg/dL, ≤55 mg/dL, ≤50 mg/dL, ≤45 mg/dL, ≤40 mg/dL, ≤35 mg/dL, ≤30 mg/dL, ≤25 mg/dL, ≤20 mg/dL, ≤15 mg/dL, or ≤10 mg/dL.

In certain embodiments, provided are methods for using an oligomeric duplex in the preparation of a medicament. In certain embodiments, pharmaceutical compositions comprising an oligomeric duplex are used for the preparation of a medicament for treating a patient suffering or susceptible to an inflammatory, cardiovascular and/or a metabolic disease, disorder, or condition. In certain embodiments, the subject has or is at risk for developing cardiovascular disease (CVD) such as hypertriglyceridemia and/or other condition selected from lipidemia (e.g., hyperlipidemia), dyslipidemia (e.g., atherogenic dyslipidemia, diabetic dyslipidemia, or mixed dyslipidemia), hyperlipoproteinemia, hyperapobetalipoproteinemia, chronic heart disease, including coronary artery disease (CAD) or any symptoms or conditions associated with a CVD, e.g., hypercholesterolemia (e.g., statin-resistant hypercholesterolemia, heterozygous or homozygous familial hypercholesterolemia) myocardial infarction (MI), acute coronary syndrome, mesenteric ischemia, superior mesenteric artery occlusion, restenosis, renal artery stenosis, angina peripheral arterial disease (PAD), calcific aortic valve disease (CAVD), aortic stenosis, aortic valve regurgitation, aortic dissection, retinal artery occlusion, atherosclerotic cardiovascular disease (ASCVD), atherosclerosis, dyslipidemia, thrombosis, cerebrovascular atherosclerosis, cerebrovascular disease, or stroke. In certain embodiments, at least one symptom of the cardiovascular injury, disease, condition, or disorder is ameliorated. In certain embodiments, the at least one symptom is selected from include, but are not limited to, angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen, and fever. In certain embodiments, administration of an oligomeric duplex provided herein (e.g., an oligomeric duplex comprising or consisting of an oligomeric antisense agent) to the subject reduces or delays the onset or progression of at least one at least one symptom of aortic stenosis.

Certain embodiments are drawn to an oligomeric duplex comprising or consisting of an oligomeric antisense agent comprising or consisting of a modified oligonucleotide having a targeting region complementary to a target region of a LPA nucleic acid, for the manufacture or preparation of a medicament for ameliorating, or delaying or preventing development or progression of a disease, disorder, condition or injury and/or for ameliorating, preventing or delaying the onset of one or more symptoms of a disease, disorder, condition or injury, wherein the disease, disorder, condition or injury is associated with cardiovascular disease (CVD) such as hypertriglyceridemia and/or other condition selected from lipidemia (e.g., hyperlipidemia), dyslipidemia (e.g., atherogenic dyslipidemia, diabetic dyslipidemia, or mixed dyslipidemia), hyperlipoproteinemia, hyperapobetalipoproteinemia, chronic heart disease, including coronary artery disease (CAD) or any symptoms or conditions associated with a CVD, e.g., hypercholesterolemia (e.g., statin-resistant hypercholesterolemia, heterozygous or homozygous familial hypercholesterolemia) myocardial infarction (MI), acute coronary syndrome, mesenteric ischemia, superior mesenteric artery occlusion, restenosis, renal artery stenosis, angina peripheral arterial disease (PAD), calcific aortic valve disease (CAVD), aortic stenosis, aortic valve regurgitation, aortic dissection, retinal artery occlusion, atherosclerotic cardiovascular disease (ASCVD), atherosclerosis, dyslipidemia, thrombosis, cerebrovascular atherosclerosis, cerebrovascular disease, or stroke. In certain embodiments, the disease is aortic stenosis In certain embodiments, an oligomeric duplex is for the manufacture or preparation of a medicament for improving one or more symptoms selected from angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen, and fever.

In certain embodiments, prophylactic administration of an oligomeric duplex or composition provided herein to a subject at risk for cardiovascular, metabolic, and/or inflammatory disease, is able to prevent, ameliorate, postpone, or delay a symptom and/or development or progression of cardiovascular, metabolic, an/or inflammatory disease progression. In certain embodiments, an oligomeric duplex is for the manufacture or preparation of a medicament for improving angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen, and fever.

In any of the methods or uses described herein, the oligomeric duplex is any oligomeric duplex (e.g., an oligomeric duplex comprising or consisting of an oligomeric antisense agent) described herein.

In certain embodiments an oligomeric duplex is administered parenterally. In certain embodiments, an oligomeric duplex is administered intravenously, subcutaneously, intramuscularly, or intrathecally.

In certain embodiments, an oligomeric duplex or composition is co-administered with a second agent or therapy. In certain embodiments, the oligomeric duplex or composition and second agent are administered concomitantly. In certain embodiments, a second agent is a glucose-lowering agent. In certain embodiments, a second agent is a LDL, TG or cholesterol lowering agent. In certain embodiments, a second agent is an anti-inflammatory agent. In certain embodiments, a second agent can be, but is not limited to, a non-steroidal anti-inflammatory drug (NSAID e.g., aspirin), niacin (e.g., Niaspan), nicotinic acid, ezetimibe, an apoB inhibitor (e.g., Mipomersen), a CETP inhibitor (e.g., Anacetrapib), an apo(a) inhibitor, a thyroid hormone analog (e.g., Eprotirome), a HMG-CoA reductase inhibitor (e.g., a statin), a fibrate (e.g., Gemfibrozil), a microsomal triglyceride transfer protein inhibitor (e.g., Lomitapide), a PCSK9 inhibitor (e.g., inclisiran), as well as therapies in development, e.g., an ANGPTL3 inhibitor, an LPA inhibitor. In certain embodiments a therapy can be, but is not limited to, Lp(a) apheresis. In certain embodiments, a second agent is an alzheimer disease drug. Agents or therapies can be co-administered or administered concomitantly. Agents or therapies can be sequentially or subsequently administered.

### VI. Pharmaceutical Compositions

In certain embodiments, described herein are pharmaceutical compositions comprising one or more oligomeric duplexes or oligomeric compounds, wherein each oligomeric duplex or compound comprises a modified oligonucleotide (e.g., oligomeric compound). In certain embodiments, the one or more oligomeric duplex or oligomeric compound each comprises an antisense agent. In certain embodiments, a pharmaceutical composition comprises a pharmaceutically acceptable diluent or carrier. In certain embodiments, a pharmaceutical composition comprises or consists of a sterile saline solution and one or more compound or duplex. In certain embodiments, the sterile saline is pharmaceutical grade saline. In certain embodiments, a pharmaceutical composition comprises or consists of one or more compound or duplex and sterile water. In certain embodiments, the sterile water is pharmaceutical grade water. In certain embodiments, a pharmaceutical composition comprises or consists of one or more compound or duplex and phosphate-buffered saline (PBS). In certain embodiments, sterile PBS is pharmaceutical grade PBS. In certain embodiments, the pH of a solution is modulated with a suitable pH-adjusting agent, for example, with acids such as hydrochloric acid and alkalis such as sodium hydroxide, to a range of from about 7.1-7.3, or to about 7.2.

In certain embodiments, a pharmaceutical composition comprises an oligomeric duplex comprising a first oligomeric compound and a second oligomeric compound and sterile saline. In certain such embodiments, a pharmaceutical composition consists of such oligomeric duplex and sterile saline. In certain embodiments, a pharmaceutical composition consists essentially of such oligomeric duplex and sterile saline. In certain embodiments, the sterile saline is sterile PBS. In certain embodiments, the sterile saline is pharmaceutical grade. In certain embodiments, the pH of a solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric duplex and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, an oligomeric duplex may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric duplex encompass any pharmaceutically acceptable salts of the oligomeric compound(s) or duplex, esters of the compound(s) or duplex, or salts of such compounds or esters. In certain embodiments, pharmaceutical compositions comprising an oligomeric compound or oligomeric duplex comprising one or more oligomeric compound, upon administration to a subject, including a human, are capable of providing (directly or indirectly) the biologically active metabolite or residue thereof. Accordingly, for example, the disclosure is also drawn to pharmaceutically acceptable salts of oligomeric compounds or oligomeric duplexes, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts.

In certain embodiments, oligomeric compounds or oligomeric duplexes are lyophilized and isolated, e.g., as sodium salts. In certain embodiments, a sodium salt of an agent or duplex is mixed with a pharmaceutically acceptable diluent. In certain embodiments, the pharmaceutically acceptable diluent comprises sterile saline, sterile water, PBS. In certain embodiments, a sodium salt of an oligomeric compound or oligomeric duplex is mixed with PBS.

Lipid moieties have been used in nucleic acid therapies in a variety of methods. In certain methods, a nucleic acid, such as an oligomeric compound or oligomeric duplex comprising oligomeric compound, is introduced into preformed liposomes or lipoplexes made of mixtures of cationic lipids and neutral lipids. In certain methods, nucleic acid complexes with mono- or poly-cationic lipids are formed without the presence of a neutral lipid. In certain embodiments, a lipid moiety is selected to increase distribution of a pharmaceutical compound to a particular cell or tissue. In certain embodiments, a lipid moiety is selected to increase distribution of a pharmaceutical compound to fat tissue. In certain embodiments, a lipid moiety is selected to increase distribution of a pharmaceutical compound to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more compounds to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition is prepared for administration of an oligomeric duplex to a subject. Suitable administration routes include, but are not limited to, oral, rectal, transmucosal, intestinal, enteral, topical, suppository, through inhalation, intrathecal, intracerebroventricular, intraperitoneal, intranasal, intraocular, intratumoral, and parenteral (e.g., intravenous, intramuscular, intrathecal, intramedullary, and subcutaneous). In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier or diluent and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, diluents, suspending agents and the like. Certain pharmaceutical compositions for injection are presented in unit dosage form, e.g., in ampoules or in multi-dose containers. Certain pharmaceutical compositions for injection are suspensions, solutions, or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein function as acids. Although such compounds may be drawn or described in protonated (free acid) form or ionized and in association with a cation (salt) form, aqueous solutions of such compounds exist in equilibrium among such forms. For example, a phosphodiester linkage of an oligonucleotide in aqueous solution exists in equilibrium among free acid, anion, and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” herein is intended to include all such forms. Drawn structures necessarily depict a single form. Nevertheless, unless otherwise indicated, such drawings are likewise intended to include corresponding forms. Herein, a structure depicting the free acid of a compound followed by the term “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, oligomeric compounds or oligomeric duplexes are in aqueous solution with sodium. In certain embodiments, oligomeric compounds or oligomeric duplexes are in aqueous solution with potassium. In certain embodiments, oligomeric compounds or oligomeric duplexes are in PBS. In certain embodiments, oligomeric compounds or oligomeric duplexes are in water. In certain such embodiments, the pH of a solution is adjusted with NaOH and/or HCl to achieve a desired pH.

Herein, a dose may be in the form of a dosage unit. For clarity, a dose (or dosage unit) of an agent (e.g., oligomeric compound, oligomeric duplex, antisense agent) in milligrams indicates the mass of the free acid form of the compound. As described herein, in aqueous solution, the free acid is in equilibrium with anionic and salt forms. However, for the purpose of calculating dose, it is assumed that the compound (e.g., oligomeric compound, oligomeric duplex, antisense agent) exists as a solvent-free, sodium-acetate free, anhydrous, free acid. In certain embodiments, where an agent (e.g., oligomeric compound, oligomeric duplex, antisense agent) is in solution comprising sodium (e.g., saline), the compound may be partially or fully de-protonated and in association with sodium ions. However, the mass of the protons is nevertheless counted toward the weight of the dose, and the mass of the sodium ions is not counted toward the weight of the dose. When an agent comprises a conjugate group, the mass of the conjugate group is included in calculating the dose of such compound. If the conjugate group also has an acid, the conjugate group is likewise assumed to be fully protonated for the purpose of calculating dose.

### VII. Compounds

Provided herein are reduced fluorine content agents and duplexes. In certain embodiments, a reduced fluorine content oligomeric compound comprises an oligonucleotide (e.g., an antisense oligomeric compound) which has a nucleobase sequence complementary to a sequence in an LPA nucleic acid, e.g., a human LPA nucleic acid (SEQ ID NO: 1), or an oligonucleotide (e.g., a sense oligomeric compound) which has a nucleobase sequence complementary to a sequence of an oligonucleotide which has a nucleobase sequence complementary to a sequence in an LPA nucleic acid, e.g., a human LPA nucleic acid (SEQ ID NO: 2). In certain embodiments, a reduced fluorine content oligomeric duplex comprises a first modified oligonucleotide (e.g., an antisense oligomeric compound), and a second modified oligonucleotide (e.g., a sense oligomeric compound). In certain embodiments, the reduced fluorine content oligomeric compounds and oligomeric duplexes provided herein may be preferable to compounds containing more fluorine atoms due to improved properties, e.g., decreased off-target actions and/or improved durability, and have LPA RNA and/or Lp(a) protein reduction activity that is comparable to or greater than that of a comparator compound containing more fluorine atoms (e.g., a compound having 20% or more, 25% or more, or 30% or more fluorine-containing nucleosides). In certain embodiments, an oligomeric compound or oligomeric duplex having reduced fluorine content provided herein comprises a modified oligonucleotide or a first modified oligonucleotide which has a nucleobase sequence complementary to a sequence in an LPA nucleic acid, having reduced fluorine content has fewer than 20%, fewer than 15%, fewer than 10%, or fewer than 5% of nucleosides comprising a fluorine atom, and/or a modified oligonucleotide or a second modified oligonucleotide which has a nucleobase sequence complementary to the first oligonucleotide, or to a sequence that is complementary to a sequence in an LPA nucleic acid, having reduced fluorine content has fewer than 10%, fewer than 8%, or fewer than 5% of nucleosides comprising a fluorine atom. In certain embodiments, an oligomeric compound or oligomeric duplex having reduced fluorine content provided herein comprises a modified oligonucleotide or first modified oligonucleotide having very low fluorine content has fewer than 20%, fewer than 15%, fewer than 10% or fewer than 5% of nucleosides comprising a fluorine atom. In certain embodiments, the second modified oligonucleotide of such oligomeric duplexes has a very low fluorine content has fewer than 15%, fewer than 12%, or fewer than 10% of nucleosides comprising a fluorine atom. In certain embodiments, an oligomeric compound or oligomeric duplex provided herein comprises an oligonucleotide or first modified oligonucleotide (e.g., an antisense oligomeric compound) having reduced fluorine content has fewer than 20%, fewer than 15%, fewer than 10%, or fewer than 5% of nucleosides comprising a fluorine atom, and has a nucleobase sequence that is at least 85%, at least 90%, at least 95%, or at least 99% complementary to an equal length portion of an LPA nucleic acid nucleobase sequence of SEQ ID NOs: 2. In certain embodiments, an oligomeric compound or oligomeric duplex provided herein comprises an oligonucleotide or a first modified oligonucleotide (e.g., an antisense oligomeric compound), having reduced fluorine content has fewer than 20%, fewer than 15%, fewer than 10%, or fewer than 5% of nucleosides comprising a fluorine atom, and having a nucleobase sequence of any one of SEQ ID NOs: 83-98, or 143-144. In certain embodiments, such oligomeric compounds or oligomeric duplexes comprise an oligonucleotide or second modified oligonucleotide (e.g., a sense oligomeric compound) having reduced fluorine content, wherein fewer than 20%, fewer than 15%, or fewer than 10% of nucleosides comprising a fluorine atom, comprising a nucleobase sequence complementary to the first modified oligonucleotide selected from among a nucleobase sequence of any one of SEQ ID NOs: 99-112 or 145-146. In some such embodiments, the modified oligonucleotide or first modified oligonucleotide has reduced fluorine content, wherein less than 20%, less than 15%, less than 10% or less than 5% of nucleobases comprising a fluorine atom, and comprises at least 18, at least 19, at least 20, at least 21, at least 22, or at least 23 nucleobases of a sequence complementary to the nucleobase sequence of SEQ ID NO: 2; and the modified oligonucleotide or second modified oligonucleotide has reduced fluorine content, wherein less than 20% less than 15%, or less than 10% of nucleobases comprising a fluorine atom, and comprises at least 16, at least 17, at least 18, at least 19, at least 20, or at least 21 contiguous nucleobases of the nucleobase sequence of any one of SEQ ID NOs: 99-112 or 145-146. In certain embodiments, an oligomeric compound or oligomeric duplex having reduced fluorine content provided herein comprises a modified oligonucleotide or first modified oligonucleotide having very low fluorine content, wherein fewer than 12%, fewer than 10% or fewer than 5% of nucleosides comprising a fluorine atom, and comprises at least 18, at least 19, at least 20, at least 21, at least 22, or at least 23 contiguous nucleobases of a sequence complementary to the nucleobase sequence of SEQ ID NO: 2. In certain embodiments, the second modified oligonucleotide of such oligomeric duplexes has a very low fluorine content, wherein fewer than 15%, fewer than 12%, or fewer than 10% of nucleosides comprising a fluorine atom, and comprises at least 16, at least 17, at least 18, at least 19, at least 20, or at least 21 contiguous nucleobases of the nucleobase sequence of any one of SEQ ID NOs: 99-112 or 145-146.

In certain embodiments, an oligomeric compound or oligomeric duplex having reduced fluorine content provided herein comprises a conjugate group. In some such embodiments of oligomeric duplexes provided herein, the conjugate group is attached to the first (e.g., antisense) or second (e.g., sense) modified oligonucleotide of the oligomeric duplex. In certain embodiments, the conjugate group is attached to the 5′- or 3′-end of the modified oligonucleotide of an oligomeric compound or of the first or second modified oligonucleotide of an oligomeric duplex, or the 5′- or 3′-terminal nucleoside of the modified oligonucleotide of an oligomeric compound or of the first or second modified oligonucleotide of an oligomeric duplex. In certain embodiments, the conjugate group is attached to the second modified oligonucleotide (e.g., sense oligomeric compound), for example, the 5′- or 3′-terminal nucleoside of the second modified oligonucleotide of an oligomeric duplex. In certain embodiments, the conjugate group is attached to the 5′-terminal nucleoside of the second modified oligonucleotide. In certain embodiments, the conjugate group comprises a cell-targeting moiety having affinity for the hepatic asialoglycoprotein receptor (ASGP-R). In certain embodiments, the cell-targeting moiety comprises more than one ligand, each an N-acetyl galactosamine (GalNAc). In certain embodiments, the cell-targeting moiety comprises 3 GalNAc ligands. In In certain embodiments, the conjugate group has the following structure:

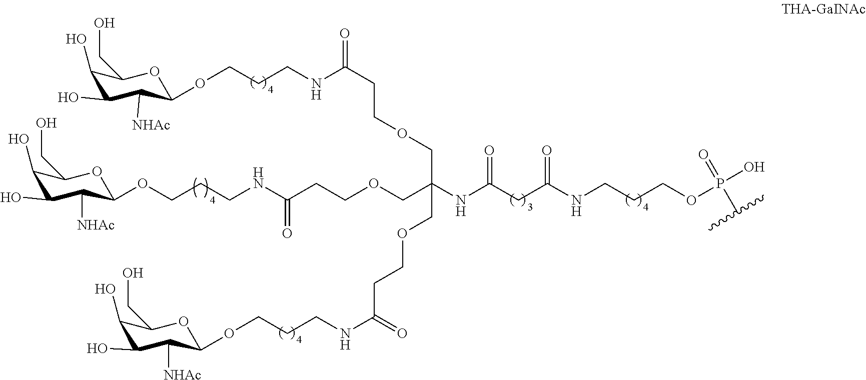

and is attached to the second modified oligonucleotide (e.g., sense oligomeric compound) of the oligomeric duplex through a phosphodiester bond, e.g., through a phosphodiester bond with the 5′-terminal nucleoside of the modified oligonucleotide of the oligomeric compound or the second modified oligonucleotide of the oligomeric duplex. In certain embodiments, the conjugate group has the following structure:

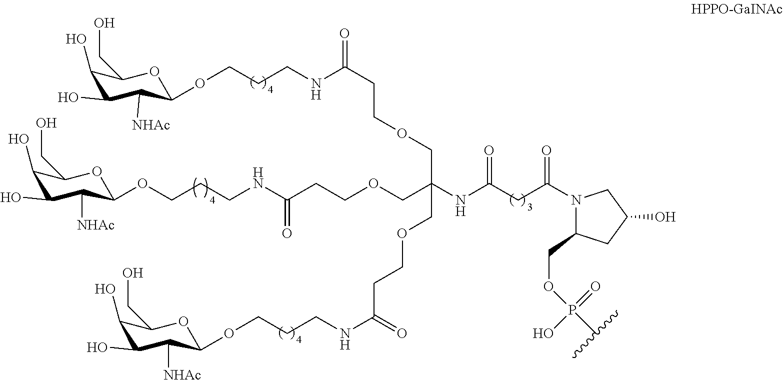

and is attached to the second modified oligonucleotide (e.g., sense oligomeric compound) of the oligomeric duplex through a phosphodiester bond, e.g., through a phosphodiester bond with the 3′-terminal nucleoside of the modified oligonucleotide of the oligomeric compound or the second modified oligonucleotide of the oligomeric duplex.
a. Compound No. 1829960

Provided herein is Compound No. 1829960, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1829960

The first oligomeric compound of Compound no. 1829960, which is Compound No. 1792676, has a first modified oligonucleotide having a nucleobase sequence of (from 5′ to 3′) TGGAGUAUGUGCCUCGAUAACAA (SEQ ID NO: 85), wherein the first oligomeric compound of Compound 1829960 is represented by the following chemical notation: vP-TesGfsGyoAyoGyoUdsAyoUyoGeoUyoGyoCyoCyoUdsCyoGdsAyoUyoAyoAyoCysAesAe (SEQ ID NO: 5); wherein
A=an adenine nucleobase,
C=an cytosine nucleobase,
G=a guanine nucleobase,
T=a thymine nucleobase,
U=a uracil nucleobase,
d=a 2′-β-D-deoxyribosyl sugar moiety
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1829960

The second oligomeric compound of Compound No. 1829960, which is Compound No. 1821829, has a nucleobase sequence of (from 5′ to 3′) GUUAUCGAGGCACAUACUCCA (SEQ ID NO: 100), wherein the second oligomeric compound of Compound 1829960 is represented by the following chemical notation:

(SEQ ID NO: 58)
GesUesUyoAyoUyoCyoGyoAyoGfoGdsCfoAyoCyoAcoUyoAyoCy
oUyoCysCesAe-[HPPO-GalNAc];

wherein:

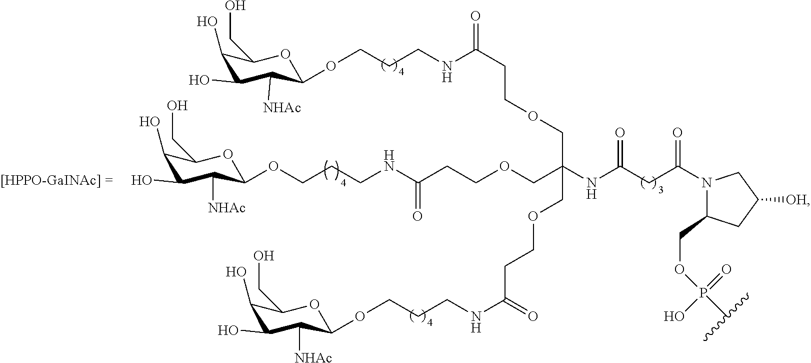

A=an adenine nucleobase,
C=an cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
d=a 2′-β-D-deoxyribosyl sugar moiety,
o=a phosphodiester internucleoside linkage, and
s=a phosphorothioate internucleoside linkage.

In certain embodiments, Compound No. 1829960 is in the form of an anion or a salt thereof, for example, a sodium salt. In certain embodiments, the oligomeric duplex is in anionic form in a solution. In certain embodiments, Compound No. 1829960 is a sodium salt or a potassium salt.

The following chemical structure is one structural representation of Compound No. 1829960:

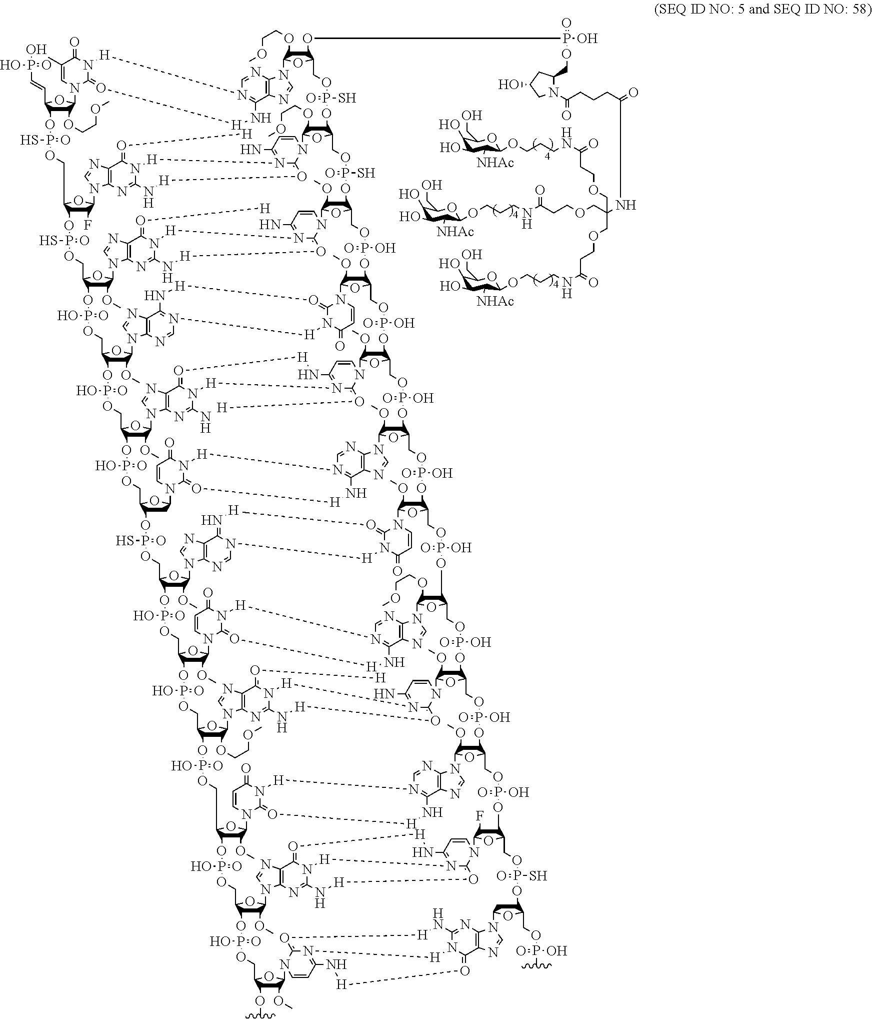

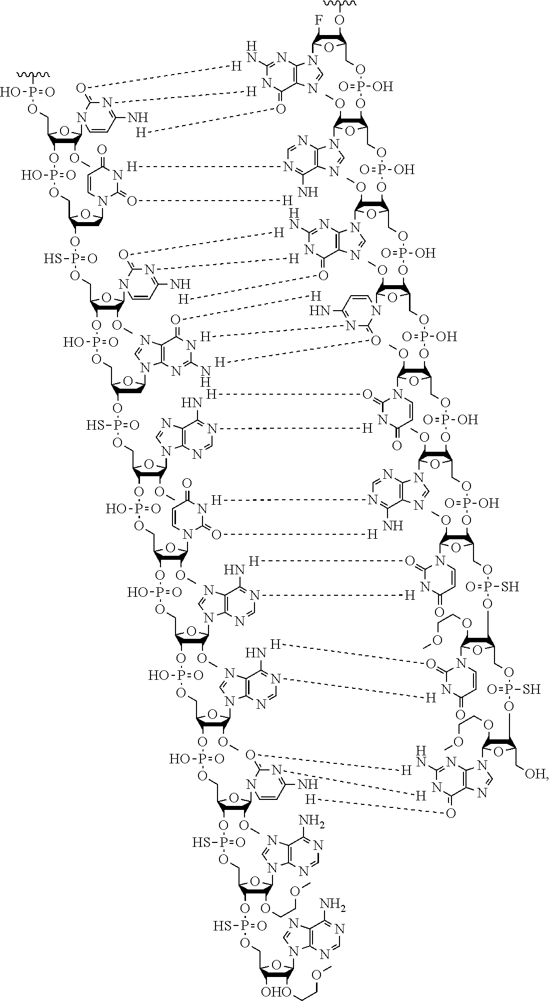

or an ion or a salt thereof

The following chemical structure represents Compound No. 1829960 as a sodium salt:

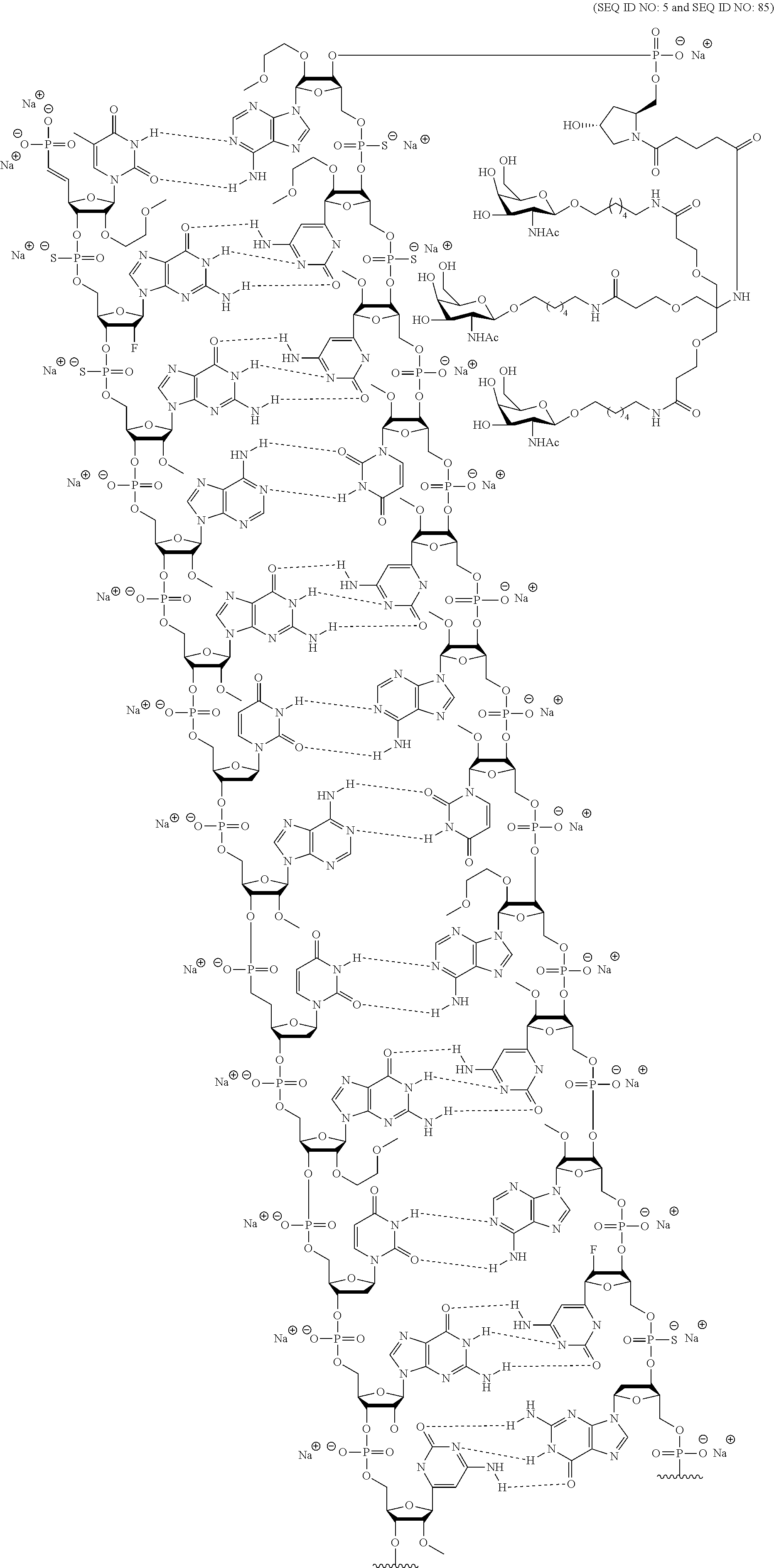

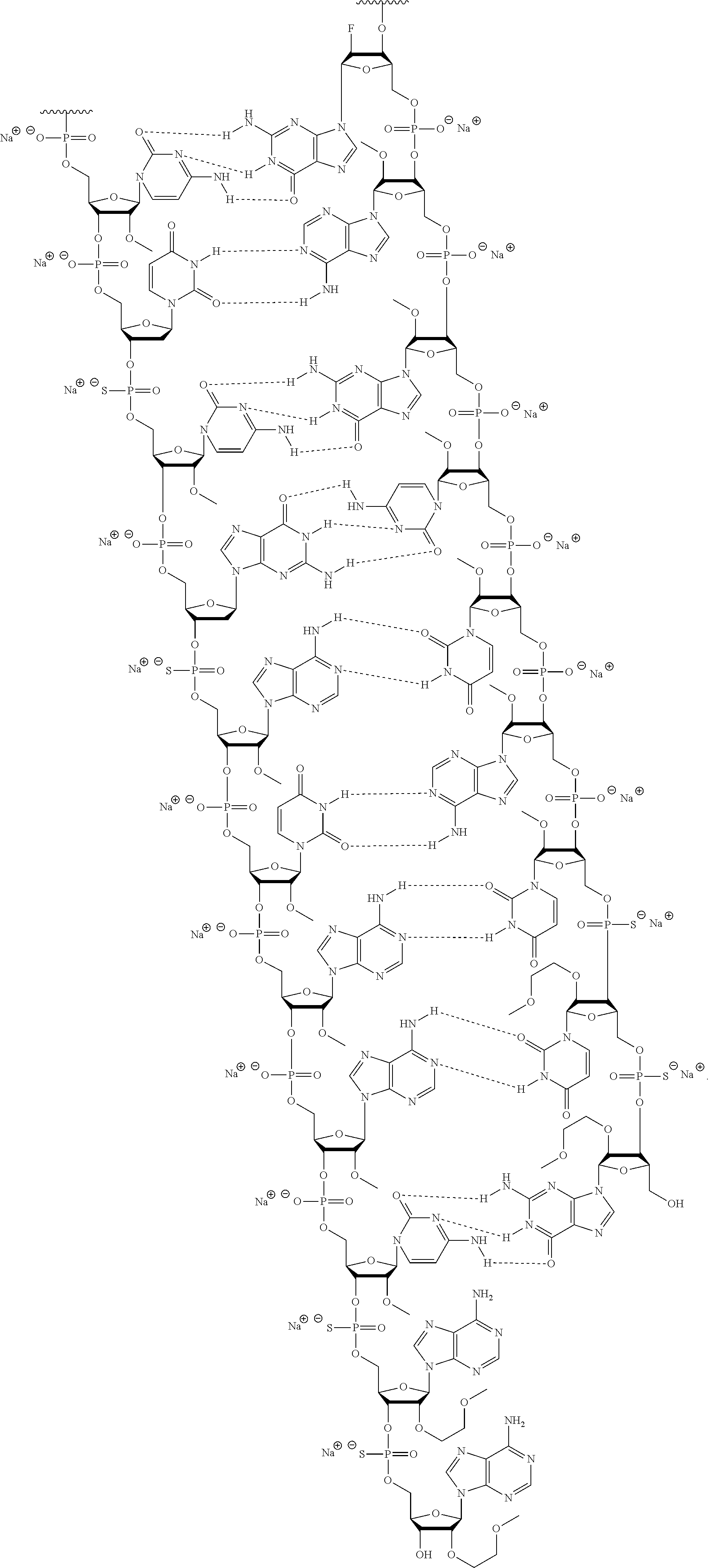

The following chemical structure represents Compound No. 1829960 ion in solution:

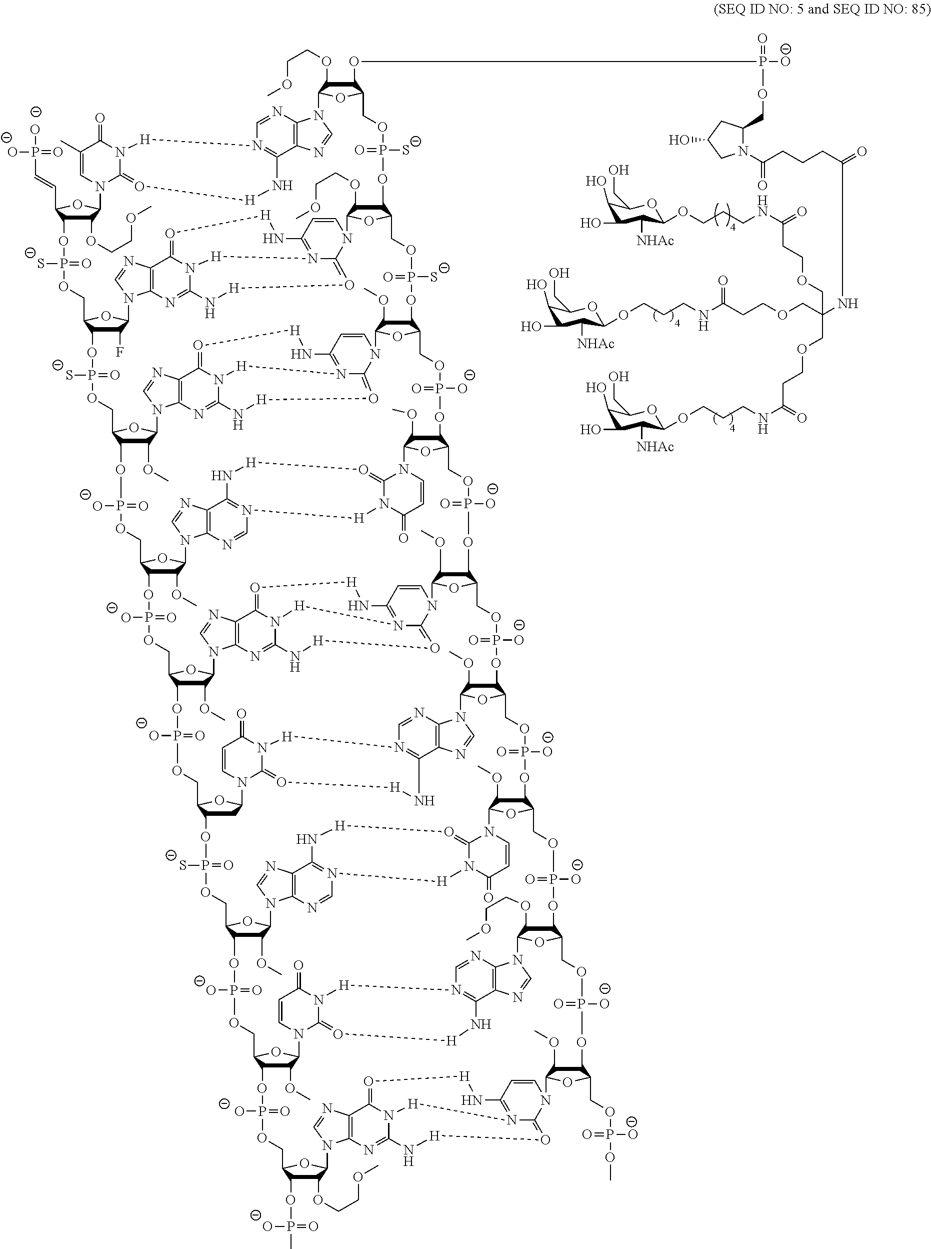

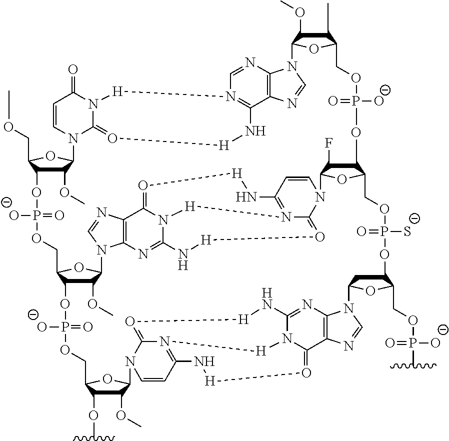

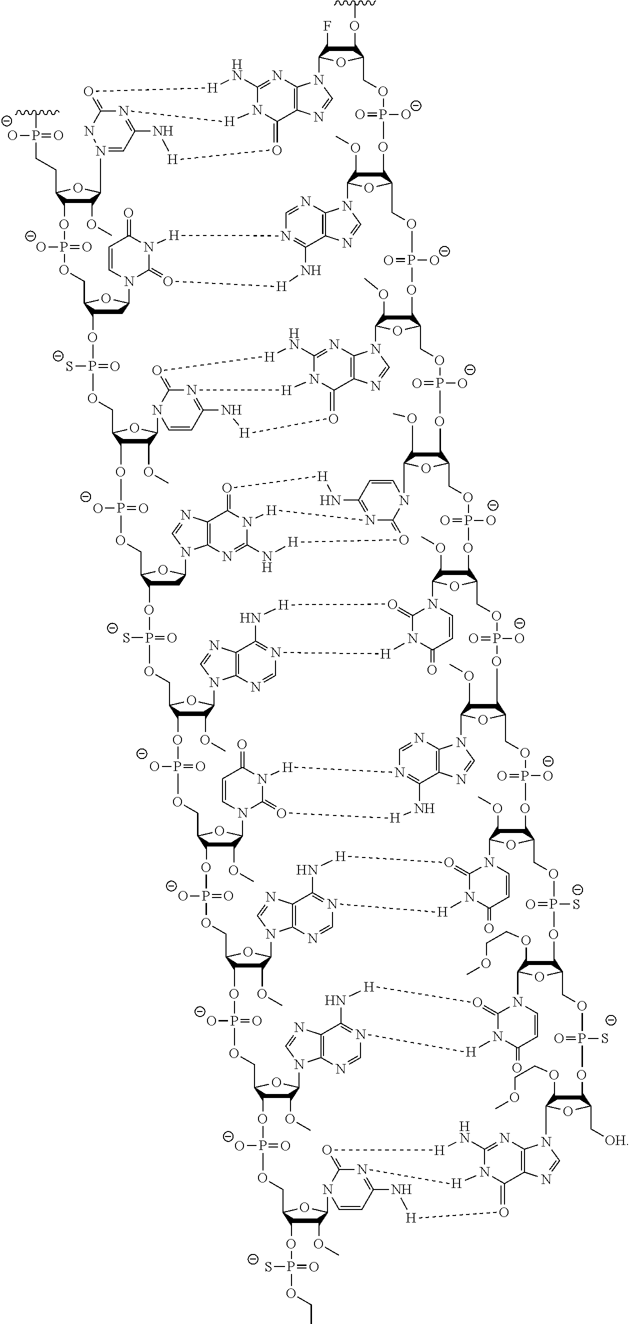

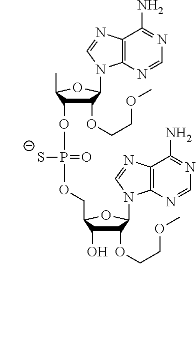

In certain embodiments, provided herein are oligomeric duplexes comprising or consisting of a first oligomeric compound containing a first modified oligonucleotide and a second oligomeric compound containing a second modified oligonucleotide as described in a. above for Compound 1829960, however optionally includes the conjugate group as depicted, which may be present or absent, and/or located at a different site (e.g., the 5′end), and furthermore independently may comprise a different cell targeting conjugate group.

b. Compound No. 1839410

Provided herein is Compound No. 1839410, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1839410

The first oligomeric compound of Compound No. 1839410, which is Compound No. 1839377, has a first modified oligonucleotide having a nucleobase sequence of (from 5′ to 3′) TGCCUCGAUAACUCUGUCCAUAA (SEQ ID NO: 87), wherein the first oligomeric compound of Compound 1839410 is represented by the following chemical notation: VP-vP-TesGfsCyoCyoUyoCfoGxoAyoUyoAyoAyoCyoUyoCfoUyoGyoUyoCyoCyoAyoUysAesAe (SEQ ID NO: 26); wherein
A=an adenine nucleobase,
C=an cytosine nucleobase,
G=a guanine nucleobase,
T=a thymine nucleobase,
U=a uracil nucleobase,
x=a 2′-β-D-deoxyxylosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1839410

The second oligomeric compound of Compound No. 1839410, which is Compound No. 1814996, has a nucleobase sequence of (from 5′ to 3′) AUGGACAGAGUUAUCGAGGCA (SEQ ID NO: 99), the second oligomeric compound of Compound 1839410 is represented by the following chemical notation: [THA-GalNAc]-AesUesGyoGyoAyoCyoAyoGyoAfoGfoUfoUyoAyoUeoCyoGyoAyoGyoGysCesAe (SEQ ID NO: 53); wherein:

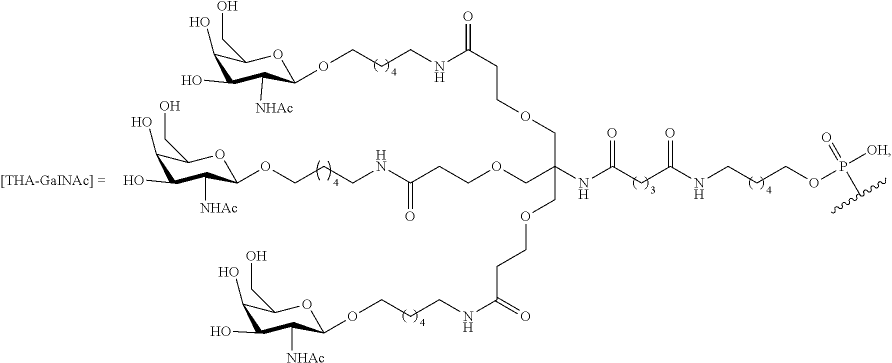

A=an adenine nucleobase,
C=an cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, Compound No. 1839410 is in the form of an anion or a salt thereof, for example, a sodium salt. In certain embodiments, the oligomeric duplex is in anionic form in a solution. In certain embodiments, Compound No. 1839410 is a sodium salt or a potassium salt.

The following chemical structure is one structural representation of Compound No. 1839410:

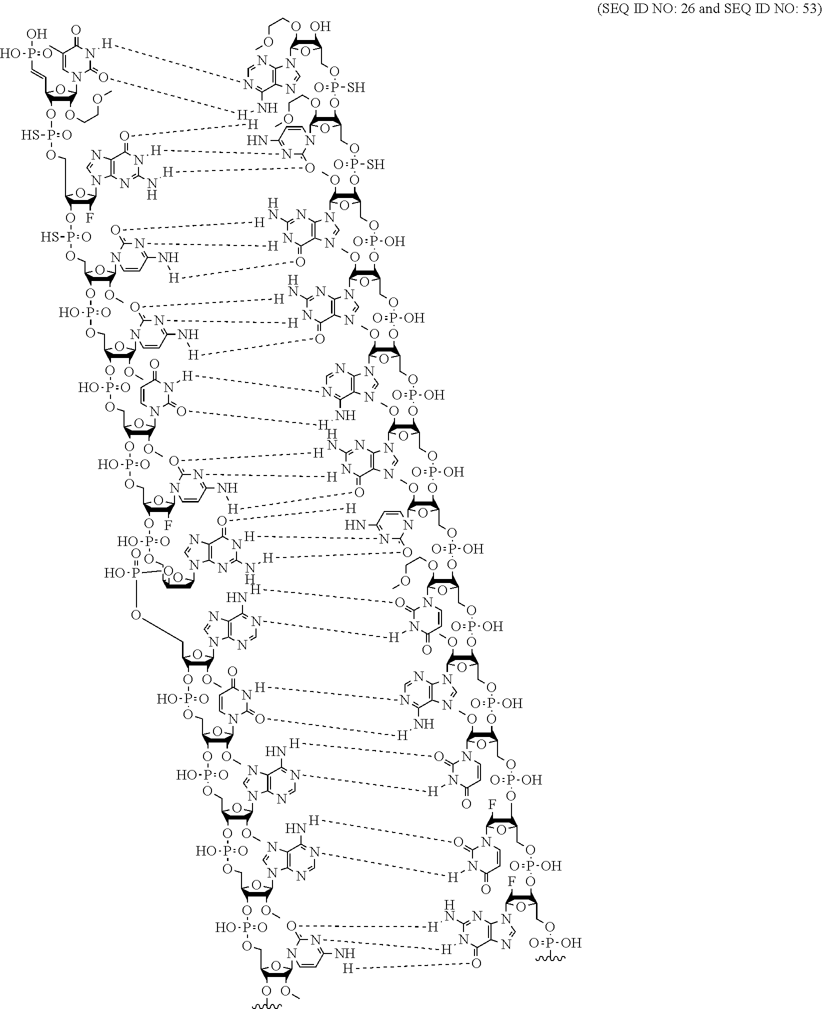

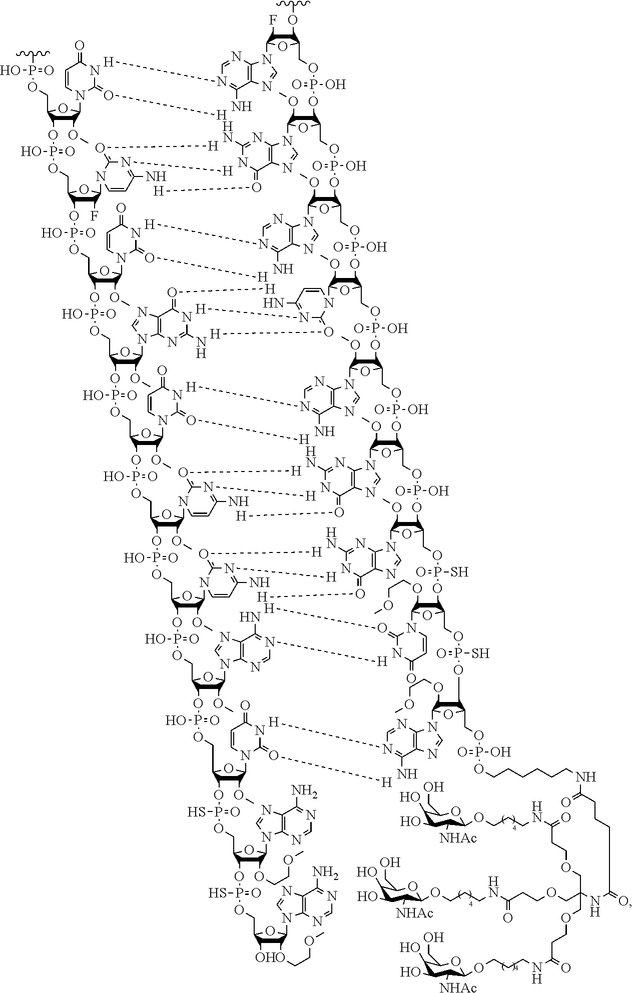

or an ion or a salt thereof.

The following chemical structure represents Compound No. 1839410 in sodium form:

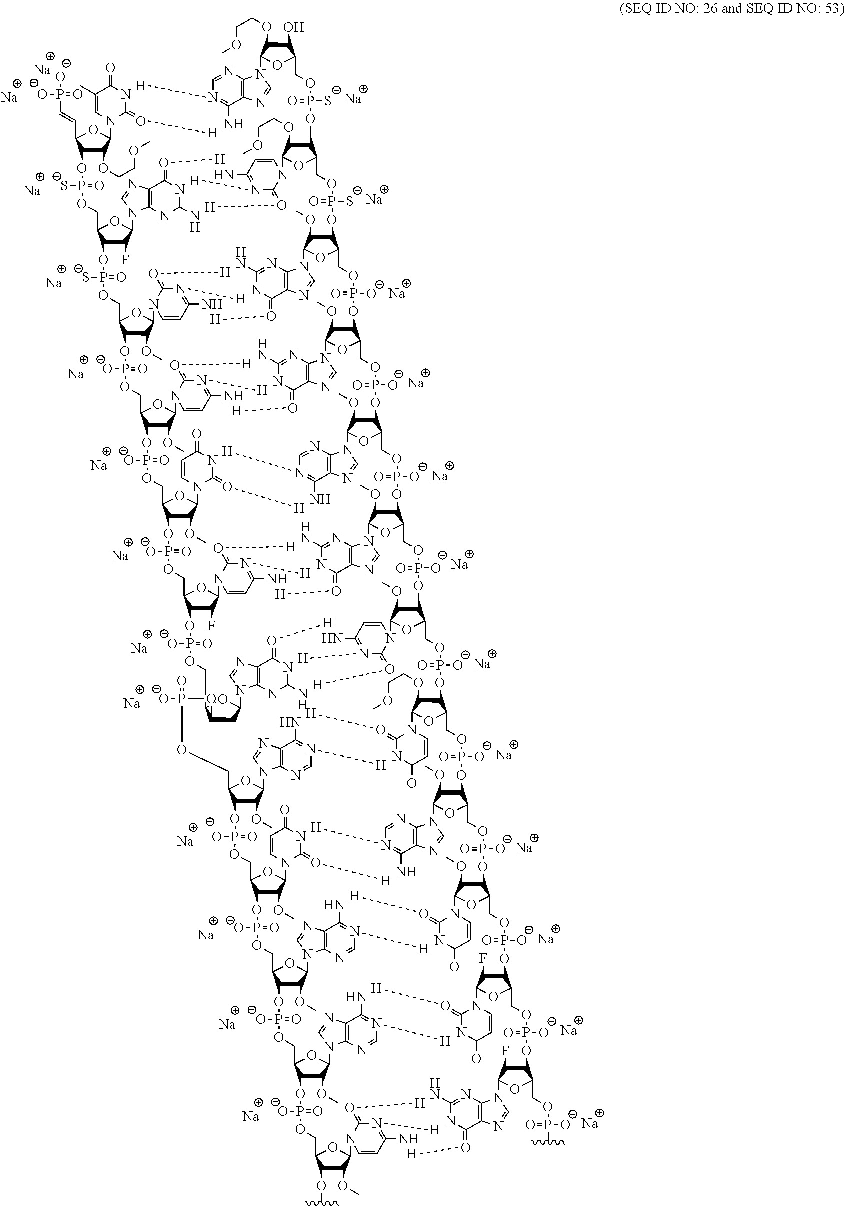

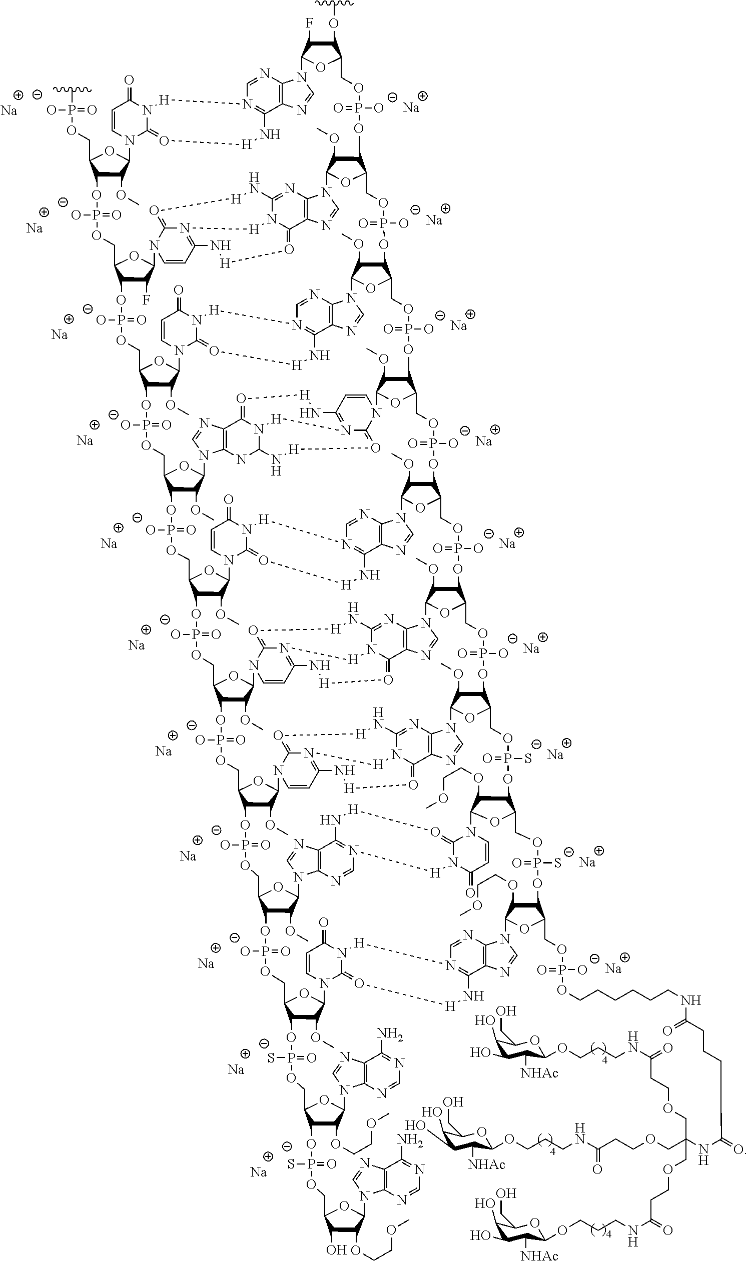

The following chemical structure represents Compound No. 1839410 ion in solution:

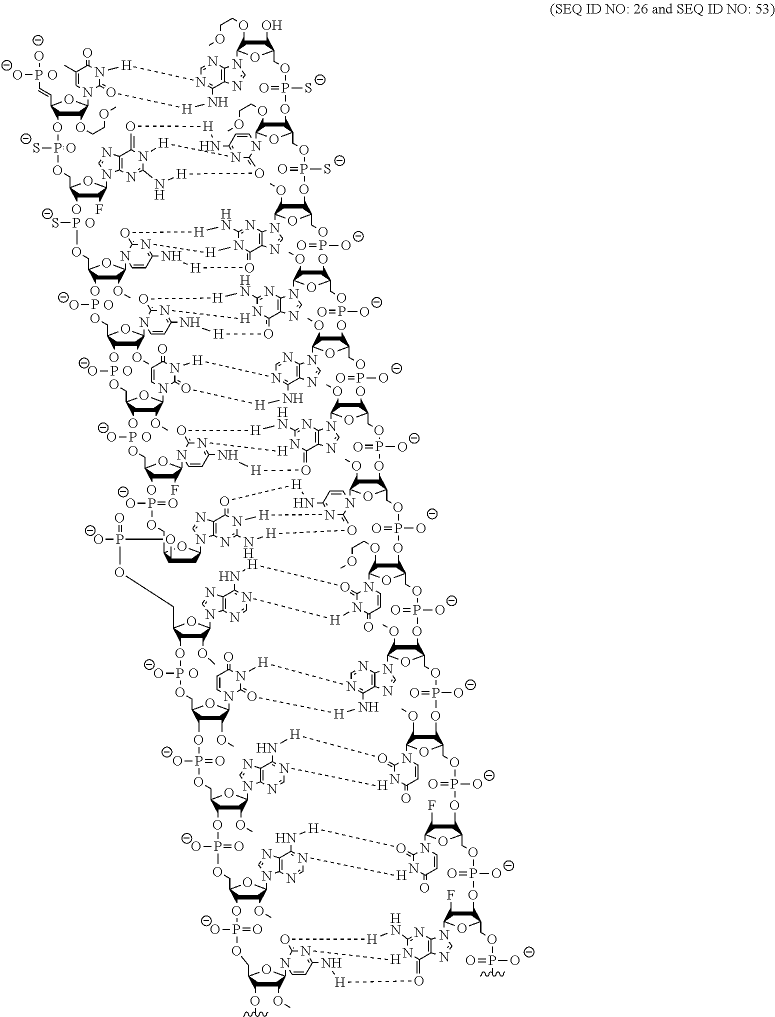

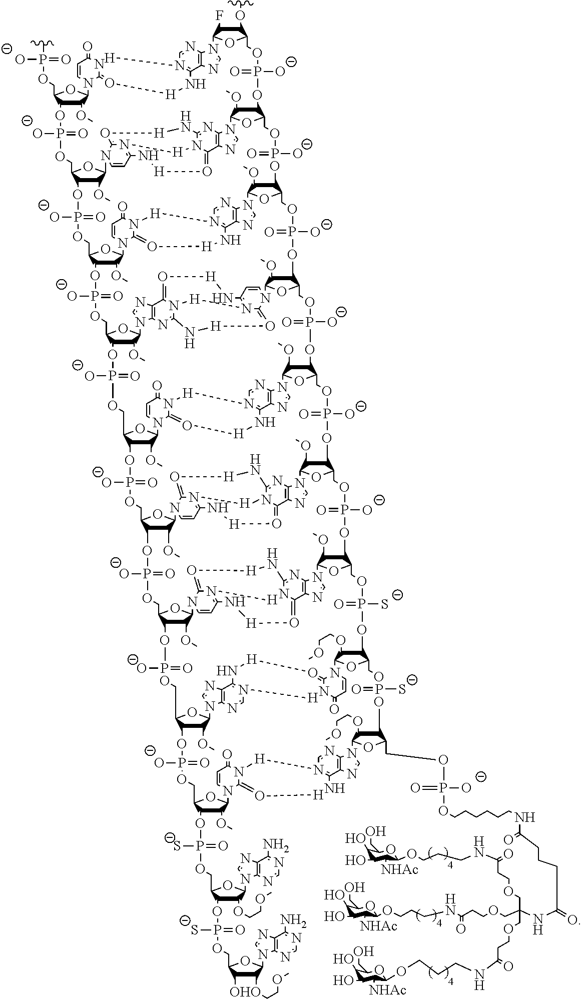

In certain embodiments, provided herein are oligomeric duplexes comprising or consisting of a first oligomeric compound containing a first modified oligonucleotide and a second oligomeric compound containing a second oligomeric compound as described in b. above for Compound no. 1839410, however, the second oligomeric compound optionally includes the conjugate group as depicted, which may be present or absent, and/or located at a different site (e.g., the 5′end), and furthermore independently may comprise a different cell targeting conjugate group.

c. Compound No. 1839412

Provided herein is Compound No. 1839412, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1839412

The first modified oligonucleotide of Compound no. 1839412, which is Compound No. 1839380, has a first modified oligonucleotide having a nucleobase sequence of (from 5′ to 3′) TGCCUCGAUAACUCUGUCCAUAA (SEQ ID NO: 87), wherein the first modified oligonucleotide of Compound 1839412 is represented by the following chemical notation: vP-TesGfsCyoCyoUxoCfoGyoAyoUyoAyoAyoCyoUyoCfoUyoGyoUyoCyoCyoAyoUysAesAe (SEQ ID NO: 28); wherein
A=an adenine nucleobase,
C=an cytosine nucleobase,
G=a guanine nucleobase,
T=a thymine nucleobase,
U=a uracil nucleobase,
e=a 2′-MOE sugar moiety,
f=a 2′-fluoro sugar moiety,
x=a 2′-β-D-deoxyxylosyl sugar moiety,
y=a 2′-OMe sugar moiety,
o=a phosphodiester internucleoside linkage,
s=a phosphorothioate internucleoside linkage, and
vP=a 5′ vinyl phosphonate moiety.

### Second Oligomeric Compound of Compound No. 1839412

The second oligomeric compound of Compound No. 1839412, which is Compound No. 1814996, has a nucleobase sequence of (from 5′ to 3′) AUGGACAGAGUUAUCGAGGCA (SEQ ID NO: 99), the second oligomeric compound of Compound 1839412 is represented by the following chemical notation: [THA-GalNAc]-AₑₛUₑₛG_{yo}G_{yo}A_{yo}C_{yo}A_{yo}G_{yo}A_{fo}G_{fo}U_{fo}U_{yo}A_{yo}UₑₒC_{yo}G_{yo}A_{yo}G_{yo}G_{ys}CₑₛAₑ (SEQ ID NO: 53); wherein:

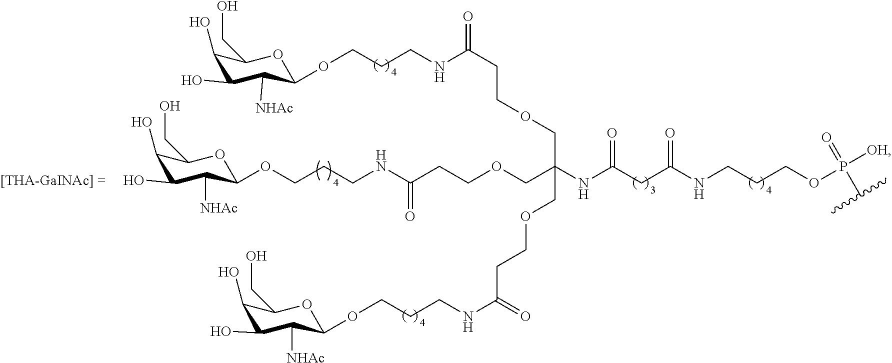

A=an adenine nucleobase,
C=an cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, Compound No. 1839412 is in the form of an anion or a salt thereof, for example, a sodium salt. In certain embodiments, the oligomeric duplex is in anionic form in a solution. In certain embodiments, Compound No. 1839412 is a sodium salt or a potassium salt.

The following chemical structure is one structural representation of Compound No. 1839412:

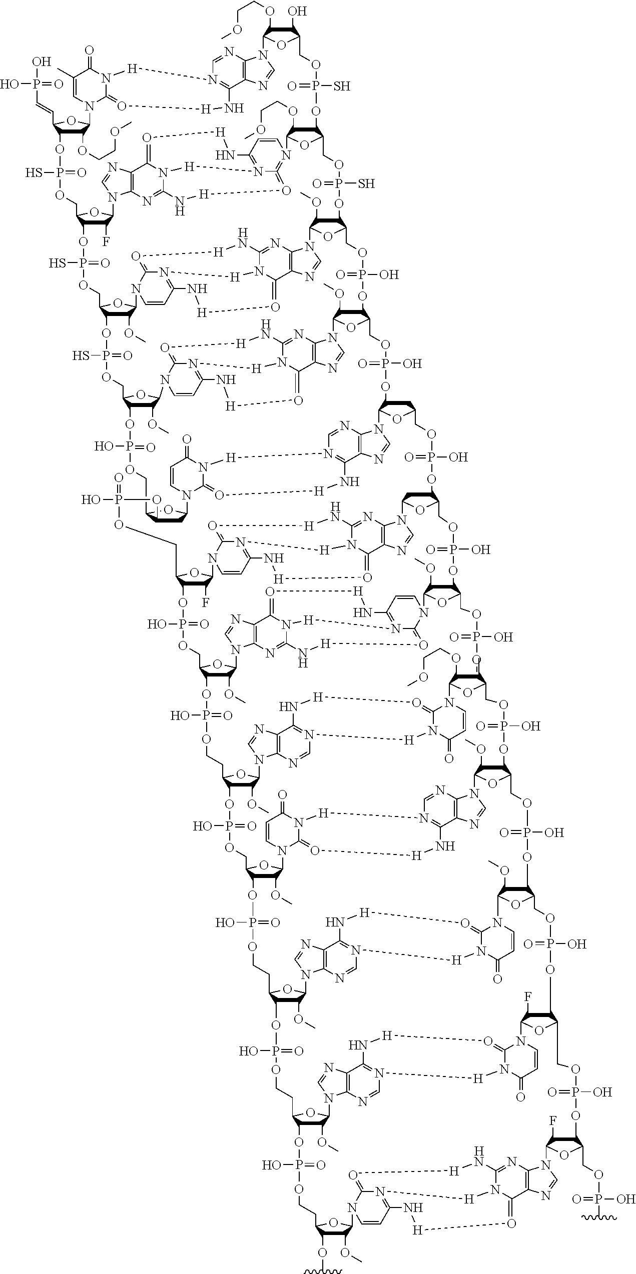

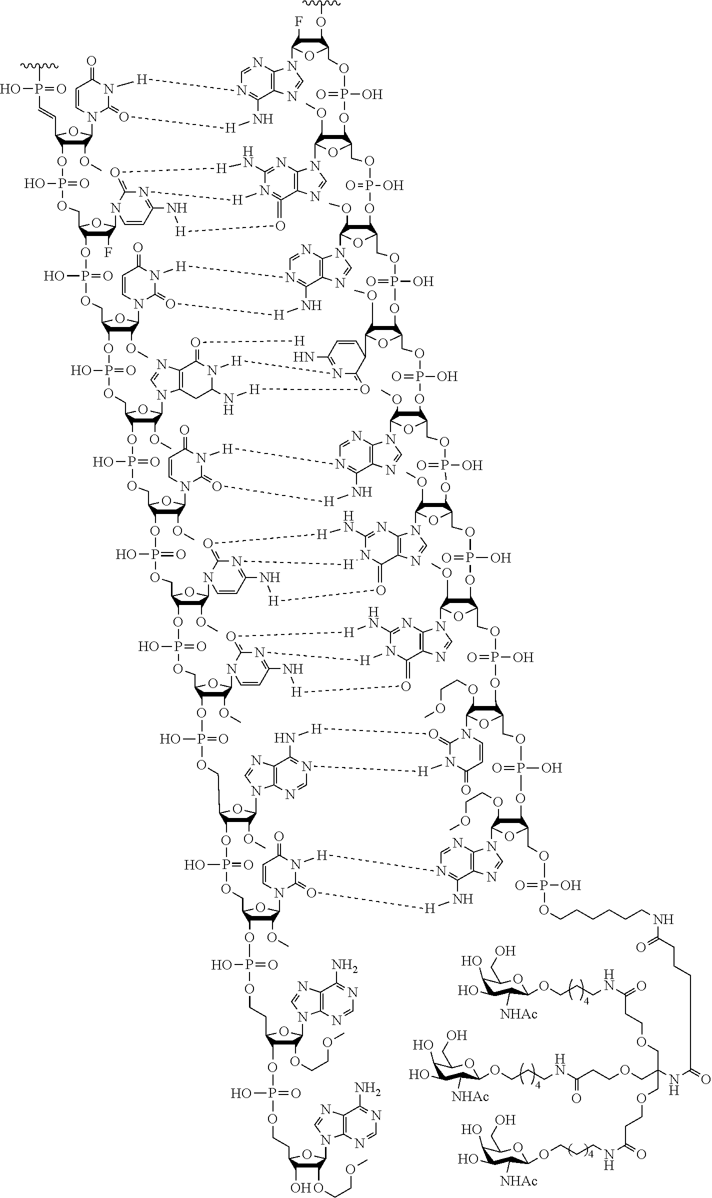

or an ion or a salt thereof.

The following chemical structure represents Compound No. 1839412 in sodium form:

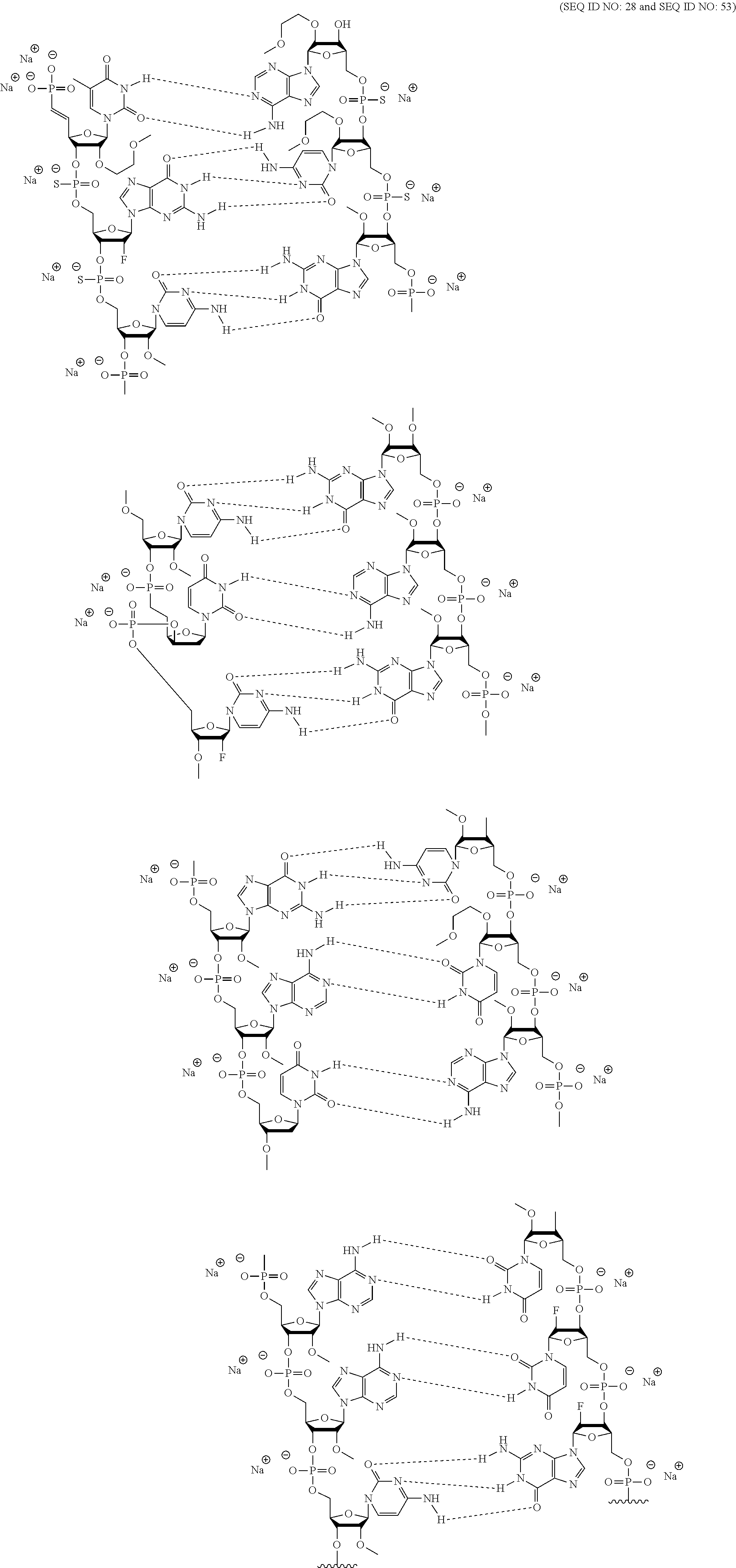

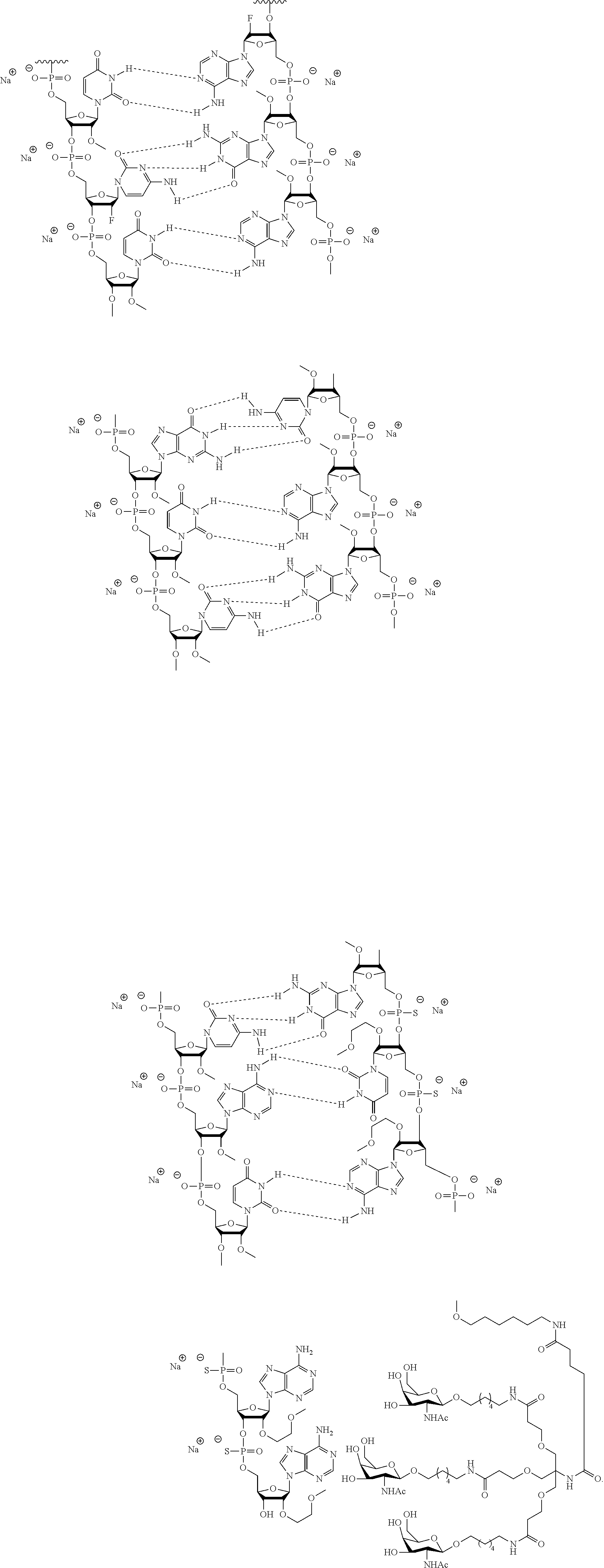

The following chemical structure represents Compound No. 1839412 ion in solution:

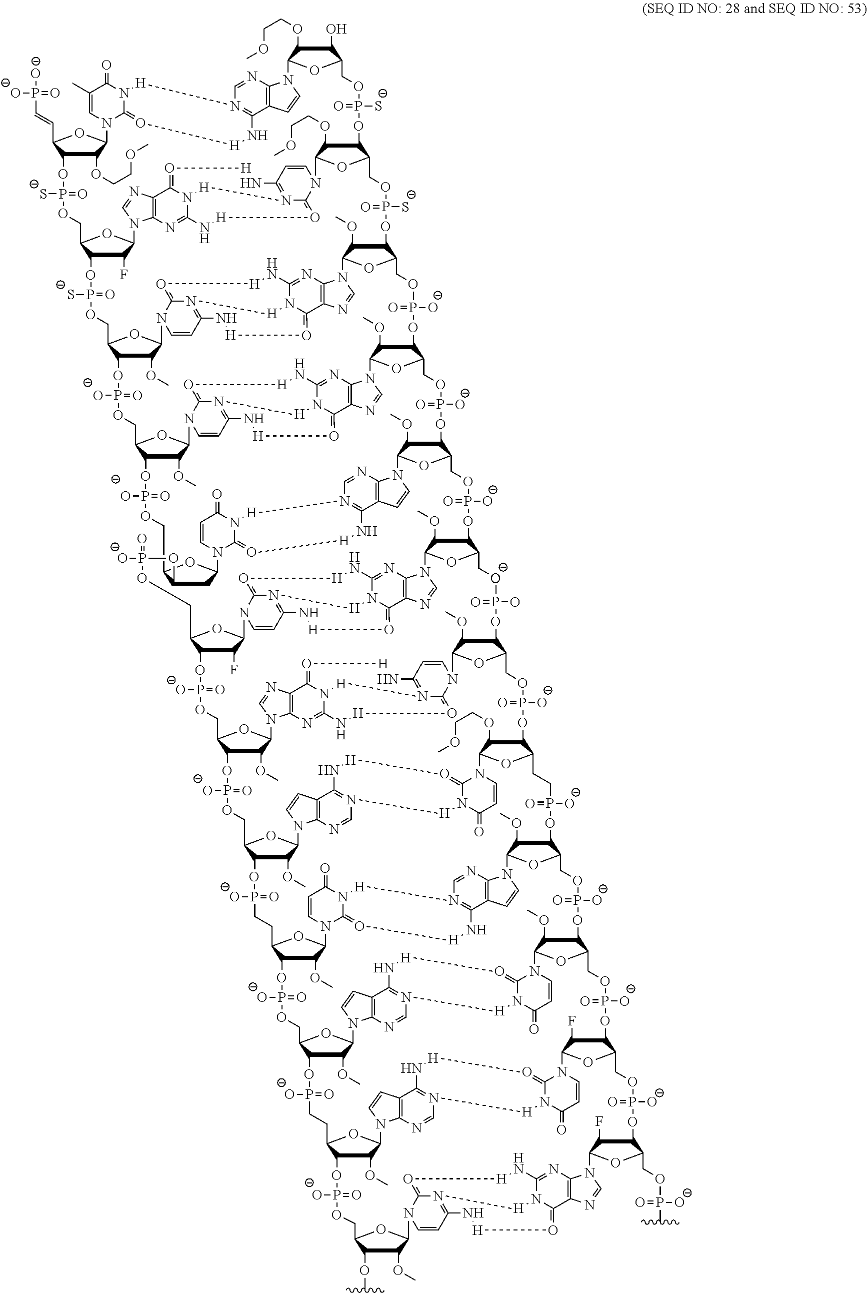

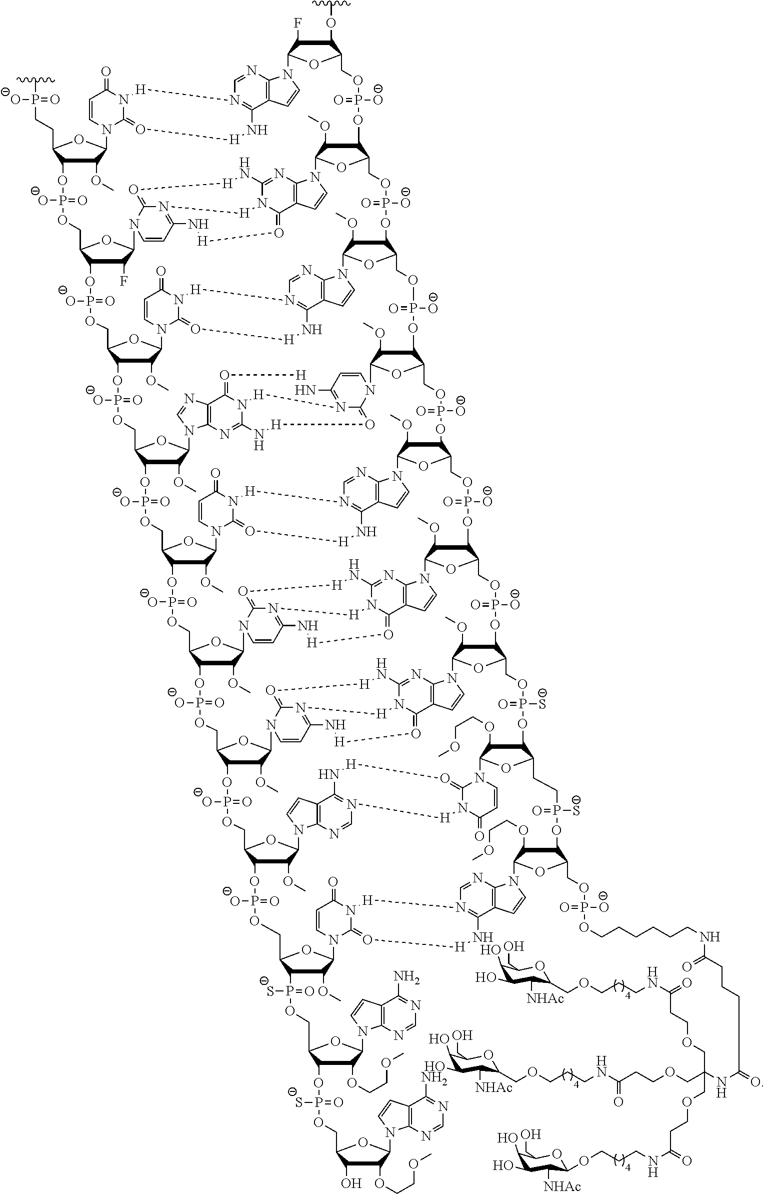

In certain embodiments, provided herein are oligomeric duplexes comprising or consisting of a first oligomeric compound containing a first modified oligonucleotide and a second oligomeric compound containing a second oligomeric compound as described in c. above for Compound no. 1839412, however, the second oligomeric compound optionally includes the conjugate group as depicted, which may be present or absent, and/or located at a different site (e.g., the 5′end), and furthermore independently may comprise a different cell targeting conjugate group.

d. Compound No. 1840071

Provided herein is Compound No. 1840071, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1840071

The first oligomeric compound of Compound no. 1840071, which is Compound No. 1792676, has a first modified oligonucleotide having a nucleobase sequence of (from 5′ to 3′) TGGAGUAUGUGCCUCGAUAACAA (SEQ ID NO: 85), wherein the first oligomeric compound of Compound 1829960 is represented by the following chemical notation: vP-TesGfsGyoAyoGyoUdsAyoUyoGeoUyoGyoCyoCyoUdsCyoGdsAyoUyoAyoAyoCysAesAe (SEQ ID NO: 5); wherein
A=an adenine nucleobase,
C=an cytosine nucleobase,
G=a guanine nucleobase,
T=a thymine nucleobase,
U=a uracil nucleobase,
d=a 2′-β-D-deoxyribosyl sugar moiety
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1840071

The second oligomeric compound of Compound No. 1840071, which is Compound No. 1839284, has a nucleobase sequence of (from 5′ to 3′) GUUAUCGAGGCACAUACUCCA (SEQ ID NO: 100), wherein the second oligomeric compound of Compound 1840071 is represented by the following chemical notation: [THA-GalNAc]-GₑₛUₑₛU_{yo}A_{yo}U_{yo}C_{yo}G_{yo}A_{yo}G_{fo}G_{ds}C_{fo}A_{yo}C_{yo}AₑₒU_{yo}A_{yo}C_{yo}U_{yo}C_{ys}CₑₛAₑ (SEQ ID NO: 66); wherein:

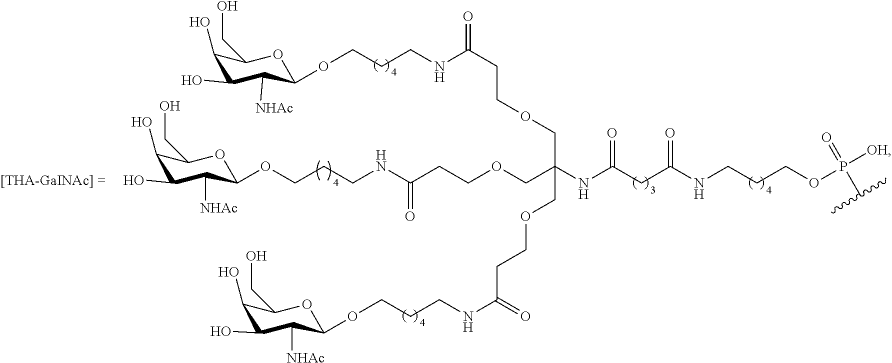

A=an adenine nucleobase,
C=an cytosine nucleobase,
G=a guanine nucleobase,
T=a thymine nucleobase,
U=a uracil nucleobase,
d=a 2′-β-D-deoxyribosyl sugar moiety
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, Compound No. 1840071 is in the form of an anion or a salt thereof, for example, a sodium salt. In certain embodiments, the oligomeric duplex is in anionic form in a solution. In certain embodiments, Compound No. 1840071 is a sodium salt or a potassium salt.

The following chemical structure is one structural representation of Compound No. 1840071:

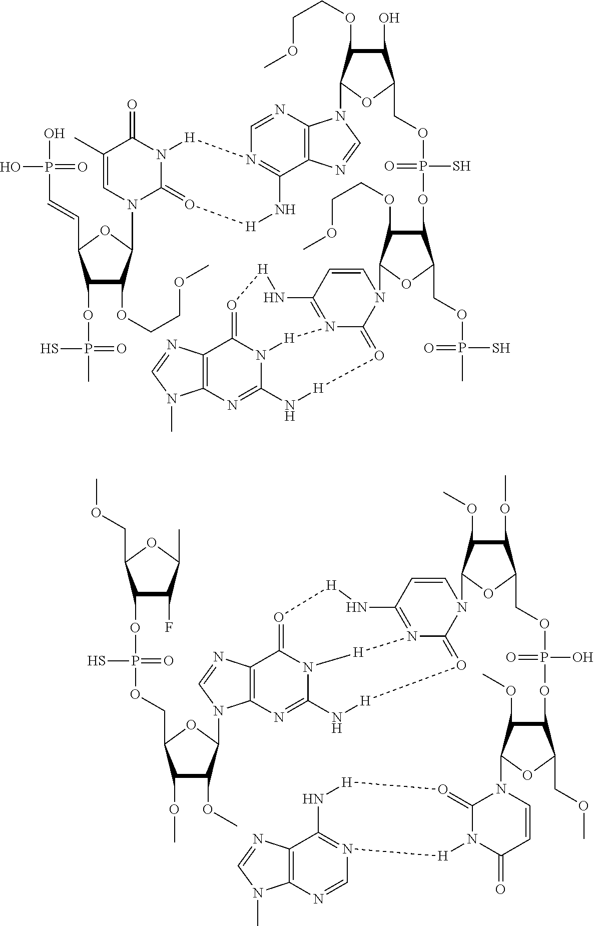

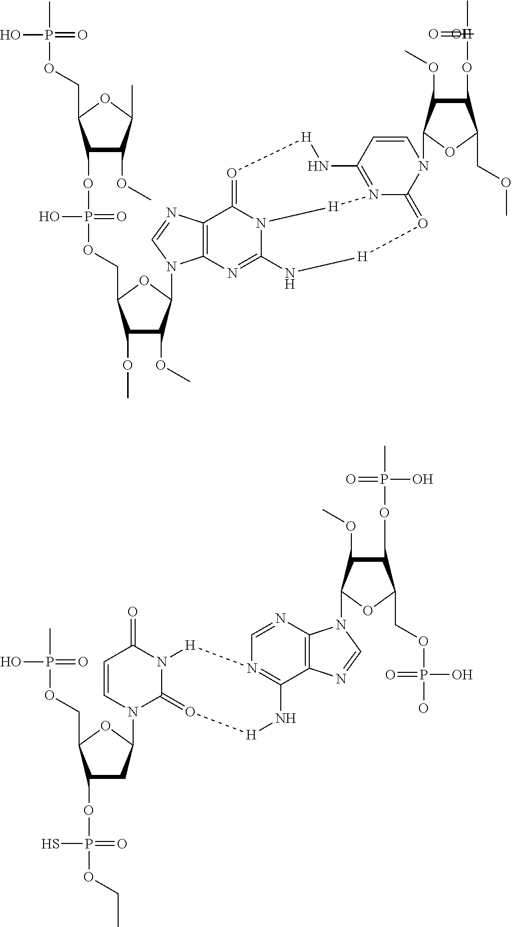

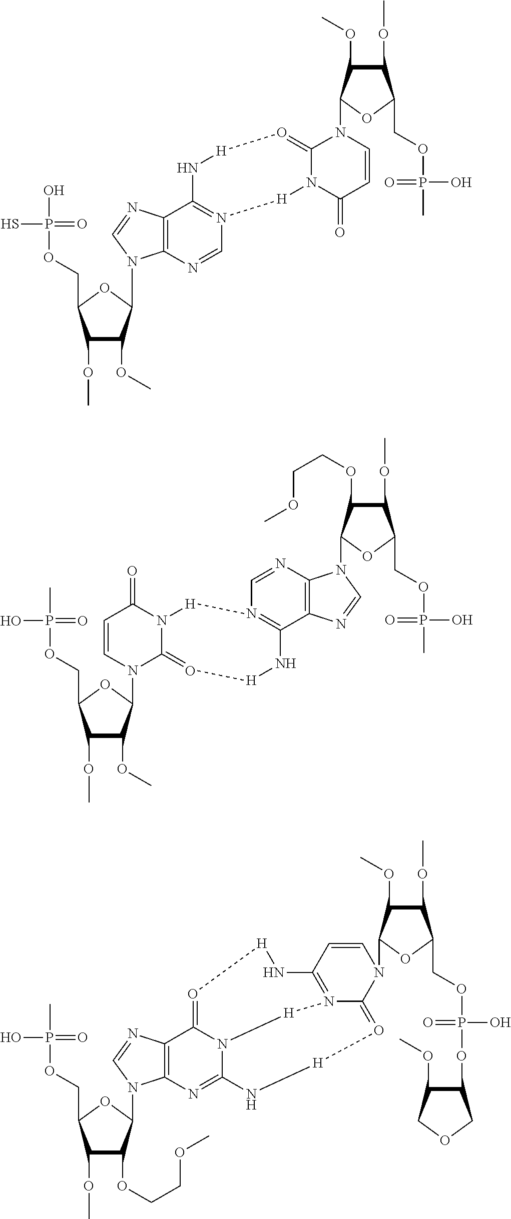

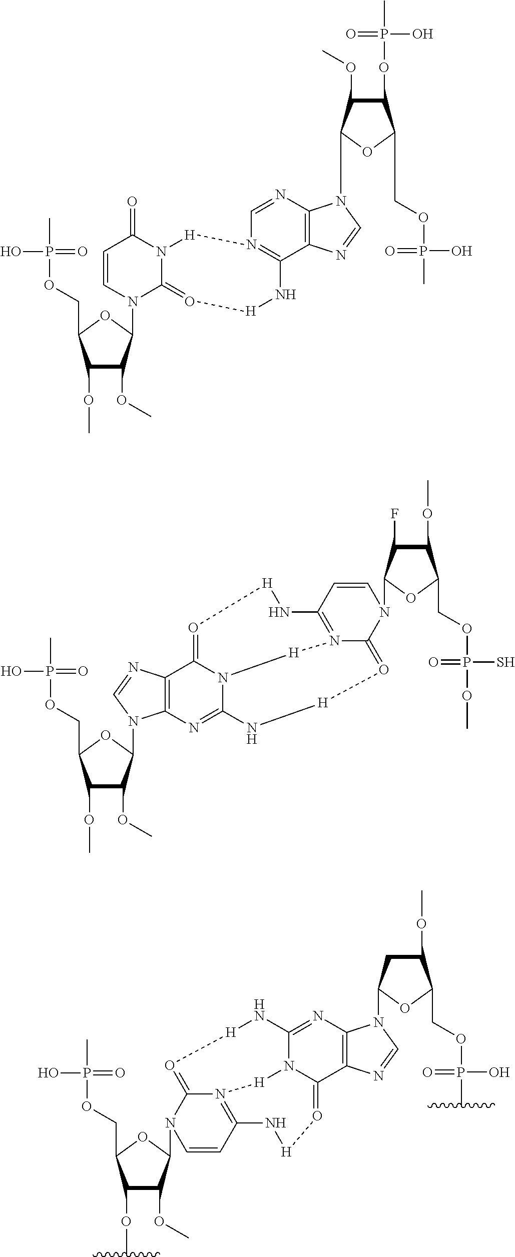

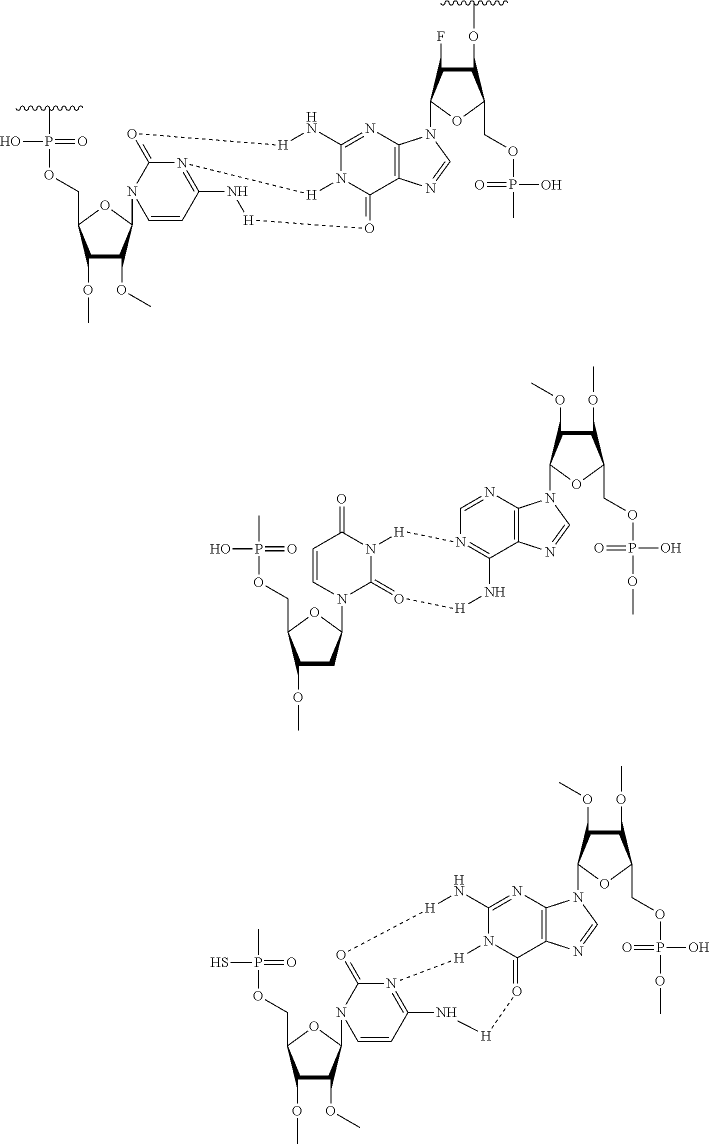

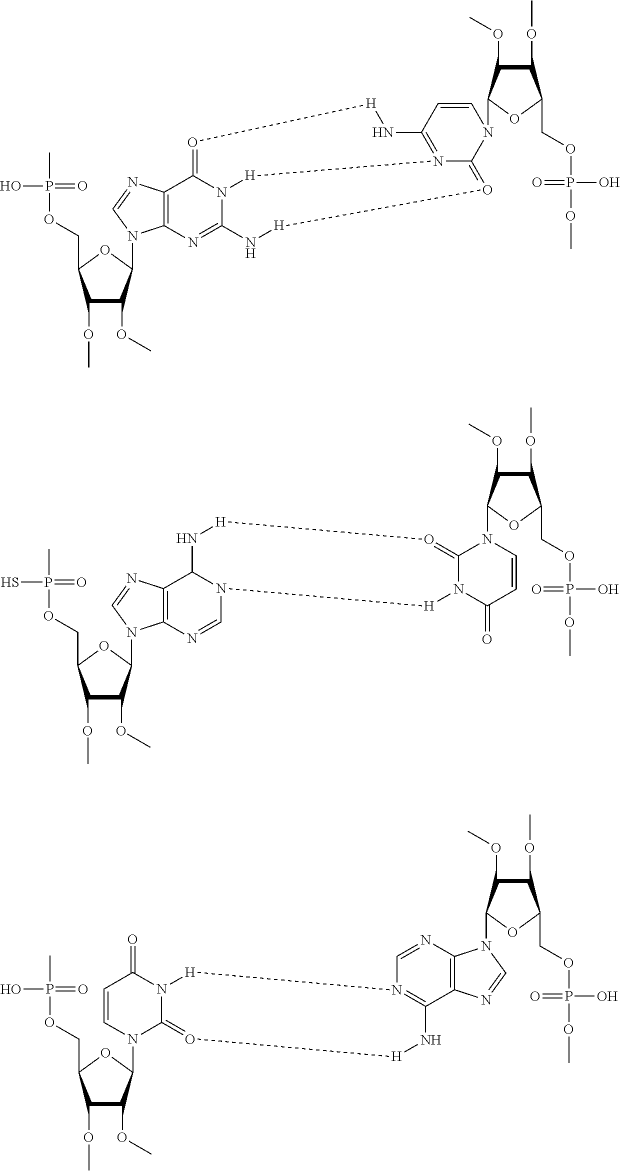

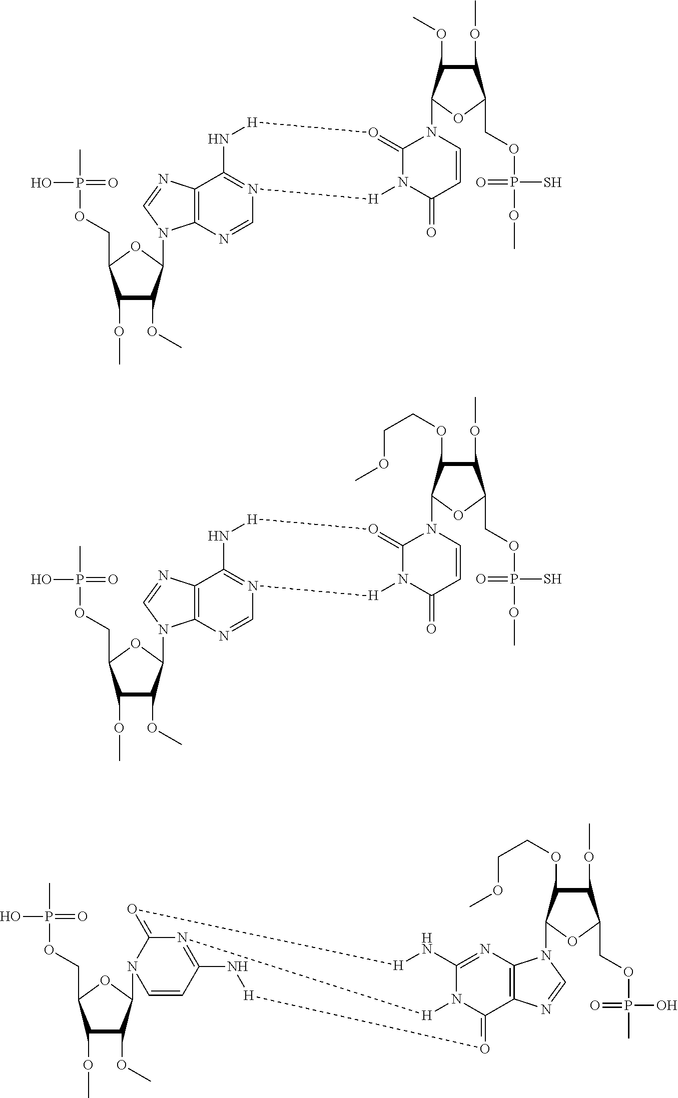

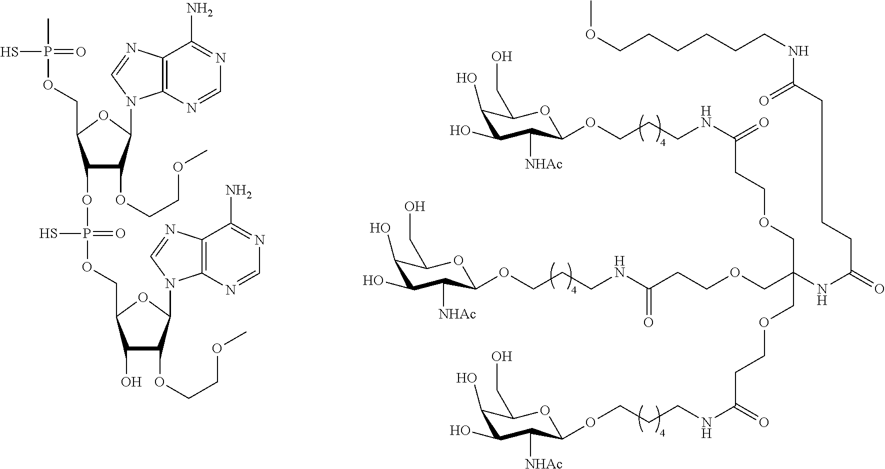

or an ion or a salt thereof.

The following chemical structure represents Compound No. 1840071 in sodium form:

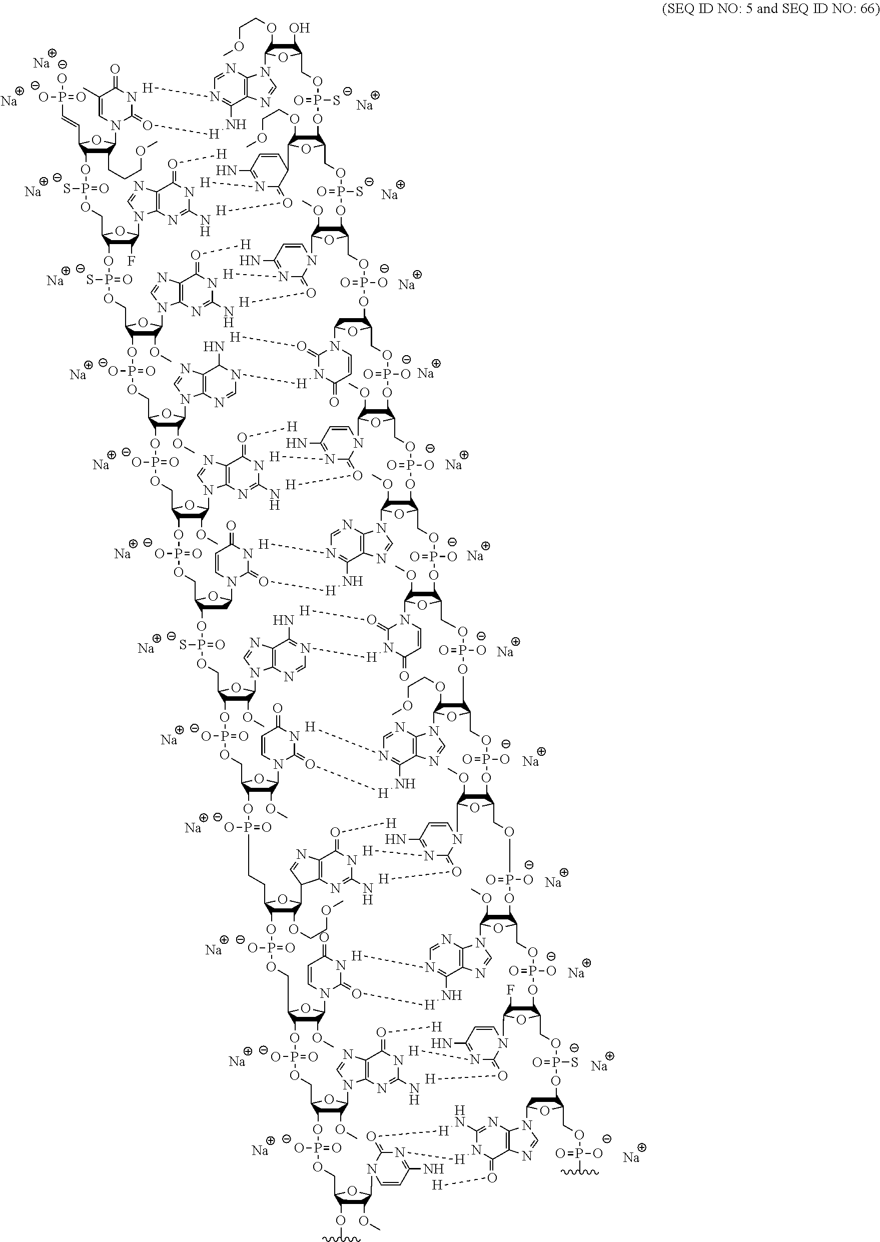

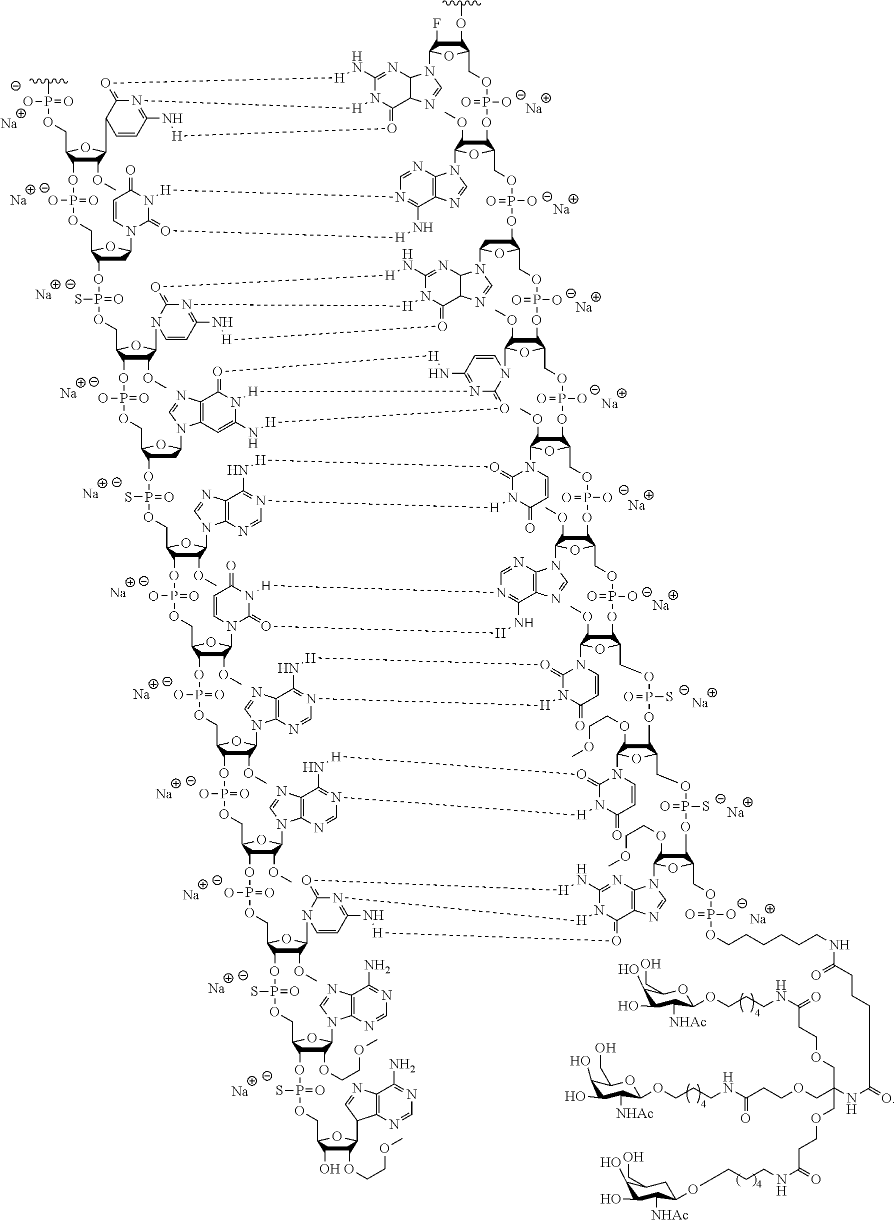

The following chemical structure represents Compound No. 1840071 ion in solution:

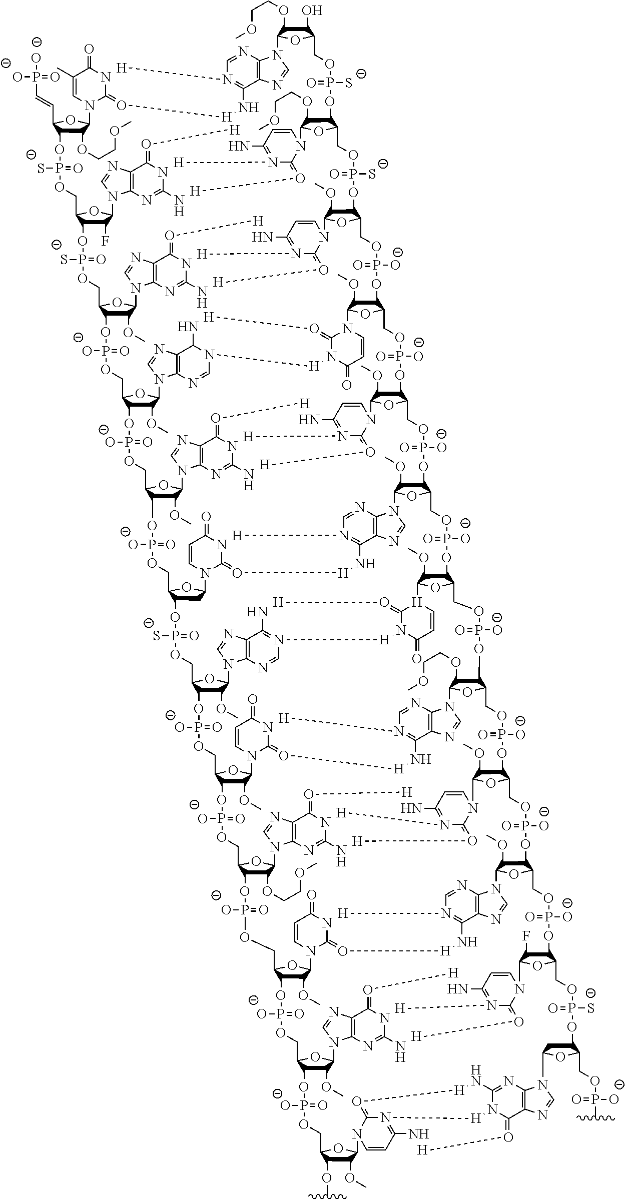

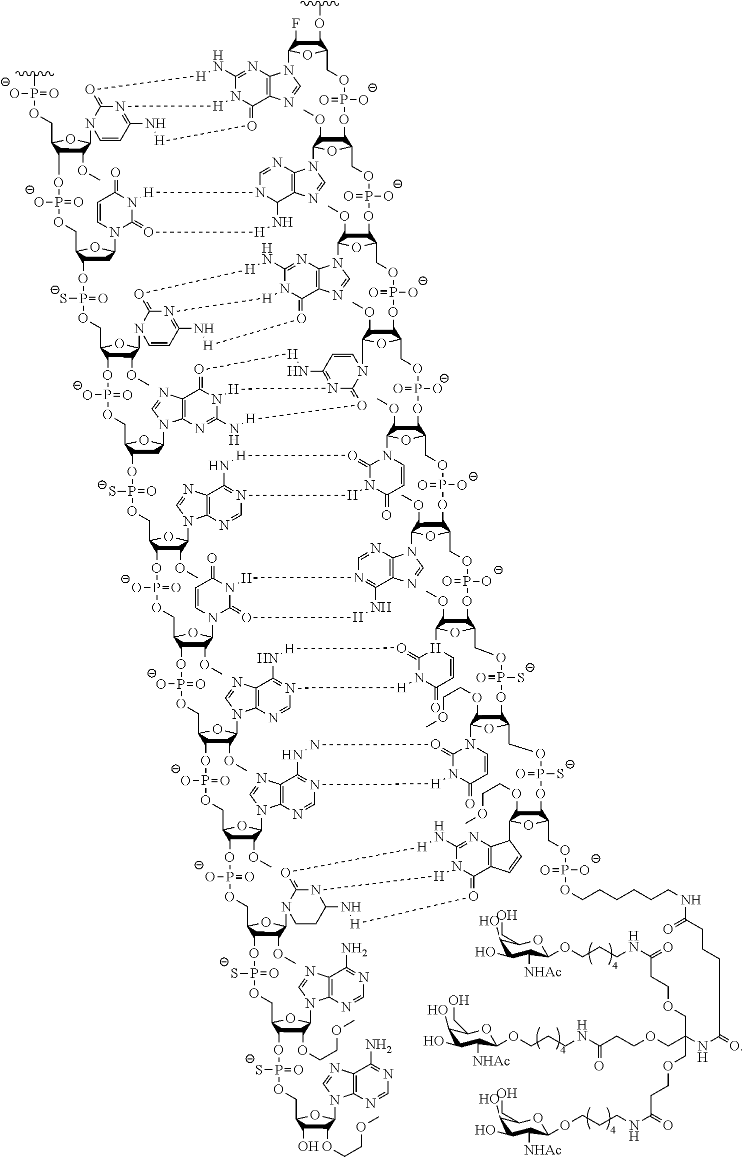

In certain embodiments, provided herein are oligomeric duplexes comprising or consisting of a first oligomeric compound containing a first modified oligonucleotide and a second oligomeric compound containing a second oligomeric compound as described in d. above for Compound no. 1840071, however, the second oligomeric compound optionally includes the conjugate group as depicted, which may be present or absent, and/or located at a different site (e.g., the 5′end), and furthermore independently may comprise a different cell targeting conjugate group.

e. Compound No. 1840453

Provided herein is Compound No. 1840453, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1840453

The first oligomeric compound of Compound No. 1840453, which is Compound No. 1838449, has a first modified oligonucleotide having a nucleobase sequence of (from 5′ to 3′) TGUGCCUCGAUAACUCUGUCCAA (SEQ ID NO:89), wherein the first modified oligonucleotide of Compound 1840453 is represented by the following chemical notation: vP-TₑₛG_{fs}U_{yo}G_{yo}C_{yo}C_{fo}U_{yo}C_{yo}G_{yo}A_{yo}U_{yo}A_{yo}A_{yo}C_{fo}U_{yo}C_{yo}U_{yo}G_{yo}U_{yo}C_{yo}C_{ys}AₑₛAₑ (SEQ ID NO: 31); wherein
A=an adenine nucleobase,
C=an cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1840453

The second oligomeric compound of Compound No. 1840453, which is Compound No. 1838398, has a nucleobase sequence of (from 5′ to 3′) GGACAGAGUUAUCGAGGCACA (SEQ ID NO: 102), wherein the second oligomeric compound of Compound 1840453 is represented by the following chemical notation: [THA-GalNAc]-GₑₛGₑₛA_{yo}C_{yo}A_{yo}G_{yo}A_{yo}G_{yo}U_{fo}U_{fo}A_{fo}U_{yo}C_{yo}GₑₒA_{yo}G_{yo}G_{yo}C_{yo}A_{ys}CₑₛAₑ (SEQ ID NO: 68); wherein:

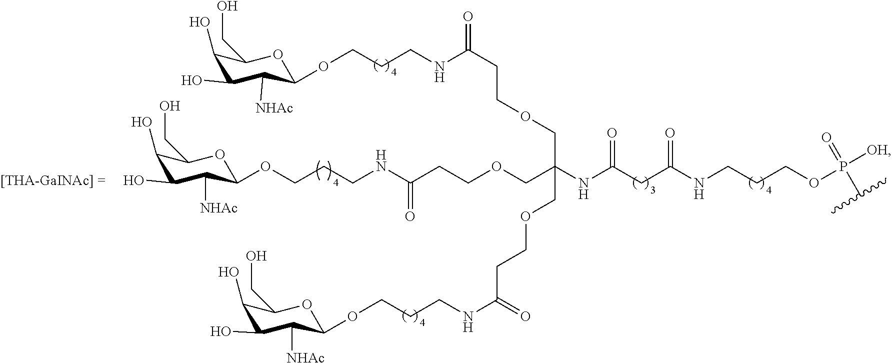

A=an adenine nucleobase,
C=an cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, Compound No. 1840453 is in the form of an anion or a salt thereof, for example, a sodium salt. In certain embodiments, the oligomeric duplex is in anionic form in a solution. In certain embodiments, Compound No. 1840453 is a sodium salt or a potassium salt.

The following chemical structure is one structural representation of Compound No. 1840453:

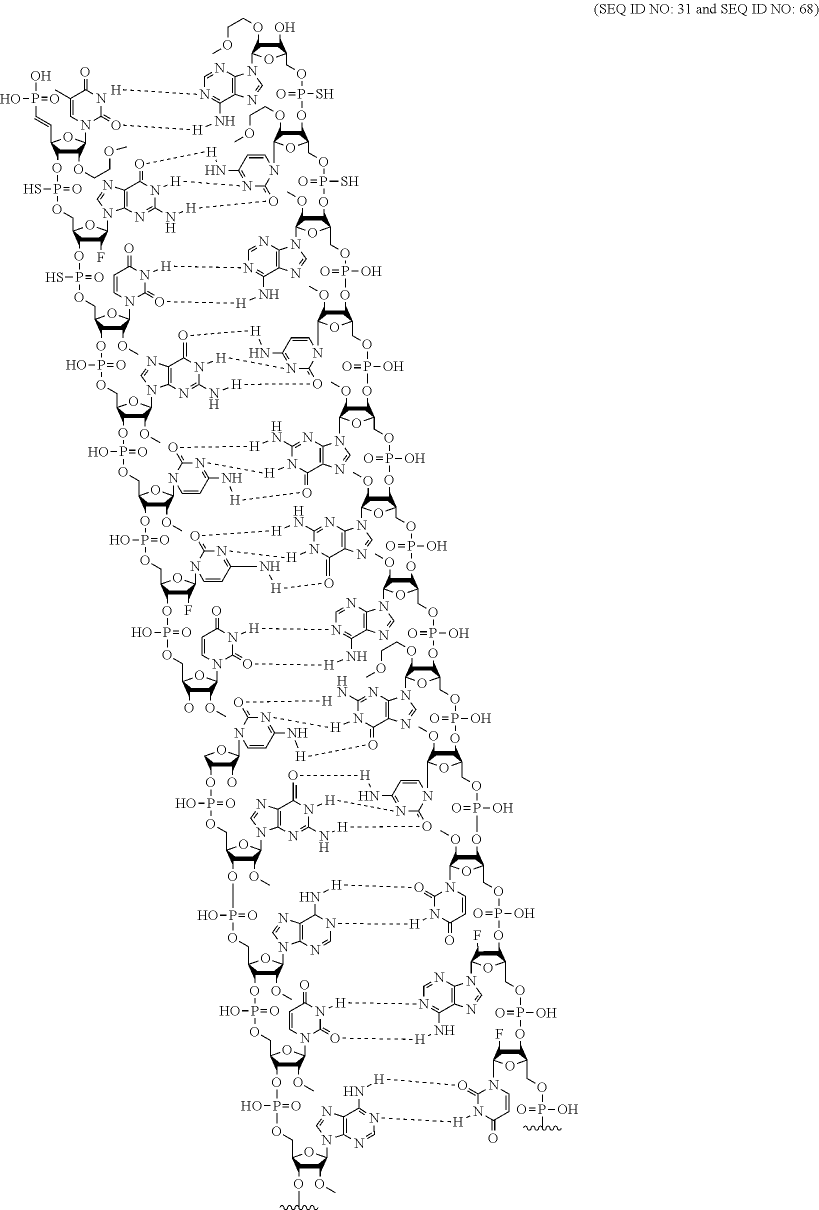

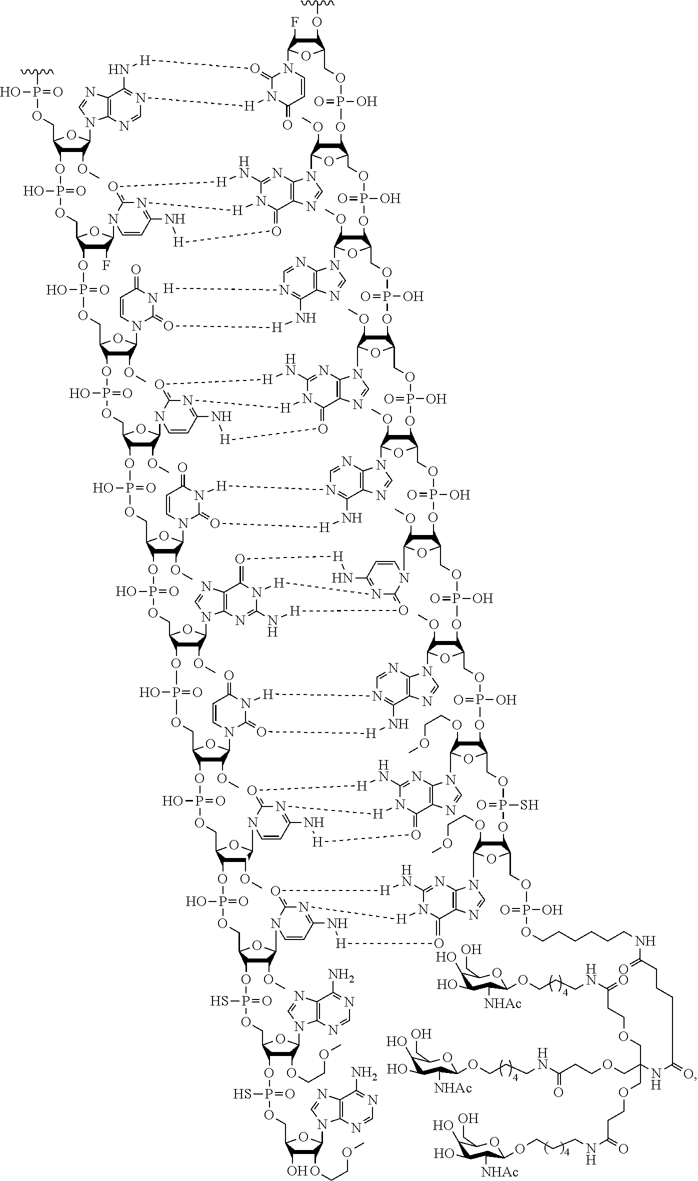

or an ion or a salt thereof.

The following chemical structure represents Compound No. 1840453 in sodium form:

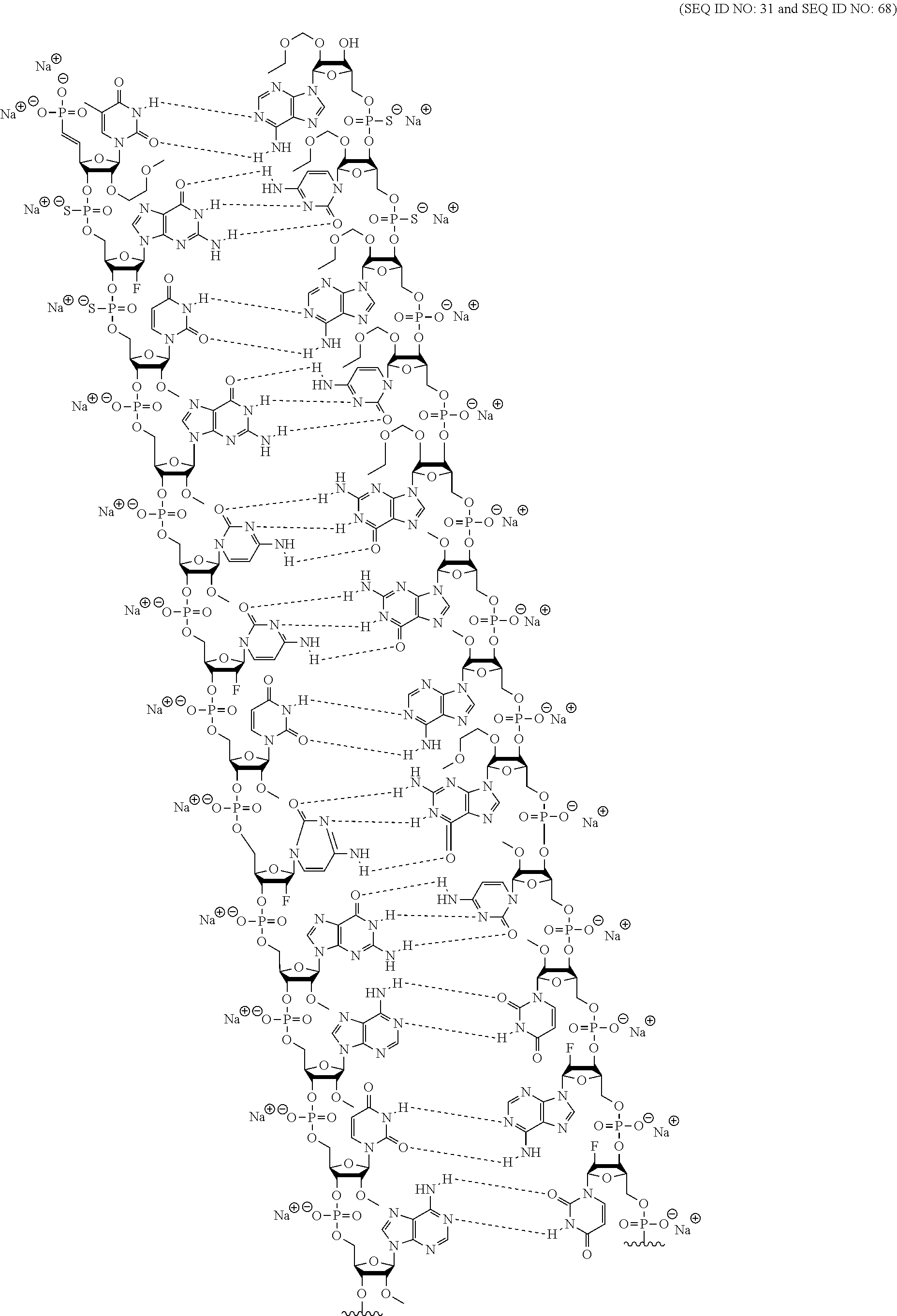

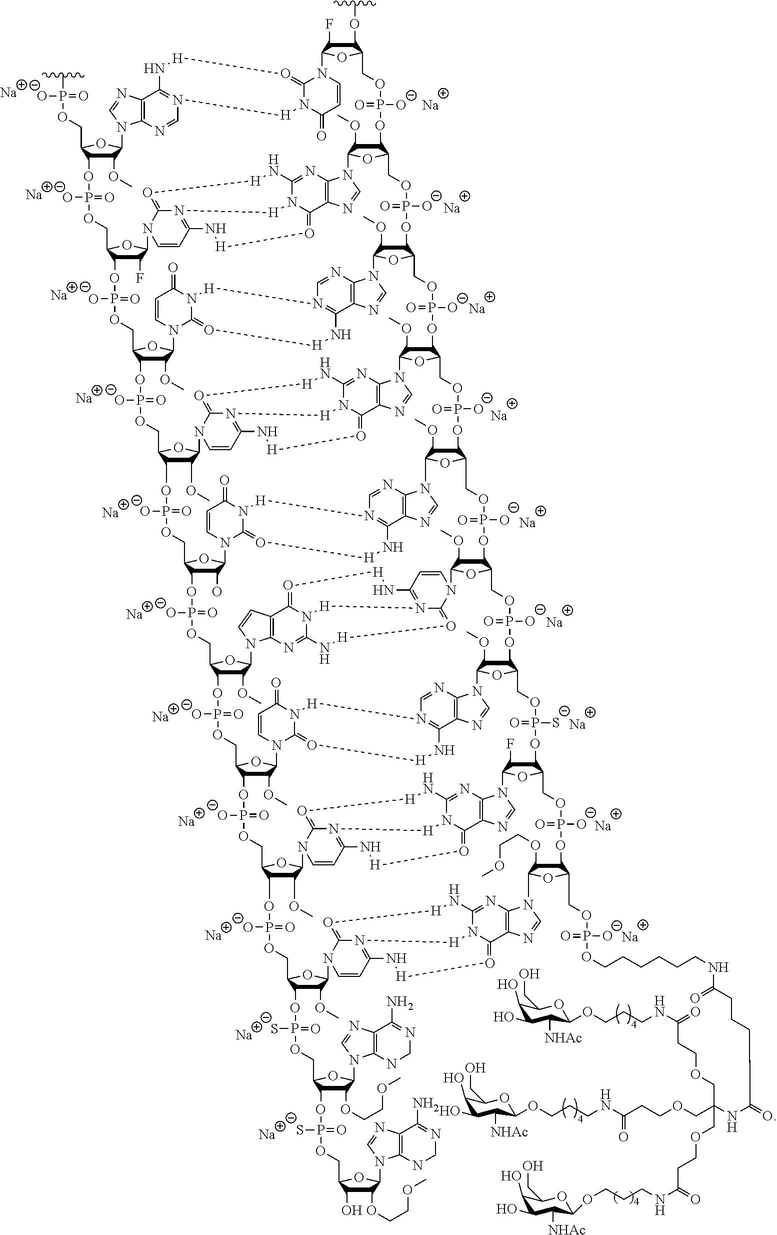

The following chemical structure represents Compound No. 1840453 ion in solution:

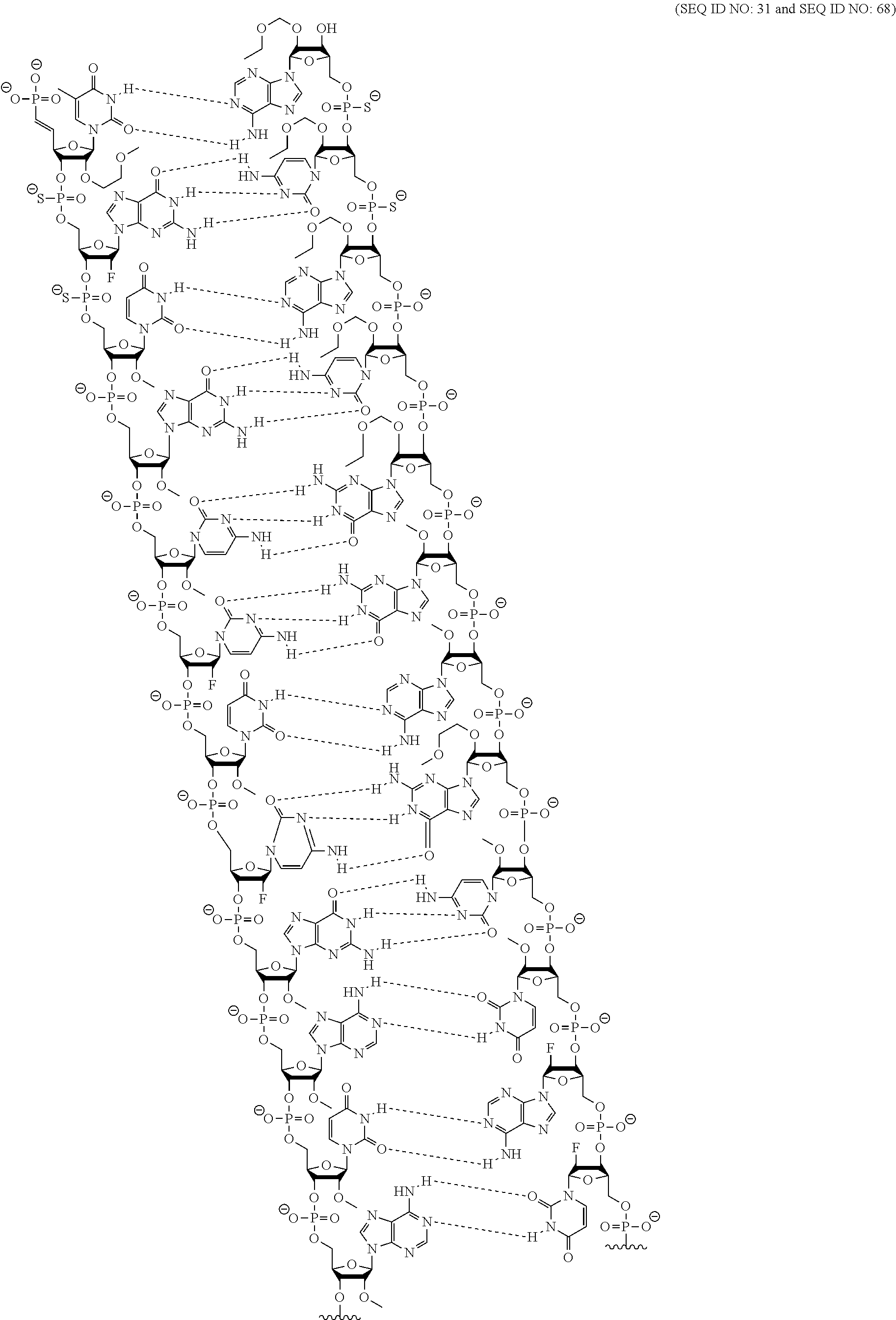

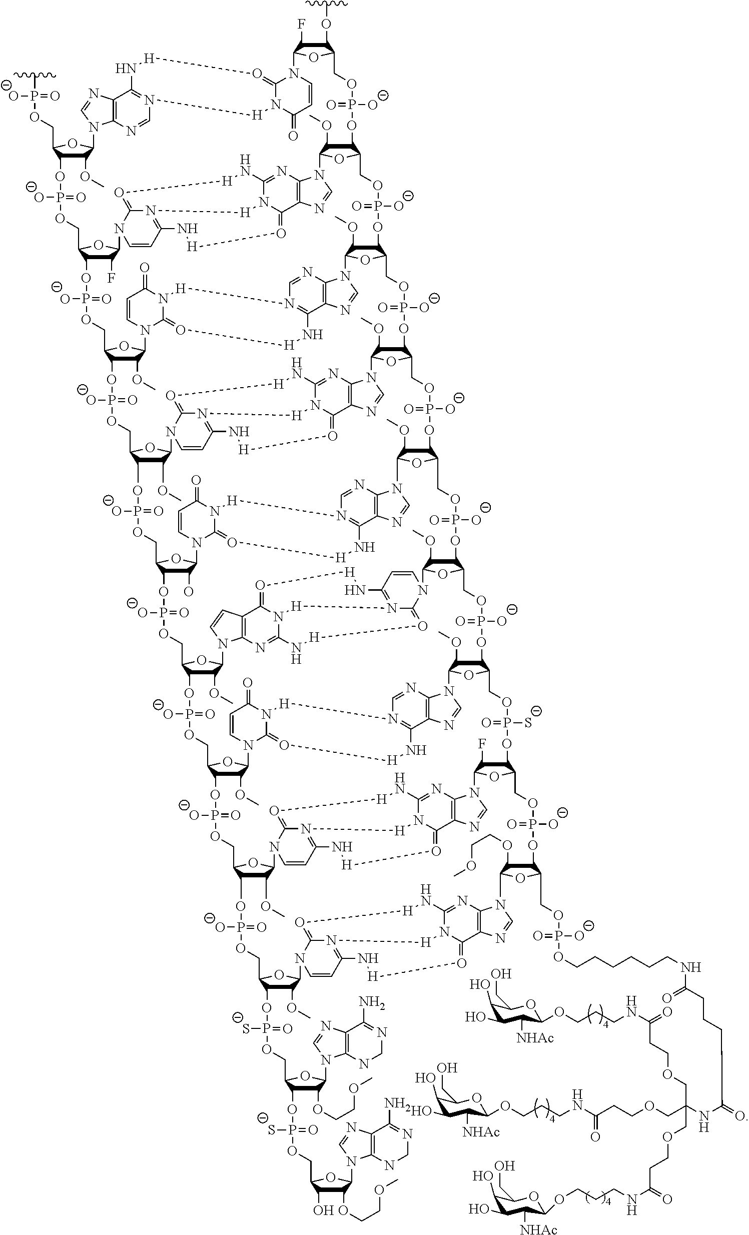

In certain embodiments, provided herein are oligomeric duplexes comprising or consisting of a first oligomeric compound containing a first modified oligonucleotide and a second oligomeric compound containing a second oligomeric compound as described in e. above for Compound no. 1840453, however, the second oligomeric compound optionally includes the conjugate group as depicted, which may be present or absent, and/or located at a different site (e.g., the 5′end), and furthermore independently may comprise a different cell targeting conjugate group.

f. Compound No. 1841135

Provided herein is Compound No. 1841135, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1841135

The first modified oligonucleotide of Compound no. 1841135, which is Compound No. 1838468, has a nucleobase sequence of (from 5′ to 3′) TUCGAUAACUCUGUCCAUUACAA (SEQ ID NO: 97), wherein the first modified oligonucleotide of Compound 1841135 is represented by the following chemical notation: vP-TₑₛU_{fs}C_{yo}G_{yo}A_{yo}U_{yo}A_{yo}A_{yo}C_{yo}U_{yo}C_{yo}U_{yo}G_{yo}U_{fo}C_{yo}C_{fo}A_{yo}U_{yo}U_{yo}A_{yo}C_{ys}AₑₛAₑ (SEQ ID NO: 45); wherein
A=an adenine nucleobase,
C=an cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1841135

The second oligomeric compound of Compound No. 1841135, which is Compound No. 1838393, has a nucleobase sequence of (from 5′ to 3′) GUAAUGGACAGAGUUAUCGAA (SEQ ID NO: 110), wherein the second oligomeric compound of Compound 1841135 is represented by the following chemical notation: [THA-GalNAc]-GₑₛUₑₛA_{yo}A_{yo}U_{yo}G_{yo}G_{yo}A_{yo}C_{fo}A_{fo}G_{fo}A_{yo}G_{yo}UₑₒU_{yo}A_{yo}U_{yo}C_{yo}G_{ys}AₑₛAₑ (SEQ ID NO: 76); wherein:

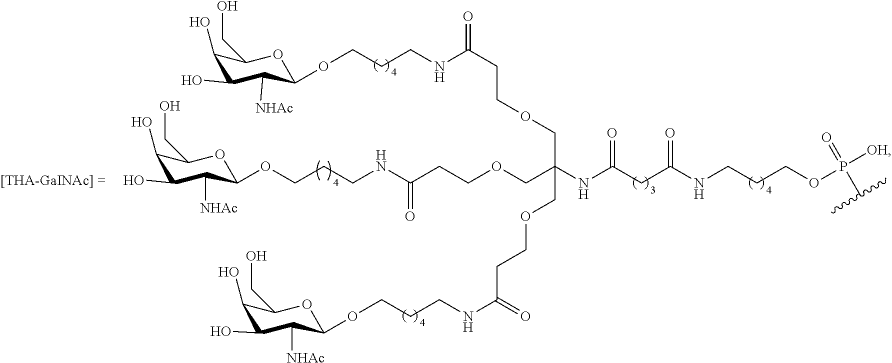

A=an adenine nucleobase,
C=an cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, Compound No. 1841135 is in the form of an anion or a salt thereof, for example, the oligomeric duplex may be in the form of a sodium salt. In certain embodiments, the oligomeric duplex is in anionic form in a solution. In certain embodiments, Compound No. 1841135 is a sodium salt or a potassium salt.

The following chemical structure is one structural representation of Compound No. 1841135.

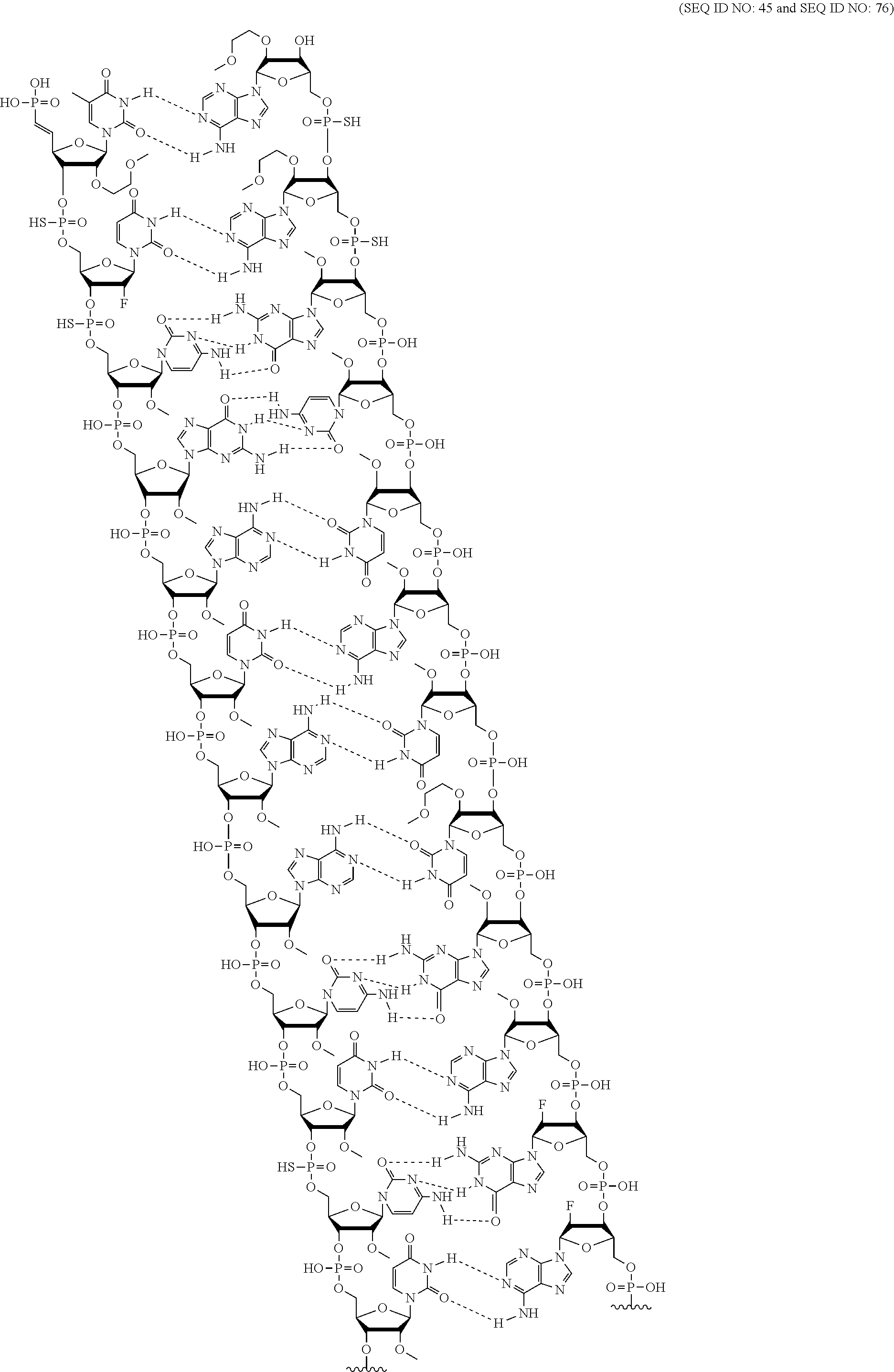

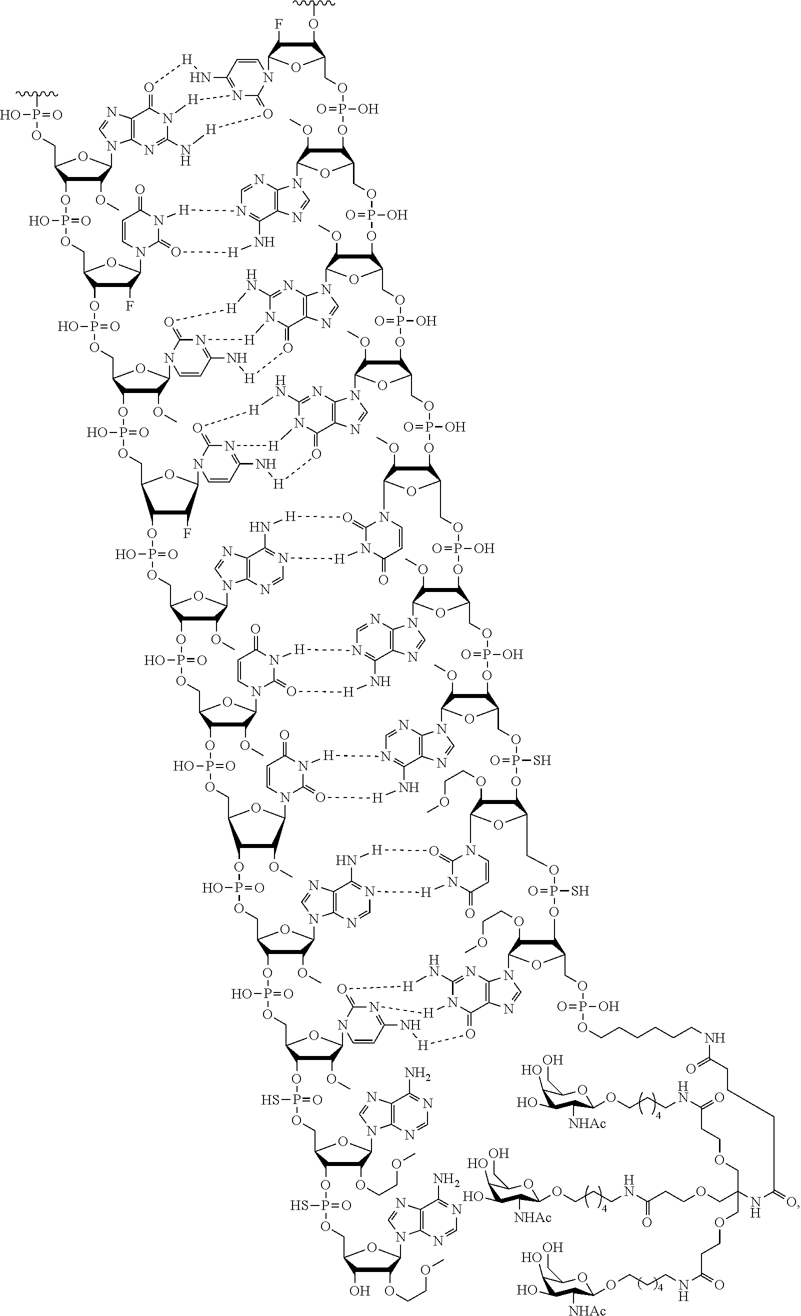

or an ion or a salt thereof

The following chemical structure represents Compound No. 1841135 in sodium form:

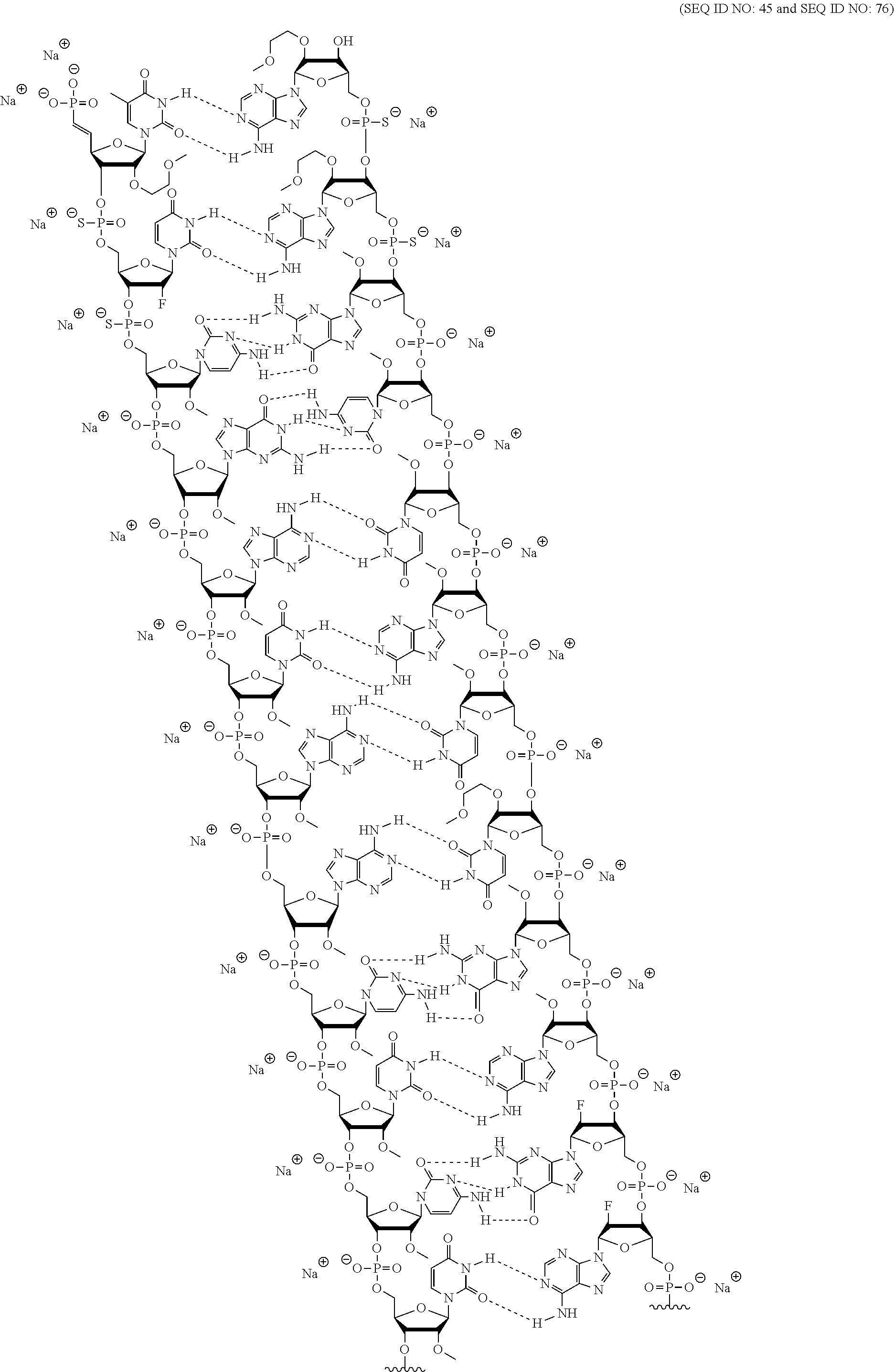

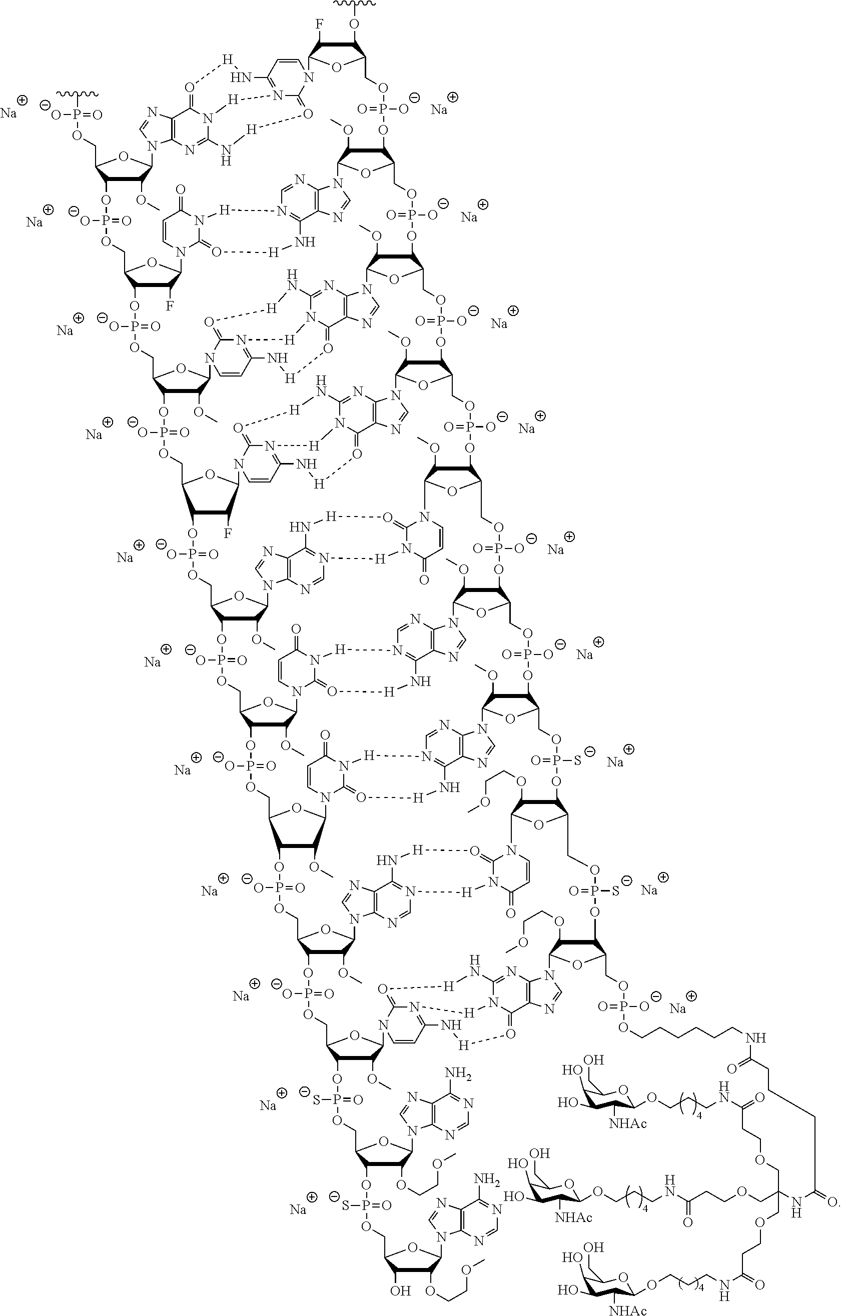

The following chemical structure represents Compound No. 1841135 ion in solution:

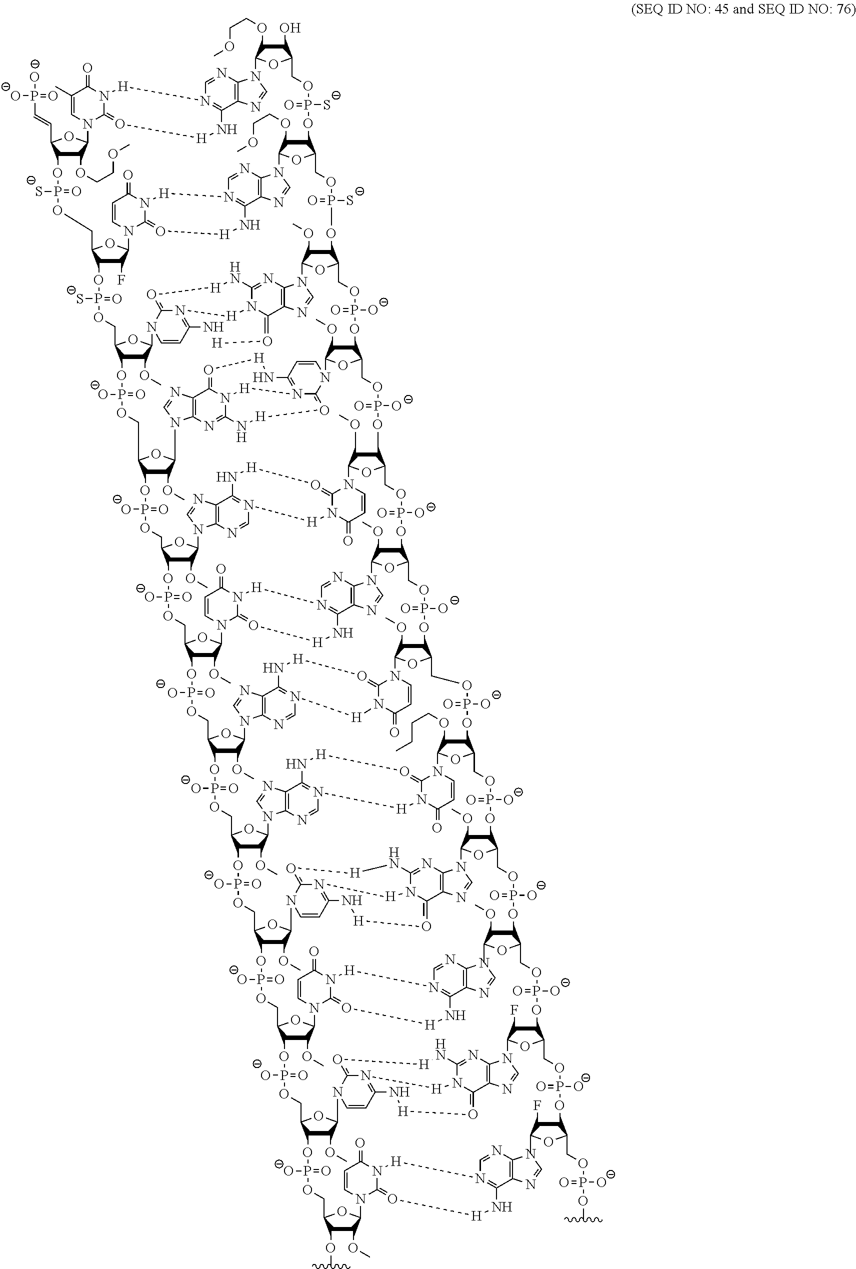

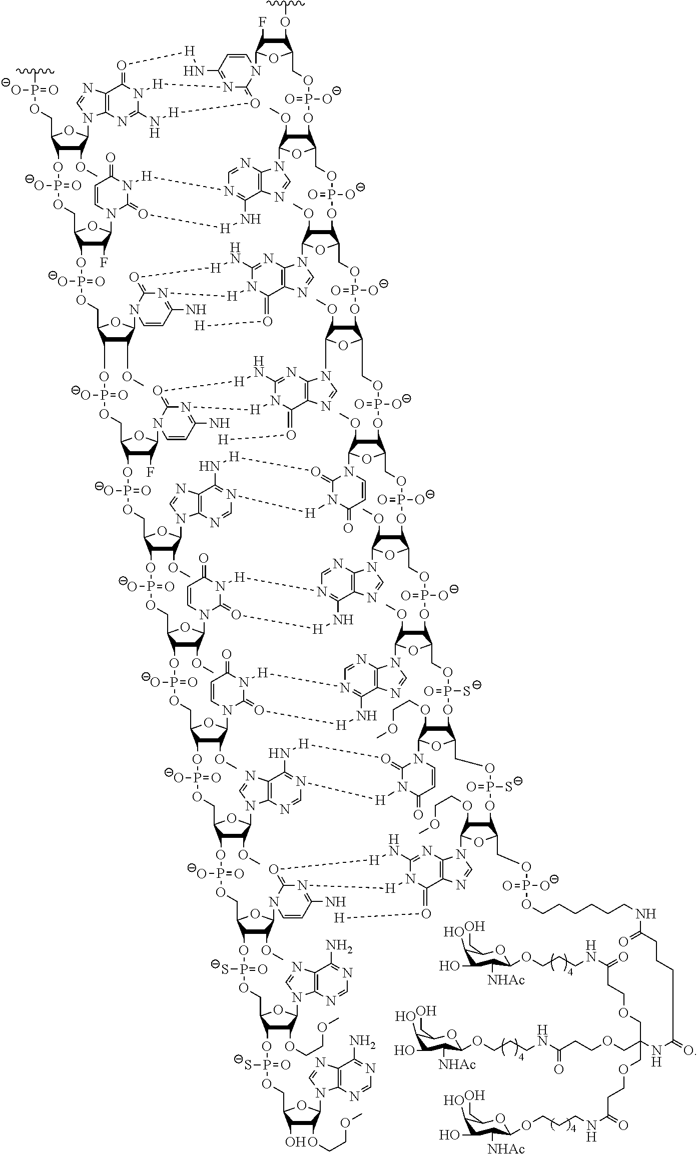

In certain embodiments, provided herein are oligomeric duplexes comprising or consisting of a first oligomeric compound containing a first modified oligonucleotide and a second oligomeric compound containing a second oligomeric compound as described in f. above for Compound no. 1841135, however, the second oligomeric compound optionally includes the conjugate group as depicted, which may be present or absent, and/or located at a different site (e.g., the 5′end), and furthermore independently may comprise a different cell targeting conjugate group.

g. Compound No. 1841136

Provided herein is Compound No. 1841136, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1841136

The first modified oligonucleotide of Compound no. 1841136, which is Compound No. 1838446, has a nucleobase sequence of (from 5′ to 3′) TUCGAUAACUCUGUCCAUUACAA (SEQ ID NO: 97), wherein the first oligomeric compound of Compound 1841136 is represented by the following chemical notation: vP-TₑₛU_{fs}C_{yo}G_{yo}A_{yo}U_{fo}A_{yo}A_{yo}C_{yo}U_{yo}C_{yo}U_{yo}G_{yo}U_{fo}C_{yo}C_{yo}A_{yo}U_{yo}U_{yo}A_{yo}C_{ys}AₑₛAₑ (SEQ ID NO: 46); wherein
A=an adenine nucleobase,
C=an cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
VP=a 5′ vinyl phosphonate moiety.

### Second Oligomeric Compound of Compound No. 1841136

The second oligomeric compound of Compound No. 1841136, which is Compound No. 1838393, has a nucleobase sequence of (from 5′ to 3′) GUAAUGGACAGAGUUAUCGAA (SEQ ID NO: 110), wherein the second oligomeric compound of Compound 1758193 is represented by the following chemical notation: [THA-GalNAc]-GₑₛUₑₛA_{yo}A_{yo}U_{yo}G_{yo}G_{yo}A_{yo}C_{fo}A_{fo}G_{fo}A_{yo}G_{yo}UₑₒU_{yo}A_{yo}U_{yo}C_{yo}G_{ys}AₑₛAₑ (SEQ ID NO: 76); wherein:

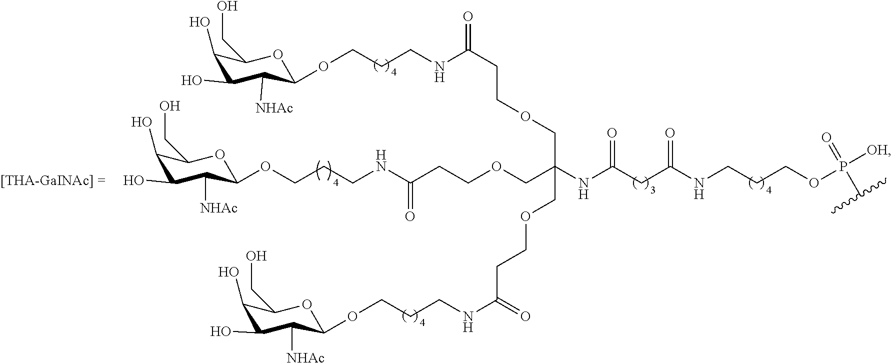

A=an adenine nucleobase,
C=an cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In certain embodiments, Compound No. 1841136 is in the form of an anion or a salt thereof, for example, the oligomeric duplex may be in the form of a sodium salt. In certain embodiments, the oligomeric duplex is in anionic form in a solution. In certain embodiments, Compound No. 1841136 is a sodium salt or a potassium salt.

The following chemical structure is one structural representation of Compound No. 1841136:

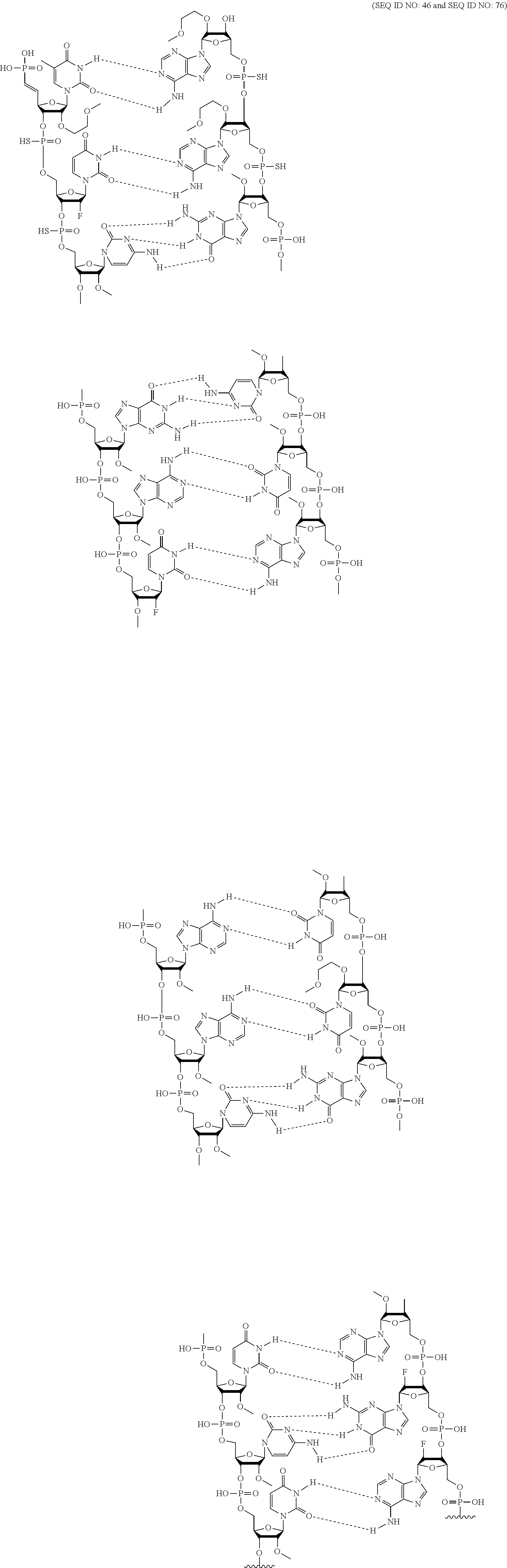

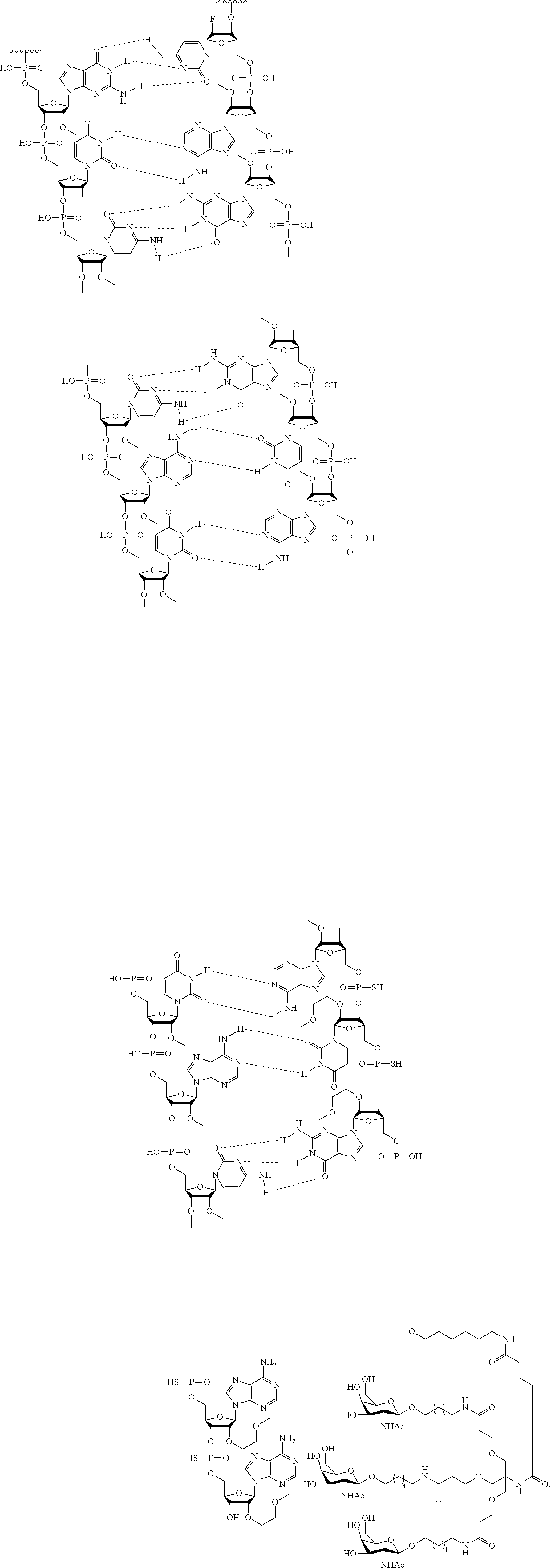

or an ion or a salt thereof.

The following chemical structure represents Compound No. 1841136 in sodium form:

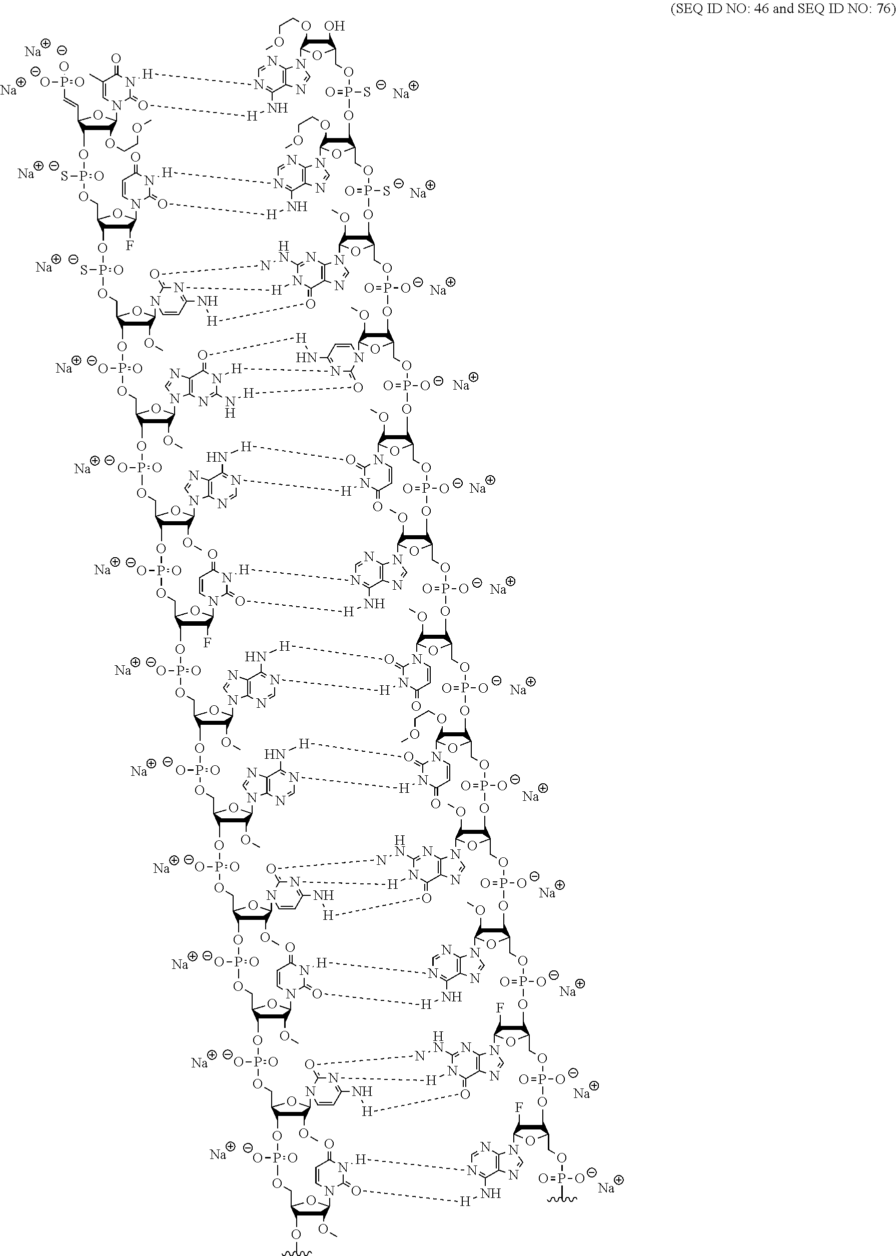

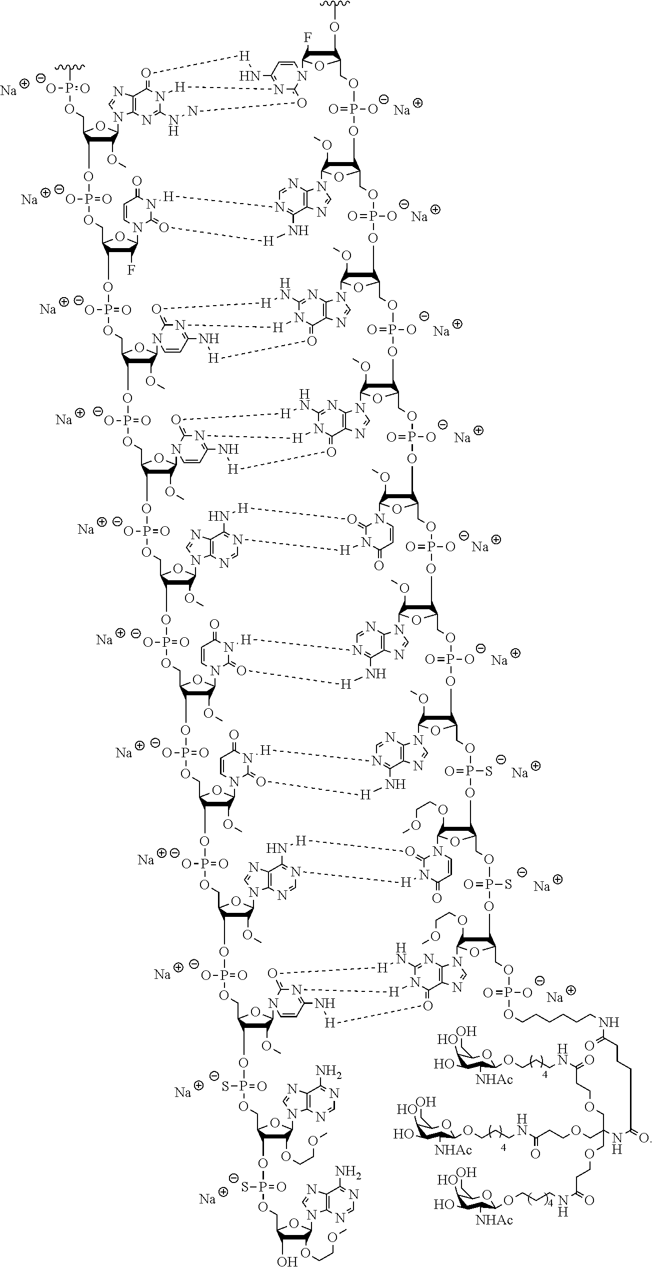

The following chemical structure represents Compound No. 1841136 ion in solution:

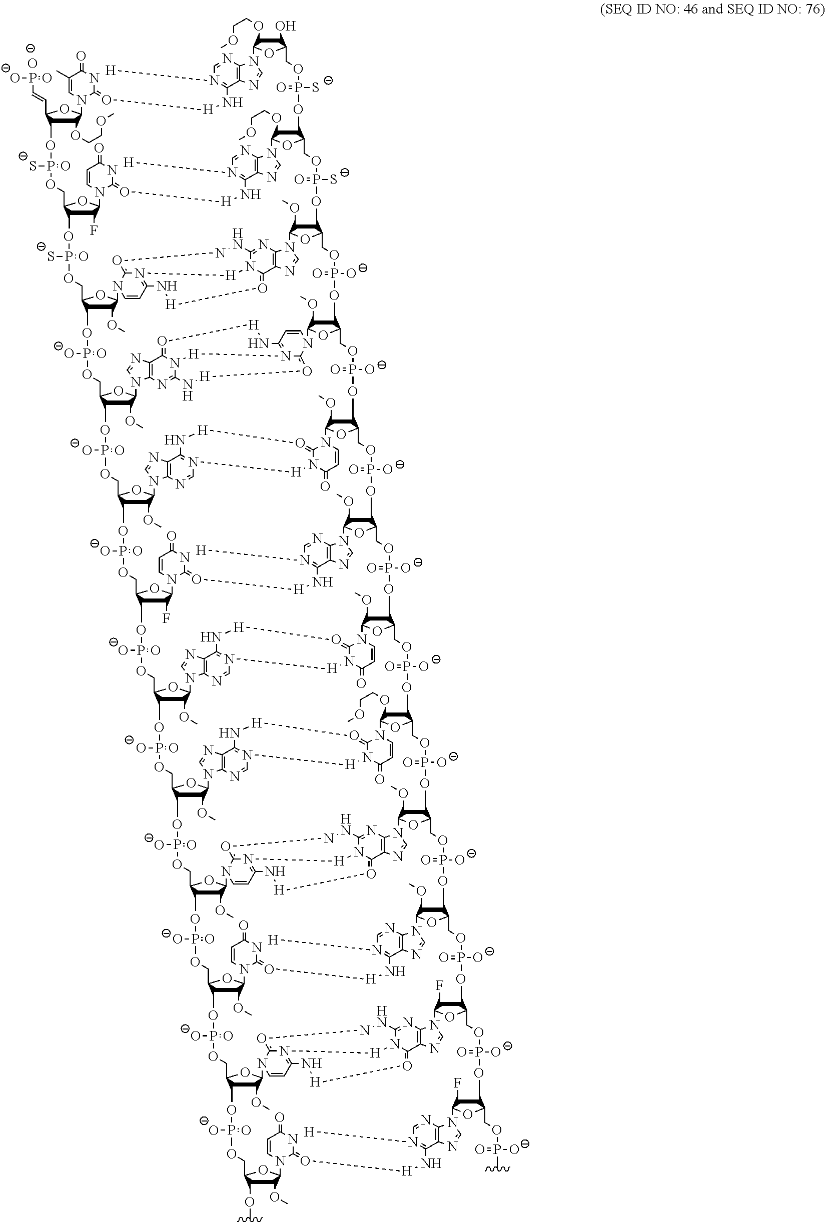

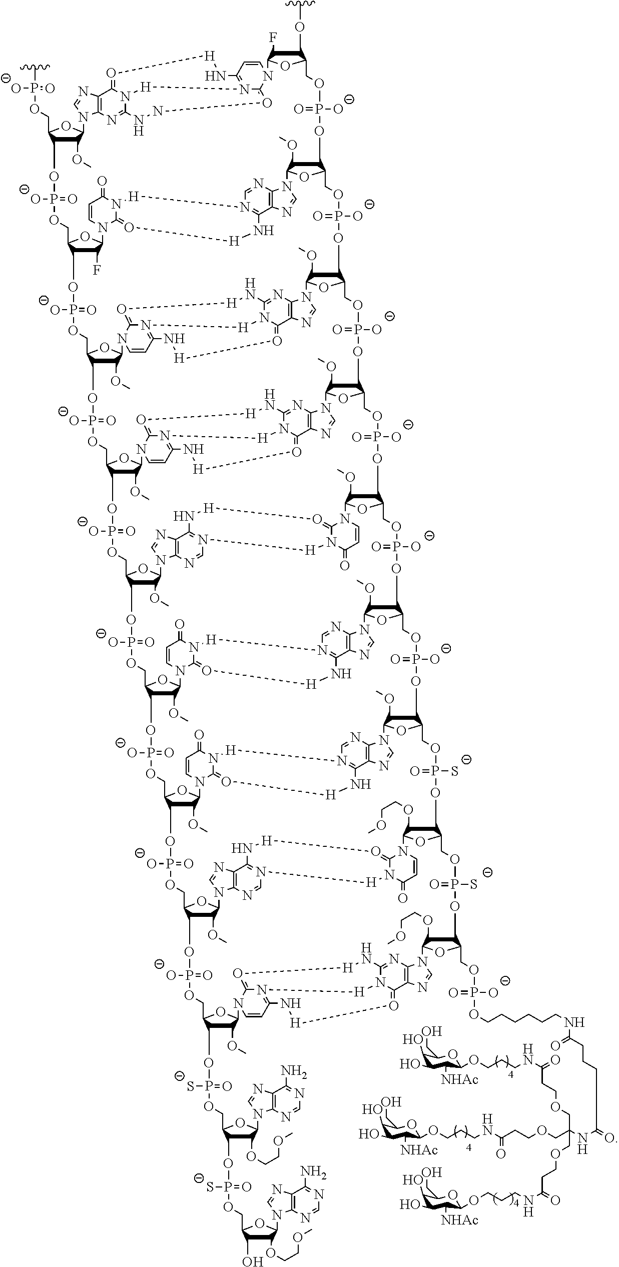

In certain embodiments, provided herein are oligomeric duplexes comprising or consisting of a first oligomeric compound containing a first modified oligonucleotide and a second oligomeric compound containing a second oligomeric compound as described in g. above for Compound no. 1841136, however, the second oligomeric compound optionally includes the conjugate group as depicted, which may be present or absent, and/or located at a different site (e.g., the 5′end), and furthermore independently may comprise a different cell targeting conjugate group.

### NONLIMITING DISCLOSURE AND INCORPORATION BY REFERENCE

Section headings used herein are for organizational purposes only and are not to be construed as limiting the subject matter described.

All documents, websites, URLs, or portions thereof, cited in this application, including, but not limited to, patents, patent applications, articles, books, and treatises, are hereby expressly incorporated-by-reference for the portions of the document discussed herein and in their entirety.

While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine(methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and oligonucleotides having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or R such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXEMPLIFICATION

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1: Design of Oligomeric Compounds

Oligomeric duplex compounds comprising antisense oligomeric compound complementary to a human LPA nucleic acid, and sense oligomeric compound complementary to an antisense oligomeric compound were designed as follows.

### Design of Antisense Oligomeric Compounds

Antisense oligomeric compounds were prepared as described in Table 1. Each antisense oligomeric compound is 23 nucleosides in length and has a sugar motif (from 5′ to 3′) as indicated, wherein each ‘e’ represents a 2′-MOE sugar moiety, each ‘y’ represents a 2′-OMe sugar moiety, each ‘f’ represents a 2′-F sugar moiety, each ‘h’ represents a 3′-fluoro-hexitol sugar moiety, each ‘d’ represents a 2′-2′-β-D-deoxyribose sugar moiety, and each ‘x’ represents a 2′-β-D-deoxyxylose sugar moiety; and each antisense oligomeric compound has an internucleoside linkage motif (from 5′ to 3′) as indicated, wherein each ‘o’ represents a phosphodiester internucleoside linkage, and each ‘s’ represents a phosphorothioate internucleoside linkage. Each antisense oligomeric compound has a vinyl phosphonate (VP—) moiety on the 5′-end.

Each antisense oligomeric compound is complementary to SEQ ID NO: 1 (GenBank Accession No. NM_005577.4). In particular, oligomeric compounds provided herein are complementary to a region of human LPA comprising nucleobases 486-530 of GenBank Accession No. NM_005577.4 GCAUCCAUGGUAAUGGACAGAGUUAUCGAGGCACAUACUCCACCA (SEQ ID NO: 2). Many compounds comprise a single mismatch at position 1 on the 5′-end of the antisense oligonucleotide, and certain compounds comprise an additional mismatch within the core 20mer sequence (excluding the 5′ end nucleobase and the two 3′ nucleobase overhang); however, due to the nature of the target LPA sequence, and the presence of kringle IV repeat sequences, an oligonucleotide may bind to multiple sites within SEQ ID NO: 1, having one or two (or more) mismatches within the core 20mer sequence (excluding the 5′ end nucleobase and the two 3′ nucleobase overhang). CPD SID indicates the SEQ ID NO: for the relevant compound, including annotation, e.g., sugar motif and internucleoside motif, and nb SID indicates the SEQ ID NO: for the nucleobase sequence of the relevant compound.

TABLE 1
Antisense oligomeric compound targeted to human LPA

| Compound | Nucleobase Sequence | Sugar Motif | Internucleoside Motif | CPD | nb |
|---|---|---|---|---|---|
| No. | (5′ to 3′) | (5′ to 3′) | (5′ to 3′) | SID | SID |
| 1749544 | TGCCUCGAUAACUCUGUCCAUCA | efyyyfyyyyyyyfyfyyyyyyy | SSOOOOOOOOOOOOOOOOOOSS | 3 | 83 |
| 1764919 | UGCCUCGAUAACUCUGUCCAUCA | yfyyyfyyyyyyyfyfyyyyyyy | SSOOOOOOOOOOOOOOOOOOSS | 4 | 84 |
| 1792676 | TGGAGUAUGUGCCUCGAUAACAA | efyyydyyeyyyydydyyyyyee | SSOOOSOOOOOOOOSOSOOOSS | 5 | 85 |
| 1792678 | TCCUCGAUAACUCUGUCCAUUAA | efyyydyyeyyyydydyyyyyee | SSOOOSOOOOOOOOSOSOOOSS | 6 | 86 |
| 1792688 | TGGAGUAUGUGCCUCGAUAACAA | efyyydyyyyyyyfyfyyyyyee | SSOOOSOOOOOOOOOOOOOOSS | 7 | 85 |
| 1792689 | TCCUCGAUAACUCUGUCCAUUAA | efyyydyyyyyyyfyfyyyyyee | SSOOOSOOOOOOOOOOOOOOSS | 8 | 86 |
| 1792699 | TCCUCGAUAACUCUGUCCAUUAA | efyyydyyeyyyyfyfyyyyyee | SSOOOSOOOOOOOOOOOOOOSS | 9 | 86 |
| 1792698 | TGGAGUAUGUGCCUCGAUAACAA | efyyydyyeyyyyfyfyyyyyee | SSOOOSOOOOOOOOOOOOOOSS | 10 | 85 |
| 1792709 | TCCUCGAUAACUCUGUCCAUUAA | ehyyyfyyyyyyyfyfyyyyyee | SSOOOOOOOOOOOOOOOOOOSS | 11 | 86 |
| 1792708 | TGGAGUAUGUGCCUCGAUAACAA | ehyyyfyyyyyyyfyfyyyyyee | SSOOOOOOOOOOOOOOOOOOSS | 12 | 85 |
| 1792718 | TGGAGUAUGUGCCUCGAUAACAA | ehyyyfyyeyyyyfyfyyyyyee | SSOOOOOOOOOOOOOOOOOOSS | 13 | 85 |
| 1792719 | TCCUCGAUAACUCUGUCCAUUAA | ehyyyfyyeyyyyfyfyyyyyee | SSOOOOOOOOOOOOOOOOOOSS | 14 | 86 |
| 1814997 | TGCCUCGAUAACUCUGUCCAUAA | efyyyyyyyyyyyfyfyyyyyee | SSOOOOOOOOOOOOOOOOOOSS | 15 | 87 |
| 1814998 | TGCCUCGAUAACUCUGUCCAUAA | efyyyfyyyyyyyfyyyyyyyee | SSOOOOOOOOOOOOOOOOOOSS | 16 | 87 |
| 1819834 | TGGAGUAUGUGCCUCGAUAACAA | efyyyyyyyyyyyfyfyyyyyee | SSOOOOOOOOOOOOOOOOOOSS | 17 | 85 |
| 1819835 | TGGAGUAUGUGCCUCGAUAACAA | efyyyfyyyyyyyfyyyyyyyee | SSOOOOOOOOOOOOOOOOOOSS | 18 | 85 |
| 1826580 | TGGAGUAUGUGCCUCGAUAACAA | efyyyyyyeyyyyfyfyyyyyee | SSOOOOOOOOOOOOOOOOOOSS | 19 | 85 |
| 1826581 | TGGAGUAUGUGCCUCGAUAACAA | efyyyfyyeyyyyfyyyyyyyee | SSOOOOOOOOOOOOOOOOOOSS | 20 | 85 |
| 1826582 | TGCCUCGAUAACUCUGUCCAUAA | efyyyyyyeyyyyfyfyyyyyee | SSOOOOOOOOOOOOOOOOOOSS | 21 | 87 |
| 1826583 | TGCCUCGAUAACUCUGUCCAUAA | efyyyfyyeyyyyfyyyyyyyee | SSOOOOOOOOOOOOOOOOOOSS | 22 | 87 |
| 1839388 | TGGAGUAUGUGCCUCGAUAACAA | efyyxfyyyyyyyfyyyyyyyee | SSOOOOOOOOOOOOOOOOOOSS | 23 | 85 |
| 1839376 | TGGAGUAUGUGCCUCGAUAACAA | efyyyfxyyyyyyfyyyyyyyee | SSOOOOOOOOOOOOOOOOOOSS | 24 | 85 |
| 1839378 | TGGAGUAUGUGCCUCGAUAACAA | efyyyxyyyyyyyfyyyyyyyee | SSOOOOOOOOOOOOOOOOOOSS | 25 | 85 |
| 1839377 | TGCCUCGAUAACUCUGUCCAUAA | efyyyfxyyyyyyfyyyyyyyee | SSOOOOOOOOOOOOOOOOOOSS | 26 | 87 |
| 1839379 | TGCCUCGAUAACUCUGUCCAUAA | efyyyxyyyyyyyfyyyyyyyee | SSOOOOOOOOOOOOOOOOOOSS | 27 | 87 |
| 1839380 | TGCCUCGAUAACUCUGUCCAUAA | efyyxfyyyyyyyfyyyyyyyee | SSOOOOOOOOOOOOOOOOOOSS | 28 | 87 |
| 1838448 | TUGCCUCGAUAACUCUGUCCAAA | efyyyfyyyyyyyfyyyyyyyee | SSOOOOOOOOOOOOOOOOOOSS | 29 | 88 |
| 1838470 | TUGCCUCGAUAACUCUGUCCAAA | efyyyyyyyyyyyfyfyyyyyee | SSOOOOOOOOOOOOOOOOOOSS | 30 | 88 |
| 1838449 | TGUGCCUCGAUAACUCUGUCCAA | efyyyfyyyyyyyfyyyyyyyee | SSOOOOOOOOOOOOOOOOOOSS | 31 | 89 |
| 1838471 | TGUGCCUCGAUAACUCUGUCCAA | efyyyyyyyyyyyfyfyyyyyee | SSOOOOOOOOOOOOOOOOOOSS | 32 | 89 |
| 1838451 | TAUGUGCCUCGAUAACUCUGUAA | efyyyfyyyyyyyfyyyyyyyee | SSOOOOOOOOOOOOOOOOOOSS | 33 | 90 |
| 1838473 | TAUGUGCCUCGAUAACUCUGUAA | efyyyyyyyyyyyfyfyyyyyee | SSOOOOOOOOOOOOOOOOOOSS | 34 | 90 |
| 1838453 | TGUAUGUGCCUCGAUAACUCUAA | efyyyfyyyyyyyfyyyyyyyee | SSOOOOOOOOOOOOOOOOOOSS | 35 | 91 |
| 1838475 | TGUAUGUGCCUCGAUAACUCUAA | efyyyyyyyyyyyfyfyyyyyee | SSOOOOOOOOOOOOOOOOOOSS | 36 | 91 |
| 1838454 | TAGUAUGUGCCUCGAUAACUCAA | efyyyfyyyyyyyfyyyyyyyee | SSOOOOOOOOOOOOOOOOOOSS | 37 | 92 |
| 1838476 | TAGUAUGUGCCUCGAUAACUCAA | efyyyyyyyyyyyfyfyyyyyee | SSOOOOOOOOOOOOOOOOOOSS | 38 | 92 |
| 1838464 | TUAACUCUGUCCAUUACCAUGAA | efyyyyyyyyyyyfyfyyyyyee | SSOOOOOOOOOOOOOOOOOOSS | 39 | 93 |
| 1838465 | TAUAACUCUGUCCAUUACCAUAA | efyyyyyyyyyyyfyfyyyyyee | SSOOOOOOOOOOOOOOOOOOSS | 40 | 94 |
| 1838472 | TUGUGCCUCGAUAACUCUGUCAA | efyyyyyyyyyyyfyfyyyyyee | SSOOOOOOOOOOOOOOOOOOSS | 41 | 95 |
| 1838450 | TUGUGCCUCGAUAACUCUGUCAA | efyyyfyyyyyyyfyyyyyyyee | SSOOOOOOOOOOOOOOOOOOSS | 42 | 95 |
| 1838469 | TCUCGAUAACUCUGUCCAUUAAA | efyyyyyyyyyyyfyfyyyyyee | SSOOOOOOOOOOOOOOOOOOSS | 43 | 96 |
| 1838447 | TCUCGAUAACUCUGUCCAUUAAA | efyyyfyyyyyyyfyyyyyyyee | SSOOOOOOOOOOOOOOOOOOSS | 44 | 96 |
| 1838468 | TUCGAUAACUCUGUCCAUUACAA | efyyyyyyyyyyyfyfyyyyyee | SSOOOOOOOOOOOOOOOOOOSS | 45 | 97 |
| 1838446 | TUCGAUAACUCUGUCCAUUACAA | efyyyfyyyyyyyfyyyyyyyee | SSOOOOOOOOOOOOOOOOOOSS | 46 | 97 |
| 1838466 | TGAUAACUCUGUCCAUUACCAAA | efyyyyyyyyyyyfyfyyyyyee | SSOOOOOOOOOOOOOOOOOOSS | 47 | 98 |
| 1792668 | TGCCUCGAUAACUCUGUCCAUAA | efyyydyyeyyyydydyyyyyee | SSOOOOOOOOOOOOOOOOOOSS | 48 | 87 |
| 1792704 | TGCCUCGAUAACUCUGUCCAUAA | ehyyyfyyyyyyyfyfyyyyyee | SSOOOOOOOOOOOOOOOOOOSS | 132 | 87 |
| 1792714 | TGCCUCGAUAACUCUGUCCAUAA | ehyyyfyyeyyyyfyfyyyyyee | SSOOOOOOOOOOOOOOOOOOSS | 133 | 87 |
| 1838442 | TUAACUCUGUCCAUUACCAUGAA | efyyyfyyyyyyyfyyyyyyyee | SSOOOOOOOOOOOOOOOOOOSS | 134 | 93 |
| 1838443 | TAUAACUCUGUCCAUUACCAUAA | efyyyfyyyyyyyfyyyyyyyee | SSOOOOOOOOOOOOOOOOOOSS | 135 | 94 |
| 1838444 | TGAUAACUCUGUCCAUUACCAAA | efyyyfyyyyyyyfyyyyyyyee | SSOOOOOOOOOOOOOOOOOOSS | 136 | 98 |
| 1838445 | TCGAUAACUCUGUCCAUUACCAA | efyyyfyyyyyyyfyyyyyyyee | SSOOOOOOOOOOOOOOOOOOSS | 137 | 143 |
| 1838467 | TCGAUAACUCUGUCCAUUACCAA | efyyyyyyyyyyyfyfyyyyyee | SSOOOOOOOOOOOOOOOOOOSS | 138 | 143 |
| 1838455 | TGAGUAUGUGCCUCGAUAACUAA | efyyyfyyyyyyyfyyyyyyyee | SSOOOOOOOOOOOOOOOOOOSS | 139 | 144 |
| 1838477 | TGAGUAUGUGCCUCGAUAACUAA | efyyyyyyyyyyyfyfyyyyyee | SSOOOOOOOOOOOOOOOOOOSS | 140 | 144 |

### Design of Sense Oligomeric Compounds

Modified sense oligomeric compounds were prepared as described in Table 2. Each sense oligomeric compound is 21 nucleosides in length and has a sugar motif (from 5′ to 3′) as indicated, wherein each ‘y’ represents a 2′-OMe sugar moiety, each ‘e’ represents a 2′-MOE sugar moiety, each ‘f’ represents a 2′-F sugar moiety, each ‘h’ represents a 3′-fluoro-hexitol sugar moiety, and each ‘x’ represents a 2′-β-D-deoxyxylose sugar moiety; and an internucleoside motif (from 5′ to 3′) as indicated, wherein each ‘o’ represents a phosphodiester internucleoside linkage, and each ‘s’ represents a phosphorothioate internucleoside linkage. Each sense oligomeric compound is complementary to an antisense oligomeric compound in Table 1.

Certain sense oligomeric compounds, indicated in the ‘Conjugate’ column of Table 2, comprise a sense modified oligonucleotide conjugated to a GalNAc moiety. In certain compounds a HPPO-GalNAc phosphoryl conjugate group is attached at the 3′-OH of the oligonucleotide (designated 3′-GalNAc in Table 2). In certain compounds a THA-GalNAc phosphoryl conjugate group is attached at the 5′-OH of the oligonucleotide (designated 5′-GalNAc in Table 2). A HPPO-GalNAc phosphoryl conjugate group is:

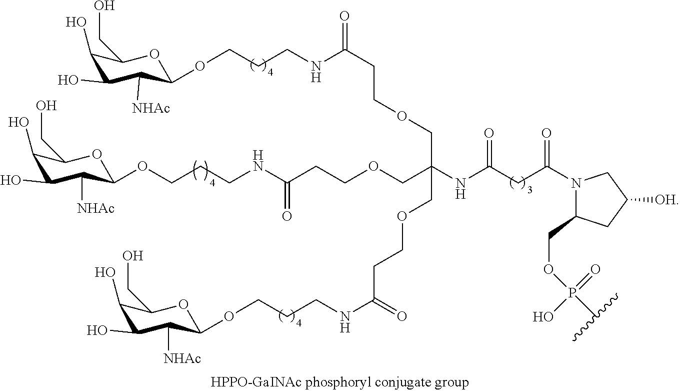

A THA-GalNAc phosphoryl conjugate group is:

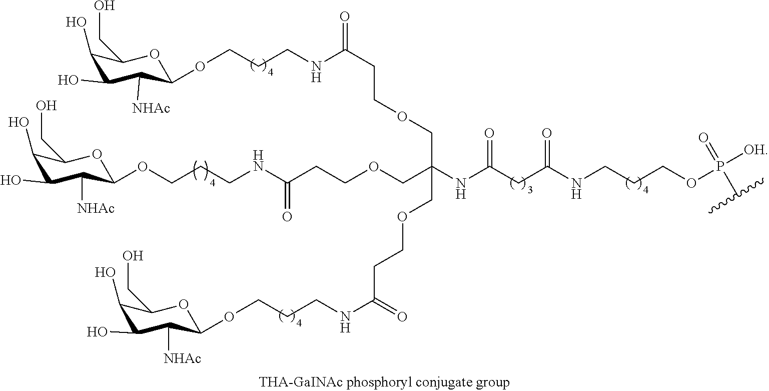

TABLE 2
Sense oligomeric compounds

| | | | Internucleoside | | | |
|---|---|---|---|---|---|---|
| Compound | Nucleobase Sequence | Sugar Motif | Linkage Motif | Conju- | CPD | nb |
| No. | (5′ to 3′) | (5′ to 3′) | (5′ to 3′) | gate | SID | SID |
| 1749524 | AUGGACAGAGUUAUCGAGGCA | yyyyyyfyfffyyyyyyyyyy | SSOOOOOOOOOOOOOOOOSS | 3′- | 49 | 99 |
| | | | | GalNAc | | |
| 1792677 | GUUAUCGAGGCACAUACUCCA | eeyyyyyyyffyyyyyyyyee | SSOOOOOOSOOOOOOOOOSS | 5′- | 50 | 100 |
| | | | | GalNAc | | |
| 1792679 | AAUGGACAGAGUUAUCGAGGA | eeyyyyyyyffyyyyyyyyee | SSOOOOOOSOOOOOOOOOSS | 5′- | 51 | 112 |
| | | | | GalNAc | | |
| 1814995 | AUGGACAGAGUUAUCGAGGCA | yyyyyyyyfffyyeyyyyyyy | SSOOOOOOOOOOOOOOOOSS | 5′- | 52 | 99 |
| | | | | GalNAc | | |
| 1814996 | AUGGACAGAGUUAUCGAGGCA | eeyyyyyyfffyyeyyyyyee | SSOOOOOOOOOOOOOOOOSS | 5′- | 53 | 99 |
| | | | | GalNAc | | |
| 1819832 | GUUAUCGAGGCACAUACUCCA | yyyyyyyyfffyyeyyyyyyy | SSOOOOOOOOOOOOOOOOSS | 3′- | 54 | 100 |
| | | | | GalNAc | | |
| 1819833 | GUUAUCGAGGCACAUACUCCA | eeyyyyyyfffyyeyyyyyee | SSOOOOOOOOOOOOOOOOSS | 3′- | 55 | 100 |
| | | | | GalNAc | | |
| 1826559 | GUUAUCGAGGCACAUACUCCA | eeyyyyyyfffyyeyyyyyee | SSOOOOOOOOOOOOOOOOSS | 5′- | 56 | 100 |
| | | | | GalNAc | | |
| 1821669 | GUUAUCGAGGCACAUACUCCA | eeyyyyyydffyyeyyyyyee | SSOOOOOOOSOOOOOOOOSS | 3′- | 57 | 100 |
| | | | | GalNAc | | |
| 1821829 | GUUAUCGAGGCACAUACUCCA | eeyyyyyyfdfyyeyyyyyee | SSOOOOOOOOSOOOOOOOSS | 3′- | 58 | 100 |
| | | | | GalNAc | | |
| 1821830 | GUUAUCGAGGCACAUACUCCA | eeyyyyyyffdyyeyyyyyee | SSOOOOOOOOOOOOOOOOSS | 3′- | 59 | 100 |
| | | | | GalNAc | | |
| 1821831 | GUUAUCGAGGCACAUACUCCA | eeyyyyyyhffyyeyyyyyee | SSOOOOOOOOOOOOOOOOSS | 3′- | 60 | 100 |
| | | | | GalNAc | | |
| 1821832 | GUUAUCGAGGCACAUACUCCA | eeyyyyyyfhfyyeyyyyyee | SSOOOOOOOOOOOOOOOOSS | 3′- | 61 | 100 |
| | | | | GalNAc | | |
| 1821833 | GUUAUCGAGGCACAUACUCCA | eeyyyyyyffhyyeyyyyyee | SSOOOOOOOOOOOOOOOOSS | 3′- | 62 | 100 |
| | | | | GalNAc | | |
| 1821835 | GUUAUCGAGGCACAUACUCCA | eeyyyyyyfxfyyeyyyyyee | SSOOOOOOOOOOOOOOOOSS | 3′- | 63 | 100 |
| | | | | GalNAc | | |
| 1821836 | GUUAUCGAGGCACAUACUCCA | eeyyyyyyffxyyeyyyyyee | SSOOOOOOOOOOOOOOOOSS | 3′- | 64 | 100 |
| | | | | GalNAc | | |
| 1821834 | GUUAUCGAGGCACAUACUCCA | eeyyyyyyxffyyeyyyyyee | SSOOOOOOOOOOOOOOOOSS | 3′- | 65 | 100 |
| | | | | GalNAc | | |
| 1839284 | GUUAUCGAGGCACAUACUCCA | eeyyyyyyfdfyyeyyyyyee | SSOOOOOOOSOOOOOOOOSS | 5′- | 66 | 100 |
| | | | | GalNAc | | |
| 1838396 | UGGACAGAGUUAUCGAGGCAA | eeyyyyyyfffyyeyyyyyee | SSOOOOOOOOOOOOOOOOSS | 5′- | 67 | 101 |
| | | | | GalNAc | | |
| 1838398 | GGACAGAGUUAUCGAGGCACA | eeyyyyyyfffyyeyyyyyee | SSOOOOOOOOOOOOOOOOSS | 5′- | 68 | 102 |
| | | | | GalNAc | | |
| 1838410 | ACAGAGUUAUCGAGGCACAUA | eeyyyyyyfffyyeyyyyyee | SSOOOOOOOOOOOOOOOOSS | 5′- | 69 | 103 |
| | | | | GalNAc | | |
| 1838404 | AGAGUUAUCGAGGCACAUACA | eeyyyyyyfffyyeyyyyyee | SSOOOOOOOOOOOOOOOOSS | 5′- | 70 | 104 |
| | | | | GalNAc | | |
| 1838406 | GAGUUAUCGAGGCACAUACUA | eeyyyyyyfffyyeyyyyyee | SSOOOOOOOOOOOOOOOOSS | 5′- | 71 | 105 |
| | | | | GalNAc | | |
| 1838377 | CAUGGUAAUGGACAGAGUUAA | eeyyyyyyfffyyeyyyyyee | SSOOOOOOOOOOOOOOOOSS | 5′- | 72 | 106 |
| | | | | GalNAc | | |
| 1838389 | AUGGUAAUGGACAGAGUUAUA | eeyyyyyyfffyyeyyyyyee | SSOOOOOOOOOOOOOOOOSS | 5′- | 73 | 107 |
| | | | | GalNAc | | |
| 1838400 | GACAGAGUUAUCGAGGCACAA | eeyyyyyyfffyyeyyyyyee | SSOOOOOOOOOOOOOOOOSS | 5′- | 74 | 108 |
| | | | | GalNAc | | |
| 1838395 | UAAUGGACAGAGUUAUCGAGA | eeyyyyyyfffyyeyyyyyee | SSOOOOOOOOOOOOOOOOSS | 5′- | 75 | 109 |
| | | | | GalNAc | | |
| 1838393 | GUAAUGGACAGAGUUAUCGAA | eeyyyyyyfffyyeyyyyyee | SSOOOOOOOOOOOOOOOOSS | 5′- | 76 | 110 |
| | | | | GalNAc | | |
| 1838390 | UGGUAAUGGACAGAGUUAUCA | eeyyyyyyfffyyeyyyyyee | SSOOOOOOOOOOOOOOOOSS | 5′- | 77 | 111 |
| | | | | GalNAc | | |
| 1841270 | GUUAUCGAGGCACAUACUCCA | eeyyyyyyffdyyeyyyyyee | SSOOOOOOOOSOOOOOOOSS | 5′- | 78 | 100 |
| | | | | GalNAc | | |
| 1841096 | GUUAUCGAGGCACAUACUCCA | eeyyyyyydffyyeyyyyyee | SSOOOOOOSOOOOOOOOOSS | 5′- | 79 | 100 |
| | | | | GalNAc | | |
| 1841271 | GUUAUCGAGGCACAUACUCCA | eeyyyyyyfhfyyeyyyyyee | SSOOOOOOOOOOOOOOOOSS | 5′- | 80 | 100 |
| | | | | GalNAc | | |
| 1841369 | GUUAUCGAGGCACAUACUCCA | eeyyyyyyffhyyeyyyyyee | SSOOOOOOOOOOOOOOOOSS | 5′- | 81 | 100 |
| | | | | GalNAc | | |
| 1792669 | AUGGACAGAGUUAUCGAGGCA | eeyyyyyyyffyyyyyyyyee | SSOOOOOOOSOOOOOOOOSS | 5′- | 82 | 99 |
| | | | | GalNAc | | |
| 1838392 | GGUAAUGGACAGAGUUAUCGA | eeyyyyyyfffyyeyyyyyee | SSOOOOOOOOOOOOOOOOSS | 5′- | 141 | 145 |
| | | | | GalNAc | | |
| 1838408 | AGUUAUCGAGGCACAUACUCA | eeyyyyyyfffyyeyyyyyee | SSOOOOOOOOOOOOOOOOSS | 5′- | 142 | 146 |
| | | | | GalNAc | | |

### Example 2: Design of Oligomeric Duplexes that Target Human LPA

Oligomeric duplexes were prepared using antisense oligomeric compounds and sense oligomeric compounds described in Example 1. Oligomeric duplex compound numbers and corresponding antisense and sense compound numbers are set forth in Table 3.

TABLE 3
Oligomeric duplexes targeted to human LPA

| Compound | Antisense | Sense |
|---|---|---|
| No. | Compound No. | Compound No. |
| 1749546 | 1749544 | 1749524 |
| 1764923 | 1764919 | 1749524 |
| 1792737 | 1792668 | 1792669 |
| 1792748 | 1792676 | 1792677 |
| 1792749 | 1792678 | 1792679 |
| 1792778 | 1792688 | 1792677 |
| 1792779 | 1792689 | 1792679 |
| 1792808 | 1792699 | 1792679 |
| 1792809 | 1792698 | 1792677 |
| 1792825 | 1792704 | 1792669 |
| 1792838 | 1792709 | 1792679 |
| 1792839 | 1792708 | 1792677 |
| 1792855 | 1792714 | 1792669 |
| 1792867 | 1792718 | 1792677 |
| 1792869 | 1792719 | 1792679 |
| 1820728 | 1814997 | 1814995 |
| 1820731 | 1814997 | 1814996 |
| 1820734 | 1814998 | 1814995 |
| 1820737 | 1814998 | 1814996 |
| 1824827 | 1819834 | 1819832 |
| 1824839 | 1819835 | 1819832 |
| 1824840 | 1819834 | 1819833 |
| 1824842 | 1819835 | 1819833 |
| 1826328 | 1819834 | 1792677 |
| 1826331 | 1819835 | 1792677 |
| 1826337 | 1814997 | 1792679 |
| 1826340 | 1814998 | 1792679 |
| 1826343 | 1792678 | 1814995 |
| 1826346 | 1814998 | 1792679 |
| 1826573 | 1819835 | 1826559 |
| 1829932 | 1819835 | 1821669 |
| 1829951 | 1819835 | 1821829 |
| 1829952 | 1819835 | 1821830 |
| 1829953 | 1819835 | 1821831 |
| 1829954 | 1819835 | 1821832 |
| 1829955 | 1819835 | 1821833 |
| 1829956 | 1819835 | 1821835 |
| 1829957 | 1819835 | 1821836 |
| 1829958 | 1819835 | 1821834 |
| 1829959 | 1792676 | 1821832 |
| 1829960 | 1792676 | 1821829 |
| 1829961 | 1792676 | 1821833 |
| 1829962 | 1792676 | 1821830 |
| 1829963 | 1792676 | 1821669 |
| 1829964 | 1792676 | 1821831 |
| 1829965 | 1792676 | 1821834 |
| 1829966 | 1792676 | 1821835 |
| 1829967 | 1792676 | 1821836 |
| 1831890 | 1826580 | 1792677 |
| 1831892 | 1826581 | 1792677 |
| 1831895 | 1826580 | 1819833 |
| 1831896 | 1826581 | 1819833 |
| 1831897 | 1826582 | 1814996 |
| 1831898 | 1826583 | 1814996 |
| 1839407 | 1839388 | 1826559 |
| 1839408 | 1839376 | 1826559 |
| 1839409 | 1839378 | 1826559 |
| 1839410 | 1839377 | 1814996 |
| 1839411 | 1839379 | 1814996 |
| 1839412 | 1839380 | 1814996 |
| 1839601 | 1826581 | 1826559 |
| 1840071 | 1792676 | 1839284 |
| 1840201 | 1838448 | 1838396 |
| 1840202 | 1838470 | 1838396 |
| 1840453 | 1838449 | 1838398 |
| 1840454 | 1838471 | 1838398 |
| 1840892 | 1838451 | 1838410 |
| 1840893 | 1838473 | 1838410 |
| 1841023 | 1838453 | 1838404 |
| 1841095 | 1838475 | 1838404 |
| 1841097 | 1838454 | 1838406 |
| 1841125 | 1838476 | 1838406 |
| 1841126 | 1838464 | 1838377 |
| 1841127 | 1838465 | 1838389 |
| 1841128 | 1838472 | 1838400 |
| 1841129 | 1838450 | 1838400 |
| 1841133 | 1838469 | 1838395 |
| 1841134 | 1838447 | 1838395 |
| 1841135 | 1838468 | 1838393 |
| 1841136 | 1838446 | 1838393 |
| 1841137 | 1838466 | 1838390 |
| 1843479 | 1792676 | 1841270 |
| 1843480 | 1792676 | 1841096 |
| 1843481 | 1792676 | 1841271 |
| 1843482 | 1792676 | 1841369 |
| 1858685 | 1838442 | 1838377 |
| 1859018 | 1838443 | 1838389 |
| 1859347 | 1838444 | 1838390 |
| 1859802 | 1838445 | 1838392 |
| 1861908 | 1838455 | 1838408 |
| 1861909 | 1838467 | 1838392 |
| 1861910 | 1838477 | 1838408 |

### Example 3: Effect of Oligomeric Duplexes Targeted to Human LPA in Primary Human Hepatocytes

### 3A. Single Dose Inhibition of Human LPA

Oligomeric duplexes were tested for single dose effects on LPA RNA in vitro in experiments that had the same culture conditions. Primary human hepatocytes (sourced from BioIVT, M0095-P, lot ZFW) were treated with oligomeric duplex at a concentration of 100 nM or 5,000 nM by free uptake at a density of 20,000 cells per well. After a 72-hour treatment period, total RNA was isolated from the cells and LPA RNA levels were measured by quantitative real-time RTPCR using human primer-probe set hAPO(a)₁₂ kB (forward sequence CCACAGTGGCCCCGGT (SEQ ID NO: 113); reverse sequence ACAGGGCTTTTCTCAGGTGGT (SEQ ID NO: 114); probe sequence CCAAGCACAGAGGCTCCTTCTGAACAAG (SEQ ID NO: 115). LPA RNA levels were normalized to total RNA content, as measured by RIBOGREENW. Reduction of LPA RNA is presented in the table below as percent LPA RNA relative to the amount of LPA RNA in untreated control cells (%˜UTC). Each separate experiment described in this example is presented in separate sub-Tables 4A-4B.

TABLE 4
Effect of oligomeric duplex on human
LPA RNA in primary human hepatocytes

| | |
|---|---|
| 4A. 100 nM | 4B. 5,000 nM |

| | LPA RNA | | LPA RNA |
|---|---|---|---|
| Compound No. | (% control) | Compound No. | (% control) |
| 1749546 | 45 | 1764923 | 37 |

### 3B: Dose Dependent Inhibition of Human LPA

i. Primary Human Hepatocytes

a. Oligomeric duplexes from Example 4 were tested at various doses in primary human hepatocytes. The oligomeric duplexes were evaluated in experiments that had the same culture conditions, and results are depicted in Table 5, with different experiments in each of sub-Tables 5A-5B.

Primary human hepatocytes (sourced from BioIVT, M0095-P, lot ZFW) plated at a density of 20,000 cells per well were treated using free uptake with various concentrations of modified oligonucleotide as specified in Table 5. After a treatment period of 48 hours, total RNA was isolated from the cells and LPA RNA levels were measured by quantitative real-time RTPCR. Human LPA primer-probe set hAPO(a)₁₂ kB (described herein above) was used to measure RNA levels as described above. LPA RNA levels were normalized to total RNA content, as measured by RIBOGREEN®. Reduction of LPA RNA is presented in Table 5 as percent LPA RNA, relative to the amount of LPA RNA in untreated control cells (% UTC). The half maximal inhibitory concentration (IC₅₀) of each modified oligonucleotide was calculated using a linear regression on a log/linear plot of the data in Excel and is also presented in Table 5. N. C. refers to IC₅₀ values that could not be reliably calculated.

TABLE 5
Dose-dependent reduction of human LPA RNA in primary human hepatocytes

| | Compound | LPA RNA (% UTC) | | | | | | IC₅₀ |
|---|---|---|---|---|---|---|---|---|
| | No. | 0.1 nM | 1 nM | 10 nM | 100 nM | 1,000 nM | 10,000 nM | (μM) |
| 5A. | 1749546 | 96 | 51 | 33 | 35 | 24 | 8 | 0.002 |

| | | LPA RNA (% UTC) | | | | IC₅₀ |
|---|---|---|---|---|---|---|
| | Compound No. | 5 nM | 50 nM | 500 nM | 5,000 nM | (μM) |
| 5B. | 1764923 | 75 | 76 | 70 | 49 | N.C |

b. Oligomeric duplexes described in Example 2 were tested at various doses in primary human hepatocytes in a series of experiments that had the same culture conditions, and results are depicted in Table 6, with different experiments in each of sub-Tables 6A-6D.

Compound AD03851 is a compound targeting LPA previously disclosed in International Patent Application Publication No. WO2017/059223, which is incorporated herein by reference.

Compound No. 1787751, comprising antisense Compound No. 1758680, and sense Compound No. 1758681, was prepared and analyzed alongside compounds described above. Antisense Compound No. 1758680 (SEQ ID NO: 116) has a nucleobase sequence (from 5′ to 3′) of UCGUAUAACAAUAAGGGGCUG; a sugar motif (from 5′ to 3′) of yfyfyfyyyyyfyfyfyfyfy, and an internucleoside linkage motif (from 5′ to 3′) of ssooooooooooooooooss; and sense Compound No. 1758681 (SEQ ID NO: 117) has a nucleobase sequence (from 5′ to 3′) of CAGCCCCUUAUUGUUAUACGA; a sugar motif (from 5′ to 3′) of yyyyyyyyfffyyyyyyyyyy, and an internucleoside linkage motif (from 5′ to 3′) of soooooooooooooooooos; wherein each “y” represents a 2′-OMe sugar moiety, and each “f” represents a 2′-F sugar moiety, and wherein each “s” represents a phosphorothioate internucleoside linkage, and each “o” represents a phosphodiester internucleoside linkage. Sense oligonucleotide Compound No. 1758681 has a THA-GalNAc moiety conjugated to the 5′-end of the oligonucleotide via a phosphorothioate bond, as described above. Compound No. 1787751 is similar to compound AD03851, and differs from AD03851 in two changes: first, Compound No. 1787751 comprises a THA-GalNAc conjugate (as described herein) on the 5′-end of the sense oligonucleotide, whereas AD03851 comprises an NAG25 conjugate; and second, 1787751 comprises a 2′-OMe sugar moiety at the 3′-end of the sense oligonucleotide, whereas AD03851 comprises an inverted 2′-deoxyribosyl sugar moiety modification at the 3′-end of the sense oligonucleotide. The NAG25 phosphorothioate moiety conjugated to the 5′-end ofthe oligonucleotide via a phosphorothioate bond, as previously described, is shown below:

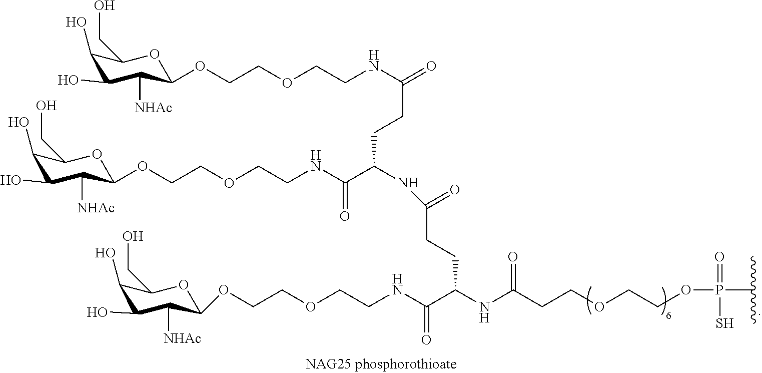

Primary human hepatocytes (sourced from BioIVT, M0095-P, lot ZFW) plated at a density of 35,000 cells per well were treated using free uptake with various concentrations of modified oligonucleotide as specified in Table 6. After a treatment period of 96 hours, total RNA was isolated from the cells and human LPA RNA levels were measured by quantitative real-time RTPCR using primer-probe set hAPO(a)12 kB as described in Example 3A herein. LPA RNA levels were normalized to HPRT RNA levels, as measured by primer-probe set RTS35336 (forward sequence TTGTTGTAGGATATGCCCTTGA (SEQ ID NO: 118); reverse sequence GCGATGTCAATAGGACTCCAG (SEQ ID NO: 119); probe sequence AGCCTAAGATGAGAGTTCAAGTTGAGTTTGG (SEQ ID NO. 120). Reduction of LPA RNA is presented in Table 6 as percent LPA RNA, relative to the amount of LPA RNA in untreated control cells (% UTC). The half maximal inhibitory concentration (IC₅₀) of each modified oligonucleotide was calculated in GraphPad Prism 10 (GraphPad Software, San Diego, CA). “N. D.” represents points for which there is no data. “N. C.” represents IC₅₀ values that could not be reliably calculated.

TABLE 6
Dose-dependent reduction of human LPA RNA in primary human hepatocytes
6A.

| | LPA RNA (% UTC) | | | | | | | | | | |
|---|---|---|---|---|---|---|---|---|---|---|---|
| Compound | 10000 | 1000 | 100 | 10 | 1 | 0.1 | 0.01 | 0.001 | 0.0001 | 0.00001 | IC₅₀ |
| No. | nM | nM | nM | nM | nM | nM | nM | nM | nM | nM | (nM) |
| 1841133 | 39 | 26 | 42 | 66 | 85 | 93 | 98 | 122 | 93 | 105 | 118 |
| 1841135 | 29 | 39 | 46 | 72 | 80 | 98 | 89 | 83 | 91 | 86 | 185 |
| 1841137 | 30 | 44 | 49 | 59 | 74 | 88 | 92 | 82 | 96 | 87 | 153 |
| 1841125 | 95 | 104 | 109 | 101 | 112 | 86 | 90 | 95 | 103 | 98 | N.C. |
| 1841127 | 40 | 60 | 79 | 73 | 81 | 86 | 95 | 88 | 104 | 92 | 4692 |
| 1841129 | 33 | 42 | 52 | 58 | 74 | 87 | 86 | 89 | 91 | 100 | 171 |
| 1840892 | 53 | 58 | 65 | 78 | 92 | 95 | 94 | 94 | 89 | 106 | 8420 |
| 1841023 | 93 | 94 | 85 | 91 | 91 | 86 | 87 | 87 | 83 | 97 | N.C. |
| 1841097 | 73 | 86 | 80 | 80 | 93 | 90 | 93 | 102 | 82 | 98 | N.C. |
| 1840071 | 46 | 64 | 66 | 111 | 90 | 103 | 102 | 87 | 96 | 91 | 4863 |
| 1840202 | 21 | 21 | 40 | 41 | 71 | 78 | 86 | 88 | 98 | 95 | 11 |
| 1840454 | 43 | 47 | 48 | 57 | 81 | 95 | 82 | 99 | 73 | 94 | 505 |
| 1841134 | 33 | 34 | 51 | 70 | 91 | 96 | 102 | 104 | 86 | 121 | 227 |
| 1841136 | 41 | 48 | 52 | 83 | 84 | 80 | 94 | 77 | 80 | 106 | 1240 |
| 1841126 | 23 | 30 | 39 | 60 | 86 | 100 | 90 | 92 | 93 | 111 | 62 |
| 1841128 | 21 | 32 | 38 | 62 | 69 | 95 | 89 | 89 | 92 | 90 | 42 |
| 1840893 | 47 | 46 | 57 | 77 | 84 | 93 | 90 | 98 | 91 | 98 | 1360 |
| 1841095 | 77 | 95 | 75 | 79 | 89 | 95 | 101 | 89 | 80 | 85 | N.C. |
| 1840201 | 29 | 32 | 42 | 50 | 73 | 105 | 88 | 108 | 97 | 102 | 55 |
| 1840453 | 44 | 50 | 46 | 61 | 76 | 80 | 95 | 95 | 88 | 94 | 515 |

6B.

| | LPA RNA (% UTC) | | | | | | | | | | |
|---|---|---|---|---|---|---|---|---|---|---|---|
| Compound | 10000 | 1000 | 100 | 10 | 1 | 0.1 | 0.01 | 0.001 | 0.0001 | 0.00001 | IC₅₀ |
| No. | nM | nM | nM | nM | nM | nM | nM | nM | nM | nM | (nM) |
| 1831895 | 66 | 48 | 57 | 71 | 81 | 79 | 88 | 95 | 108 | 85 | N.C. |
| 1829951 | 42 | 36 | 65 | 65 | 71 | 82 | 81 | 79 | 80 | 90 | 625 |
| 1829963 | 85 | 91 | 105 | 83 | 84 | 92 | 76 | 81 | 100 | 91 | N.C. |
| 1831890 | 72 | 67 | 81 | 93 | 82 | 85 | 94 | 89 | 101 | 98 | N.C. |
| 1829962 | 67 | 53 | 60 | 66 | 79 | 82 | 78 | 95 | 87 | 93 | N.C. |
| 1824840 | 52 | 58 | 63 | 83 | 117 | 125 | 109 | 91 | 107 | 131 | 4086 |
| 1831898 | 13 | 20 | 28 | 38 | 62 | 82 | 87 | 99 | 85 | 108 | 5 |
| 1829952 | 50 | 46 | 62 | 67 | 87 | 76 | 86 | 68 | 78 | 86 | N.C. |
| 1820737 | 27 | 31 | 32 | 48 | 64 | 90 | 90 | 85 | 87 | 94 | 18 |
| 1829960 | 72 | 71 | 69 | 74 | 81 | 80 | 91 | 92 | 97 | 89 | N.C. |
| 1829957 | 41 | 50 | 73 | 81 | 75 | 80 | 82 | 87 | 99 | 91 | 3800 |
| 1839407 | 69 | 67 | 58 | 74 | 91 | N.D. | 97 | 106 | 90 | 99 | N.C. |
| 1831897 | 16 | 20 | 25 | 41 | 74 | 88 | 73 | 76 | 88 | 115 | 6 |
| 1826573 | 42 | 33 | 46 | 55 | 72 | 89 | 89 | 82 | 93 | 88 | 115 |
| 1829955 | 44 | 51 | 66 | 71 | 99 | 71 | 85 | 106 | 85 | 92 | N.C. |
| 1829961 | 59 | 60 | 79 | 69 | 88 | 73 | 96 | 90 | 82 | 89 | N.C. |
| 1839408 | 46 | 34 | 25 | 58 | 65 | 89 | 91 | 74 | 100 | 80 | 53 |
| 1831896 | 59 | 49 | 52 | 69 | 70 | 86 | 81 | 80 | 87 | 101 | 5536 |
| 1831892 | 74 | 67 | 49 | 74 | 70 | 81 | 59 | 67 | 77 | 83 | N.C. |
| 1839410 | 57 | 15 | 32 | 43 | 74 | 79 | 78 | 82 | 96 | 114 | 23 |
| 1829956 | 75 | 72 | 70 | 83 | 88 | 81 | 88 | 90 | 106 | 103 | N.C. |
| 1829959 | 85 | 68 | 93 | 88 | 81 | 90 | 83 | 89 | 71 | 107 | N.C. |
| 1792748 | 36 | 8 | 71 | 66 | 88 | 86 | 88 | 91 | 91 | 103 | 145 |
| 1839411 | 98 | 39 | 44 | 68 | 76 | 101 | 85 | 89 | 100 | 92 | N.C |
| 1839409 | 65 | 56 | 52 | 76 | 70 | 80 | 76 | 109 | 96 | 133 | N.C. |
| 1839412 | 28 | 23 | 28 | 33 | 53 | 73 | 86 | 95 | 97 | 95 | 3 |
| 1826328 | 80 | 71 | 74 | 77 | 72 | 86 | 84 | 87 | 87 | 85 | N.C. |

6C.

| | LPA RNA (% UTC) | | | | | | | | | | |
|---|---|---|---|---|---|---|---|---|---|---|---|
| Compound | 10000 | 1000 | 100 | 10 | 1 | 0.1 | 0.01 | 0.001 | 0.0001 | 0.00001 | IC₅₀ |
| No. | nM | nM | nM | nM | nM | nM | nM | nM | nM | nM | (nM) |
| 1820737 | 16 | 19 | 19 | 39 | 62 | 73 | 95 | 95 | 93 | 108 | 4 |
| 1824842 | 4 | 23 | 46 | 51 | 62 | 83 | 90 | 91 | 101 | 104 | 15 |
| 1792748 | 1 | 3 | 40 | 84 | 96 | 99 | 82 | 91 | 85 | 97 | 61 |
| 1792749 | 3 | 22 | 50 | 66 | 89 | 83 | 85 | 89 | 102 | 91 | 53 |
| 1787751 | 82 | 86 | 92 | 87 | 98 | 101 | 103 | 111 | 97 | 111 | N.C. |
| 1792737 | 2 | 14 | 41 | 72 | 99 | 87 | 87 | 101 | 90 | 94 | 52 |
| 1792855 | 89 | 93 | 85 | 81 | 89 | 93 | 87 | 98 | 93 | 90 | N.C. |

6D.

| | LPA RNA (% UTC) | | | | | | | | | | |
|---|---|---|---|---|---|---|---|---|---|---|---|
| Compound | 6000 | 600 | 60 | 6 | 0.6 | 0.06 | 0.006 | 0.0006 | 0.00006 | 0.000006 | IC₅₀ |
| No. | nM | nM | nM | nM | nM | nM | nM | nM | nM | nM | (nM) |
| 1792749 | 3 | 14 | 34 | 42 | 52 | 78 | 69 | 27 | 93 | 187 | 0.87 |
| 1787751 | 5 | 16 | 16 | 34 | 28 | 52 | 113 | 107 | 91 | 91 | 0.33 |

### Example 4: Effect of Oligomeric Duplexes in Transgenic Primary Mouse Hepatocytes

### 4A. Single Dose Inhibition of Human LPA in Transgenic Primary Mouse Hepatocytes

Oligomeric duplexes were tested for single dose effects on LPA RNA in vitro in experiments that had the same culture conditions. Each separate experiment is presented in a separate sub-table 7A or 7B in Table 7 below. Primary hepatocytes were isolated from transgenic mice previously described in Frazer K, et. al., The apolipoprotein (a) gene is regulated by sex hormones and acute-phase inducers in YAC transgenic mice; Nature Genetics, 1995, 9: 424-431. Transgenic primary mouse hepatocytes were treated with oligomeric duplex at a concentration of 500 nM (Table 7A) or 5000 nM (Table 7B) by free uptake at a density of 20,000 cells per well. After a treatment period of 24 hours, total RNA was isolated from the cells and LPA RNA levels were measured by quantitative real-time RTPCR using human primer-probe set hAPO(a) 12 kB (described herein above). LPA RNA levels were normalized to total RNA content, as measured by RIBOGREENW. Reduction of LPA RNA is presented in the table below as percent LPA RNA relative to the amount of LPA RNA in untreated control cells (% UTC).

TABLE 7
Effect of oligomeric duplexes on human LPA
RNA in transgenic primary mouse hepatocytes

| | |
|---|---|
| 7A. 500 nM | 7B. 5000 nM |

| | LPA RNA | | LPA RNA |
|---|---|---|---|
| Compound No. | (% control) | Compound No. | (% control) |
| 1749546 | 31 | 1764923 | 6 |

### 4B. Dose Dependent Inhibition ofLPA in Transgenic Primary Mouse Hepatocvtes

Oligomeric duplexes and comparator compound were tested at various doses in transgenic primary mouse hepatocytes. Primary hepatocytes were isolated from transgenic mice previously described in Frazer K, et. al., The apolipoprotein (a) gene is regulated by sex hormones and acute-phase inducers in YAC transgenic mice; Nature Genetics, 1995, 9: 424-431. Transgenic primary hepatocytes plated at a density of 20,000 cells per well (Table 8) or 30,000 cells per well (Table 9A and Table 9B3) and treated using free uptake with various concentrations of oligomeric duplexes as specified in Table 8 and Table 9 below. Transgenic primary hepatocytes at a density of 30,000 cells/well were treated using electroporation with various concentrations of oligomeric compound as specific in Table 10 below. After a treatment period of 24 hours, total RNA was isolated from the cells and human LPA RNA levels were measured by quantitative real-time RTPCR using LPA primer-probe set hAPO(a)₁₂ kB (described herein above) to measure RNA levels as described above. LPA RNA levels were normalized to total RNA content, as measured by RIBOGREEN®. Reduction of LPA RNA is presented in Tables 8-10 below as percent LPA RNA, relative to the amount of LPA RNA in untreated control cells (% UTC). The half maximal inhibitory concentration (IC₅₀) was calculated using a linear regression on a log/linear plot of the data in Excel and is also presented in Table 8, or in GraphPad Prism 10 (GraphPad Software, San Diego, CA) and is presented in Tables 9-10. “N. C.” represents IC₅₀ values that could not be reliably calculated.

TABLE 8
Dose-dependent reduction of human LPA
RNA in primary mouse hepatocytes

| | LPA RNA (% UTC) | | | | IC₅₀ |
|---|---|---|---|---|---|
| Compound No. | 5 nM | 50 nM | 500 nM | 5,000 nM | (μM) |
| 1764923 | 61 | 70 | 44 | 14 | 0.11 |

TABLE 9
Dose-dependent reduction of human LPA RNA in primary mouse hepatocytes

| | LPA RNA (% UTC) | | | | | | | | | | |
|---|---|---|---|---|---|---|---|---|---|---|---|
| Compound | 5000 | 1000 | 200 | 40 | 8 | 1.6 | 0.32 | 0.064 | 0.0128 | 0.00256 | IC₅₀ |
| No. | nM | nM | nM | nM | nM | nM | nM | nM | nM | nM | (nM) |

9A

| | | | | | | | | | | | |
|---|---|---|---|---|---|---|---|---|---|---|---|
| 1841133 | 8 | 11 | 11 | 28 | 43 | 73 | 82 | 86 | 90 | 88 | 5.99 |
| 1841125 | 68 | 73 | 71 | 92 | 108 | 112 | 117 | 125 | 102 | 111 | N.C. |
| 1840892 | 18 | 26 | 37 | 56 | 96 | 103 | 89 | 109 | 98 | 90 | 119 |
| 1840071 | 8 | 11 | 14 | 22 | 36 | 63 | 90 | 93 | 114 | 78 | 4.54 |
| 1841134 | 5 | 7 | 13 | 24 | 51 | 60 | 89 | 111 | 99 | 92 | 6.85 |
| 1841126 | 2 | 4 | 6 | 9 | 25 | 49 | 85 | 92 | 83 | 94 | 1.81 |
| 1840893 | 12 | 14 | 21 | 38 | 58 | 80 | 102 | 87 | 98 | 102 | 19.1 |
| 1840201 | 4 | 5 | 9 | 13 | 34 | 67 | 86 | 100 | 105 | 97 | 3.81 |
| 1841135 | 3 | 3 | 4 | 7 | 15 | 40 | 73 | 84 | 87 | 86 | 0.88 |
| 1841127 | 4 | 4 | 5 | 10 | 31 | 63 | 75 | 85 | 87 | 90 | 2.19 |
| 1841023 | 9 | 14 | 19 | 40 | 70 | 83 | 87 | 114 | 94 | 90 | 24.0 |
| 1840202 | 3 | 4 | 4 | 9 | 20 | 43 | 62 | 79 | 79 | 72 | 0.56 |
| 1841136 | 3 | 3 | 4 | 9 | 19 | 38 | 74 | 89 | 81 | 84 | 0.94 |
| 1841128 | 3 | 3 | 6 | 17 | 37 | 66 | 85 | 79 | 77 | 81 | 2.82 |
| 1841095 | 6 | 8 | 13 | 20 | 42 | 69 | 98 | 85 | 92 | 93 | 5.88 |
| 1840453 | 9 | 7 | 10 | 21 | 37 | 81 | 87 | 97 | 99 | 106 | 6.11 |
| 1841137 | 2 | 2 | 3 | 8 | 18 | 51 | 81 | 96 | 101 | 110 | 1.65 |
| 1841129 | 4 | 5 | 9 | 18 | 38 | 62 | 88 | 88 | 79 | 87 | 3.45 |
| 1841097 | 27 | 30 | 43 | 66 | 81 | 100 | 101 | 86 | 83 | 85 | 188 |
| 1840454 | 6 | 6 | 9 | 11 | 26 | 46 | 77 | 82 | 79 | 79 | 1.18 |
| 1829956 | 11 | 14 | 15 | 21 | 48 | 61 | 74 | 91 | 84 | 78 | 4.00 |
| 1829960 | 6 | 8 | 10 | 18 | 26 | 52 | 78 | 74 | 80 | 77 | 1.27 |
| 1787751 | 12 | 13 | 15 | 39 | 60 | 81 | 94 | 99 | 93 | 87 | 18.0 |
| 1829961 | 13 | 17 | 21 | 45 | 60 | 82 | 84 | 91 | 97 | 75 | 22.0 |
| 1829962 | 7 | 10 | 9 | 15 | 41 | 52 | 81 | 92 | 78 | 85 | 2.63 |

| 9B | | | | | | | | | | | |
|---|---|---|---|---|---|---|---|---|---|---|---|
| | LPA RNA (% UTC) | | | | | | | | | | |
| Duplex | 5000 | 1000 | 200 | 40 | 8 | 1.6 | 0.32 | 0.064 | 0.0128 | 0.00256 | IC₅₀ |
| No. | nM | nM | nM | nM | nM | nM | nM | nM | nM | nM | (nM) |
| 1820737 | 7 | 9 | 14 | 39 | 54 | 80 | 76 | 94 | 91 | 94 | 12 |
| 1839410 | 6 | 7 | 16 | 31 | 49 | 68 | 96 | 92 | 89 | 124 | 9 |
| 1839411 | 16 | 19 | 30 | 50 | 70 | 88 | 91 | 90 | 119 | 118 | 48 |
| 1839412 | 9 | 19 | 22 | 34 | 54 | 79 | 113 | 104 | 102 | 106 | 18 |
| 1787751 | 25 | 24 | 38 | 60 | 83 | 107 | 98 | 109 | 114 | 112 | 127 |

TABLE 10
Dose-dependent reduction of human LPA RNA in primary mouse hepatocytes

| | LPA RNA (% UTC) | | | | | | | | | | |
|---|---|---|---|---|---|---|---|---|---|---|---|
| Compound | 5000 | 1000 | 200 | 40 | 8 | 1.6 | 0.32 | 0.064 | 0.0128 | 0.00256 | IC₅₀ |
| No. | nM | nM | nM | nM | nM | nM | nM | nM | nM | nM | (nM) |
| 1841133 | 6 | 6 | 7 | 10 | 11 | 17 | 45 | 91 | 101 | 104 | 0.33 |
| 1841125 | 37 | 50 | 67 | 66 | 83 | 90 | 98 | 104 | 96 | 95 | 937 |
| 1840892 | 9 | 10 | 12 | 15 | 29 | 56 | 86 | 102 | 115 | 98 | 2.83 |
| 1840071 | 7 | 11 | 10 | 13 | 14 | 16 | 43 | 71 | 85 | 102 | 0.23 |
| 1841134 | 5 | 7 | 11 | 11 | 14 | 22 | 65 | 79 | 94 | 102 | 0.51 |
| 1841126 | 3 | 6 | 9 | 8 | 10 | 14 | 26 | 62 | 77 | 89 | 0.10 |
| 1840893 | 8 | 10 | 13 | 14 | 21 | 36 | 66 | 83 | 86 | 91 | 0.83 |
| 1840201 | 9 | 8 | 9 | 10 | 10 | 14 | 29 | 68 | 88 | 91 | 0.14 |
| 1841135 | 6 | 7 | 8 | 9 | 11 | 13 | 19 | 69 | 91 | 106 | 0.12 |
| 1841127 | 11 | 10 | 11 | 12 | 12 | 14 | 33 | 90 | 93 | 91 | 0.23 |
| 1841023 | 17 | 17 | 16 | 19 | 34 | 51 | 80 | 74 | 104 | 107 | 2.70 |
| 1840202 | 7 | 9 | 10 | 10 | 10 | 16 | 33 | 81 | 111 | 111 | 0.21 |
| 1841136 | 6 | 11 | 9 | 10 | 11 | 13 | 23 | 46 | 83 | 98 | 0.07 |
| 1841128 | 6 | 9 | 10 | 10 | 8 | 14 | 37 | 65 | 84 | 89 | 0.15 |
| 1841095 | 18 | 13 | 12 | 13 | 14 | 22 | 72 | 87 | 103 | 86 | 0.68 |
| 1840453 | 15 | 12 | 9 | 14 | 13 | 18 | 39 | 91 | 122 | 101 | 0.28 |
| 1841137 | 6 | 6 | 6 | 7 | 9 | 12 | 31 | 70 | 97 | 118 | 0.16 |
| 1841129 | 15 | 21 | 16 | 15 | 15 | 23 | 55 | 88 | 115 | 126 | 0.50 |
| 1841097 | 30 | 41 | 46 | 61 | 75 | 75 | 76 | 97 | 115 | 101 | 193 |
| 1840454 | 14 | 27 | 24 | 18 | 18 | 22 | 44 | 69 | 110 | 95 | 0.41 |
| 1829956 | 25 | 25 | 23 | 25 | 25 | 39 | 65 | 65 | 72 | 91 | 0.92 |
| 1829960 | 12 | 15 | 19 | 20 | 18 | 20 | 41 | 61 | 83 | 87 | 0.21 |
| 1787751 | 22 | 27 | 29 | 31 | 32 | 41 | 137 | 124 | 135 | 136 | 6.34 |
| 1829961 | 17 | 30 | 27 | 33 | 32 | 70 | 108 | 114 | 120 | 133 | 11.8 |
| 1829962 | 20 | 32 | 30 | 34 | 28 | 45 | 91 | 80 | 96 | 113 | 6.64 |

### Example 5: Activity of Oligomeric Duplexes Targeting Human LPA in LPA Transgenic Mice

### 5A. Single Dose Inhibition of Human LPA in Transgenic Primary Mouse

Oligomeric duplexes described above were analyzed along with a comparator compound (1787751) for their effects on LPA mRNA and apo(a) protein in human LPA transgenic mice. Each separate experiment is presented in a separate sub-table 1A-11C in Table 11 below. Groups of two female LPA transgenic mice each received a single subcutaneous injection of oligomeric duplex at a dose of 2 mg/kg. One group of 3 LPA transgenic mice received a single subcutaneous injection of PBS. Mice were sacrificed at Day 7 and RNA was extracted from liver tissue for quantitative real time RTPCR analysis of LPA RNA using human primer probe set hAPO(a)₁₂ kBTS (described herein above). LPA RNA levels were normalized to total RNA content, as measured by RIBOGREEN®. Reduction of LPA RNA is presented as percent LPA RNA relative to the amount of LPA in tissue from PBS control animals (0 control).

To evaluate effects of oligomeric duplexes on apo(a) protein levels, blood plasma was collected prior to treatment and on Day 7 when the mice were sacrificed and analyzed using an automated clinical chemistry analyzer (Hitachi Olympus AU400c, Melville, NY). apo(a) protein levels were analyzed with an Lp(a) assay kit from Randox (Catalog 4LP2757). The results were averaged for each group of mice and are presented in the tables below as percent apo(a), relative to the amount of apo(a) present at baseline (% baseline).

TABLE 11
Reduction of LPA in LPA transgenic mice

| 11A. | Liver LPA RNA | apo(a) Plasma Protein |
|---|---|---|
| Compound No. | (% control) | (% Baseline) |
| PBS | 100 | 67 |
| 1787751 | 29 | 24 |
| 1792749 | 10 | 12 |
| 1792779 | 79 | 27 |
| 1792838 | 179 | 104 |
| 1792867 | 88 | 53 |
| 11B. | Liver LPA RNA | apo(a) Plasma Protein |
| Compound No. | (% control) | (% Baseline) |
| PBS | 1001 | 126 |
| 1787751 | 13 | 15 |
| 1792748 | 19 | 28 |
| 1792839 | 51 | 33 |
| 11C. | Liver LPA RNA | apo(a) Plasma Protein |
| Compound No. | (% control) | (% Baseline) |
| PBS | 100 | 115 |
| 1787751 | 21 | 26 |
| 1792748 | 22 | 26 |
| 1792749 | 14 | 22 |
| 1792869 | 47 | 47 |
| 1820728 | 7 | 12 |
| 1792855 | 24 | 25 |
| 1820731 | 9 | 11 |
| 1820734 | 12 | 18 |
| 1820737 | 23 | 12 |
| 1824827 | 14 | 8 |
| 1824839 | 32 | 32 |
| 1824840 | 27 | 10 |
| 1824842 | 21 | 9 |

### 5B. Single Dose Inhibition of Human LPA in LPA Transgenic Mouse

Oligomeric duplexes described above were analyzed for their effects on apo(a) protein in human LPA transgenic mice (described herein above). Groups of 3 female LPA transgenic mice each received a single subcutaneous injection of oligomeric duplex compound at a dose of 3 mg/kg. One group of 3 female LPA transgenic mice received a single subcutaneous injection of PBS. Blood plasma was collected 1 day prior to treatment and at 6 weeks post treatment on Day 42 and apo(a) protein levels were analyzed by ELISA array with the Abcam ELISA kit (ab212165). Results were averaged for each group of mice and presented in Table 12 as percent apo(a) protein, relative to the amount of apo(a) protein present at baseline (% baseline).

TABLE 12
Reduction of apo(a) protein levels in LPA transgenic mice

| | apo(a) Plasma Protein |
|---|---|
| Compound No. | (% Baseline) |
| PBS | 125 |
| 1858685 | 18 |
| 1859018 | 7 |
| 1859347 | 18 |
| 1859802 | 6 |
| 1861909 | 4 |
| 1861910 | 2 |

### 5C. Duration of Inhibition of Human LPA in Transgenic Mouse

Oligomeric duplexes and comparator compound no. 1787751 were analyzed for their effects on LPA mRNA and apo(a) protein in human LPA transgenic mice. Groups of 3 female LPA transgenic mice each received a single subcutaneous injection of oligomeric duplex at a dose of 3 mg/kg. One group of 3 female LPA transgenic mice received a single subcutaneous injection of PBS.

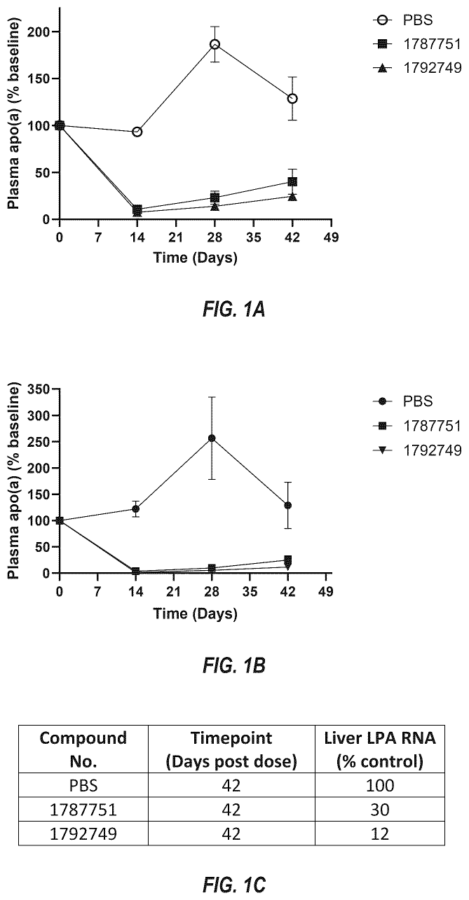

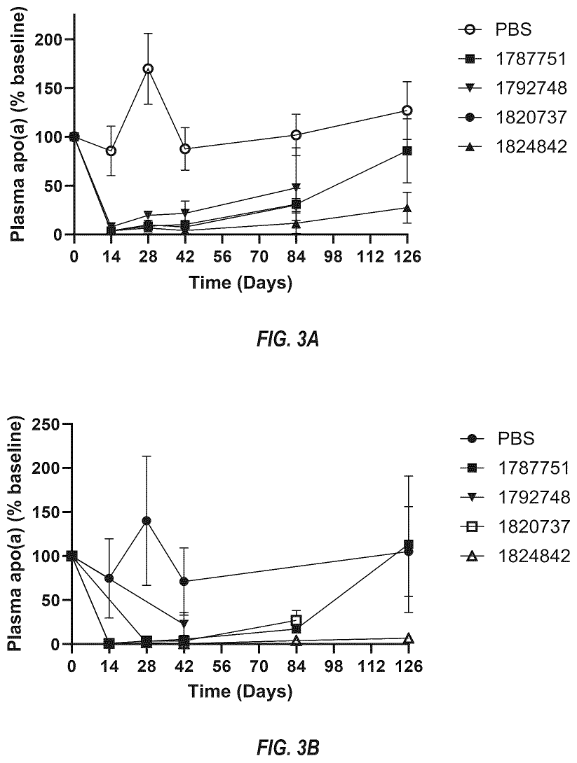

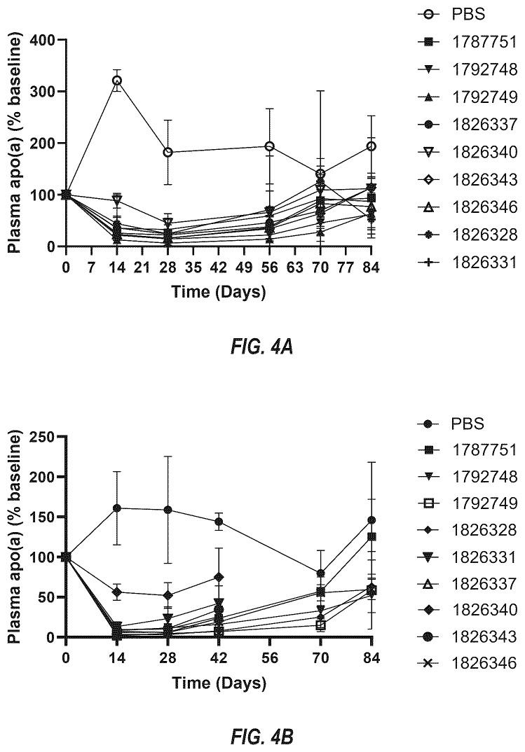

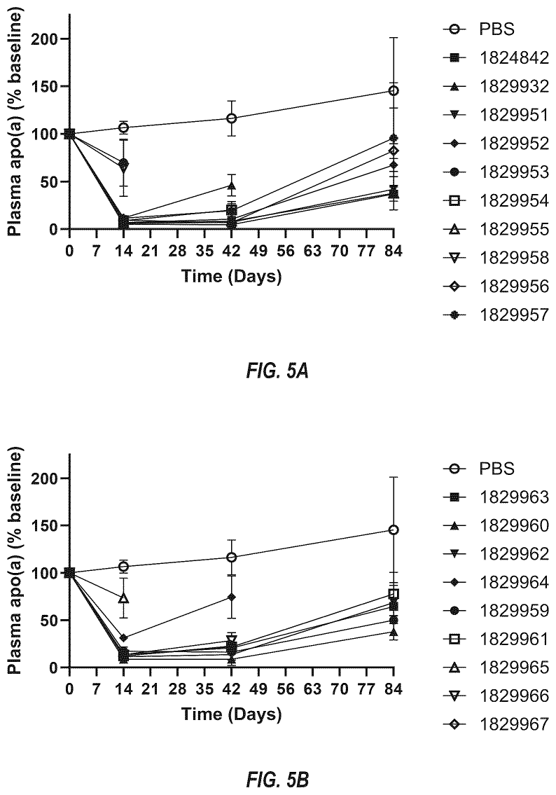

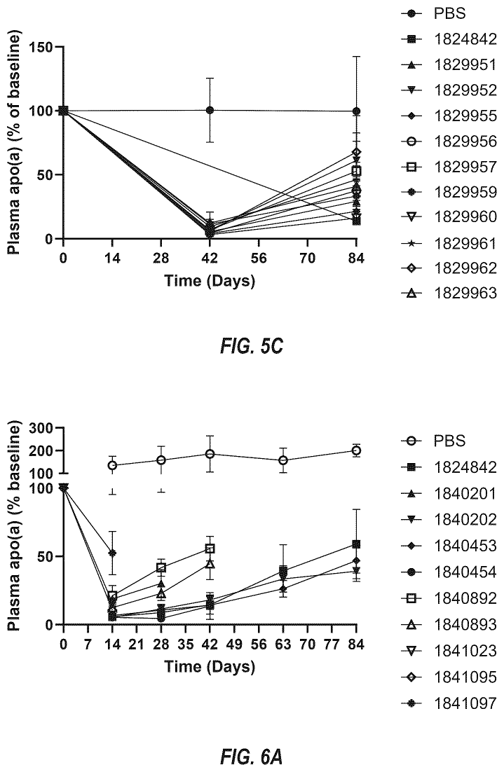

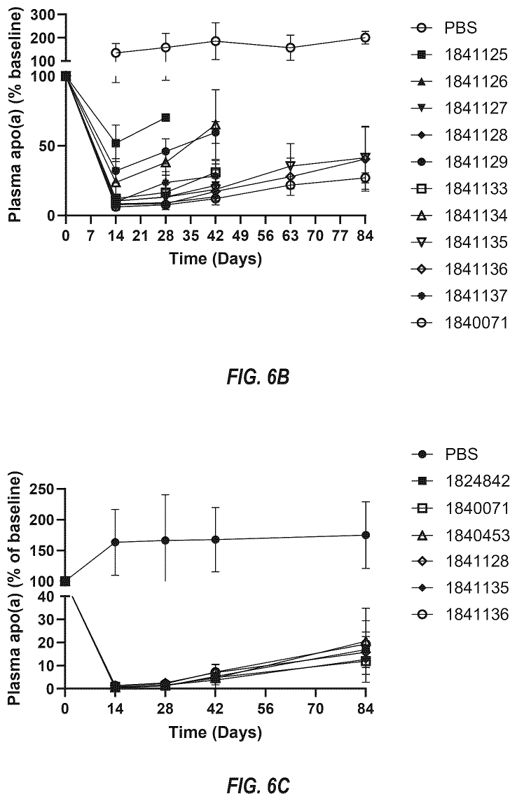

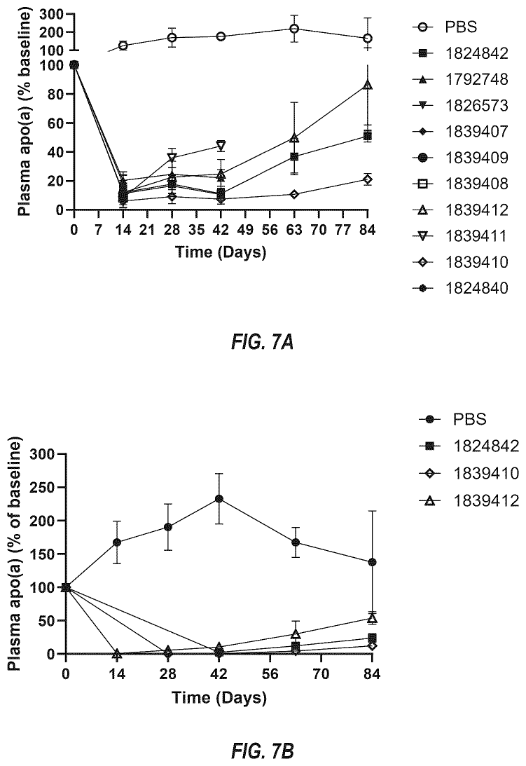

To evaluate the duration of action of oligomeric duplexes on apo(a) protein levels, blood plasma was collected between Day −3 and Day 1 prior to treatment and at various timepoints as indicated in FIGS. 1-7 and apo(a) protein levels analyzed using an assay kit from Randox (Catalog #LP2757) and an automated clinical chemistry analyzer (Hitachi Olympus AU400c, Melville, NY) (see FIGS. 1A, 2A, 3A, 4A, 5A, 5B, 6A, 6B, 7A) or by ELISA with the Abcam ELISA kit (ab212165) (see FIGS. 1B, 2B, 3B, 4B, 5B, 6B, 7B). Results were averaged for each group of mice and are presented in FIGS. 1A-1B through FIGS. 7A-7B as percent apo(a) relative to the amount of apo(a) present at baseline, as measured at pre-treatment (% baseline). N. C. indicates values that were not calculated. In some instances, values were below the Lower Limit of Quantification (LLOQ). The symbol “t” indicates one or more samples were LLOQ. N. D. indicates values that could not be determined as all the samples were below the LLOQ.

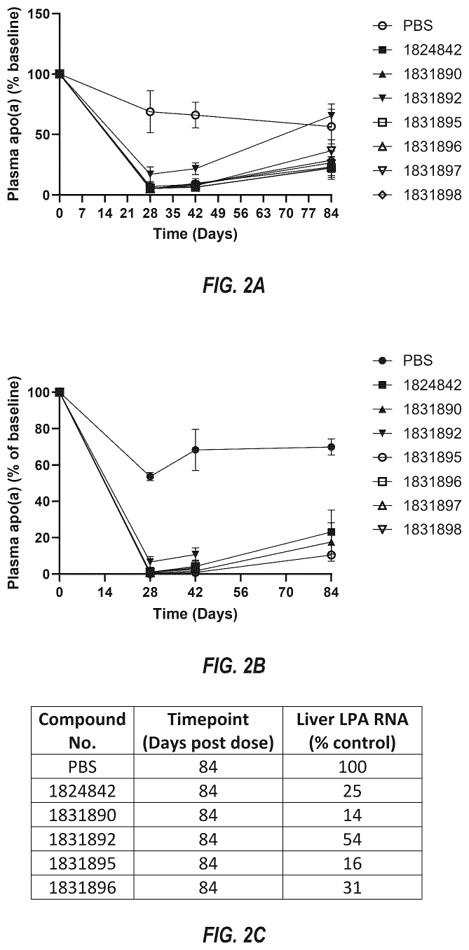

Mice were sacrificed at various timepoints post injection on either Day 28, Day 42, Day 84, or Day 126. In some instances (see FIG. 1C and FIG. 2C), RNA was extracted from liver tissue for quantitative real time RTPCR analysis of LPA RNA using human primer probe set hAPO(a)₁₂ kBTS (described herein above). LPA RNA levels were normalized to total RNA content, as measured by RIBOGREEN®. Reduction of LPA RNA is presented as percent LPA RNA relative to the amount of LPA in tissue from PBS control animals (% control). N. C. indicates values that were not calculated.

### Example 6: Tolerability of Oligomeric Duplexes

Wild type BALB/C mice (Charles River Laboratory) were treated with oligomeric compounds described above and evaluated for changes in the levels of various plasma chemistry markers. Groups of four male BALB/C mice each were injected subcutaneously once a week for two weeks (for a total of three treatments at days 1, 7, and 14) with either 30 or 100 mg/kg of oligomeric compound as described in tables 13A-13D below. One group of four male BALB/C mice was injected with PBS. Mice were euthanized at day 21.

To evaluate the effect of modified oligonucleotides on liver function, plasma levels of alanine aminotransferase (ALT) and aspartate aminotransferase (AST) were measured on the day the mice were sacrificed (Day 21) using an automated clinical chemistry analyzer (Hitachi Olympus AU400c, Melville, NY). Results were averaged for each group of mice and are presented in Table 13 below. Each experiment is identified in separate sub-tables 13A-13D below. Additional compounds 1840202, 1841133, 1841134, 1841135, and 1841136, similarly tested at 100 mg/kg in separate experiments, were well tolerated and did not demonstrate elevated plasma chemistry markers (data not shown). Compound 1840071 was similarly evaluated by treating groups of mice once a week for five weeks (for a total of six treatments) with 100 mg/kg, and plasma ALT and AST were measured on Day 21 and on the day the mice were sacrificed (Day 42); and compound 1840071 was well tolerated and did not result in elevated ALT or AST (data not shown).

TABLE 13
Plasma chemistry markers in BALB/C mice
13A.

| | | |
|---|---|---|
| | | Plasma clinical |
| Compound | Dose | chemistry |

| No. | (mg/kg) | ALT (U/L) | AST (U/L) |
|---|---|---|---|
| PBS | — | 22 | 70 |
| 1792749 | 30 | 149 | 199 |
| | 100 | 162 | 194 |

| 13B. | | 13C. | | |
|---|---|---|---|---|
| Compound | Plasma | Compound | Plasma | |
| No. | clinical | No. | clinical | |
| (100 | chemistry | (100 | chemistry | |
| mg/kg) | ALT (U/L) | mg/kg) | ALT (U/L) | AST (U/L) |
| PBS | 22 | PBS | 27 | 61 |
| 1792748 | 29 | 1839410 | 26 | 39 |
| 1824842 | 26 | 1839411 | 28 | 47 |
| 1820737 | 956 | 1839412 | 29 | 76 |

13D.

Plasma clinical
chemistry

| Compound No. | ALT | AST |
|---|---|---|
| (100 mg/kg) | (U/L) | (U/L) |
| PBS | 41 | 55 |
| 1841126 | 93 | 249 |
| 1841127 | 27 | 62 |
| 1841137 | 37 | 70 |
| 1840201 | 135 | 133 |
| 1840453 | 90 | 145 |
| 1840454 | 179 | 315 |
| 1841128 | 19 | 40 |
| 1841129 | 21 | 39 |
| 1840892 | 69 | 64 |
| 1840893 | 30 | 43 |
| 1841097 | 395 | 218 |
| 1841125 | 30 | 57 |

### Example 7: Inhibition of Human Plasminogen

Oligomeric compounds selected from examples above were tested at various doses in Huh-7 cells in a series of experiments performed under the same culture conditions to determine effects on plasminogen RNA (PLG). Results are depicted in Table 14, with different experiments in each of sub-Tables 14A/14B through 14D. In each of separate experiments, Huh-7 cells were plated at a density of 30,000 cells/well, and treated using 24-hour electroporation at 160V with ten serial dilutions of oligomeric compounds ranging from 20,000 nM to 0.00002 nM (Table 14A/14B and 14D) and 16,000 nM to 0.0016 nM (Table 14C). After a treatment period of 24 hours, total RNA was isolated from the cells and PLG RNA levels were measured by quantitative real-time RTPCR. Human PLG primer-probe sets RTS3442 (forward sequence ACCATGTCTGGACTGGAATGC, (SEQ ID NO: 121); reverse sequence CTTCTTCAGGTTCTTGTTTGGAAAT (SEQ ID NO: 122); probe sequence ACTCTCAGAGCCCACACGCTCATGG (SEQ ID NO. 123)) and RTS3443 (forward sequence GCGGTGGGAGTACTGTAAGATACC (SEQ ID NO: 124); reverse sequence GTGCTGTGGGAGCCAATTG (SEQ ID NO: 125); probe sequence CTGTGACTCCTCCCCAGTATCCACGGA (SEQ ID NO. 126)) were used to measure RNA levels as described above. PLG RNA levels were normalized to total RNA content, as measured by RIBOGREEN®.

Compound No. 1744547, targeted to ACTN1 RNA, was included in the experiments as a control, and the effect on ACTN1 RNA levels was evaluated. Compound No. 1744547 comprises antisense Compound No. 1744535 (SEQ ID NO: 127), which has a nucleobase sequence (from 5′ to 3′) of TAAGUGAGCUAGCAAACACACAU, a sugar motif (from 5′ to 3′) of efyyyfyyyyyyyfyfyyyyyyy, and an internucleoside linkage motif (from 5′ to 3′) of ssooooooooooooooooooss; and sense Compound No. 1744544 (SEQ ID NO: 128) which has a nucleobase sequence (from 5′ to 3′) of GUGUGUUUGCUAGCUCACUUA; a sugar motif (from 5′ to 3′) of yyyyyyfyfffyyyyyyyyyy, and an internucleoside linkage motif (from 5′ to 3′) of ssooooooooooooooooss; wherein each “y” represents a 2′-OMe sugar moiety, each “f” represents a 2′-F sugar moiety, and each “e” represents a cEt sugar moiety, and wherein each “s” represents a phosphorothioate internucleoside linkage, and each “o” represents a phosphodiester internucleoside linkage; and sense oligonucleotide Compound No. 1744544 has an HPPO-GalNAc moiety conjugated to the 3′-end of the oligonucleotide via a phosphodiester bond. ACTN1 RNA levels were measured by quantitative real-time RTPCR using primer-probe set HTS5001 (forward sequence CAATATGGCGGGCACCAA (SEQ ID NO: 129); reverse sequence GGTCCCATTTGCCATTGATC (SEQ ID NO: 130); probe sequence CCCTACACAACCATCACGCCTCAGG (SEQ ID NO. 131)). ACTN1 RNA levels were normalized to total RNA content, as measured by RIBOGREEN®.

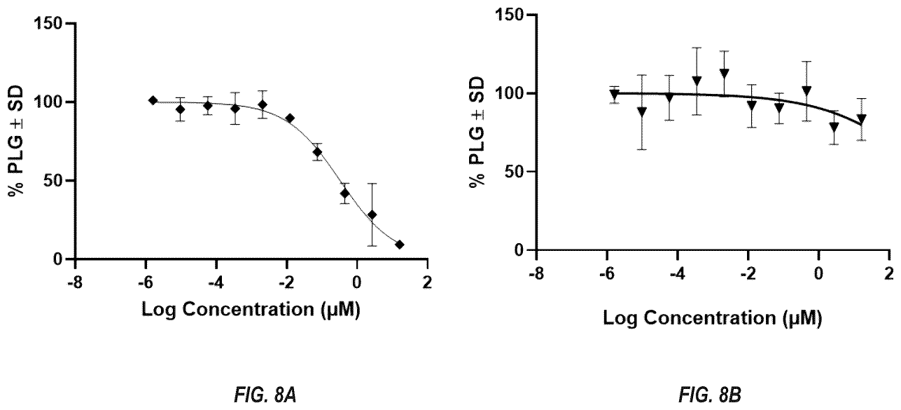

PLG RNA was measured and determined as a percent of PLG RNA in untreated controls cells (% UTC). In addition to control sample treated with ACTN1 targeted compound indicated in the results in Table 14, ACTN1 RNA was also measured as internal controls in samples treated with LPA targeted compounds, and no reduction in ACTN1 RNA was detected (data not shown). The IC₅₀ value of each modified oligonucleotide was calculated using GraphPad Prism 10 software (GraphPad Software, San Diego, CA) and is presented in Table 14. “N. C.” represents IC₅₀ values that were above the highest concentration evaluated (16 mM or 20 mM). Exemplary results for compounds demonstrating PLG reduction and no significant reduction of PLG are shown in FIGS. 8A-8B. FIG. 8A depicts results of PLG levels measured after treatment with compound 1826573; and FIG. 8B depicts results of PLG levels measured after treatment with compound 1840071.

Compounds 1841133, 1841125, 1840892, 1841134, 1841126, 1840893, 1840201, 1841135, 1841127, 1841023, 1840202, 1841136, 1841128, 1841095, 1840453, 1841137, 1841129, 1841097, 1840454, 1829960 and 1829956 were evaluated in similar experiments as those depicted in Table 14C, under slightly varied conditions of 24 hour electroporation of cells plated at density of 25000 cells/well; and compounds demonstrating significant LPA RNA inhibition did not demonstrate significant PLG RNA reduction activity, with the exception of 1829956 which demonstrated slightly more knock down of PLG RNA than in the experiment in Table 14C (data not shown).

TABLE 14
Reduction of human PLG RNA

| 14A. | | | 14B. | | |
|---|---|---|---|---|---|
| Primer | | PLG | Primer | | PLG |
| Probe | Compound | IC₅₀ | Probe | Compound | IC₅₀ |
| Set | Number | (μM) | Set | Number | (μM) |
| RTS3442 | 1831895 | 0.09 | RTS3443 | 1831895 | 0.08 |
| | 1831890 | 1.73 | | 1831890 | 0.58 |
| | 1831898 | N.C. | | 1831898 | N.C. |
| | 1829960 | N.C. | | 1829960 | N.C. |
| | 1831897 | N.C. | | 1831897 | N.C. |
| | 1829955 | 0.59 | | 1829955 | 0.25 |
| | 1831896 | 0.48 | | 1831896 | 0.21 |
| | 1829956 | N.C. | | 1829956 | N.C. |
| | 1829951 | 0.09 | | 1829951 | 0.1 |
| | 1829962 | N.C. | | 1829962 | N.C. |
| | 1829952 | 0.7 | | 1829952 | N.C. |
| | 1829957 | 0.77 | | 1829957 | 0.24 |
| | 1826328 | 3.21 | | 1826328 | 1.03 |
| | 1829961 | N.C. | | 1829961 | N.C. |
| | 1831892 | 3.02 | | 1831892 | 2.43 |
| RTS3442 | 1829959 | N.C. | RTS3443 | 1829959 | N.C. |
| | 1829963 | N.C. | | 1829963 | N.C. |
| | 1787751 | N.C. | | 1787751 | N.C. |
| | 1792748 | N.C. | | 1792748 | N.C. |
| | 1824840 | 0.05 | | 1824840 | 0.13 |
| | 1820737 | N.C | | 1820737 | N.C. |
| | 1839407 | 0.44 | | 1839407 | 1.12 |
| | 1826573 | 0.33 | | 1826573 | 0.76 |
| | 1839408 | 0.28 | | 1839408 | 1.21 |
| | 1839410 | N.C. | | 1839410 | N.C. |
| | 1839411 | N.C. | | 1839411 | N.C. |
| | 1839409 | 0.19 | | 1839409 | 0.11 |
| | 1839412 | N.C. | | 1839412 | N.C. |
| | 1744547 | N.C. | | 1744547 | N.C. |
| HTS5001 | 1744547 | 0.14 | | | |
| 14C | | | 14D | | |
| Primer | | PLG | Primer | | PLG |
| Probe | Compound | IC₅₀ | Probe | Compound | IC₅₀ |
| Set1 | Number | (μM) | Set | Number | (μM) |
| RTS3442 | 1792749 | N.C. | RTS3442 | 1840071 | N.C. |
| | 1824842 | N.C. | | 1744547 | N.C. |
| | 1826328 | 16.66 | RTS3443 | 1840071 | N.C. |
| | 1787751 | N.C. | | 1744547 | N.C. |
| | | | HTS5001 | 1744547 | 0.049 |

### Example 8: Reduction of Human LPA in LPA Transgenic Mice

Oligomeric duplexes were analyzed alongside competitor compounds or surrogate for effects on LPA mRNA and apo(a) protein in human LPA transgenic mice (described herein above). Groups of 4 female LPA transgenic mice each received a single subcutaneous injection of oligomeric duplex compound at various doses as indicated. One group of 4 female LPA transgenic mice received a single subcutaneous injection of PBS as a negative control.

Compound AD03851, is a compound targeting LPA previously disclosed in International Patent Application Publication No. WO2017/059223, which is incorporated herein by reference. Compound No. 1851494 is a surrogate for compound LPA-3291-M1, a nicked hairpin compound targeting LPA previously disclosed in International Patent Application Publication No. WO2022/032288, which is incorporated herein by reference, and differs from LPA-329-M1 in two changes: first, Compound No. 1851494 comprises a vinyl phosphonate moiety on the 5′-end of the antisense oligonucleotide, whereas LPA-329-M1 comprises a methyl methylene phosphonate (MeMOP) moiety on the on the 5′-end of the antisense oligonucleotide; and second, Compound No. 1851494 comprises a THA-GalNAc conjugate (as described herein) on the 5′-end of the sense oligonucleotide, whereas LPA-329-M1 comprises 2′-GalNAc adenosine (AdemA) nucleosides at positions 28, 29, and 30 of the sense oligonucleotide.

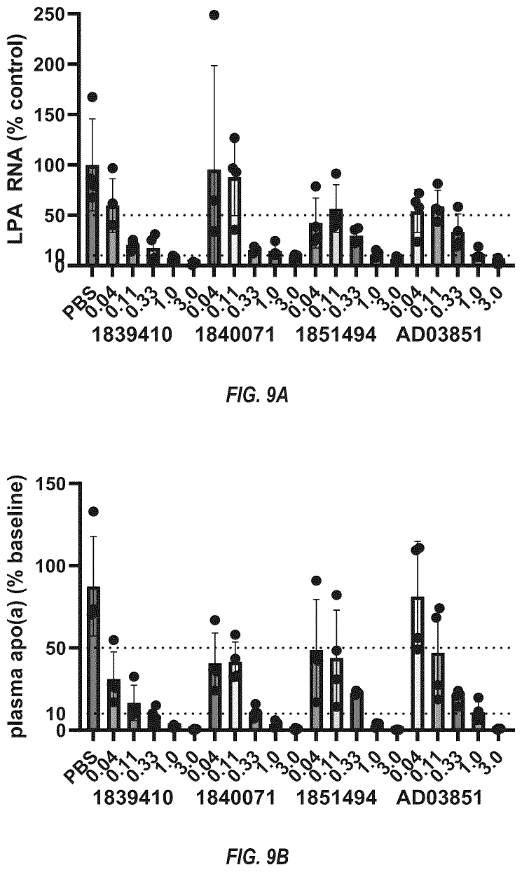

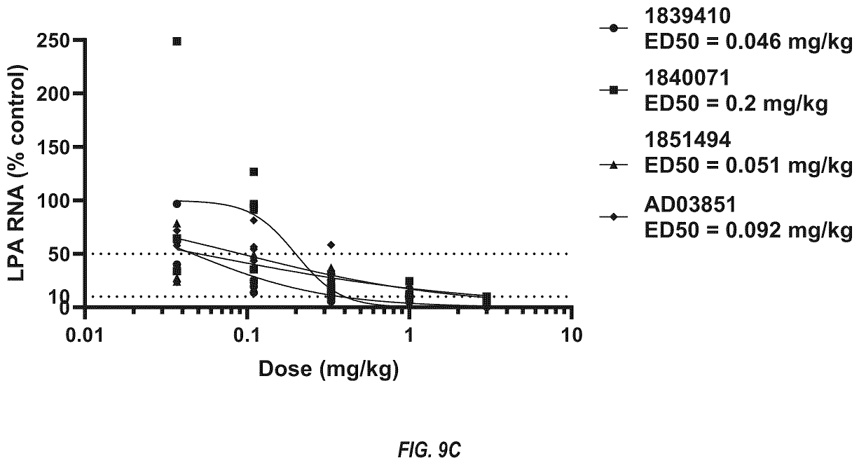

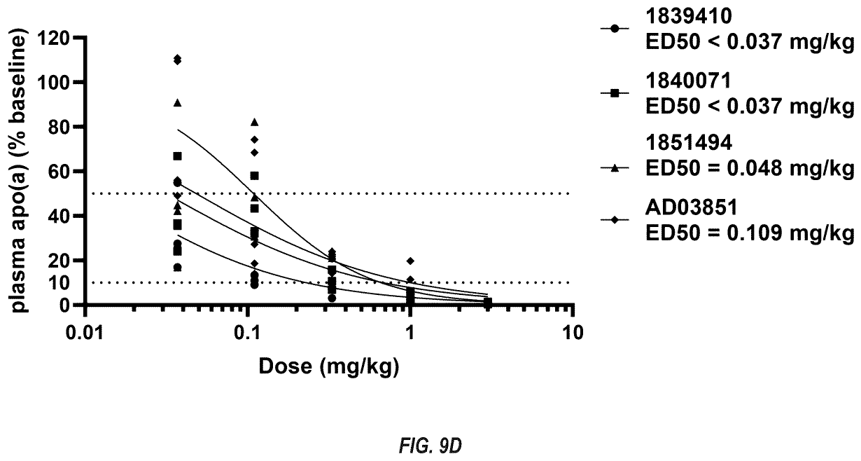

LPA RNA and apo(a) protein were evaluated pre- and post-treatment to determine dose dependent effects of compounds. Results are depicted in FIG. 9A-9D. 2 weeks post treatment, mice were sacrificed, and RNA extracted from liver tissue for quantitative real time RTPCR analysis of LPA RNA using human primer probe set hAPO(a)₁₂ kBTS (described above). LPA RNA levels were normalized to total RNA content, as measured by RIBOGREEN®. Reduction of LPA RNA is presented in FIG. 9A and FIG. 9C as percent LPA RNA relative to the amount of LPA RNA in tissue from PBS control animals (% control). ED50 were calculated and presented in Table 15. For evaluation of apo(a) protein levels, blood plasma was collected 1 day prior to treatment on Day −1 and on Day 14 when the mice were sacrificed. apo(a) protein levels were analyzed by ELISA array with the Abcam ELISA kit (ab212165). Results were averaged for each group of mice and are presented in FIG. 9B and FIG. 9D as percent apo(a) protein relative to the amount of apo(a) protein present at baseline (% baseline). ED50 were calculated and presented in Table 15.

TABLE 15
Reduction of LPA and effect on apo(a)
levels in LPA transgenic mice

| | Liver LPA RNA | apo(a) Plasma Protein |
|---|---|---|
| Compound No. | ED50 (mg/kg) | ED50 (mg/kg) |
| 1839410 | 0.05 | 0.01 |
| 1840071 | 0.20 | 0.03 |
| 1851494 | 0.05 | 0.05 |
| AD03851 | 0.09 | 0.11 |

### Example 9: Reduction of Human LPA in LPA Transgenic Mice, Duration of Action

Oligomeric duplexes and comparator compounds were analyzed for effect on apo(a) protein in human LPA transgenic mice (described herein above). Compound AD03851, a compound targeting LPA was previously disclosed in International Patent Application Publication No WO2017/059223, which is incorporated herein by reference. Compound C21S, is a surrogate for Conjugate 21, a compound targeting LPA previously disclosed in International Patent Application Publication No WO2020/099476, which is incorporated herein by reference, and differs from Conjugate 21 in that C21S has a different GalNAc3 ligand conjugated to the siRNA than Conjugate 21.

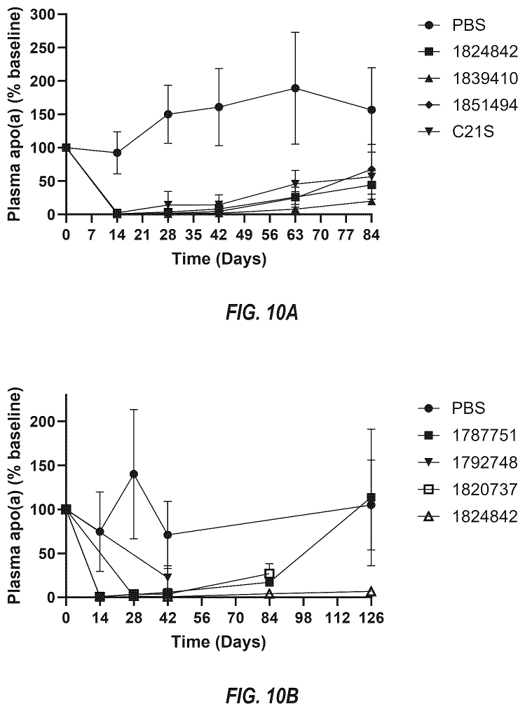

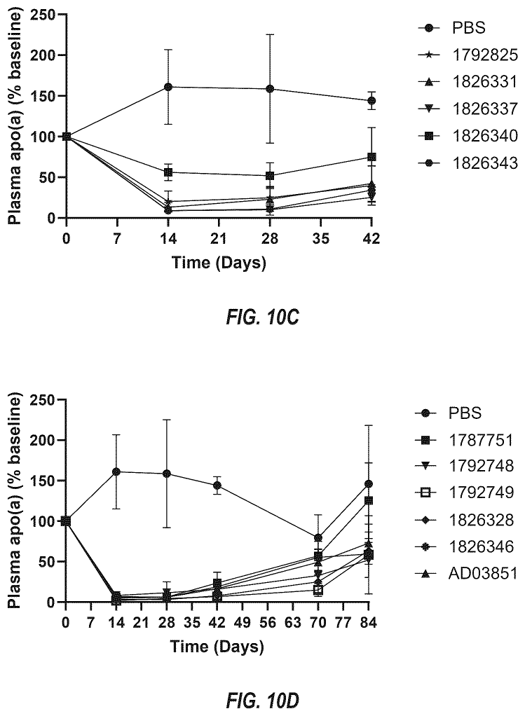

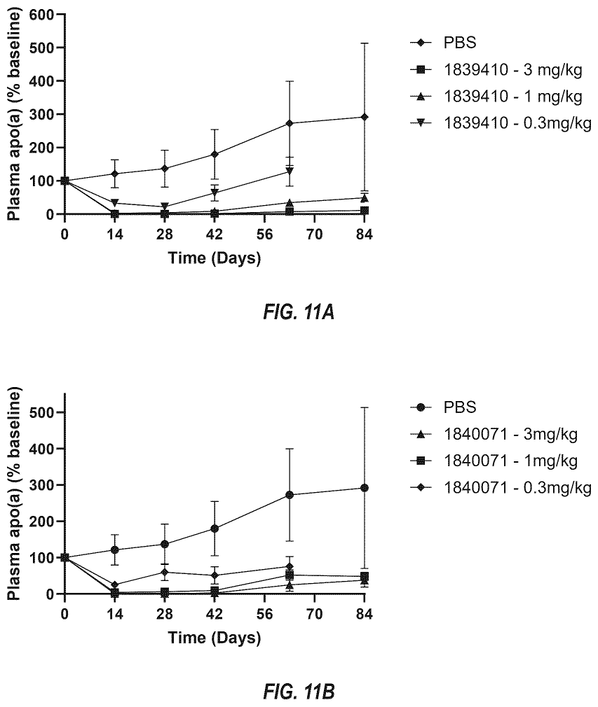

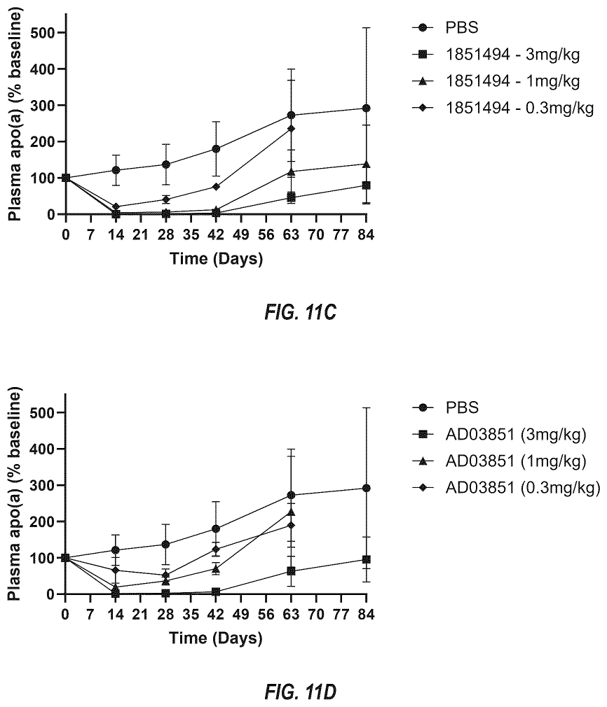

Groups of 3-9 female LPA transgenic mice each received a single subcutaneous injection of compound at various doses. One group of 3-4 female LPA transgenic mice received a single subcutaneous injection of PBS as a negative control. Duration of action ofthe compounds was evaluated by measuring apo(a) protein levels over time following compound administration. Blood plasma was collected 1 day prior to treatment on Day −1 and at various timepoints and apo(a) protein levels were analyzed by ELISA array with the Abcam ELISA kit (ab212165). Results were averaged for each group of mice and are presented in FIGS. 10A-10D and FIGS. 11A-11D. FIGS. 10A-10D depict results of a single 3 mg/kg dose of compound over time as a percent apo(a) protein relative to the amount of apo(a) protein present at baseline (% baseline). FIGS. 10C and 10D depict results of one experiment in two separate graphs due to the number of compounds. In some instances, values were below the level of Quantification (LLOQ), values were not calculated, or values could not be determined. In some instances, values were below the level of Quantification (LLOQ), values were not calculated, or values could not be determined. FIGS. 11A-11D depict results of various doses of compounds over time as a percent apo(a) protein relative to the amount of apo(a) protein present at baseline (% baseline).

1-104. (canceled)

105. An oligomeric duplex comprising:
i) an oligomeric compound according to the following chemical notation:
(SEQ ID NO: 26)
vP-TesGfsCyoCyoUyoCfoGxoAyoUyoAyoAyoCyoUyoCfoUyoGy
oUyoCyoCyoAyoUysAesAe,
wherein:
A=an adenine nucleobase, C=a cytosine nucleobase, G=a guanine nucleobase, T=a thymine nucleobase, U=a uracil nucleobase, x=a 2′-β-D-deoxyxylosyl sugar moiety, e=a 2′-O-methoxyethyl (MOE) sugar moiety, f=a 2′-fluoro sugar moiety, y=a 2′-O-methyl (OMe) sugar moiety, o=a phosphodiester internucleoside linkage, s=a phosphorothioate internucleoside linkage, and VP=a 5′ vinyl phosphonate moiety; and
ii) an oligomeric compound according to the following chemical notation:
(SEQ ID NO: 53)
[THA-GalNAc]-AesUesGyoGyoAyoCyoAyoGyoAfoGfoUfoUyoA
yoUeoCyoGyoAyoGyoGysCesAe;
wherein: 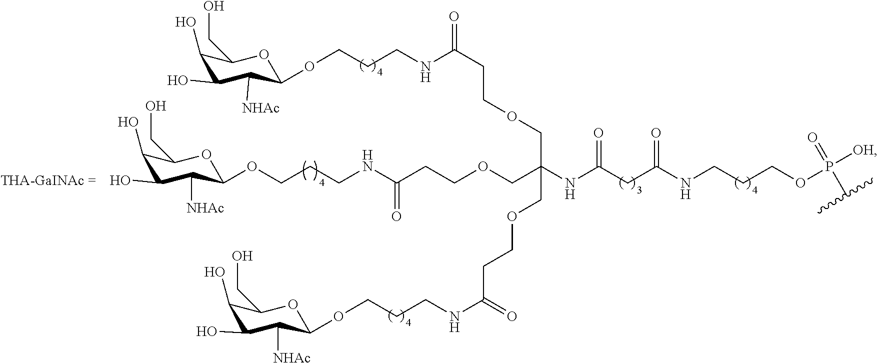
A=an adenine nucleobase, C=a cytosine nucleobase, G=a guanine nucleobase, T=a thymine nucleobase, U=a uracil nucleobase, e=a 2′-MOE sugar moiety, f=a 2′-fluoro sugar moiety, y=a 2′-OMe sugar moiety, o=a phosphodiester internucleoside linkage, and s=a phosphorothioate internucleoside linkage.

106. An oligomeric duplex according to the chemical structure: 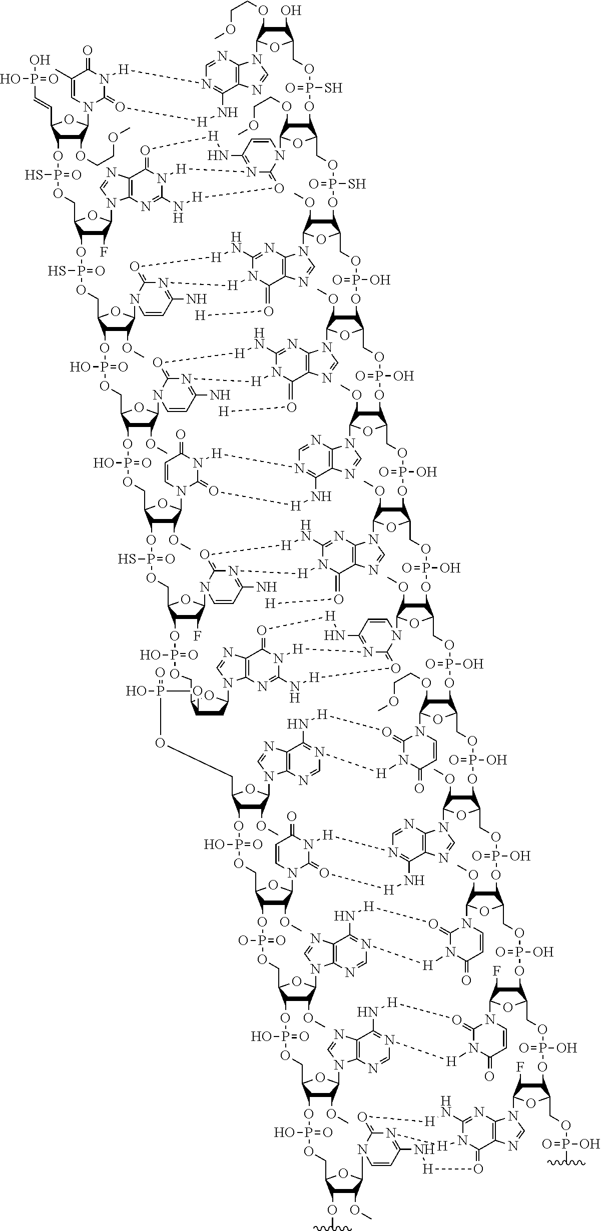 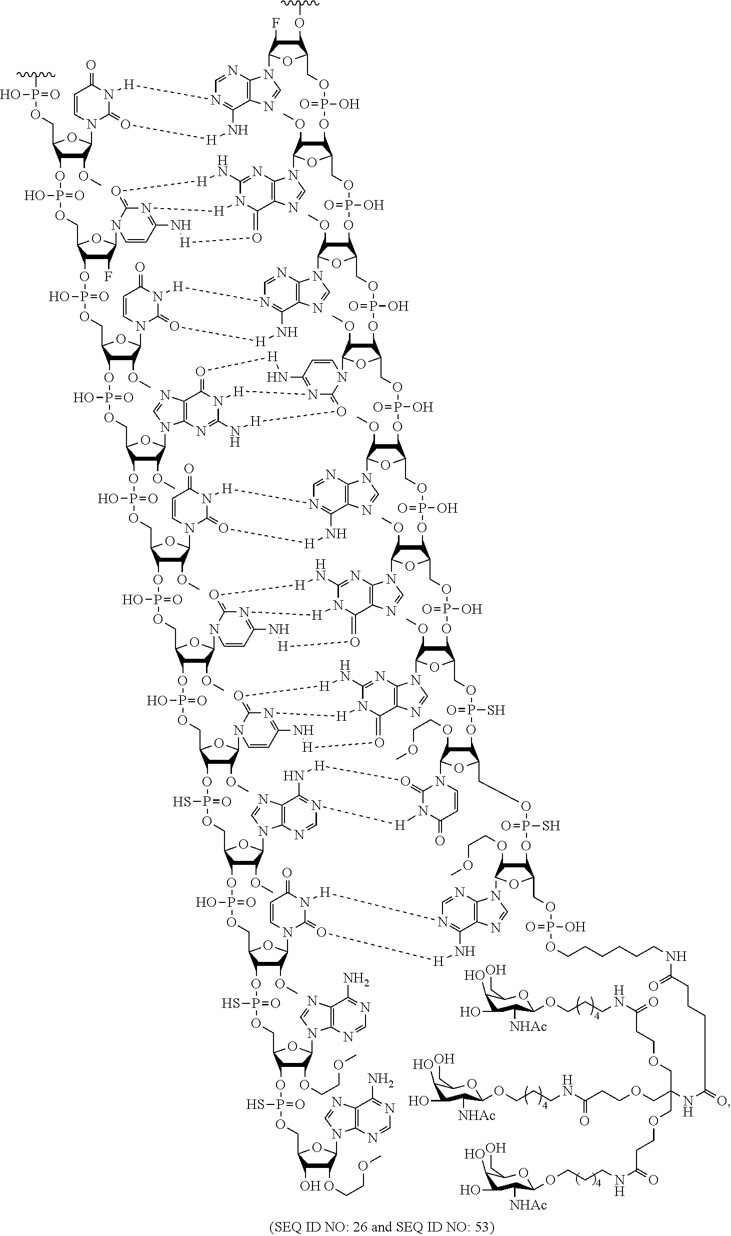
or an anionic form or salt thereof.

107. The oligomeric duplex of claim 106, wherein the salt is a sodium salt.

108. The oligomeric duplex of claim 106, wherein the salt is a potassium salt.

109. An oligomeric duplex according to the following chemical structure:

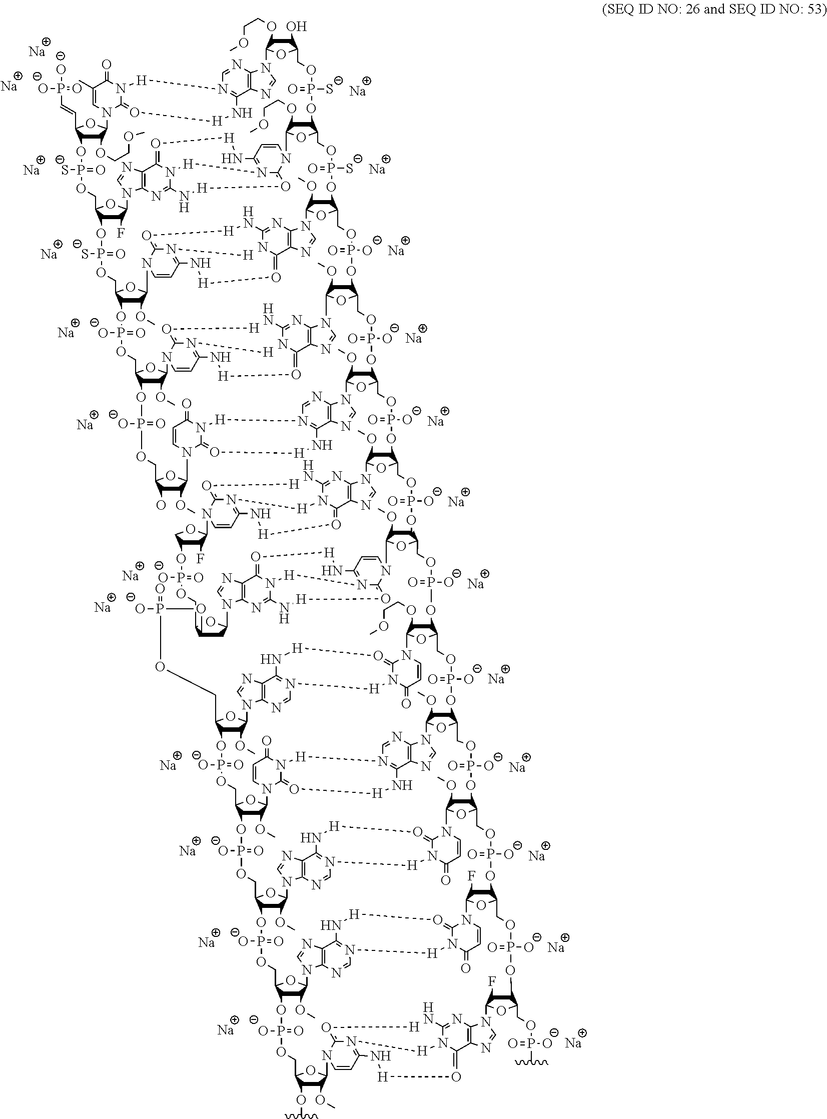

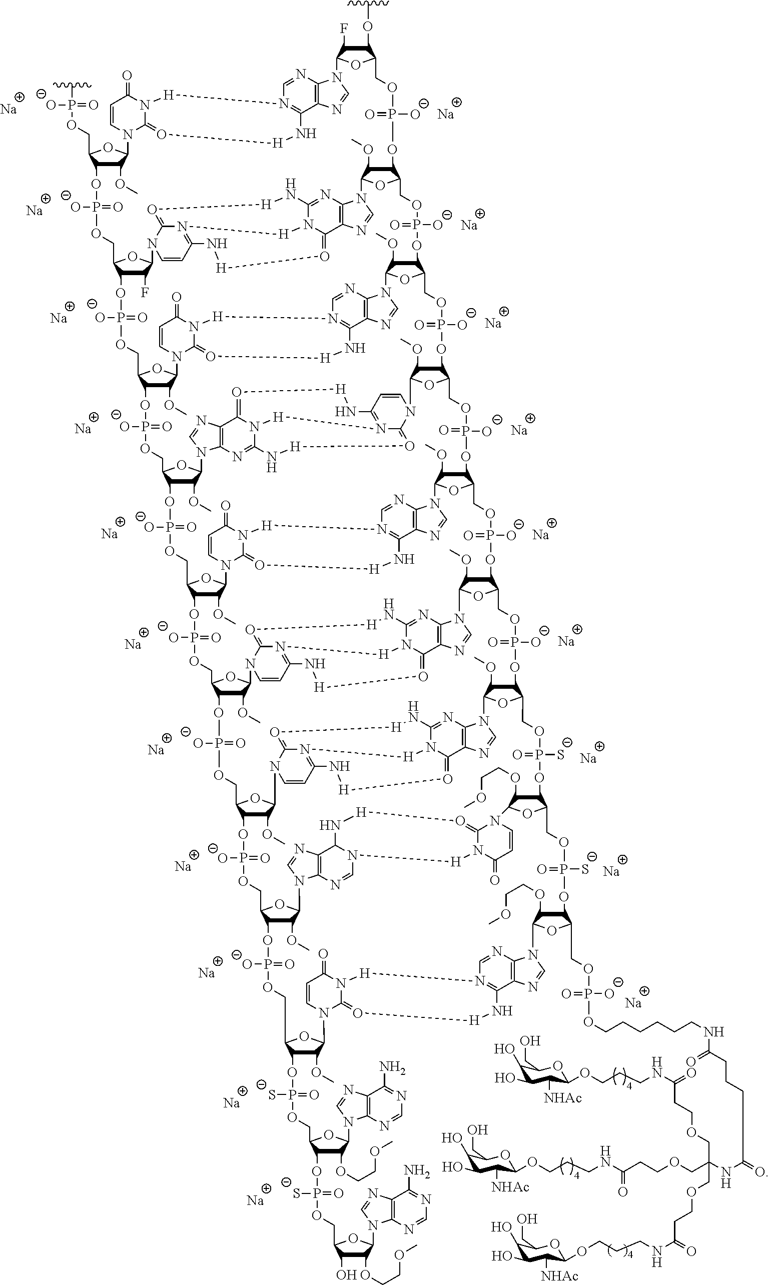

110. An oligomeric duplex according to the following chemical structure:

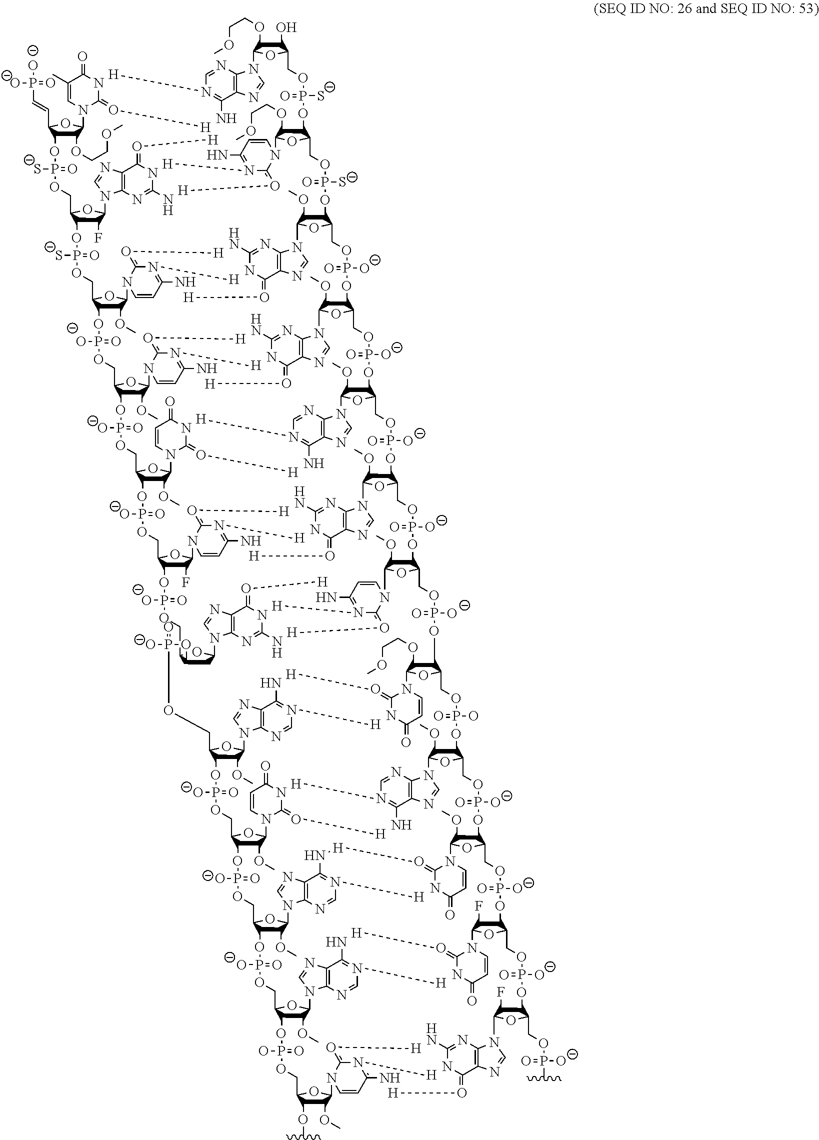

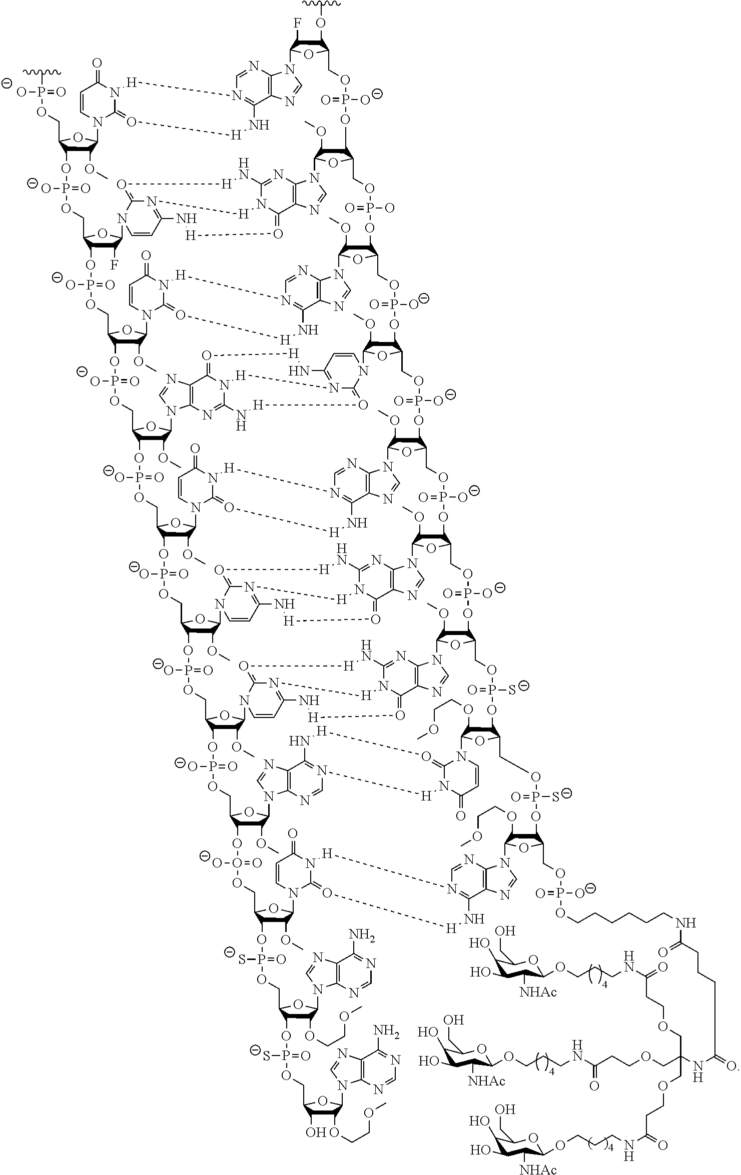

111. A pharmaceutical composition comprising the oligomeric duplex of claim 106 and a pharmaceutically acceptable diluent or carrier.

112. A pharmaceutical composition comprising the oligomeric duplex of claim 109 and a pharmaceutically acceptable diluent or carrier.

113. A pharmaceutical composition comprising the oligomeric duplex of claim 110 and a pharmaceutically acceptable diluent or carrier.

114. The pharmaceutical composition of claim 111, wherein the pharmaceutically acceptable diluent is water or phosphate-buffered saline.

115. The pharmaceutical composition of claim 112, wherein the pharmaceutically acceptable diluent is water or phosphate-buffered saline.

116. The pharmaceutical composition of claim 113, wherein the pharmaceutically acceptable diluent is water or phosphate-buffered saline.


SEQUENCE LISTING (146 sequences)

SEQ ID NO: 1 - RNA, 6431 nt, Homo sapiens
  source 1..6431: mol_type = mRNA
gtaagtcaacaatgtcctgggattgggacacactttctgggcactgctggccagtcccaaaatggaacataaggaagtggttcttctacttcttttatttctgaaatcagcagcacctgagcaaagccatgtggtccaggattgctaccatggtgatggacagagttatcgaggcacgtactccaccactgtcacaggaaggacctgccaagcttggtcatctatgacaccacatcaacataataggaccacagaaaactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcaaaggcctggggtgcaggagtgctaccatggtaatggacagagttatcgaggcacatactccaccactgtcacaggaagaacctgccaagcttggtcatctatgacaccacactcgcatagtcggaccccagaatactacccaaatgctggcttgatcatgaactactgcaggaatccagatgctgtggcagctccttattgttatacgagggatcccggtgtcaggtgggagtactgcaacctgacgcaatgctcagacgcagaagggactgccgtcgcgcctccgactgttaccccggttccaagcctagaggctccttccgaacaagcaccgactgagcagaggcctggggtgcaggagtgctaccacggtaatggacagagttatcgaggcacatactccaccactgtcactggaagaacctgccaagcttggtcatctatgacaccacactcgcatagtcggaccccagaatactacccaaatgctggcttgatcatgaactactgcaggaatccagatcctgtggcagccccttattgttatacgagggatcccagtgtcaggtgggagtactgcaacctgacacaatgctcagacgcagaagggactgccgtcgcgcctccaactattaccccgattccaagcctagaggctccttctgaacaagcaccaactgagcaaaggcctggggtgcaggagtgctaccacggaaatggacagagttatcaaggcacatacttcattactgtcacaggaagaacctgccaagcttggtcatctatgacaccacactcgcatagtcggaccccagcatactacccaaatgctggcttgatcaagaactactgccgaaatccagatcctgtggcagccccttggtgttatacaacagatcccagtgtcaggtgggagtactgcaacctgacacgatgctcagatgcagaatggactgccttcgtccctccgaatgttattctggctccaagcctagaggctttttttgaacaagcactgactgaggaaacccccggggtacaggactgctactaccattatggacagagttaccgaggcacatactccaccactgtcacaggaagaacttgccaagcttggtcatctatgacaccacaccagcatagtcggaccccagaaaactacccaaatgctggcctgaccaggaactactgcaggaatccagatgctgagattcgcccttggtgttacaccatggatcccagtgtcaggtgggagtactgcaacctgacacaatgcctggtgacagaatcaagtgtccttgcaactctcacggtggtcccagatccaagcacagaggcttcttctgaagaagcaccaacggagcaaagccccggggtccaggattgctaccatggtgatggacagagttatcgaggctcattctctaccactgtcacaggaaggacatgtcagtcttggtcctctatgacaccacactggcatcagaggacaacagaatattatccaaatggtggcctgaccaggaactactgcaggaatccagatgctgagattagtccttggtgttataccatggatcccaatgtcagatgggagtactgcaacctgacacaatgtccagtgacagaatcaagtgtccttgcgacgtccacggctgtttctgaacaagcaccaacggagcaaagccccacagtccaggactgctaccatggtgatggacagagttatcgaggctcattctccaccactgttacaggaaggacatgtcagtcttggtcctctatgacaccacactggcatcagagaaccacagaatactacccaaatggtggcctgaccaggaactactgcaggaatccagatgctgagattcgcccttggtgttataccatggatcccagtgtcagatgggagtactgcaacctgacgcaatgtccagtgatggaatcaactctcctcacaactcccacggtggtcccagttccaagcacagagcttccttctgaagaagcaccaactgaaaacagcactggggtccaggactgctaccgaggtgatggacagagttatcgaggcacactctccaccactatcacaggaagaacatgtcagtcttggtcgtctatgacaccacattggcatcggaggatcccattatactatccaaatgctggcctgaccaggaactactgcaggaatccagatgctgagattcgcccttggtgttacaccatggatcccagtgtcaggtgggagtactgcaacctgacacgatgtccagtgacagaatcgagtgtcctcacaactcccacagtggccccggttccaagcacagaggctccttctgaacaagcaccacctgagaaaagccctgtggtccaggattgctaccatggtgatggacggagttatcgaggcatatcctccaccactgtcacaggaaggacctgtcaatcttggtcatctatgataccacactggcatcagaggaccccagaaaactacccaaatgctggcctgaccgagaactactgcaggaatccagattctgggaaacaaccctggtgttacacaaccgatccgtgtgtgaggtgggagtactgcaatctgacacaatgctcagaaacagaatcaggtgtcctagagactcccactgttgttccagttccaagcatggaggctcattctgaagcagcaccaactgagcaaacccctgtggtccggcagtgctaccatggtaatggccagagttatcgaggcacattctccaccactgtcacaggaaggacatgtcaatcttggtcatccatgacaccacaccggcatcagaggaccccagaaaactacccaaatgatggcctgacaatgaactactgcaggaatccagatgccgatacaggcccttggtgttttaccatggaccccagcatcaggtgggagtactgcaacctgacgcgatgctcagacacagaagggactgtggtcgctcctccgactgtcatccaggttccaagcctagggcctccttctgaacaagactgtatgtttgggaatgggaaaggataccggggcaagaaggcaaccactgttactgggacgccatgccaggaatgggctgcccaggagccccatagacacagcacgttcattccagggacaaataaatgggcaggtctggaaaaaaattactgccgtaaccctgatggtgacatcaatggtccctggtgctacacaatgaatccaagaaaactttttgactactgtgatatccctctctgtgcatcctcttcatttgattgtgggaagcctcaagtggagccgaagaaatgtcctggaagcattgtaggggggtgtgtggcccacccacattcctggccctggcaagtcagtctcagaacaaggtttggaaagcacttctgtggaggcaccttaatatccccagagtgggtgctgactgctgctcactgcttgaagaagtcctcaaggccttcatcctacaaggtcatcctgggtgcacaccaagaagtgaacctcgaatctcatgttcaggaaatagaagtgtctaggctgttcttggagcccacacaagcagatattgccttgctaaagctaagcaggcctgccgtcatcactgacaaagtaatgccagcttgtctgccatccccagactacatggtcaccgccaggactgaatgttacatcactggctggggagaaacccaaggtacctttgggactggccttctcaaggaagcccagctccttgttattgagaatgaagtgtgcaatcactataagtatatttgtgctgagcatttggccagaggcactgacagttgccagggtgacagtggagggcctctggtttgcttcgagaaggacaaatacattttacaaggagtcacttcttggggtcttggctgtgcacgccccaataagcctggtgtctatgctcgtgtttcaaggtttgttacttggattgagggaatgatgagaaataattaattggacgggagacagagtgaagcatcaacctacttagaagctgaaacgtgggtaaggatttagcatgctggaaataatagacagcaatcaaacgaagacactgttcccagctaccagctatgccaaaccttggcatttttggtatttttgtgtataagcttttaaggtctgactgacaaattctgtattaaggtgtcatagctatgacatttgttaaaaataaactctgcacttattttgatttgaa

SEQ ID NO: 2 - DNA, 45 bp, synthetic construct
  source 1..45: mol_type = other DNA
gctaccatggtaatggacagagttatcgaggcacatactccacca

SEQ ID NO: 3 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-fluoro guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methyl cytidine 3'-phosphorothioate
  modified_base 23: mod_base = OTHER; note = 2'-O-methyl adenosine
tgcctcgataactctgtccatca

SEQ ID NO: 4 - RNA, 23 nt, synthetic construct
  source 1..23: mol_type = other RNA
  modified_base 1: mod_base = OTHER; note = 2'-O-methyl uridine 3'-phosphorothioate 5' vinyl phosphonate
  modified_base 2: mod_base = OTHER; note = 2'-fluoro guanosine 3'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-fluoro guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methyl cytidine 3'-phosphorothioate
  modified_base 23: mod_base = OTHER; note = 2'-O-methyl adenosine
tgcctcgataactctgtccatca

SEQ ID NO: 5 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-deoxyuridine 3'-phosphorothioate
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methoxyethyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-deoxyuridine 3'-phosphorothioate
  modified_base 15: mod_base = OTHER; note = 2'-O-methyl cytidine 3'-phosphodiester
  modified_base 16: mod_base = OTHER; note = 2'-deoxyguanosine 3'-phosphorothioate
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 6 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro cytidine 3'-phosphorothioate
  modified_base 3: mod_base = OTHER; note = 2'-O-methyl cytidine 3'-phosphodiester
  modified_base 4: mod_base = OTHER; note = 2'-O-methyl uridine 3'-phosphodiester
  modified_base 5: mod_base = OTHER; note = 2'-O-methyl cytidine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-deoxyuridine 3'-phosphorothioate
  modified_base 15: mod_base = OTHER; note = 2'-O-methyl guanosine 3'-phosphodiester
  modified_base 16: mod_base = OTHER; note = 2'-deoxyuridine 3'-phosphorothioate
  modified_base 17..18: mod_base = OTHER; note = 2'-O-methyl cytidine 3'-phosphodiester
  modified_base 19: mod_base = OTHER; note = 2'-O-methyl adenosine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcctcgataactctgtccattaa

SEQ ID NO: 7 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-deoxyuridine 3'-phosphorothioate
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-fluoro guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 8 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro cytidine 3'-phosphorothioate
  modified_base 3: mod_base = OTHER; note = 2'-O-methyl cytidine 3'-phosphodiester
  modified_base 4: mod_base = OTHER; note = 2'-O-methyl uridine 3'-phosphodiester
  modified_base 5: mod_base = OTHER; note = 2'-O-methyl cytidine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9..10: mod_base = OTHER; note = 2'-O-methyl adenosine 3'-phosphodiester
  modified_base 11: mod_base = OTHER; note = 2'-O-methyl cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..18: mod_base = OTHER; note = 2'-O-methyl cytidine 3'-phosphodiester
  modified_base 19: mod_base = OTHER; note = 2'-O-methyl adenosine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcctcgataactctgtccattaa

SEQ ID NO: 9 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro cytidine 3'-phosphorothioate
  modified_base 3: mod_base = OTHER; note = 2'-O-methyl cytidine 3'-phosphodiester
  modified_base 4: mod_base = OTHER; note = 2'-O-methyl uridine 3'-phosphodiester
  modified_base 5: mod_base = OTHER; note = 2'-O-methyl cytidine 3'-phosphodiester
  modified_base 6: mod_base = OTHER; note = 2'-deoxyguanosine 3'-phosphorothioate
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..18: mod_base = OTHER; note = 2'-O-methyl cytidine 3'-phosphodiester
  modified_base 19: mod_base = OTHER; note = 2'-O-methyl adenosine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcctcgataactctgtccattaa

SEQ ID NO: 10 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-deoxyuridine 3'-phosphorothioate
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methoxyethyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-fluoro guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 11 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 3'-fluoro hexitol nucleic acid cytidine 4'-phosphorothioate
  modified_base 3: mod_base = OTHER; note = 2'-O-methyl cytidine 3'-phosphodiester
  modified_base 4: mod_base = OTHER; note = 2'-O-methyl uridine 3'-phosphodiester
  modified_base 5: mod_base = OTHER; note = 2'-O-methyl cytidine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9..10: mod_base = OTHER; note = 2'-O-methyl adenosine 3'-phosphodiester
  modified_base 11: mod_base = OTHER; note = 2'-O-methyl cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..18: mod_base = OTHER; note = 2'-O-methyl cytidine 3'-phosphodiester
  modified_base 19: mod_base = OTHER; note = 2'-O-methyl adenosine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcctcgataactctgtccattaa

SEQ ID NO: 12 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 3'-fluoro hexitol nucleic acid guanosine 4'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-fluoro uridine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-fluoro guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 13 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 3'-fluoro hexitol nucleic acid guanosine 4'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-fluoro uridine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methoxyethyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-fluoro guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 14 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 3'-fluoro hexitol nucleic acid cytidine 4'-phosphorothioate
  modified_base 3: mod_base = OTHER; note = 2'-O-methyl cytidine 3'-phosphodiester
  modified_base 4: mod_base = OTHER; note = 2'-O-methyl uridine 3'-phosphodiester
  modified_base 5: mod_base = OTHER; note = 2'-O-methyl cytidine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methoxyethyl adenosine 3'-phosphodiester
  modified_base 10: mod_base = OTHER; note = 2'-O-methyl adenosine 3'-phosphodiester
  modified_base 11: mod_base = OTHER; note = 2'-O-methyl cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17..18: mod_base = OTHER; note = 2'-O-methyl cytidine 3'-phosphodiester
  modified_base 19: mod_base = OTHER; note = 2'-O-methyl adenosine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcctcgataactctgtccattaa

SEQ ID NO: 15 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-fluoro guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgcctcgataactctgtccataa

SEQ ID NO: 16 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgcctcgataactctgtccataa

SEQ ID NO: 17 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-O-methyl uridine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-fluoro guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 18 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-fluoro uridine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-O-methyl guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 19 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-O-methyl uridine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methoxyethyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-fluoro guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 20 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-fluoro uridine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methoxyethyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-O-methyl guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 21 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methoxyethyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-fluoro guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgcctcgataactctgtccataa

SEQ ID NO: 22 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methoxyethyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgcctcgataactctgtccataa

SEQ ID NO: 23 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-beta-D-deoxyxylose guanosine 3'-phosphodiester
  modified_base 6: mod_base = OTHER; note = 2'-fluoro uridine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-O-methyl guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 24 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-fluoro uridine 3'-phosphodiester
  modified_base 7: mod_base = OTHER; note = 2'-beta-D-deoxyxylose adenosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-O-methyl guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 25 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-beta-D-deoxyxylose uridine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12..13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-O-methyl guanosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19..20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tggagtatgtgcctcgataacaa

SEQ ID NO: 26 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro cytidine 3'-phosphodiester
  modified_base 7: mod_base = OTHER; note = 2'-beta-D-deoxyxylose guanosine 3'-phosphodiester
  modified_base 8: mod_base = OTHER; note = 2'-O-methyl adenosine 3'-phosphodiester
  modified_base 9: mod_base = OTHER; note = 2'-O-methyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgcctcgataactctgtccataa

SEQ ID NO: 27 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-beta-D-deoxyxylose cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgcctcgataactctgtccataa

SEQ ID NO: 28 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3..4: mod_base = OTHER; note = 2'-O-methyl cytidine 3'-phosphodiester
  modified_base 5: mod_base = OTHER; note = 2'-beta-D-deoxyxylose uridine 3'-phosphodiester
  modified_base 6: mod_base = OTHER; note = 2'-fluoro cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-O-methyl guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgcctcgataactctgtccataa

SEQ ID NO: 29 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro uridine 3'-phosphorothioate
  modified_base 3: mod_base = OTHER; note = 2'-O-methyl guanosine 3'-phosphodiester
  modified_base 4..5: mod_base = OTHER; note = 2'-O-methyl cytidine 3'-phosphodiester
  modified_base 6: mod_base = OTHER; note = 2'-fluoro uridine 3'-phosphodiester
  modified_base 7: mod_base = OTHER; note = 2'-O-methyl cytidine 3'-phosphodiester
  modified_base 8: mod_base = OTHER; note = 2'-O-methyl guanosine 3'-phosphodiester
  modified_base 9: mod_base = OTHER; note = 2'-O-methyl adenosine 3'-phosphodiester
  modified_base 10: mod_base = OTHER; note = 2'-O-methyl uridine 3'-phosphodiester
  modified_base 11..12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-O-methyl uridine 3'-phosphodiester
  modified_base 17: mod_base = OTHER; note = 2'-O-methyl guanosine 3'-phosphodiester
  modified_base 18: mod_base = OTHER; note = 2'-O-methyl uridine 3'-phosphodiester
  modified_base 19..20: mod_base = OTHER; note = 2'-O-methyl cytidine 3'-phosphodiester
  modified_base 21: mod_base = OTHER; note = 2'-O-methyl adenosine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
ttgcctcgataactctgtccaaa

SEQ ID NO: 30 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro uridine 3'-phosphorothioate
  modified_base 3: mod_base = OTHER; note = 2'-O-methyl guanosine 3'-phosphodiester
  modified_base 4..5: mod_base = OTHER; note = 2'-O-methyl cytidine 3'-phosphodiester
  modified_base 6: mod_base = OTHER; note = 2'-O-methyl uridine 3'-phosphodiester
  modified_base 7: mod_base = OTHER; note = 2'-O-methyl cytidine 3'-phosphodiester
  modified_base 8: mod_base = OTHER; note = 2'-O-methyl guanosine 3'-phosphodiester
  modified_base 9: mod_base = OTHER; note = 2'-O-methyl adenosine 3'-phosphodiester
  modified_base 10: mod_base = OTHER; note = 2'-O-methyl uridine 3'-phosphodiester
  modified_base 11..12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-fluoro uridine 3'-phosphodiester
  modified_base 17: mod_base = OTHER; note = 2'-O-methyl guanosine 3'-phosphodiester
  modified_base 18: mod_base = OTHER; note = 2'-O-methyl uridine 3'-phosphodiester
  modified_base 19..20: mod_base = OTHER; note = 2'-O-methyl cytidine 3'-phosphodiester
  modified_base 21: mod_base = OTHER; note = 2'-O-methyl adenosine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
ttgcctcgataactctgtccaaa

SEQ ID NO: 31 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl uridine 3'-phosphodiester
  modified_base 4: mod_base = OTHER; note = 2'-O-methyl guanosine 3'-phosphodiester
  modified_base 5: mod_base = OTHER; note = 2'-O-methyl cytidine 3'-phosphodiester
  modified_base 6: mod_base = OTHER; note = 2'-fluoro cytidine 3'-phosphodiester
  modified_base 7: mod_base = OTHER; note = 2'-O-methyl uridine 3'-phosphodiester
  modified_base 8: mod_base = OTHER; note = 2'-O-methyl cytidine 3'-phosphodiester
  modified_base 9: mod_base = OTHER; note = 2'-O-methyl guanosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12..13: mod_base = OTHER; note = 2'-O-methyl adenosine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl uridine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphodiester
  modified_base 20: mod_base = OTHER; note = 2'-O-methyl cytidine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgtgcctcgataactctgtccaa

SEQ ID NO: 32 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl uridine 3'-phosphodiester
  modified_base 4: mod_base = OTHER; note = 2'-O-methyl guanosine 3'-phosphodiester
  modified_base 5..6: mod_base = OTHER; note = 2'-O-methyl cytidine 3'-phosphodiester
  modified_base 7: mod_base = OTHER; note = 2'-O-methyl uridine 3'-phosphodiester
  modified_base 8: mod_base = OTHER; note = 2'-O-methyl cytidine 3'-phosphodiester
  modified_base 9: mod_base = OTHER; note = 2'-O-methyl guanosine 3'-phosphodiester
  modified_base 10: mod_base = OTHER; note = 2'-O-methyl adenosine 3'-phosphodiester
  modified_base 11: mod_base = OTHER; note = 2'-O-methyl uridine 3'-phosphodiester
  modified_base 12..13: mod_base = OTHER; note = 2'-O-methyl adenosine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-fluoro cytidine 3'-phosphodiester
  modified_base 17: mod_base = OTHER; note = 2'-O-methyl uridine 3'-phosphodiester
  modified_base 18: mod_base = OTHER; note = 2'-O-methyl guanosine 3'-phosphodiester
  modified_base 19: mod_base = OTHER; note = 2'-O-methyl uridine 3'-phosphodiester
  modified_base 20: mod_base = OTHER; note = 2'-O-methyl cytidine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgtgcctcgataactctgtccaa

SEQ ID NO: 33 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro adenosine 3'-phosphorothioate
  modified_base 3: mod_base = OTHER; note = 2'-O-methyl uridine 3'-phosphodiester
  modified_base 4: mod_base = OTHER; note = 2'-O-methyl guanosine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7..8: mod_base = OTHER; note = 2'-O-methyl cytidine 3'-phosphodiester
  modified_base 9: mod_base = OTHER; note = 2'-O-methyl uridine 3'-phosphodiester
  modified_base 10: mod_base = OTHER; note = 2'-O-methyl cytidine 3'-phosphodiester
  modified_base 11: mod_base = OTHER; note = 2'-O-methyl guanosine 3'-phosphodiester
  modified_base 12: mod_base = OTHER; note = 2'-O-methyl adenosine 3'-phosphodiester
  modified_base 13: mod_base = OTHER; note = 2'-O-methyl uridine 3'-phosphodiester
  modified_base 14: mod_base = OTHER; note = 2'-fluoro adenosine 3'-phosphodiester
  modified_base 15: mod_base = OTHER; note = 2'-O-methyl adenosine 3'-phosphodiester
  modified_base 16: mod_base = OTHER; note = 2'-O-methyl cytidine 3'-phosphodiester
  modified_base 17: mod_base = OTHER; note = 2'-O-methyl uridine 3'-phosphodiester
  modified_base 18: mod_base = OTHER; note = 2'-O-methyl cytidine 3'-phosphodiester
  modified_base 19: mod_base = OTHER; note = 2'-O-methyl uridine 3'-phosphodiester
  modified_base 20: mod_base = OTHER; note = 2'-O-methyl guanosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tatgtgcctcgataactctgtaa

SEQ ID NO: 34 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro adenosine 3'-phosphorothioate
  modified_base 3: mod_base = OTHER; note = 2'-O-methyl uridine 3'-phosphodiester
  modified_base 4: mod_base = OTHER; note = 2'-O-methyl guanosine 3'-phosphodiester
  modified_base 5: mod_base = OTHER; note = 2'-O-methyl uridine 3'-phosphodiester
  modified_base 6: mod_base = OTHER; note = 2'-O-methyl guanosine 3'-phosphodiester
  modified_base 7..8: mod_base = OTHER; note = 2'-O-methyl cytidine 3'-phosphodiester
  modified_base 9: mod_base = OTHER; note = 2'-O-methyl uridine 3'-phosphodiester
  modified_base 10: mod_base = OTHER; note = 2'-O-methyl cytidine 3'-phosphodiester
  modified_base 11: mod_base = OTHER; note = 2'-O-methyl guanosine 3'-phosphodiester
  modified_base 12: mod_base = OTHER; note = 2'-O-methyl adenosine 3'-phosphodiester
  modified_base 13: mod_base = OTHER; note = 2'-O-methyl uridine 3'-phosphodiester
  modified_base 14: mod_base = OTHER; note = 2'-fluoro adenosine 3'-phosphodiester
  modified_base 15: mod_base = OTHER; note = 2'-O-methyl adenosine 3'-phosphodiester
  modified_base 16: mod_base = OTHER; note = 2'-fluoro cytidine 3'-phosphodiester
  modified_base 17: mod_base = OTHER; note = 2'-O-methyl uridine 3'-phosphodiester
  modified_base 18: mod_base = OTHER; note = 2'-O-methyl cytidine 3'-phosphodiester
  modified_base 19: mod_base = OTHER; note = 2'-O-methyl uridine 3'-phosphodiester
  modified_base 20: mod_base = OTHER; note = 2'-O-methyl guanosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tatgtgcctcgataactctgtaa

SEQ ID NO: 35 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl uridine 3'-phosphodiester
  modified_base 8: mod_base = OTHER; note = 2'-O-methyl guanosine 3'-phosphodiester
  modified_base 9..10: mod_base = OTHER; note = 2'-O-methyl cytidine 3'-phosphodiester
  modified_base 11: mod_base = OTHER; note = 2'-O-methyl uridine 3'-phosphodiester
  modified_base 12: mod_base = OTHER; note = 2'-O-methyl cytidine 3'-phosphodiester
  modified_base 13: mod_base = OTHER; note = 2'-O-methyl guanosine 3'-phosphodiester
  modified_base 14: mod_base = OTHER; note = 2'-fluoro adenosine 3'-phosphodiester
  modified_base 15: mod_base = OTHER; note = 2'-O-methyl uridine 3'-phosphodiester
  modified_base 16..17: mod_base = OTHER; note = 2'-O-methyl adenosine 3'-phosphodiester
  modified_base 18: mod_base = OTHER; note = 2'-O-methyl cytidine 3'-phosphodiester
  modified_base 19: mod_base = OTHER; note = 2'-O-methyl uridine 3'-phosphodiester
  modified_base 20: mod_base = OTHER; note = 2'-O-methyl cytidine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgtatgtgcctcgataactctaa

SEQ ID NO: 36 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl guanosine 3'-phosphodiester
  modified_base 7: mod_base = OTHER; note = 2'-O-methyl uridine 3'-phosphodiester
  modified_base 8: mod_base = OTHER; note = 2'-O-methyl guanosine 3'-phosphodiester
  modified_base 9..10: mod_base = OTHER; note = 2'-O-methyl cytidine 3'-phosphodiester
  modified_base 11: mod_base = OTHER; note = 2'-O-methyl uridine 3'-phosphodiester
  modified_base 12: mod_base = OTHER; note = 2'-O-methyl cytidine 3'-phosphodiester
  modified_base 13: mod_base = OTHER; note = 2'-O-methyl guanosine 3'-phosphodiester
  modified_base 14: mod_base = OTHER; note = 2'-fluoro adenosine 3'-phosphodiester
  modified_base 15: mod_base = OTHER; note = 2'-O-methyl uridine 3'-phosphodiester
  modified_base 16: mod_base = OTHER; note = 2'-fluoro adenosine 3'-phosphodiester
  modified_base 17: mod_base = OTHER; note = 2'-O-methyl adenosine 3'-phosphodiester
  modified_base 18: mod_base = OTHER; note = 2'-O-methyl cytidine 3'-phosphodiester
  modified_base 19: mod_base = OTHER; note = 2'-O-methyl uridine 3'-phosphodiester
  modified_base 20: mod_base = OTHER; note = 2'-O-methyl cytidine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgtatgtgcctcgataactctaa

SEQ ID NO: 37 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro adenosine 3'-phosphorothioate
  modified_base 3: mod_base = OTHER; note = 2'-O-methyl guanosine 3'-phosphodiester
  modified_base 4: mod_base = OTHER; note = 2'-O-methyl uridine 3'-phosphodiester
  modified_base 5: mod_base = OTHER; note = 2'-O-methyl adenosine 3'-phosphodiester
  modified_base 6: mod_base = OTHER; note = 2'-fluoro uridine 3'-phosphodiester
  modified_base 7: mod_base = OTHER; note = 2'-O-methyl guanosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10..11: mod_base = OTHER; note = 2'-O-methyl cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro guanosine 3'-phosphodiester
  modified_base 15: mod_base = OTHER; note = 2'-O-methyl adenosine 3'-phosphodiester
  modified_base 16: mod_base = OTHER; note = 2'-O-methyl uridine 3'-phosphodiester
  modified_base 17..18: mod_base = OTHER; note = 2'-O-methyl adenosine 3'-phosphodiester
  modified_base 19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tagtatgtgcctcgataactcaa

SEQ ID NO: 38 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro adenosine 3'-phosphorothioate
  modified_base 3: mod_base = OTHER; note = 2'-O-methyl guanosine 3'-phosphodiester
  modified_base 4: mod_base = OTHER; note = 2'-O-methyl uridine 3'-phosphodiester
  modified_base 5: mod_base = OTHER; note = 2'-O-methyl adenosine 3'-phosphodiester
  modified_base 6: mod_base = OTHER; note = 2'-O-methyl uridine 3'-phosphodiester
  modified_base 7: mod_base = OTHER; note = 2'-O-methyl guanosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10..11: mod_base = OTHER; note = 2'-O-methyl cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro guanosine 3'-phosphodiester
  modified_base 15: mod_base = OTHER; note = 2'-O-methyl adenosine 3'-phosphodiester
  modified_base 16: mod_base = OTHER; note = 2'-fluoro uridine 3'-phosphodiester
  modified_base 17..18: mod_base = OTHER; note = 2'-O-methyl adenosine 3'-phosphodiester
  modified_base 19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tagtatgtgcctcgataactcaa

SEQ ID NO: 39 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro uridine 3'-phosphorothioate
  modified_base 3..4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl uridine 3'-phosphodiester
  modified_base 7: mod_base = OTHER; note = 2'-O-methyl cytidine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl uridine 3'-phosphodiester
  modified_base 11..12: mod_base = OTHER; note = 2'-O-methyl cytidine 3'-phosphodiester
  modified_base 13: mod_base = OTHER; note = 2'-O-methyl adenosine 3'-phosphodiester
  modified_base 14: mod_base = OTHER; note = 2'-fluoro uridine 3'-phosphodiester
  modified_base 15: mod_base = OTHER; note = 2'-O-methyl uridine 3'-phosphodiester
  modified_base 16: mod_base = OTHER; note = 2'-fluoro adenosine 3'-phosphodiester
  modified_base 17..18: mod_base = OTHER; note = 2'-O-methyl cytidine 3'-phosphodiester
  modified_base 19: mod_base = OTHER; note = 2'-O-methyl adenosine 3'-phosphodiester
  modified_base 20: mod_base = OTHER; note = 2'-O-methyl uridine 3'-phosphodiester
  modified_base 21: mod_base = OTHER; note = 2'-O-methyl guanosine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
ttaactctgtccattaccatgaa

SEQ ID NO: 40 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro adenosine 3'-phosphorothioate
  modified_base 3: mod_base = OTHER; note = 2'-O-methyl uridine 3'-phosphodiester
  modified_base 4..5: mod_base = OTHER; note = 2'-O-methyl adenosine 3'-phosphodiester
  modified_base 6: mod_base = OTHER; note = 2'-O-methyl cytidine 3'-phosphodiester
  modified_base 7: mod_base = OTHER; note = 2'-O-methyl uridine 3'-phosphodiester
  modified_base 8: mod_base = OTHER; note = 2'-O-methyl cytidine 3'-phosphodiester
  modified_base 9: mod_base = OTHER; note = 2'-O-methyl uridine 3'-phosphodiester
  modified_base 10: mod_base = OTHER; note = 2'-O-methyl guanosine 3'-phosphodiester
  modified_base 11: mod_base = OTHER; note = 2'-O-methyl uridine 3'-phosphodiester
  modified_base 12..13: mod_base = OTHER; note = 2'-O-methyl cytidine 3'-phosphodiester
  modified_base 14: mod_base = OTHER; note = 2'-fluoro adenosine 3'-phosphodiester
  modified_base 15: mod_base = OTHER; note = 2'-O-methyl uridine 3'-phosphodiester
  modified_base 16: mod_base = OTHER; note = 2'-fluoro uridine 3'-phosphodiester
  modified_base 17: mod_base = OTHER; note = 2'-O-methyl adenosine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tataactctgtccattaccataa

SEQ ID NO: 41 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro uridine 3'-phosphorothioate
  modified_base 3: mod_base = OTHER; note = 2'-O-methyl guanosine 3'-phosphodiester
  modified_base 4: mod_base = OTHER; note = 2'-O-methyl uridine 3'-phosphodiester
  modified_base 5: mod_base = OTHER; note = 2'-O-methyl guanosine 3'-phosphodiester
  modified_base 6..7: mod_base = OTHER; note = 2'-O-methyl cytidine 3'-phosphodiester
  modified_base 8: mod_base = OTHER; note = 2'-O-methyl uridine 3'-phosphodiester
  modified_base 9: mod_base = OTHER; note = 2'-O-methyl cytidine 3'-phosphodiester
  modified_base 10: mod_base = OTHER; note = 2'-O-methyl guanosine 3'-phosphodiester
  modified_base 11: mod_base = OTHER; note = 2'-O-methyl adenosine 3'-phosphodiester
  modified_base 12: mod_base = OTHER; note = 2'-O-methyl uridine 3'-phosphodiester
  modified_base 13: mod_base = OTHER; note = 2'-O-methyl adenosine 3'-phosphodiester
  modified_base 14: mod_base = OTHER; note = 2'-fluoro adenosine 3'-phosphodiester
  modified_base 15: mod_base = OTHER; note = 2'-O-methyl cytidine 3'-phosphodiester
  modified_base 16: mod_base = OTHER; note = 2'-fluoro uridine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl guanosine 3'-phosphodiester
  modified_base 20: mod_base = OTHER; note = 2'-O-methyl uridine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
ttgtgcctcgataactctgtcaa

SEQ ID NO: 42 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro uridine 3'-phosphorothioate
  modified_base 3: mod_base = OTHER; note = 2'-O-methyl guanosine 3'-phosphodiester
  modified_base 4: mod_base = OTHER; note = 2'-O-methyl uridine 3'-phosphodiester
  modified_base 5: mod_base = OTHER; note = 2'-O-methyl guanosine 3'-phosphodiester
  modified_base 6: mod_base = OTHER; note = 2'-fluoro cytidine 3'-phosphodiester
  modified_base 7: mod_base = OTHER; note = 2'-O-methyl cytidine 3'-phosphodiester
  modified_base 8: mod_base = OTHER; note = 2'-O-methyl uridine 3'-phosphodiester
  modified_base 9: mod_base = OTHER; note = 2'-O-methyl cytidine 3'-phosphodiester
  modified_base 10: mod_base = OTHER; note = 2'-O-methyl guanosine 3'-phosphodiester
  modified_base 11: mod_base = OTHER; note = 2'-O-methyl adenosine 3'-phosphodiester
  modified_base 12: mod_base = OTHER; note = 2'-O-methyl uridine 3'-phosphodiester
  modified_base 13: mod_base = OTHER; note = 2'-O-methyl adenosine 3'-phosphodiester
  modified_base 14: mod_base = OTHER; note = 2'-fluoro adenosine 3'-phosphodiester
  modified_base 15: mod_base = OTHER; note = 2'-O-methyl cytidine 3'-phosphodiester
  modified_base 16: mod_base = OTHER; note = 2'-O-methyl uridine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl guanosine 3'-phosphodiester
  modified_base 20: mod_base = OTHER; note = 2'-O-methyl uridine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
ttgtgcctcgataactctgtcaa

SEQ ID NO: 43 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro cytidine 3'-phosphorothioate
  modified_base 3: mod_base = OTHER; note = 2'-O-methyl uridine 3'-phosphodiester
  modified_base 4: mod_base = OTHER; note = 2'-O-methyl cytidine 3'-phosphodiester
  modified_base 5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl uridine 3'-phosphodiester
  modified_base 8..9: mod_base = OTHER; note = 2'-O-methyl adenosine 3'-phosphodiester
  modified_base 10: mod_base = OTHER; note = 2'-O-methyl cytidine 3'-phosphodiester
  modified_base 11: mod_base = OTHER; note = 2'-O-methyl uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro guanosine 3'-phosphodiester
  modified_base 15: mod_base = OTHER; note = 2'-O-methyl uridine 3'-phosphodiester
  modified_base 16: mod_base = OTHER; note = 2'-fluoro cytidine 3'-phosphodiester
  modified_base 17: mod_base = OTHER; note = 2'-O-methyl cytidine 3'-phosphodiester
  modified_base 18: mod_base = OTHER; note = 2'-O-methyl adenosine 3'-phosphodiester
  modified_base 19..20: mod_base = OTHER; note = 2'-O-methyl uridine 3'-phosphodiester
  modified_base 21: mod_base = OTHER; note = 2'-O-methyl adenosine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tctcgataactctgtccattaaa

SEQ ID NO: 44 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro cytidine 3'-phosphorothioate
  modified_base 3: mod_base = OTHER; note = 2'-O-methyl uridine 3'-phosphodiester
  modified_base 4: mod_base = OTHER; note = 2'-O-methyl cytidine 3'-phosphodiester
  modified_base 5: mod_base = OTHER; note = 2'-O-methyl guanosine 3'-phosphodiester
  modified_base 6: mod_base = OTHER; note = 2'-fluoro adenosine 3'-phosphodiester
  modified_base 7: mod_base = OTHER; note = 2'-O-methyl uridine 3'-phosphodiester
  modified_base 8..9: mod_base = OTHER; note = 2'-O-methyl adenosine 3'-phosphodiester
  modified_base 10: mod_base = OTHER; note = 2'-O-methyl cytidine 3'-phosphodiester
  modified_base 11: mod_base = OTHER; note = 2'-O-methyl uridine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro guanosine 3'-phosphodiester
  modified_base 15: mod_base = OTHER; note = 2'-O-methyl uridine 3'-phosphodiester
  modified_base 16..17: mod_base = OTHER; note = 2'-O-methyl cytidine 3'-phosphodiester
  modified_base 18: mod_base = OTHER; note = 2'-O-methyl adenosine 3'-phosphodiester
  modified_base 19..20: mod_base = OTHER; note = 2'-O-methyl uridine 3'-phosphodiester
  modified_base 21: mod_base = OTHER; note = 2'-O-methyl adenosine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tctcgataactctgtccattaaa

SEQ ID NO: 45 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro uridine 3'-phosphorothioate
  modified_base 3: mod_base = OTHER; note = 2'-O-methyl cytidine 3'-phosphodiester
  modified_base 4: mod_base = OTHER; note = 2'-O-methyl guanosine 3'-phosphodiester
  modified_base 5: mod_base = OTHER; note = 2'-O-methyl adenosine 3'-phosphodiester
  modified_base 6: mod_base = OTHER; note = 2'-O-methyl uridine 3'-phosphodiester
  modified_base 7..8: mod_base = OTHER; note = 2'-O-methyl adenosine 3'-phosphodiester
  modified_base 9: mod_base = OTHER; note = 2'-O-methyl cytidine 3'-phosphodiester
  modified_base 10: mod_base = OTHER; note = 2'-O-methyl uridine 3'-phosphodiester
  modified_base 11: mod_base = OTHER; note = 2'-O-methyl cytidine 3'-phosphodiester
  modified_base 12: mod_base = OTHER; note = 2'-O-methyl uridine 3'-phosphodiester
  modified_base 13: mod_base = OTHER; note = 2'-O-methyl guanosine 3'-phosphodiester
  modified_base 14: mod_base = OTHER; note = 2'-fluoro uridine 3'-phosphodiester
  modified_base 15: mod_base = OTHER; note = 2'-O-methyl cytidine 3'-phosphodiester
  modified_base 16: mod_base = OTHER; note = 2'-fluoro cytidine 3'-phosphodiester
  modified_base 17: mod_base = OTHER; note = 2'-O-methyl adenosine 3'-phosphodiester
  modified_base 18..19: mod_base = OTHER; note = 2'-O-methyl uridine 3'-phosphodiester
  modified_base 20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
ttcgataactctgtccattacaa

SEQ ID NO: 46 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro uridine 3'-phosphorothioate
  modified_base 3: mod_base = OTHER; note = 2'-O-methyl cytidine 3'-phosphodiester
  modified_base 4: mod_base = OTHER; note = 2'-O-methyl guanosine 3'-phosphodiester
  modified_base 5: mod_base = OTHER; note = 2'-O-methyl adenosine 3'-phosphodiester
  modified_base 6: mod_base = OTHER; note = 2'-fluoro uridine 3'-phosphodiester
  modified_base 7..8: mod_base = OTHER; note = 2'-O-methyl adenosine 3'-phosphodiester
  modified_base 9: mod_base = OTHER; note = 2'-O-methyl cytidine 3'-phosphodiester
  modified_base 10: mod_base = OTHER; note = 2'-O-methyl uridine 3'-phosphodiester
  modified_base 11: mod_base = OTHER; note = 2'-O-methyl cytidine 3'-phosphodiester
  modified_base 12: mod_base = OTHER; note = 2'-O-methyl uridine 3'-phosphodiester
  modified_base 13: mod_base = OTHER; note = 2'-O-methyl guanosine 3'-phosphodiester
  modified_base 14: mod_base = OTHER; note = 2'-fluoro uridine 3'-phosphodiester
  modified_base 15..16: mod_base = OTHER; note = 2'-O-methyl cytidine 3'-phosphodiester
  modified_base 17: mod_base = OTHER; note = 2'-O-methyl adenosine 3'-phosphodiester
  modified_base 18..19: mod_base = OTHER; note = 2'-O-methyl uridine 3'-phosphodiester
  modified_base 20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
ttcgataactctgtccattacaa

SEQ ID NO: 47 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl adenosine 3'-phosphodiester
  modified_base 4: mod_base = OTHER; note = 2'-O-methyl uridine 3'-phosphodiester
  modified_base 5..6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl uridine 3'-phosphodiester
  modified_base 9: mod_base = OTHER; note = 2'-O-methyl cytidine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro cytidine 3'-phosphodiester
  modified_base 15: mod_base = OTHER; note = 2'-O-methyl adenosine 3'-phosphodiester
  modified_base 16: mod_base = OTHER; note = 2'-fluoro uridine 3'-phosphodiester
  modified_base 17: mod_base = OTHER; note = 2'-O-methyl uridine 3'-phosphodiester
  modified_base 18: mod_base = OTHER; note = 2'-O-methyl adenosine 3'-phosphodiester
  modified_base 19..20: mod_base = OTHER; note = 2'-O-methyl cytidine 3'-phosphodiester
  modified_base 21: mod_base = OTHER; note = 2'-O-methyl adenosine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgataactctgtccattaccaaa

SEQ ID NO: 48 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-deoxycytidine 3'-phosphorothioate
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methoxyethyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-deoxycytidine 3'-phosphorothioate
  modified_base 15: mod_base = OTHER; note = 2'-O-methyl uridine 3'-phosphodiester
  modified_base 16: mod_base = OTHER; note = 2'-deoxyguanosine 3'-phosphorothioate
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgcctcgataactctgtccataa

SEQ ID NO: 49 - RNA, 21 nt, synthetic construct
  source 1..21: mol_type = other RNA
  modified_base 1: mod_base = OTHER; note = 2'-O-methyl adenosine 3'-phosphorothioate
  modified_base 2: mod_base = OTHER; note = 2'-O-methyl uridine 3'-phosphorothioate
  modified_base 3..4: mod_base = OTHER; note = 2'-O-methyl guanosine 3'-phosphodiester
  modified_base 5: mod_base = OTHER; note = 2'-O-methyl adenosine 3'-phosphodiester
  modified_base 6: mod_base = OTHER; note = 2'-O-methyl cytidine 3'-phosphodiester
  modified_base 7: mod_base = OTHER; note = 2'-fluoro adenosine 3'-phosphodiester
  modified_base 8: mod_base = OTHER; note = 2'-O-methyl guanosine 3'-phosphodiester
  modified_base 9: mod_base = OTHER; note = 2'-fluoro adenosine 3'-phosphodiester
  modified_base 10: mod_base = OTHER; note = 2'-fluoro guanosine 3'-phosphodiester
  modified_base 11: mod_base = OTHER; note = 2'-fluoro uridine 3'-phosphodiester
  modified_base 12: mod_base = OTHER; note = 2'-O-methyl uridine 3'-phosphodiester
  modified_base 13: mod_base = OTHER; note = 2'-O-methyl adenosine 3'-phosphodiester
  modified_base 14: mod_base = OTHER; note = 2'-O-methyl uridine 3'-phosphodiester
  modified_base 15: mod_base = OTHER; note = 2'-O-methyl cytidine 3'-phosphodiester
  modified_base 16: mod_base = OTHER; note = 2'-O-methyl guanosine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl guanosine 3'-phosphorothioate
  modified_base 20: mod_base = OTHER; note = 2'-O-methyl cytidine 3'-phosphorothioate
  modified_base 21: mod_base = OTHER; note = 2'-O-methyl adenosine, conjugated at the 3' end to trishexylamino-N-Acetylgalactosamine-6-hydroxypropyl phosphodiester conjugate
atggacagagttatcgaggca

SEQ ID NO: 50 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro guanosine 3'-phosphorothioate
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
gttatcgaggcacatactcca

SEQ ID NO: 51 - RNA, 21 nt, synthetic construct
  source 1..21: mol_type = other RNA
  modified_base 1: mod_base = OTHER; note = 2'-O-methoxyethyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl adenosine 3'-phosphorothioate
  modified_base 3: mod_base = OTHER; note = 2'-O-methyl uridine 3'-phosphodiester
  modified_base 4..5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro adenosine 3'-phosphorothioate
  modified_base 11: mod_base = OTHER; note = 2'-fluoro guanosine 3'-phosphodiester
  modified_base 12..13: mod_base = OTHER; note = 2'-O-methyl uridine 3'-phosphodiester
  modified_base 14: mod_base = OTHER; note = 2'-O-methyl adenosine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl guanosine 3'-phosphodiester
  modified_base 18: mod_base = OTHER; note = 2'-O-methyl adenosine 3'-phosphodiester
  modified_base 19: mod_base = OTHER; note = 2'-O-methyl guanosine 3'-phosphorothioate
  modified_base 20: mod_base = OTHER; note = 2'-O-methoxyethyl guanosine 3'-phosphorothioate
  modified_base 21: mod_base = OTHER; note = 2'-O-methoxyethyl adenosine
aatggacagagttatcgagga

SEQ ID NO: 52 - RNA, 21 nt, synthetic construct
  source 1..21: mol_type = other RNA
  modified_base 1: mod_base = OTHER; note = 2'-O-methyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methyl uridine 3'-phosphorothioate
  modified_base 3..4: mod_base = OTHER; note = 2'-O-methyl guanosine 3'-phosphodiester
  modified_base 5: mod_base = OTHER; note = 2'-O-methyl adenosine 3'-phosphodiester
  modified_base 6: mod_base = OTHER; note = 2'-O-methyl cytidine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-fluoro adenosine 3'-phosphodiester
  modified_base 10: mod_base = OTHER; note = 2'-fluoro guanosine 3'-phosphodiester
  modified_base 11: mod_base = OTHER; note = 2'-fluoro uridine 3'-phosphodiester
  modified_base 12: mod_base = OTHER; note = 2'-O-methyl uridine 3'-phosphodiester
  modified_base 13: mod_base = OTHER; note = 2'-O-methyl adenosine 3'-phosphodiester
  modified_base 14: mod_base = OTHER; note = 2'-O-methoxyethyl uridine 3'-phosphodiester
  modified_base 15: mod_base = OTHER; note = 2'-O-methyl cytidine 3'-phosphodiester
  modified_base 16: mod_base = OTHER; note = 2'-O-methyl guanosine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl guanosine 3'-phosphorothioate
  modified_base 20: mod_base = OTHER; note = 2'-O-methyl cytidine 3'-phosphorothioate
  modified_base 21: mod_base = OTHER; note = 2'-O-methyl adenosine
atggacagagttatcgaggca

SEQ ID NO: 53 - RNA, 21 nt, synthetic construct
  source 1..21: mol_type = other RNA
  modified_base 1: mod_base = OTHER; note = 2'-O-methoxyethyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3..4: mod_base = OTHER; note = 2'-O-methyl guanosine 3'-phosphodiester
  modified_base 5: mod_base = OTHER; note = 2'-O-methyl adenosine 3'-phosphodiester
  modified_base 6: mod_base = OTHER; note = 2'-O-methyl cytidine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-fluoro adenosine 3'-phosphodiester
  modified_base 10: mod_base = OTHER; note = 2'-fluoro guanosine 3'-phosphodiester
  modified_base 11: mod_base = OTHER; note = 2'-fluoro uridine 3'-phosphodiester
  modified_base 12: mod_base = OTHER; note = 2'-O-methyl uridine 3'-phosphodiester
  modified_base 13: mod_base = OTHER; note = 2'-O-methyl adenosine 3'-phosphodiester
  modified_base 14: mod_base = OTHER; note = 2'-O-methoxyethyl uridine 3'-phosphodiester
  modified_base 15: mod_base = OTHER; note = 2'-O-methyl cytidine 3'-phosphodiester
  modified_base 16: mod_base = OTHER; note = 2'-O-methyl guanosine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl guanosine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
atggacagagttatcgaggca

SEQ ID NO: 54 - RNA, 21 nt, synthetic construct
  source 1..21: mol_type = other RNA
  modified_base 1: mod_base = OTHER; note = 2'-O-methyl guanosine 3'-phosphorothioate
  modified_base 2: mod_base = OTHER; note = 2'-O-methyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9..10: mod_base = OTHER; note = 2'-fluoro guanosine 3'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19..20: mod_base = OTHER; note = 2'-O-methyl cytidine 3'-phosphorothioate
  modified_base 21: mod_base = OTHER; note = 2'-O-methyl adenosine, conjugated at the 3' end to trishexylamino-N-Acetylgalactosamine-6-hydroxypropyl phosphodiester conjugate
gttatcgaggcacatactcca

SEQ ID NO: 55 - RNA, 21 nt, synthetic construct
  source 1..21: mol_type = other RNA
  modified_base 1: mod_base = OTHER; note = 2'-O-methoxyethyl guanosine 3'-phosphorothioate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9..10: mod_base = OTHER; note = 2'-fluoro guanosine 3'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine, conjugated at the 3' end to trishexylamino-N-Acetylgalactosamine-6-hydroxypropyl phosphodiester conjugate
gttatcgaggcacatactcca

SEQ ID NO: 56 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9..10: mod_base = OTHER; note = 2'-fluoro guanosine 3'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
gttatcgaggcacatactcca

SEQ ID NO: 57 - RNA, 21 nt, synthetic construct
  source 1..21: mol_type = other RNA
  modified_base 1: mod_base = OTHER; note = 2'-O-methoxyethyl guanosine 3'-phosphorothioate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-deoxyguanosine 3'-phosphorothioate
  modified_base 10: mod_base = OTHER; note = 2'-fluoro guanosine 3'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine, conjugated at the 3' end to trishexylamino-N-Acetylgalactosamine-6-hydroxypropyl phosphodiester conjugate
gttatcgaggcacatactcca

SEQ ID NO: 58 - RNA, 21 nt, synthetic construct
  source 1..21: mol_type = other RNA
  modified_base 1: mod_base = OTHER; note = 2'-O-methoxyethyl guanosine 3'-phosphorothioate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-deoxyguanosine 3'-phosphorothioate
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine, conjugated at the 3' end to trishexylamino-N-Acetylgalactosamine-6-hydroxypropyl phosphodiester conjugate
gttatcgaggcacatactcca

SEQ ID NO: 59 - RNA, 21 nt, synthetic construct
  source 1..21: mol_type = other RNA
  modified_base 1: mod_base = OTHER; note = 2'-O-methoxyethyl guanosine 3'-phosphorothioate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9..10: mod_base = OTHER; note = 2'-fluoro guanosine 3'-phosphodiester
  modified_base 11: mod_base = OTHER; note = 2'-deoxycytidine 3'-phosphorothioate
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine, conjugated at the 3' end to trishexylamino-N-Acetylgalactosamine-6-hydroxypropyl phosphodiester conjugate
gttatcgaggcacatactcca

SEQ ID NO: 60 - RNA, 21 nt, synthetic construct
  source 1..21: mol_type = other RNA
  modified_base 1: mod_base = OTHER; note = 2'-O-methoxyethyl guanosine 3'-phosphorothioate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 3'-fluoro hexitol nucleic acid guanosine 4'-phosphodiester
  modified_base 10: mod_base = OTHER; note = 2'-fluoro guanosine 3'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine, conjugated at the 3' end to trishexylamino-N-Acetylgalactosamine-6-hydroxypropyl phosphodiester conjugate
gttatcgaggcacatactcca

SEQ ID NO: 61 - RNA, 21 nt, synthetic construct
  source 1..21: mol_type = other RNA
  modified_base 1: mod_base = OTHER; note = 2'-O-methoxyethyl guanosine 3'-phosphorothioate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 3'-fluoro hexitol nucleic acid guanosine 4'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine, conjugated at the 3' end to trishexylamino-N-Acetylgalactosamine-6-hydroxypropyl phosphodiester conjugate
gttatcgaggcacatactcca

SEQ ID NO: 62 - RNA, 21 nt, synthetic construct
  source 1..21: mol_type = other RNA
  modified_base 1: mod_base = OTHER; note = 2'-O-methoxyethyl guanosine 3'-phosphorothioate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9..10: mod_base = OTHER; note = 2'-fluoro guanosine 3'-phosphodiester
  modified_base 11: mod_base = OTHER; note = 3'-fluoro hexitol nucleic acid cytidine 4'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine, conjugated at the 3' end to trishexylamino-N-Acetylgalactosamine-6-hydroxypropyl phosphodiester conjugate
gttatcgaggcacatactcca

SEQ ID NO: 63 - RNA, 21 nt, synthetic construct
  source 1..21: mol_type = other RNA
  modified_base 1: mod_base = OTHER; note = 2'-O-methoxyethyl guanosine 3'-phosphorothioate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-beta-D-deoxyxylose guanosine 3'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine, conjugated at the 3' end to trishexylamino-N-Acetylgalactosamine-6-hydroxypropyl phosphodiester conjugate
gttatcgaggcacatactcca

SEQ ID NO: 64 - RNA, 21 nt, synthetic construct
  source 1..21: mol_type = other RNA
  modified_base 1: mod_base = OTHER; note = 2'-O-methoxyethyl guanosine 3'-phosphorothioate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9..10: mod_base = OTHER; note = 2'-fluoro guanosine 3'-phosphodiester
  modified_base 11: mod_base = OTHER; note = 2'-beta-D-deoxyxylose cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine, conjugated at the 3' end to trishexylamino-N-Acetylgalactosamine-6-hydroxypropyl phosphodiester conjugate
gttatcgaggcacatactcca

SEQ ID NO: 65 - RNA, 21 nt, synthetic construct
  source 1..21: mol_type = other RNA
  modified_base 1: mod_base = OTHER; note = 2'-O-methoxyethyl guanosine 3'-phosphorothioate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-beta-D-deoxyxylose guanosine 3'-phosphodiester
  modified_base 10: mod_base = OTHER; note = 2'-fluoro guanosine 3'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine, conjugated at the 3' end to trishexylamino-N-Acetylgalactosamine-6-hydroxypropyl phosphodiester conjugate
gttatcgaggcacatactcca

SEQ ID NO: 66 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-deoxyguanosine 3'-phosphorothioate
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
gttatcgaggcacatactcca

SEQ ID NO: 67 - RNA, 21 nt, synthetic construct
  source 1..21: mol_type = other RNA
  modified_base 1: mod_base = OTHER; note = 2'-O-methoxyethyl uridine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-O-methyl adenosine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10..11: mod_base = OTHER; note = 2'-fluoro uridine 3'-phosphodiester
  modified_base 12: mod_base = OTHER; note = 2'-O-methyl adenosine 3'-phosphodiester
  modified_base 13: mod_base = OTHER; note = 2'-O-methyl uridine 3'-phosphodiester
  modified_base 14: mod_base = OTHER; note = 2'-O-methoxyethyl cytidine 3'-phosphodiester
  modified_base 15: mod_base = OTHER; note = 2'-O-methyl guanosine 3'-phosphodiester
  modified_base 16: mod_base = OTHER; note = 2'-O-methyl adenosine 3'-phosphodiester
  modified_base 17..18: mod_base = OTHER; note = 2'-O-methyl guanosine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
tggacagagttatcgaggcaa

SEQ ID NO: 68 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl guanosine 3'-phosphorothioate
  modified_base 3: mod_base = OTHER; note = 2'-O-methyl adenosine 3'-phosphodiester
  modified_base 4: mod_base = OTHER; note = 2'-O-methyl cytidine 3'-phosphodiester
  modified_base 5: mod_base = OTHER; note = 2'-O-methyl adenosine 3'-phosphodiester
  modified_base 6: mod_base = OTHER; note = 2'-O-methyl guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9..10: mod_base = OTHER; note = 2'-fluoro uridine 3'-phosphodiester
  modified_base 11: mod_base = OTHER; note = 2'-fluoro adenosine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-O-methoxyethyl guanosine 3'-phosphodiester
  modified_base 15: mod_base = OTHER; note = 2'-O-methyl adenosine 3'-phosphodiester
  modified_base 16..17: mod_base = OTHER; note = 2'-O-methyl guanosine 3'-phosphodiester
  modified_base 18: mod_base = OTHER; note = 2'-O-methyl cytidine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
ggacagagttatcgaggcaca

SEQ ID NO: 69 - RNA, 21 nt, synthetic construct
  source 1..21: mol_type = other RNA
  modified_base 1: mod_base = OTHER; note = 2'-O-methoxyethyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl cytidine 3'-phosphorothioate
  modified_base 3: mod_base = OTHER; note = 2'-O-methyl adenosine 3'-phosphodiester
  modified_base 4: mod_base = OTHER; note = 2'-O-methyl guanosine 3'-phosphodiester
  modified_base 5: mod_base = OTHER; note = 2'-O-methyl adenosine 3'-phosphodiester
  modified_base 6: mod_base = OTHER; note = 2'-O-methyl guanosine 3'-phosphodiester
  modified_base 7..8: mod_base = OTHER; note = 2'-O-methyl uridine 3'-phosphodiester
  modified_base 9: mod_base = OTHER; note = 2'-fluoro adenosine 3'-phosphodiester
  modified_base 10: mod_base = OTHER; note = 2'-fluoro uridine 3'-phosphodiester
  modified_base 11: mod_base = OTHER; note = 2'-fluoro cytidine 3'-phosphodiester
  modified_base 12: mod_base = OTHER; note = 2'-O-methyl guanosine 3'-phosphodiester
  modified_base 13: mod_base = OTHER; note = 2'-O-methyl adenosine 3'-phosphodiester
  modified_base 14: mod_base = OTHER; note = 2'-O-methoxyethyl guanosine 3'-phosphodiester
  modified_base 15: mod_base = OTHER; note = 2'-O-methyl guanosine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl cytidine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl uridine 3'-phosphorothioate
  modified_base 21: mod_base = OTHER; note = 2'-O-methoxyethyl adenosine
acagagttatcgaggcacata

SEQ ID NO: 70 - RNA, 21 nt, synthetic construct
  source 1..21: mol_type = other RNA
  modified_base 1: mod_base = OTHER; note = 2'-O-methoxyethyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl guanosine 3'-phosphorothioate
  modified_base 3: mod_base = OTHER; note = 2'-O-methyl adenosine 3'-phosphodiester
  modified_base 4: mod_base = OTHER; note = 2'-O-methyl guanosine 3'-phosphodiester
  modified_base 5..6: mod_base = OTHER; note = 2'-O-methyl uridine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-fluoro cytidine 3'-phosphodiester
  modified_base 10: mod_base = OTHER; note = 2'-fluoro guanosine 3'-phosphodiester
  modified_base 11: mod_base = OTHER; note = 2'-fluoro adenosine 3'-phosphodiester
  modified_base 12..13: mod_base = OTHER; note = 2'-O-methyl guanosine 3'-phosphodiester
  modified_base 14: mod_base = OTHER; note = 2'-O-methoxyethyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
agagttatcgaggcacataca

SEQ ID NO: 71 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl adenosine 3'-phosphorothioate
  modified_base 3: mod_base = OTHER; note = 2'-O-methyl guanosine 3'-phosphodiester
  modified_base 4..5: mod_base = OTHER; note = 2'-O-methyl uridine 3'-phosphodiester
  modified_base 6: mod_base = OTHER; note = 2'-O-methyl adenosine 3'-phosphodiester
  modified_base 7: mod_base = OTHER; note = 2'-O-methyl uridine 3'-phosphodiester
  modified_base 8: mod_base = OTHER; note = 2'-O-methyl cytidine 3'-phosphodiester
  modified_base 9: mod_base = OTHER; note = 2'-fluoro guanosine 3'-phosphodiester
  modified_base 10: mod_base = OTHER; note = 2'-fluoro adenosine 3'-phosphodiester
  modified_base 11: mod_base = OTHER; note = 2'-fluoro guanosine 3'-phosphodiester
  modified_base 12: mod_base = OTHER; note = 2'-O-methyl guanosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl cytidine 3'-phosphodiester
  modified_base 16: mod_base = OTHER; note = 2'-O-methyl adenosine 3'-phosphodiester
  modified_base 17: mod_base = OTHER; note = 2'-O-methyl uridine 3'-phosphodiester
  modified_base 18: mod_base = OTHER; note = 2'-O-methyl adenosine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl uridine 3'-phosphorothioate
  modified_base 21: mod_base = OTHER; note = 2'-O-methoxyethyl adenosine
gagttatcgaggcacatacta

SEQ ID NO: 72 - RNA, 21 nt, synthetic construct
  source 1..21: mol_type = other RNA
  modified_base 1: mod_base = OTHER; note = 2'-O-methoxyethyl cytidine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl adenosine 3'-phosphorothioate
  modified_base 3: mod_base = OTHER; note = 2'-O-methyl uridine 3'-phosphodiester
  modified_base 4..5: mod_base = OTHER; note = 2'-O-methyl guanosine 3'-phosphodiester
  modified_base 6: mod_base = OTHER; note = 2'-O-methyl uridine 3'-phosphodiester
  modified_base 7..8: mod_base = OTHER; note = 2'-O-methyl adenosine 3'-phosphodiester
  modified_base 9: mod_base = OTHER; note = 2'-fluoro uridine 3'-phosphodiester
  modified_base 10..11: mod_base = OTHER; note = 2'-fluoro guanosine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl guanosine 3'-phosphodiester
  modified_base 16: mod_base = OTHER; note = 2'-O-methyl adenosine 3'-phosphodiester
  modified_base 17: mod_base = OTHER; note = 2'-O-methyl guanosine 3'-phosphodiester
  modified_base 18: mod_base = OTHER; note = 2'-O-methyl uridine 3'-phosphodiester
  modified_base 19: mod_base = OTHER; note = 2'-O-methyl uridine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
catggtaatggacagagttaa

SEQ ID NO: 73 - RNA, 21 nt, synthetic construct
  source 1..21: mol_type = other RNA
  modified_base 1: mod_base = OTHER; note = 2'-O-methoxyethyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3..4: mod_base = OTHER; note = 2'-O-methyl guanosine 3'-phosphodiester
  modified_base 5: mod_base = OTHER; note = 2'-O-methyl uridine 3'-phosphodiester
  modified_base 6..7: mod_base = OTHER; note = 2'-O-methyl adenosine 3'-phosphodiester
  modified_base 8: mod_base = OTHER; note = 2'-O-methyl uridine 3'-phosphodiester
  modified_base 9..10: mod_base = OTHER; note = 2'-fluoro guanosine 3'-phosphodiester
  modified_base 11: mod_base = OTHER; note = 2'-fluoro adenosine 3'-phosphodiester
  modified_base 12: mod_base = OTHER; note = 2'-O-methyl cytidine 3'-phosphodiester
  modified_base 13: mod_base = OTHER; note = 2'-O-methyl adenosine 3'-phosphodiester
  modified_base 14: mod_base = OTHER; note = 2'-O-methoxyethyl guanosine 3'-phosphodiester
  modified_base 15: mod_base = OTHER; note = 2'-O-methyl adenosine 3'-phosphodiester
  modified_base 16: mod_base = OTHER; note = 2'-O-methyl guanosine 3'-phosphodiester
  modified_base 17..18: mod_base = OTHER; note = 2'-O-methyl uridine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl uridine 3'-phosphorothioate
  modified_base 21: mod_base = OTHER; note = 2'-O-methoxyethyl adenosine
atggtaatggacagagttata

SEQ ID NO: 74 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl adenosine 3'-phosphorothioate
  modified_base 3: mod_base = OTHER; note = 2'-O-methyl cytidine 3'-phosphodiester
  modified_base 4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-O-methyl adenosine 3'-phosphodiester
  modified_base 7: mod_base = OTHER; note = 2'-O-methyl guanosine 3'-phosphodiester
  modified_base 8: mod_base = OTHER; note = 2'-O-methyl uridine 3'-phosphodiester
  modified_base 9: mod_base = OTHER; note = 2'-fluoro uridine 3'-phosphodiester
  modified_base 10: mod_base = OTHER; note = 2'-fluoro adenosine 3'-phosphodiester
  modified_base 11: mod_base = OTHER; note = 2'-fluoro uridine 3'-phosphodiester
  modified_base 12: mod_base = OTHER; note = 2'-O-methyl cytidine 3'-phosphodiester
  modified_base 13: mod_base = OTHER; note = 2'-O-methyl guanosine 3'-phosphodiester
  modified_base 14: mod_base = OTHER; note = 2'-O-methoxyethyl adenosine 3'-phosphodiester
  modified_base 15..16: mod_base = OTHER; note = 2'-O-methyl guanosine 3'-phosphodiester
  modified_base 17: mod_base = OTHER; note = 2'-O-methyl cytidine 3'-phosphodiester
  modified_base 18: mod_base = OTHER; note = 2'-O-methyl adenosine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
gacagagttatcgaggcacaa

SEQ ID NO: 75 - RNA, 21 nt, synthetic construct
  source 1..21: mol_type = other RNA
  modified_base 1: mod_base = OTHER; note = 2'-O-methoxyethyl uridine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl adenosine 3'-phosphorothioate
  modified_base 3: mod_base = OTHER; note = 2'-O-methyl adenosine 3'-phosphodiester
  modified_base 4: mod_base = OTHER; note = 2'-O-methyl uridine 3'-phosphodiester
  modified_base 5..6: mod_base = OTHER; note = 2'-O-methyl guanosine 3'-phosphodiester
  modified_base 7: mod_base = OTHER; note = 2'-O-methyl adenosine 3'-phosphodiester
  modified_base 8: mod_base = OTHER; note = 2'-O-methyl cytidine 3'-phosphodiester
  modified_base 9: mod_base = OTHER; note = 2'-fluoro adenosine 3'-phosphodiester
  modified_base 10: mod_base = OTHER; note = 2'-fluoro guanosine 3'-phosphodiester
  modified_base 11: mod_base = OTHER; note = 2'-fluoro adenosine 3'-phosphodiester
  modified_base 12: mod_base = OTHER; note = 2'-O-methyl guanosine 3'-phosphodiester
  modified_base 13: mod_base = OTHER; note = 2'-O-methyl uridine 3'-phosphodiester
  modified_base 14: mod_base = OTHER; note = 2'-O-methoxyethyl uridine 3'-phosphodiester
  modified_base 15: mod_base = OTHER; note = 2'-O-methyl adenosine 3'-phosphodiester
  modified_base 16: mod_base = OTHER; note = 2'-O-methyl uridine 3'-phosphodiester
  modified_base 17: mod_base = OTHER; note = 2'-O-methyl cytidine 3'-phosphodiester
  modified_base 18: mod_base = OTHER; note = 2'-O-methyl guanosine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl guanosine 3'-phosphorothioate
  modified_base 21: mod_base = OTHER; note = 2'-O-methoxyethyl adenosine
taatggacagagttatcgaga

SEQ ID NO: 76 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3..4: mod_base = OTHER; note = 2'-O-methyl adenosine 3'-phosphodiester
  modified_base 5: mod_base = OTHER; note = 2'-O-methyl uridine 3'-phosphodiester
  modified_base 6..7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-fluoro cytidine 3'-phosphodiester
  modified_base 10: mod_base = OTHER; note = 2'-fluoro adenosine 3'-phosphodiester
  modified_base 11: mod_base = OTHER; note = 2'-fluoro guanosine 3'-phosphodiester
  modified_base 12: mod_base = OTHER; note = 2'-O-methyl adenosine 3'-phosphodiester
  modified_base 13: mod_base = OTHER; note = 2'-O-methyl guanosine 3'-phosphodiester
  modified_base 14: mod_base = OTHER; note = 2'-O-methoxyethyl uridine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl uridine 3'-phosphodiester
  modified_base 18: mod_base = OTHER; note = 2'-O-methyl cytidine 3'-phosphodiester
  modified_base 19: mod_base = OTHER; note = 2'-O-methyl guanosine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
gtaatggacagagttatcgaa

SEQ ID NO: 77 - RNA, 21 nt, synthetic construct
  source 1..21: mol_type = other RNA
  modified_base 1: mod_base = OTHER; note = 2'-O-methoxyethyl uridine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl guanosine 3'-phosphorothioate
  modified_base 3: mod_base = OTHER; note = 2'-O-methyl guanosine 3'-phosphodiester
  modified_base 4: mod_base = OTHER; note = 2'-O-methyl uridine 3'-phosphodiester
  modified_base 5..6: mod_base = OTHER; note = 2'-O-methyl adenosine 3'-phosphodiester
  modified_base 7: mod_base = OTHER; note = 2'-O-methyl uridine 3'-phosphodiester
  modified_base 8: mod_base = OTHER; note = 2'-O-methyl guanosine 3'-phosphodiester
  modified_base 9: mod_base = OTHER; note = 2'-fluoro guanosine 3'-phosphodiester
  modified_base 10: mod_base = OTHER; note = 2'-fluoro adenosine 3'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl guanosine 3'-phosphodiester
  modified_base 14: mod_base = OTHER; note = 2'-O-methoxyethyl adenosine 3'-phosphodiester
  modified_base 15: mod_base = OTHER; note = 2'-O-methyl guanosine 3'-phosphodiester
  modified_base 16..17: mod_base = OTHER; note = 2'-O-methyl uridine 3'-phosphodiester
  modified_base 18: mod_base = OTHER; note = 2'-O-methyl adenosine 3'-phosphodiester
  modified_base 19: mod_base = OTHER; note = 2'-O-methyl uridine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
tggtaatggacagagttatca

SEQ ID NO: 78 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9..10: mod_base = OTHER; note = 2'-fluoro guanosine 3'-phosphodiester
  modified_base 11: mod_base = OTHER; note = 2'-deoxycytidine 3'-phosphorothioate
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
gttatcgaggcacatactcca

SEQ ID NO: 79 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-deoxyguanosine 3'-phosphorothioate
  modified_base 10: mod_base = OTHER; note = 2'-fluoro guanosine 3'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
gttatcgaggcacatactcca

SEQ ID NO: 80 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 3'-fluoro hexitol nucleic acid guanosine 4'-phosphodiester
  modified_base 11: mod_base = OTHER; note = 2'-fluoro cytidine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
gttatcgaggcacatactcca

SEQ ID NO: 81 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-O-methyl cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9..10: mod_base = OTHER; note = 2'-fluoro guanosine 3'-phosphodiester
  modified_base 11: mod_base = OTHER; note = 3'-fluoro hexitol nucleic acid cytidine 4'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-O-methoxyethyl adenosine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
gttatcgaggcacatactcca

SEQ ID NO: 82 - RNA, 21 nt, synthetic construct
  source 1..21: mol_type = other RNA
  modified_base 1: mod_base = OTHER; note = 2'-O-methoxyethyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl uridine 3'-phosphorothioate
  modified_base 3..4: mod_base = OTHER; note = 2'-O-methyl guanosine 3'-phosphodiester
  modified_base 5: mod_base = OTHER; note = 2'-O-methyl adenosine 3'-phosphodiester
  modified_base 6: mod_base = OTHER; note = 2'-O-methyl cytidine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methyl adenosine 3'-phosphodiester
  modified_base 10: mod_base = OTHER; note = 2'-fluoro guanosine 3'-phosphorothioate
  modified_base 11: mod_base = OTHER; note = 2'-fluoro uridine 3'-phosphodiester
  modified_base 12: mod_base = OTHER; note = 2'-O-methyl uridine 3'-phosphodiester
  modified_base 13: mod_base = OTHER; note = 2'-O-methyl adenosine 3'-phosphodiester
  modified_base 14: mod_base = OTHER; note = 2'-O-methyl uridine 3'-phosphodiester
  modified_base 15: mod_base = OTHER; note = 2'-O-methyl cytidine 3'-phosphodiester
  modified_base 16: mod_base = OTHER; note = 2'-O-methyl guanosine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl guanosine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
atggacagagttatcgaggca

SEQ ID NO: 83 - RNA, 23 nt, synthetic construct
  source 1..23: mol_type = other RNA
  modified_base 1: mod_base = OTHER; note = thymine
tgcctcgataactctgtccatca

SEQ ID NO: 84 - RNA, 23 nt, synthetic construct
  source 1..23: mol_type = other RNA
tgcctcgataactctgtccatca

SEQ ID NO: 85 - RNA, 23 nt, synthetic construct
  source 1..23: mol_type = other RNA
  modified_base 1: mod_base = OTHER; note = thymine
tggagtatgtgcctcgataacaa

SEQ ID NO: 86 - RNA, 23 nt, synthetic construct
  source 1..23: mol_type = other RNA
  modified_base 1: mod_base = OTHER; note = thymine
tcctcgataactctgtccattaa

SEQ ID NO: 87 - RNA, 23 nt, synthetic construct
  source 1..23: mol_type = other RNA
  modified_base 1: mod_base = OTHER; note = thymine
tgcctcgataactctgtccataa

SEQ ID NO: 88 - RNA, 23 nt, synthetic construct
  source 1..23: mol_type = other RNA
  modified_base 1: mod_base = OTHER; note = thymine
ttgcctcgataactctgtccaaa

SEQ ID NO: 89 - RNA, 23 nt, synthetic construct
  source 1..23: mol_type = other RNA
  modified_base 1: mod_base = OTHER; note = thymine
tgtgcctcgataactctgtccaa

SEQ ID NO: 90 - RNA, 23 nt, synthetic construct
  source 1..23: mol_type = other RNA
  modified_base 1: mod_base = OTHER; note = thymine
tatgtgcctcgataactctgtaa

SEQ ID NO: 91 - RNA, 23 nt, synthetic construct
  source 1..23: mol_type = other RNA
  modified_base 1: mod_base = OTHER; note = thymine
tgtatgtgcctcgataactctaa

SEQ ID NO: 92 - RNA, 23 nt, synthetic construct
  source 1..23: mol_type = other RNA
  modified_base 1: mod_base = OTHER; note = thymine
tagtatgtgcctcgataactcaa

SEQ ID NO: 93 - RNA, 23 nt, synthetic construct
  source 1..23: mol_type = other RNA
  modified_base 1: mod_base = OTHER; note = thymine
ttaactctgtccattaccatgaa

SEQ ID NO: 94 - RNA, 23 nt, synthetic construct
  source 1..23: mol_type = other RNA
  modified_base 1: mod_base = OTHER; note = thymine
tataactctgtccattaccataa

SEQ ID NO: 95 - RNA, 23 nt, synthetic construct
  source 1..23: mol_type = other RNA
  modified_base 1: mod_base = OTHER; note = thymine
ttgtgcctcgataactctgtcaa

SEQ ID NO: 96 - RNA, 23 nt, synthetic construct
  source 1..23: mol_type = other RNA
  modified_base 1: mod_base = OTHER; note = thymine
tctcgataactctgtccattaaa

SEQ ID NO: 97 - RNA, 23 nt, synthetic construct
  source 1..23: mol_type = other RNA
  modified_base 1: mod_base = OTHER; note = thymine
ttcgataactctgtccattacaa

SEQ ID NO: 98 - RNA, 23 nt, synthetic construct
  source 1..23: mol_type = other RNA
  modified_base 1: mod_base = OTHER; note = thymine
tgataactctgtccattaccaaa

SEQ ID NO: 99 - RNA, 21 nt, synthetic construct
  source 1..21: mol_type = other RNA
atggacagagttatcgaggca

SEQ ID NO: 100 - RNA, 21 nt, synthetic construct
  source 1..21: mol_type = other RNA
gttatcgaggcacatactcca

SEQ ID NO: 101 - RNA, 21 nt, synthetic construct
  source 1..21: mol_type = other RNA
tggacagagttatcgaggcaa

SEQ ID NO: 102 - RNA, 21 nt, synthetic construct
  source 1..21: mol_type = other RNA
ggacagagttatcgaggcaca

SEQ ID NO: 103 - RNA, 21 nt, synthetic construct
  source 1..21: mol_type = other RNA
acagagttatcgaggcacata

SEQ ID NO: 104 - RNA, 21 nt, synthetic construct
  source 1..21: mol_type = other RNA
agagttatcgaggcacataca

SEQ ID NO: 105 - RNA, 21 nt, synthetic construct
  source 1..21: mol_type = other RNA
gagttatcgaggcacatacta

SEQ ID NO: 106 - RNA, 21 nt, synthetic construct
  source 1..21: mol_type = other RNA
catggtaatggacagagttaa

SEQ ID NO: 107 - RNA, 21 nt, synthetic construct
  source 1..21: mol_type = other RNA
atggtaatggacagagttata

SEQ ID NO: 108 - RNA, 21 nt, synthetic construct
  source 1..21: mol_type = other RNA
gacagagttatcgaggcacaa

SEQ ID NO: 109 - RNA, 21 nt, synthetic construct
  source 1..21: mol_type = other RNA
taatggacagagttatcgaga

SEQ ID NO: 110 - RNA, 21 nt, synthetic construct
  source 1..21: mol_type = other RNA
gtaatggacagagttatcgaa

SEQ ID NO: 111 - RNA, 21 nt, synthetic construct
  source 1..21: mol_type = other RNA
tggtaatggacagagttatca

SEQ ID NO: 112 - RNA, 21 nt, synthetic construct
  source 1..21: mol_type = other RNA
aatggacagagttatcgagga

SEQ ID NO: 113 - DNA, 16 bp, synthetic construct
  source 1..16: mol_type = other DNA
ccacagtggccccggt

SEQ ID NO: 114 - DNA, 21 bp, synthetic construct
  source 1..21: mol_type = other DNA
acagggcttttctcaggtggt

SEQ ID NO: 115 - DNA, 28 bp, synthetic construct
  source 1..28: mol_type = other DNA
ccaagcacagaggctccttctgaacaag

SEQ ID NO: 116 - RNA, 21 nt, synthetic construct
  source 1..21: mol_type = other RNA
  modified_base 1: mod_base = OTHER; note = 2'-O-methyl uridine 3'-phosphorothioate
  modified_base 2: mod_base = OTHER; note = 2'-fluoro cytidine 3'-phosphorothioate
  modified_base 3: mod_base = OTHER; note = 2'-O-methyl guanosine 3'-phosphodiester
  modified_base 4: mod_base = OTHER; note = 2'-fluoro uridine 3'-phosphodiester
  modified_base 5: mod_base = OTHER; note = 2'-O-methyl adenosine 3'-phosphodiester
  modified_base 6: mod_base = OTHER; note = 2'-fluoro uridine 3'-phosphodiester
  modified_base 7..8: mod_base = OTHER; note = 2'-O-methyl adenosine 3'-phosphodiester
  modified_base 9: mod_base = OTHER; note = 2'-O-methyl cytidine 3'-phosphodiester
  modified_base 10..11: mod_base = OTHER; note = 2'-O-methyl adenosine 3'-phosphodiester
  modified_base 12: mod_base = OTHER; note = 2'-fluoro uridine 3'-phosphodiester
  modified_base 13: mod_base = OTHER; note = 2'-O-methyl adenosine 3'-phosphodiester
  modified_base 14: mod_base = OTHER; note = 2'-fluoro adenosine 3'-phosphodiester
  modified_base 15: mod_base = OTHER; note = 2'-O-methyl guanosine 3'-phosphodiester
  modified_base 16: mod_base = OTHER; note = 2'-fluoro guanosine 3'-phosphodiester
  modified_base 17: mod_base = OTHER; note = 2'-O-methyl guanosine 3'-phosphodiester
  modified_base 18: mod_base = OTHER; note = 2'-fluoro guanosine 3'-phosphodiester
  modified_base 19: mod_base = OTHER; note = 2'-O-methyl cytidine 3'-phosphorothioate
  modified_base 20: mod_base = OTHER; note = 2'-fluoro uridine 3'-phosphorothioate
  modified_base 21: mod_base = OTHER; note = 2'-O-methyl guanosine
tcgtataacaataaggggctg

SEQ ID NO: 117 - RNA, 21 nt, synthetic construct
  source 1..21: mol_type = other RNA
  modified_base 1: mod_base = OTHER; note = 2'-O-methyl cytidine 3'-phosphorothioate, conjugated at the 5' end to trishexylamino-N-Acetylgalactosamine-6-aminohexyl phosphorothioate conjugate
  modified_base 2: mod_base = OTHER; note = 2'-O-methyl adenosine 3'-phosphodiester
  modified_base 3: mod_base = OTHER; note = 2'-O-methyl guanosine 3'-phosphodiester
  modified_base 4..7: mod_base = OTHER; note = 2'-O-methyl cytidine 3'-phosphodiester
  modified_base 8: mod_base = OTHER; note = 2'-O-methyl uridine 3'-phosphodiester
  modified_base 9: mod_base = OTHER; note = 2'-fluoro uridine 3'-phosphodiester
  modified_base 10: mod_base = OTHER; note = 2'-fluoro adenosine 3'-phosphodiester
  modified_base 11: mod_base = OTHER; note = 2'-fluoro uridine 3'-phosphodiester
  modified_base 12: mod_base = OTHER; note = 2'-O-methyl uridine 3'-phosphodiester
  modified_base 13: mod_base = OTHER; note = 2'-O-methyl guanosine 3'-phosphodiester
  modified_base 14..15: mod_base = OTHER; note = 2'-O-methyl uridine 3'-phosphodiester
  modified_base 16: mod_base = OTHER; note = 2'-O-methyl adenosine 3'-phosphodiester
  modified_base 17: mod_base = OTHER; note = 2'-O-methyl uridine 3'-phosphodiester
  modified_base 18: mod_base = OTHER; note = 2'-O-methyl adenosine 3'-phosphodiester
  modified_base 19: mod_base = OTHER; note = 2'-O-methyl cytidine 3'-phosphodiester
  modified_base 20: mod_base = OTHER; note = 2'-O-methyl guanosine 3'-phosphorothioate
  modified_base 21: mod_base = OTHER; note = 2'-O-methyl adenosine
cagccccttattgttatacga

SEQ ID NO: 118 - DNA, 22 bp, synthetic construct
  source 1..22: mol_type = other DNA
ttgttgtaggatatgcccttga

SEQ ID NO: 119 - DNA, 21 bp, synthetic construct
  source 1..21: mol_type = other DNA
gcgatgtcaataggactccag

SEQ ID NO: 120 - DNA, 31 bp, synthetic construct
  source 1..31: mol_type = other DNA
agcctaagatgagagttcaagttgagtttgg

SEQ ID NO: 121 - DNA, 21 bp, synthetic construct
  source 1..21: mol_type = other DNA
accatgtctggactggaatgc

SEQ ID NO: 122 - DNA, 25 bp, synthetic construct
  source 1..25: mol_type = other DNA
cttcttcaggttcttgtttggaaat

SEQ ID NO: 123 - DNA, 25 bp, synthetic construct
  source 1..25: mol_type = other DNA
actctcagagcccacacgctcatgg

SEQ ID NO: 124 - DNA, 24 bp, synthetic construct
  source 1..24: mol_type = other DNA
gcggtgggagtactgtaagatacc

SEQ ID NO: 125 - DNA, 19 bp, synthetic construct
  source 1..19: mol_type = other DNA
gtgctgtgggagccaattg

SEQ ID NO: 126 - DNA, 27 bp, synthetic construct
  source 1..27: mol_type = other DNA
ctgtgactcctccccagtatccacgga

SEQ ID NO: 127 - RNA, 23 nt, synthetic construct
  source 1..23: mol_type = other RNA
  modified_base 1: mod_base = OTHER; note = 2'-O-methoxyethyl thymidine 3'-phosphorothioate
  modified_base 2: mod_base = OTHER; note = 2'-fluoro adenosine 3'-phosphorothioate
  modified_base 3: mod_base = OTHER; note = 2'-O-methyl adenosine 3'-phosphodiester
  modified_base 4: mod_base = OTHER; note = 2'-O-methyl guanosine 3'-phosphodiester
  modified_base 5: mod_base = OTHER; note = 2'-O-methyl uridine 3'-phosphodiester
  modified_base 6: mod_base = OTHER; note = 2'-fluoro guanosine 3'-phosphodiester
  modified_base 7: mod_base = OTHER; note = 2'-O-methyl adenosine 3'-phosphodiester
  modified_base 8: mod_base = OTHER; note = 2'-O-methyl guanosine 3'-phosphodiester
  modified_base 9: mod_base = OTHER; note = 2'-O-methyl cytidine 3'-phosphodiester
  modified_base 10: mod_base = OTHER; note = 2'-O-methyl uridine 3'-phosphodiester
  modified_base 11: mod_base = OTHER; note = 2'-O-methyl adenosine 3'-phosphodiester
  modified_base 12: mod_base = OTHER; note = 2'-O-methyl guanosine 3'-phosphodiester
  modified_base 13: mod_base = OTHER; note = 2'-O-methyl cytidine 3'-phosphodiester
  modified_base 14: mod_base = OTHER; note = 2'-fluoro adenosine 3'-phosphodiester
  modified_base 15: mod_base = OTHER; note = 2'-O-methyl adenosine 3'-phosphodiester
  modified_base 16: mod_base = OTHER; note = 2'-fluoro adenosine 3'-phosphodiester
  modified_base 17: mod_base = OTHER; note = 2'-O-methyl cytidine 3'-phosphodiester
  modified_base 18: mod_base = OTHER; note = 2'-O-methyl adenosine 3'-phosphodiester
  modified_base 19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl cytidine 3'-phosphorothioate
  modified_base 22: mod_base = OTHER; note = 2'-O-methyl adenosine 3'-phosphorothioate
  modified_base 23: mod_base = OTHER; note = 2'-O-methyl uridine
taagtgagctagcaaacacacat

SEQ ID NO: 128 - RNA, 21 nt, synthetic construct
  source 1..21: mol_type = other RNA
  modified_base 1: mod_base = OTHER; note = 2'-O-methyl guanosine 3'-phosphorothioate
  modified_base 2: mod_base = OTHER; note = 2'-O-methyl uridine 3'-phosphorothioate
  modified_base 3: mod_base = OTHER; note = 2'-O-methyl guanosine 3'-phosphodiester
  modified_base 4: mod_base = OTHER; note = 2'-O-methyl uridine 3'-phosphodiester
  modified_base 5: mod_base = OTHER; note = 2'-O-methyl guanosine 3'-phosphodiester
  modified_base 6: mod_base = OTHER; note = 2'-O-methyl uridine 3'-phosphodiester
  modified_base 7: mod_base = OTHER; note = 2'-fluoro uridine 3'-phosphodiester
  modified_base 8: mod_base = OTHER; note = 2'-O-methyl uridine 3'-phosphodiester
  modified_base 9: mod_base = OTHER; note = 2'-fluoro guanosine 3'-phosphodiester
  modified_base 10: mod_base = OTHER; note = 2'-fluoro cytidine 3'-phosphodiester
  modified_base 11: mod_base = OTHER; note = 2'-fluoro uridine 3'-phosphodiester
  modified_base 12: mod_base = OTHER; note = 2'-O-methyl adenosine 3'-phosphodiester
  modified_base 13: mod_base = OTHER; note = 2'-O-methyl guanosine 3'-phosphodiester
  modified_base 14: mod_base = OTHER; note = 2'-O-methyl cytidine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17: mod_base = OTHER; note = 2'-O-methyl adenosine 3'-phosphodiester
  modified_base 18: mod_base = OTHER; note = 2'-O-methyl cytidine 3'-phosphodiester
  modified_base 19..20: mod_base = OTHER; note = 2'-O-methyl uridine 3'-phosphorothioate
  modified_base 21: mod_base = OTHER; note = 2'-O-methyl adenosine, conjugated at the 3' end to trishexylamino-N-Acetylgalactosamine-6-hydroxypropyl phosphodiester conjugate
gtgtgtttgctagctcactta

SEQ ID NO: 129 - DNA, 18 bp, synthetic construct
  source 1..18: mol_type = other DNA
caatatggcgggcaccaa

SEQ ID NO: 130 - DNA, 20 bp, synthetic construct
  source 1..20: mol_type = other DNA
ggtcccatttgccattgatc

SEQ ID NO: 131 - DNA, 25 bp, synthetic construct
  source 1..25: mol_type = other DNA
ccctacacaaccatcacgcctcagg

SEQ ID NO: 132 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 3'-fluoro hexitol nucleic acid guanosine 4'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-fluoro guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgcctcgataactctgtccataa

SEQ ID NO: 133 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 3'-fluoro hexitol nucleic acid guanosine 4'-phosphorothioate
  modified_base 3..4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-fluoro cytidine 3'-phosphodiester
  modified_base 7: mod_base = OTHER; note = 2'-O-methyl guanosine 3'-phosphodiester
  modified_base 8: mod_base = OTHER; note = 2'-O-methyl adenosine 3'-phosphodiester
  modified_base 9: mod_base = OTHER; note = 2'-O-methoxyethyl uridine 3'-phosphodiester
  modified_base 10..11: mod_base = OTHER; note = 2'-O-methyl adenosine 3'-phosphodiester
  modified_base 12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl uridine 3'-phosphodiester
  modified_base 16: mod_base = OTHER; note = 2'-fluoro guanosine 3'-phosphodiester
  modified_base 17: mod_base = OTHER; note = 2'-O-methyl uridine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgcctcgataactctgtccataa

SEQ ID NO: 134 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro uridine 3'-phosphorothioate
  modified_base 3..4: mod_base = OTHER; note = 2'-O-methyl adenosine 3'-phosphodiester
  modified_base 5: mod_base = OTHER; note = 2'-O-methyl cytidine 3'-phosphodiester
  modified_base 6: mod_base = OTHER; note = 2'-fluoro uridine 3'-phosphodiester
  modified_base 7: mod_base = OTHER; note = 2'-O-methyl cytidine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl uridine 3'-phosphodiester
  modified_base 11..12: mod_base = OTHER; note = 2'-O-methyl cytidine 3'-phosphodiester
  modified_base 13: mod_base = OTHER; note = 2'-O-methyl adenosine 3'-phosphodiester
  modified_base 14: mod_base = OTHER; note = 2'-fluoro uridine 3'-phosphodiester
  modified_base 15: mod_base = OTHER; note = 2'-O-methyl uridine 3'-phosphodiester
  modified_base 16: mod_base = OTHER; note = 2'-O-methyl adenosine 3'-phosphodiester
  modified_base 17..18: mod_base = OTHER; note = 2'-O-methyl cytidine 3'-phosphodiester
  modified_base 19: mod_base = OTHER; note = 2'-O-methyl adenosine 3'-phosphodiester
  modified_base 20: mod_base = OTHER; note = 2'-O-methyl uridine 3'-phosphodiester
  modified_base 21: mod_base = OTHER; note = 2'-O-methyl guanosine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
ttaactctgtccattaccatgaa

SEQ ID NO: 135 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro adenosine 3'-phosphorothioate
  modified_base 3: mod_base = OTHER; note = 2'-O-methyl uridine 3'-phosphodiester
  modified_base 4..5: mod_base = OTHER; note = 2'-O-methyl adenosine 3'-phosphodiester
  modified_base 6: mod_base = OTHER; note = 2'-fluoro cytidine 3'-phosphodiester
  modified_base 7: mod_base = OTHER; note = 2'-O-methyl uridine 3'-phosphodiester
  modified_base 8: mod_base = OTHER; note = 2'-O-methyl cytidine 3'-phosphodiester
  modified_base 9: mod_base = OTHER; note = 2'-O-methyl uridine 3'-phosphodiester
  modified_base 10: mod_base = OTHER; note = 2'-O-methyl guanosine 3'-phosphodiester
  modified_base 11: mod_base = OTHER; note = 2'-O-methyl uridine 3'-phosphodiester
  modified_base 12..13: mod_base = OTHER; note = 2'-O-methyl cytidine 3'-phosphodiester
  modified_base 14: mod_base = OTHER; note = 2'-fluoro adenosine 3'-phosphodiester
  modified_base 15..16: mod_base = OTHER; note = 2'-O-methyl uridine 3'-phosphodiester
  modified_base 17: mod_base = OTHER; note = 2'-O-methyl adenosine 3'-phosphodiester
  modified_base 18..19: mod_base = OTHER; note = 2'-O-methyl cytidine 3'-phosphodiester
  modified_base 20: mod_base = OTHER; note = 2'-O-methyl adenosine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tataactctgtccattaccataa

SEQ ID NO: 136 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl adenosine 3'-phosphodiester
  modified_base 4: mod_base = OTHER; note = 2'-O-methyl uridine 3'-phosphodiester
  modified_base 5: mod_base = OTHER; note = 2'-O-methyl adenosine 3'-phosphodiester
  modified_base 6: mod_base = OTHER; note = 2'-fluoro adenosine 3'-phosphodiester
  modified_base 7: mod_base = OTHER; note = 2'-O-methyl cytidine 3'-phosphodiester
  modified_base 8: mod_base = OTHER; note = 2'-O-methyl uridine 3'-phosphodiester
  modified_base 9: mod_base = OTHER; note = 2'-O-methyl cytidine 3'-phosphodiester
  modified_base 10: mod_base = OTHER; note = 2'-O-methyl uridine 3'-phosphodiester
  modified_base 11: mod_base = OTHER; note = 2'-O-methyl guanosine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro cytidine 3'-phosphodiester
  modified_base 15: mod_base = OTHER; note = 2'-O-methyl adenosine 3'-phosphodiester
  modified_base 16..17: mod_base = OTHER; note = 2'-O-methyl uridine 3'-phosphodiester
  modified_base 18: mod_base = OTHER; note = 2'-O-methyl adenosine 3'-phosphodiester
  modified_base 19..20: mod_base = OTHER; note = 2'-O-methyl cytidine 3'-phosphodiester
  modified_base 21: mod_base = OTHER; note = 2'-O-methyl adenosine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgataactctgtccattaccaaa

SEQ ID NO: 137 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro cytidine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6: mod_base = OTHER; note = 2'-fluoro adenosine 3'-phosphodiester
  modified_base 7: mod_base = OTHER; note = 2'-O-methyl adenosine 3'-phosphodiester
  modified_base 8: mod_base = OTHER; note = 2'-O-methyl cytidine 3'-phosphodiester
  modified_base 9: mod_base = OTHER; note = 2'-O-methyl uridine 3'-phosphodiester
  modified_base 10: mod_base = OTHER; note = 2'-O-methyl cytidine 3'-phosphodiester
  modified_base 11: mod_base = OTHER; note = 2'-O-methyl uridine 3'-phosphodiester
  modified_base 12: mod_base = OTHER; note = 2'-O-methyl guanosine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl cytidine 3'-phosphodiester
  modified_base 16: mod_base = OTHER; note = 2'-O-methyl adenosine 3'-phosphodiester
  modified_base 17..18: mod_base = OTHER; note = 2'-O-methyl uridine 3'-phosphodiester
  modified_base 19: mod_base = OTHER; note = 2'-O-methyl adenosine 3'-phosphodiester
  modified_base 20: mod_base = OTHER; note = 2'-O-methyl cytidine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcgataactctgtccattaccaa

SEQ ID NO: 138 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro cytidine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5: mod_base = OTHER; note = 2'-O-methyl uridine 3'-phosphodiester
  modified_base 6..7: mod_base = OTHER; note = 2'-O-methyl adenosine 3'-phosphodiester
  modified_base 8: mod_base = OTHER; note = 2'-O-methyl cytidine 3'-phosphodiester
  modified_base 9: mod_base = OTHER; note = 2'-O-methyl uridine 3'-phosphodiester
  modified_base 10: mod_base = OTHER; note = 2'-O-methyl cytidine 3'-phosphodiester
  modified_base 11: mod_base = OTHER; note = 2'-O-methyl uridine 3'-phosphodiester
  modified_base 12: mod_base = OTHER; note = 2'-O-methyl guanosine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl cytidine 3'-phosphodiester
  modified_base 16: mod_base = OTHER; note = 2'-fluoro adenosine 3'-phosphodiester
  modified_base 17..18: mod_base = OTHER; note = 2'-O-methyl uridine 3'-phosphodiester
  modified_base 19: mod_base = OTHER; note = 2'-O-methyl adenosine 3'-phosphodiester
  modified_base 20: mod_base = OTHER; note = 2'-O-methyl cytidine 3'-phosphodiester
  modified_base 21: mod_base = OTHER; note = 2'-O-methyl cytidine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tcgataactctgtccattaccaa

SEQ ID NO: 139 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl adenosine 3'-phosphodiester
  modified_base 4: mod_base = OTHER; note = 2'-O-methyl guanosine 3'-phosphodiester
  modified_base 5: mod_base = OTHER; note = 2'-O-methyl uridine 3'-phosphodiester
  modified_base 6: mod_base = OTHER; note = 2'-fluoro adenosine 3'-phosphodiester
  modified_base 7: mod_base = OTHER; note = 2'-O-methyl uridine 3'-phosphodiester
  modified_base 8: mod_base = OTHER; note = 2'-O-methyl guanosine 3'-phosphodiester
  modified_base 9: mod_base = OTHER; note = 2'-O-methyl uridine 3'-phosphodiester
  modified_base 10: mod_base = OTHER; note = 2'-O-methyl guanosine 3'-phosphodiester
  modified_base 11..12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl guanosine 3'-phosphodiester
  modified_base 16: mod_base = OTHER; note = 2'-O-methyl adenosine 3'-phosphodiester
  modified_base 17: mod_base = OTHER; note = 2'-O-methyl uridine 3'-phosphodiester
  modified_base 18..19: mod_base = OTHER; note = 2'-O-methyl adenosine 3'-phosphodiester
  modified_base 20: mod_base = OTHER; note = 2'-O-methyl cytidine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgagtatgtgcctcgataactaa

SEQ ID NO: 140 - RNA, 23 nt, synthetic construct
  source 1..23: mol_type = other RNA
  modified_base 1: mod_base = OTHER; note = 2'-O-methoxyethyl thymidine 3'-phosphorothioate 5' vinyl phosphonate
  modified_base 2: mod_base = OTHER; note = 2'-fluoro guanosine 3'-phosphorothioate
  modified_base 3: mod_base = OTHER; note = 2'-O-methyl adenosine 3'-phosphodiester
  modified_base 4: mod_base = OTHER; note = 2'-O-methyl guanosine 3'-phosphodiester
  modified_base 5: mod_base = OTHER; note = 2'-O-methyl uridine 3'-phosphodiester
  modified_base 6: mod_base = OTHER; note = 2'-O-methyl adenosine 3'-phosphodiester
  modified_base 7: mod_base = OTHER; note = 2'-O-methyl uridine 3'-phosphodiester
  modified_base 8: mod_base = OTHER; note = 2'-O-methyl guanosine 3'-phosphodiester
  modified_base 9: mod_base = OTHER; note = 2'-O-methyl uridine 3'-phosphodiester
  modified_base 10: mod_base = OTHER; note = 2'-O-methyl guanosine 3'-phosphodiester
  modified_base 11..12: mod_base = OTHER; note = 2'-O-methyl cytidine 3'-phosphodiester
  modified_base 13: mod_base = OTHER; note = 2'-O-methyl uridine 3'-phosphodiester
  modified_base 14: mod_base = OTHER; note = 2'-fluoro cytidine 3'-phosphodiester
  modified_base 15: mod_base = OTHER; note = 2'-O-methyl guanosine 3'-phosphodiester
  modified_base 16: mod_base = OTHER; note = 2'-fluoro adenosine 3'-phosphodiester
  modified_base 17: mod_base = OTHER; note = 2'-O-methyl uridine 3'-phosphodiester
  modified_base 18..19: mod_base = OTHER; note = 2'-O-methyl adenosine 3'-phosphodiester
  modified_base 20: mod_base = OTHER; note = 2'-O-methyl cytidine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tgagtatgtgcctcgataactaa

SEQ ID NO: 141 - RNA, 21 nt, synthetic construct
  source 1..21: mol_type = other RNA
  modified_base 1: mod_base = OTHER; note = 2'-O-methoxyethyl guanosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl guanosine 3'-phosphorothioate
  modified_base 3: mod_base = OTHER; note = 2'-O-methyl uridine 3'-phosphodiester
  modified_base 4..5: mod_base = OTHER; note = 2'-O-methyl adenosine 3'-phosphodiester
  modified_base 6: mod_base = OTHER; note = 2'-O-methyl uridine 3'-phosphodiester
  modified_base 7..8: mod_base = OTHER; note = 2'-O-methyl guanosine 3'-phosphodiester
  modified_base 9: mod_base = OTHER; note = 2'-fluoro adenosine 3'-phosphodiester
  modified_base 10: mod_base = OTHER; note = 2'-fluoro cytidine 3'-phosphodiester
  modified_base 11: mod_base = OTHER; note = 2'-fluoro adenosine 3'-phosphodiester
  modified_base 12: mod_base = OTHER; note = 2'-O-methyl guanosine 3'-phosphodiester
  modified_base 13: mod_base = OTHER; note = 2'-O-methyl adenosine 3'-phosphodiester
  modified_base 14: mod_base = OTHER; note = 2'-O-methoxyethyl guanosine 3'-phosphodiester
  modified_base 15..16: mod_base = OTHER; note = 2'-O-methyl uridine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphorothioate
  modified_base 20: mod_base = OTHER; note = 2'-O-methoxyethyl guanosine 3'-phosphorothioate
  modified_base 21: mod_base = OTHER; note = 2'-O-methoxyethyl adenosine
ggtaatggacagagttatcga

SEQ ID NO: 142 - RNA, 21 nt, synthetic construct
  source 1..21: mol_type = other RNA
  modified_base 1: mod_base = OTHER; note = 2'-O-methoxyethyl adenosine 3'-phosphorothioate, conjugated at the 5' end to trishexylamino-N-Acetylgalactosamine-6-aminohexyl phosphodiester conjugate
  modified_base 2: mod_base = OTHER; note = 2'-O-methoxyethyl guanosine 3'-phosphorothioate
  modified_base 3..4: mod_base = OTHER; note = 2'-O-methyl uridine 3'-phosphodiester
  modified_base 5: mod_base = OTHER; note = 2'-O-methyl adenosine 3'-phosphodiester
  modified_base 6: mod_base = OTHER; note = 2'-O-methyl uridine 3'-phosphodiester
  modified_base 7: mod_base = OTHER; note = 2'-O-methyl cytidine 3'-phosphodiester
  modified_base 8: mod_base = OTHER; note = 2'-O-methyl guanosine 3'-phosphodiester
  modified_base 9: mod_base = OTHER; note = 2'-fluoro adenosine 3'-phosphodiester
  modified_base 10..11: mod_base = OTHER; note = 2'-fluoro guanosine 3'-phosphodiester
  modified_base 12: mod_base = OTHER; note = 2'-O-methyl cytidine 3'-phosphodiester
  modified_base 13: mod_base = OTHER; note = 2'-O-methyl adenosine 3'-phosphodiester
  modified_base 14: mod_base = OTHER; note = 2'-O-methoxyethyl cytidine 3'-phosphodiester
  modified_base 15: mod_base = OTHER; note = 2'-O-methyl adenosine 3'-phosphodiester
  modified_base 16: mod_base = OTHER; note = 2'-O-methyl uridine 3'-phosphodiester
  modified_base 17: mod_base = OTHER; note = 2'-O-methyl adenosine 3'-phosphodiester
  modified_base 18: mod_base = OTHER; note = 2'-O-methyl cytidine 3'-phosphodiester
  modified_base 19: mod_base = OTHER; note = 2'-O-methyl uridine 3'-phosphorothioate
  modified_base 20: mod_base = OTHER; note = 2'-O-methoxyethyl cytidine 3'-phosphorothioate
  modified_base 21: mod_base = OTHER; note = 2'-O-methoxyethyl adenosine
agttatcgaggcacatactca

SEQ ID NO: 143 - RNA, 23 nt, synthetic construct
  source 1..23: mol_type = other RNA
tcgataactctgtccattaccaa

SEQ ID NO: 144 - RNA, 23 nt, synthetic construct
  source 1..23: mol_type = other RNA
tgagtatgtgcctcgataactaa

SEQ ID NO: 145 - RNA, 21 nt, synthetic construct
  source 1..21: mol_type = other RNA
ggtaatggacagagttatcga

SEQ ID NO: 146 - RNA, 21 nt, synthetic construct
  source 1..21: mol_type = other RNA
agttatcgaggcacatactca